(19) United States
(12) Patent Application Publication
(10) Pub. No.: US 2019/0244683 A1
(43) Pub. Date: Aug. 8, 2019

(54) SYSTEMS AND METHODS FOR USING ELECTRONIC MEDICAL RECORDS IN CONJUNCTION WITH PATIENT APPS

(71) Applicant: Revon Systems, Inc., Crestwood, KY (US)

(72) Inventor: Cedric Francois, Louisville, KY (US)

(21) Appl. No.: 16/111,855

(22) Filed: Aug. 24, 2018

Related U.S. Application Data
(63) Continuation of application No. 14/751,301, filed on Jun. 26, 2015, now abandoned.
Application No. 14/751,301 is a continuation of application No. PCT/US13/77227, filed on Dec. 20, 2013.
(60) Provisional application No. 61/788,060, filed on Mar. 15, 2013.
(60) Provisional application No. 61/763,890, filed on Feb. 12, 2013.
(60) Provisional application No. 61/746,590, filed on Dec. 28, 2012.

(51) Int. Cl. G16H 10/20; G16H 10/60

(52) U.S. Cl. CPC G16H 10/20; G16H 10/60

(57) ABSTRACT

In some aspects, the present disclosure provides a computer program product for assembling a database comprising electronic medical records (EMRs). In certain embodiments, a EMR comprises at least one active diagnosis module (ADM). In some embodiments, the database is searchable based on at least some ADM content. Other aspects and features of the present disclosure are described herein.

### BACKGROUND

Medical records have traditionally been written on paper and maintained in folders. These folders are often divided into multiple sections, with new information added to each section as relevant over time. Retrieving paper records when needed may be time-consuming, particularly if they have been archived off-site. Patients may have multiple medical records generated at different medical facilities at which they have received care. For example, a patient's primary care provider may not have ready access to a medical record generated at a hospital where a patient received surgery. Another problematic feature of traditional medical records is the use of handwriting by health care providers, which may at times be difficult to decipher. Standard electronic medical records (EMRs) may offer, among other things, the possibility of increased accessibility and legibility. While standard EMRs undoubtedly offer many potential benefits, the entry of accurate and comprehensive information regarding a patient into a standard EMR may be burdensome.

### SUMMARY

In some aspects, the disclosure provides a computer program product for assembling a database comprising electronic medical records (EMRs). An “electronic medical record” may sometimes be referred to as an “electronic health record”, “electronic health care record”, “electronic patient record”, or various similar terms. Such terms may be considered equivalent and interchangeable.

In some aspects a computer program product for assembling a database comprising electronic medical records (EMRs) or modules thereof is provided, the computer program product comprising a computer-readable medium encoded with computer-executable instructions for performing a method comprising: (a) receiving from a contributor a dataset comprising health information regarding an individual; and (b) providing an incentive to the contributor or the contributor's designee. In some embodiments, the database or computer program product may be at least partly owned by a business entity. In some embodiments, a business entity, which may at least partly own the database or computer program product, may provide an incentive. In some embodiments, health information is checked, e.g., to determine whether it meets a set of predetermined criteria. In some embodiments, an incentive is provided following verification that the health information meets a set of predetermined criteria. In some embodiments, a dataset meeting said predetermined criteria may be deemed adequate to assemble an EMR or a module thereof. In some embodiments an incentive is issued following receipt of a request from a subscriber to access or analyze an EMR or a module thereof, wherein the EMR or module is assembled at least in part from data contributed by the contributor.

In some embodiments, an EMR or database contains one or more active diagnosis modules (ADMs). In some embodiments an incentive is issued following receipt of a request from a subscriber to access or analyze an ADM, wherein the ADM is assembled at least in part from data contributed by the contributor. In some embodiments an incentive comprises the privilege of prescribing experimental therapies. In some embodiments an incentive may comprise a share, e.g., a share in a business entity that at least in part owns or controls or administers the database or computer program product. In some embodiments a contributor is a health care provider (HCP). A HCP may be a physician, a nurse, a clinical research coordinator, or any other professional providing health services to patients. In some embodiments a contributor is a HCP and the health information pertains to a patient of the HCP. In some embodiments, a form comprising predetermined fields for entering health information is provided to a contributor. In some embodiments, completion of at least some fields of said form is checked following submission. In some embodiments, a dataset is analyzed to determine whether it contains health information that meets predetermined criteria, e.g., criteria indicating that the dataset is adequate to assemble an EMR or a module thereof; and the dataset is accepted or rejected based at least in part on said analyzing. In some embodiments a computer program product analyzes a proposed definitive diagnosis (e.g., in a submitted health information dataset) to determine whether it satisfies a set of predetermined criteria and, if so, updates a status from “tentative” to “definitive”.

In some embodiments a computer program product provides feedback to the contributor regarding the dataset, said feedback optionally comprising: (i) informing the contributor whether the dataset was accepted or rejected; (ii) informing the contributor of a rejected dataset of one or more reason(s) why the dataset was rejected; (iii) informing a contributor of or to a deficient proposed definitive ADM why the proposed definitive ADM was deemed deficient; (iv) suggesting a diagnostic test; (v) suggesting a therapeutic; (vi) suggesting referral to a colleague. In some embodiments a computer program product maintains, e.g., in one or more computers, account data pertaining at least in part to datasets received from contributors. In some embodiments a computer program product maintains, e.g., in one or more computers, account data of outstanding shares of the business entity or other forms of compensation, e.g. cash or other forms of credits. In some embodiments there may be multiple contributors, and the account data may include an account for each contributor.

In some embodiments, a method may further comprise electronically providing to a contributor account data regarding said contributor's account. In some embodiments, said account data is provided in response to a request from the contributor. In some embodiments said request is received from a portable electronic device, said account data is provided to the portable electronic device. In some embodiments a portable electronic device comprises a cellular phone. In some embodiments a database is searchable based on at least one element of an ADM. In some embodiments a method comprises receiving a request from a subscriber; accessing the database in response to the request; and providing a response to the subscriber. In some embodiments, de-identified data from the database is accessed, retrieved, analyzed, or provided to a subscriber in response to the request. In some embodiments an EMR in the database contains one or more ADMs adapted for identifying or enrolling subjects in a clinical trial and/or for gathering data pertaining to a clinical trial.

In some aspects, a computer or computer readable medium comprising a computer program product, e.g., as set forth herein, is provided.

In some aspects, an electronic device providing (e.g., having stored on computer-readable medium thereof and/or having computer-executable instruction steps contained therein) an application operative to interface with a computer that maintains a database or account data as set forth herein, is provided. In some embodiments a device is a portable electronic device. In some embodiments an application may allow a contributor to access at least some of his or her account data upon request. In some embodiments an electronic device provides an application that allows a contributor to purchase and sell shares in the business entity and, e.g., track the share price over time.

In some aspects, a database, tangibly embodied in a non-transitory computer-readable medium, is provided, wherein the database comprises: a plurality of ADMs, wherein an ADM comprises at least a tentative diagnosis or a definitive diagnosis, wherein a definitive diagnosis has been determined to satisfy at least one predetermined criterion.

In some embodiments a diagnosis is a conventional disease diagnosis. In some embodiments an ADM comprises a diagnosis status. In some embodiments an ADM comprises a tentative diagnosis and a definitive diagnosis. In some embodiments an ADM comprises a molecular diagnosis, wherein said molecular diagnosis has been determined to be consistent with a conventional diagnosis. In some embodiments an ADM comprises at least one diagnostic test and, e.g., a result thereof. In some embodiments an ADM comprises at least one diagnostic test suggested at least in part based on a tentative diagnosis. In some embodiments an ADM comprises at least one diagnostic test and a result thereof, and wherein the definitive diagnosis has been confirmed as definitive based at least in part on a result of said diagnostic. In some embodiments an ADM comprises at least one therapeutic, and wherein said therapeutic has been confirmed as appropriate for the definitive diagnosis. In some embodiments an ADM comprises at least one therapeutic, and wherein the therapeutic has been confirmed as appropriate for the patient. In some embodiments an ADM comprises an indication of the presence, absence, or characteristic(s) of at least one symptom, sign, complication, or outcome of the definitive diagnosis.

In some embodiments an ADM does not comprise a patient name. In some embodiments an ADM does not comprise a patient social security number. In some embodiments an ADM does not comprise protected health information. In some embodiments at least some information in an ADM is selected from a set of predetermined options. In some embodiments at least some information in an ADM is selected from numerical values. In some embodiments at least 50%, 60%, 70%, 80%, 90%, or more of the data items in an ADM are selected from a set of predetermined options or numerical values.

In some aspects, one or more non-transitory computer-readable media is provided, comprising: a database, wherein the database comprises a database as set forth herein, and wherein the one or more non-transitory computer-readable media store instructions that, when executed by one or more processors, cause the one or more processors to: access the database to determine a response to a user's query. In some embodiments, said instructions further cause the one or more processors to transmit a result to a user. In some aspects, one or more non-transitory computer-readable media is provided, comprising: a database, wherein the database comprises a database as set forth herein, and wherein the one or more non-transitory computer-readable media store instructions that, when executed by one or more processors, cause the one or more processors to: access the database to retrieve or analyze at least one ADM or an element thereof from the database in response to a user's query. In some embodiments, said instructions further cause the one or more processors to transmit a result to a user. In some embodiments instructions further cause the one or more processors to update account information for a contributor after accessing an EMR or ADM stored in the database, wherein the EMR or ADM comprises information submitted by the contributor, wherein said account information is optionally stored in an account database. In some embodiments, one or more non-transitory computer-readable media comprises a first medium and a second medium, and wherein the first medium comprises a database and the second medium comprises the instructions to retrieve or analyze data from the database. In some embodiments the instructions form part of a computer program used to access the database and the instructions are stored separately from the database.

In some aspects, a method comprising accessing a database as set forth herein to create, retrieve, analyze, or modify an EMR or ADM is provided. In some aspects, a method comprising accessing a database as set forth herein to respond to a query by a user is provided. In some embodiments the user is a subscriber.

In some aspects, one or more non-transitory computer-readable media comprising at least one ADM is provided, wherein an ADM comprises at least a tentative diagnosis or a definitive diagnosis, wherein a definitive diagnosis has been determined to satisfy at least one predetermined criterion. In some embodiments a diagnosis is a conventional disease diagnosis. In some embodiments an ADM comprises a diagnosis status. In some embodiments an ADM comprises a tentative diagnosis and a definitive diagnosis. In some embodiments an ADM comprises a molecular diagnosis, wherein said molecular diagnosis has been determined to be consistent with a conventional diagnosis.

In some embodiments an ADM comprises at least one diagnostic test and, e.g., a result thereof. In some embodiments an ADM comprises at least one diagnostic test suggested at least in part based on a tentative diagnosis.

In some embodiments an ADM comprises at least one diagnostic test and a result thereof, and wherein the definitive diagnosis has been confirmed as definitive based at least in part on a result of said diagnostic. In some embodiments an ADM comprises at least one therapeutic, and wherein said therapeutic has been confirmed as appropriate for the definitive diagnosis. In some embodiments an ADM comprises at least one therapeutic, and wherein the therapeutic has been confirmed as appropriate for the patient.

In some embodiments an ADM comprises an indication of the presence, absence, or characteristic(s) of at least one symptom, sign, complication, or outcome of the definitive diagnosis. In some embodiments a symptom, sign, complication or outcome is a key symptom, sign, complication or outcome. In some embodiments an ADM does not comprise a patient name. In some embodiments an ADM does not comprise a patient social security number. In some embodiments an ADM does not comprise protected health information. In some embodiments the one or more non-transitory computer-readable media comprises a plurality (more than one) of ADMs.

In some embodiments the one or more non-transitory computer-readable media comprises a plurality of ADMs for a patient. In some embodiments the one or more non-transitory computer-readable media comprises a plurality of ADMs for different patients.

In some embodiments an ADM for a patient is associated with information identifying said patient. In some embodiments an ADM for a patient comprises a module of an EMR for said patient. In some embodiments a method is provided comprising accessing the one or more non-transitory computer-readable media to create, retrieve, analyze, or modify an ADM. In some embodiments said retrieval, analysis, or modification is at least in part or solely for a patient care purpose. In some embodiments said retrieval or analysis is at least in part or solely for a research purpose.

In some aspects apparatus is provided, comprising: (i) one or more processors; memory, the memory comprising: a database as set forth herein, wherein the memory stores instructions that, when executed by the one or more processors, cause the one or more processors to: (a) access the database to create, modify, or retrieve an EMR or ADM; or (b) provide a template for entry of health information, wherein said template is optionally an ADM template; (c) analyze information received from a contributor to determine whether such information at least in part meets a set of predetermined criteria for storing the information in the database; or (d) analyze content of the database; or (e) determine a response to a query. In some embodiments, said instructions cause the one or more processors to retrieve information from a plurality of EMRs in response to a query, wherein the query optionally specifies a patient characteristic, conventional disease diagnosis, molecular diagnosis, diagnostic, treatment, symptom, sign, complication, or outcome, or combination thereof. In some embodiments said instructions cause the one or more processors to analyze information from a plurality of EMRs in response to a query, wherein the query may specify a patient characteristic, conventional disease diagnosis, molecular diagnosis, diagnostic, treatment, symptom, sign, complication, or outcome, or combination thereof. In some embodiments said instructions cause the one or more processors to retrieve information from a plurality of ADMs in response to a query, wherein the query may specify a conventional disease diagnosis, molecular diagnosis, diagnostic, treatment, symptom, sign, complication, or outcome, or combination thereof. In some embodiments said instructions cause the one or more processors to analyze information from a plurality of ADMs in response to a query, wherein the query may specify a conventional disease diagnosis, molecular diagnosis, diagnostic, treatment, symptom, sign, complication, or outcome, or combination thereof. In some embodiments said memory further comprises an account database. In some embodiments said database, account database, or both, is at least in part owned or administered by a business entity. In some embodiments said database, account database, or both is at least in part owned or administered by a business entity, and said memory comprises a database comprising shareholder account information.

In some aspects apparatus is provided, comprising: one or more processors; memory, the memory comprising: a database as set forth herein, wherein the memory comprises: an account database and wherein the memory stores instructions that, when executed by the one or more processors, cause the one or more processors to: (a) update information in the account database following receipt from a contributor of health information that meets at least one set of predetermined criteria; or (b) update information in the account database following a request by a subscriber to retrieve, access, or analyze an EMR or ADM, wherein said account information optionally comprises information tracking incentives earned by contributor(s) or (c) determine an incentive due to a contributor based at least in part on one or more requests received from subscriber(s) to retrieve, access, or analyze an EMR or ADM, wherein said EMR or ADM comprises information contributed by the contributor; or (d) issue an incentive to a contributor based at least in part on receiving a request by a subscriber to retrieve, access, or analyze an EMR or ADM, wherein said EMR or ADM comprises information contributed by the contributor. In some embodiments the request causes the one or more processors to execute instructions to retrieve, access, or analyze an ADM. In some embodiments the contributor is a HCP. In some embodiments said database is at least in part owned or administered by a business entity. In some embodiments said database is at least in part owned or administered by a business entity, and said memory comprises a database comprising shareholder account information.

In some aspects apparatus is provided comprising: one or more processors; memory, the memory comprising: a database as set forth herein, and wherein the memory stores instructions that, when executed by the one or more processors, cause the one or more processors to: analyze information received from a contributor to determine whether such information at least in part meets a set of predetermined criteria for inclusion in an EMR or ADM or for storage in the database. In some embodiments said instructions cause the processor to store at least some of said information in the database if predetermined criteria are met. In some embodiments said instructions cause the processor to provide feedback based at least in part on said analysis. In some embodiments said information comprises at least a tentative diagnosis.

In some embodiments said information comprises at least a proposed definitive diagnosis.

In some embodiments said instructions cause the processor to determine whether a proposed definitive diagnosis is consistent with other information regarding a patient to whom said proposed definitive diagnosis pertains, wherein said other information optionally comprises a result of at least one diagnostic test. In some embodiments said instructions cause the processor to update the status of an ADM if a proposed definitive diagnosis is confirmed. In some embodiments said instructions cause the processor to suggest at least one diagnostic test suitable for confirming a tentative or proposed definitive diagnosis. In some embodiments said instructions cause the processor to suggest at least one treatment suitable for a tentative, proposed definitive, or definitive diagnosis. In some embodiments said instructions comprise instructions for interfacing with an application for an electronic device. In some embodiments said device is a portable electronic device. In some embodiments said application allows a user to access the database or to access user account information.

In some aspects, a method comprising assembling a database as set forth herein is provided. In some embodiments a method comprises (a) receiving health information datasets pertaining to a plurality of patients; and (b) storing at least some of said health information in the database, optionally after checking said information to determine whether it meets a set of predetermined criteria. In some embodiments a method comprise (a) receiving health information datasets from a plurality of HCPs; and (b) storing at least some of said health information in the database, optionally after checking said information to determine whether it meets a set of predetermined criteria. In some embodiments a method comprises (a) receiving health information datasets pertaining to a plurality of patients; and (b) storing at least some of said health information in the database, optionally after checking said information to determine whether it meets a set of predetermined criteria. In some embodiments a method comprises providing a template to subscribers and receiving information entered into said template, said template optionally comprising a disease-specific or discipline-specialized template.

In some embodiments a method comprises providing an incentive to a contributor based at least in part on submission of health information by said contributor, said incentive optionally being determined based at least in part on access of such health information by subscribers. In some embodiments a method comprises providing a subscription to said database, optionally in exchange for a fee. In some aspects a method comprises retrieving, accessing, analyzing, or modifying information in said database, e.g., in response to a query from a user.

In some aspects, a database comprising EMRs is provided, said database being usable by HCPs for health care purposes and usable in addition for at least one non-health care purpose (e.g., a research purpose). In some aspects, health information is provided at least in part in modules comprising de-identified health information. A HCP may access such module(s), e.g., in the context of providing health care to a patient. An individual who is not an HCP of the patient may access or retrieve data from said module(s), e.g., for research purpose(s). In some embodiments an EMRs in such a database is a set of one or more ADMs corresponding to a patient.

In some aspects, EMR systems and uses of EMRs for purposes of clinical trials and/or managed access programs are provided. In some embodiments such purposes may include subject enrollment and/or electronic data capture.

In some aspects, methods of facilitating patient care are provided. In some embodiments, a method of facilitating patient care comprises using a computer program product or apparatus described herein to enter, access, retrieve, analyze, modify, or store information that relates to a patient who is a subject or potential subject in a clinical trial or managed access program (MAP), wherein such information optionally comprises screening data or trial-specific clinical trial data. In some embodiments the method comprises entering a tentative diagnosis of a disease for a patient, entering sufficient data to establish a confirmed diagnosis of a disease for the patient, and entering sufficient data to determine that the patient is eligible or potentially eligible for a trial of an experimental therapy intended for patients having the disease. In some embodiments at least some of the data is entered from within an EMR. In some embodiments at least some of the data is entered using a module or component that is accessed from within an EMR. In some embodiments at least some of the data is entered into an ADM or ADM template, e.g., an ADM-SC. In some embodiments the data is evaluated to identify one or more trials or MAPs for which the patient is eligible or potentially eligible. In some embodiments the method further comprises enrolling or arranging for enrollment of the patient in a clinical trial or managed access program for which the patient is eligible. The patient may then be able to receive an experimental therapy that may be helpful in treating the patient's disease. In some embodiments the method further comprises providing an experimental therapy to the patient, e.g., administering or prescribing the experimental therapy, wherein the experimental therapy is provided as part of a clinical trial or MAP. In some embodiments the method further comprises providing an experimental therapy to the patient, e.g., administering or prescribing the experimental therapy, wherein the experimental therapy is a therapy that is a candidate for repositioning.

In some aspects, methods of facilitating conducting a clinical trial or managed access program are provided. In some embodiments, a method of facilitating conducting a clinical trial or managed access program comprises using a computer program product or apparatus described herein to enter, access, retrieve, analyze, modify, or store information that relates to a patient who is a subject or potential subject in a clinical trial or managed access program (MAP), wherein such information optionally comprises screening data or trial-specific clinical trial data. In some embodiments the method comprises entering a tentative diagnosis of a disease for a patient, entering sufficient data to establish a confirmed diagnosis of a disease for the patient, and entering sufficient data to determine that the patient is eligible or potentially eligible for a trial of an experimental therapy intended for patients having the disease. In some embodiments at least some of the data is entered from within an EMR. In some embodiments at least some of the data is entered using a module or component that is accessed from within an EMR. In some embodiments at least some of the data is entered into an ADM or ADM template, e.g., an ADM-SC. In some embodiments the data is evaluated to identify one or more trials or MAPs for which the patient is eligible or potentially eligible. In some embodiments the method further comprises enrolling or arranging for enrollment of the patient in a clinical trial or managed access program for which the patient is eligible. In some embodiments the data is evaluated to identify one or more therapies that is a candidate for repositioning, e.g., wherein the therapy is a candidate for repositioning for use in the disease corresponding to the ADM. In some embodiments the method further comprises facilitating making the therapy that is a candidate for repositioning available to the patient, e.g., assisting the patient or the patient's HCP to obtain the therapy. In some embodiments data pertaining to the patient after the patient receives the experimental therapy is entered into the ADM either directly or via entering the data into an EMR from which it is used to populate one or more fields of the ADM. In some embodiments a method further comprises collecting or analyzing at least some of the data pertaining to the patient after the patient receives the experimental therapy.

In some aspects, a component that provides ADM functionality to an EMR system that lacks such functionality is provided. In some embodiments an ADM component may extend functionality of a standard EMR system so that such system is able to utilize ADM templates and/or ADMs.

In some embodiments of any aspect(s) hereof, database updates, feedback, or response may be performed or provided in a timely manner. In some aspects an average time for providing feedback or response to a HCP or updating an EMR or ADM may be selected so as to not substantially interfere with or delay normal workflow of the HCP. In some aspects an average response or update time may be selected to be below a predetermined value. In some embodiments a predetermined value may be equal to or less than 1, 2, 5, 10, 15, 20, 30, 40, 45, 50, or 60 seconds for, e.g., at least some classes of actions. In some embodiments an alert pertaining at least in part to time anticipated to be required for response or update may be provided, said alert comprising, e.g., an estimated time, an indication that a response or update may take more than a predetermined time, an option to abort an update or query, etc. In some embodiments, database accesses, updates, or queries are at least in part prioritized. Prioritization may take into account, e.g., factors such as the user (e.g., whether the user is a contributor or subscriber), the nature of the action, prior response times to the user or during a session, etc. For example, an action performed in response to a contributor, e.g., a HCP, may be assigned a higher average priority than an action performed by a non-contributor.

### BRIEF DESCRIPTION OF THE SEVERAL VIEWS OF THE DRAWINGS

FIG. 1 is a block diagram showing an exemplary implementation of a system and interaction with various categories of users in accordance with some implementations.

FIG. 2 is a block diagram showing details of exemplary interactions between a EMR system and contributors in accordance with some embodiments.

FIG. 3 is a flow chart in accordance with some embodiments.

FIG. 4 is a flow chart in accordance with some embodiments.

FIG. 5 is a flow chart in accordance with some embodiments.

FIG. 6 is a flow chart in accordance with some embodiments.

FIG. 7 is a diagram showing an implementation of a cloud computing system in accordance with some embodiments.

FIG. 8 shows a screen shot illustrating creation of an ADM in accordance with some embodiments. A HCP, e.g., a clinical research coordinator, creates an Active Diagnosis Module for the patient. This may be accomplished by clicking on the link “New ADM” within the EMR. Also shown is a link for accessing historical ADMs (ADMs created prior to creation of the new ADM).

FIGS. 9A and 9B show screen shots from within an ADM in accordance with some embodiment. FIG. 9B shows requesting entry of a tentative diagnosis in accordance with some embodiments. A scrolldown list of diagnoses may be presented for selection in accordance with some embodiments.

FIG. 10 shows a screen shot illustrating the successful creation of an Active Diagnosis Module in accordance with some embodiments. Successful creation is indicated by a ribbon on top of the screen. Initially this ribbon labels the diagnosis as tentative. This screen shot shows a pink ribbon indicating that a tentative diagnosis of multiple myeloma has been entered. The clinical research coordinator or treating physician is now instructed by the ADM to fill in all information needed to confirm the diagnosis.

FIG. 11 shows a screen shot illustrating that, in accordance with certain embodiments, when all required information has been properly filled out, the ribbon on top of the screen changes color and indicates that the diagnosis has been confirmed. This screen shot shows a purple ribbon indicating that a diagnosis of multiple myeloma has been confirmed.

FIG. 12 shows a screen shot illustrating exemplary means for accessing experimental therapies functionality in an EMR system in accordance with some embodiments.

FIG. 13 shows a screen shot illustrating exemplary means for accessing experimental therapies functionality in an EMR system in accordance with some embodiments. The screen shot shows a purple ribbon indicating that a diagnosis of multiple myeloma has been confirmed.

FIG. 14 shows a screen shot illustrating exemplary appearance of a screen after experimental therapies functionality is accessed in accordance with some embodiments. Two menus are available: Clinical Trials; Expanded Use Therapies.

FIG. 15 shows a screen shot illustrating exemplary appearance of a screen after “Clinical Trials” has been selected in accordance with some embodiments. A protocol is chosen and the ADM now becomes a “Screening ADM”. The ADM-SC may contain appropriate fields to ensure that protocol requirements for enrollment are met. Once all the screening data elements are met, a request may be sent to the sponsor to enroll the patient.

FIG. 16 shows a screen shot illustrating exemplary appearance of a screen in which an ADM can be exited by clicking “Exit ADM” in the upper right corner in accordance with some embodiments.

FIG. 17 shows a screen shot illustrating exemplary appearance of a screen in which historical ADMs (ADMs created prior to the creation of a newly created ADM) can be entered again by clicking on the “Historical ADM” link in the EMR menu in accordance with some embodiments.

FIG. 18 is a schematic diagram showing interactions between an ADM-equipped EMR system or business entity and various constituencies having an interest in experimental therapies in accordance with some embodiments.

FIGS. 19A-19G show a screen shot and sequence of steps illustrating creation of a new ADM for the tentative diagnosis “Age-related Macular Degeneration—Geographic Atrophy”, completion of various ADM fields, and activation of the ADM with a definitive (confirmed) diagnosis of “Age-related Macular Degeneration—Geographic Atrophy” in accordance with some embodiments.

FIG. 20 is a schematic diagram showing interactions between ADMs for two patients and various ADM-equipped EMR systems at different health care organizations that have EMRs for those patients in accordance with some embodiments (left side of diagram). Also shown (right side of diagram) are interactions between the ADMs and the patients, and interactions between the ADMs and various entities interested in the data in the ADMs.

FIG. 21 is a schematic diagram of a system (shown in the upper part of the figure) in which an ADM database interfaces with an ADM-equipped EMR system and with an EDC system in accordance with some embodiments. Data entered into subject's EMRs is automatically copied into the ADMs. The lower part of the figure shows a conventional EDC system that requires entry of data from subject's EMRs into the EDC system by study personnel.

FIG. 22 is a schematic diagram of a system in which ADMs (ADM-EDCs) perform all electronic data capture functions for a clinical trial at three trial sites in accordance with some embodiments, eliminating the need for a separate EDC system at these sites.

FIG. 23 shows various screens of a smartphone equipped with a Dx app in accordance with some embodiments. FIG. 23 interface 2301 shows that a Dx app may be accessed via an icon, which may be present on a home screen of a smartphone. FIG. 23 interface 2302 shows that after the patient taps the “My Dx” icon a list of diseases appears on the screen. For example, FIG. 23 interface 2302 shows a disease list for a patient who has multiple myeloma, type II diabetes, and hypertension. FIG. 23 interface 2303 shows functions that become available after a patient selects a particular disease from the disease list in accordance with some embodiments.

FIG. 24 shows various screens of a smartphone equipped with a Dx app in accordance with some embodiments. FIG. 24 interface 2401 shows selection of a “Find Patients” function. FIG. 24, interfaces 2402, 2403 and 2404 show subsequent screens.

FIG. 25 shows various screens of a smartphone equipped with a Dx app in accordance with some embodiments, after a Find Patients app has been selected as in FIG. 24. FIG. 25 interface 2501 shows selecting a patient found by the Find Patients app. FIG. 25 interface 2502 shows sending a message to the patient.

FIG. 26 shows various screens of a smartphone equipped with a Dx app in accordance with some embodiments. FIG. 26 interface 2601 shows selection of a “Find Physicians” function. FIG. 26 interface 2602 shows a map presenting the location of various physicians and that selecting a particular physician results in display of the physician's name and rating. FIG. 26 interface 2603 shows the physician's address and affiliation and shows providing options to visit the physician's website or make an appointment with the physician.

FIG. 27 shows various screens of a smartphone with a Dx app in accordance with some embodiments. The interfaces in FIG. 27 show a sequence of screens relating to an Experimental Therapies function in accordance with some embodiments.

FIG. 28 shows various screens of a smartphone equipped with a Dx app in accordance with some embodiments. FIG. 28 interface 2801 shows accessing an ADM that has an alert. FIG. 28 interface 2802 shows the content of the alert.

FIG. 29 shows various screens of a smartphone equipped with a Dx app in accordance with some embodiments. FIG. 29 interface 2901 shows accessing an ADM that has expired. FIG. 29 interface 2902 shows an alert notifying the patient that the ADM has expired and advising the patient how to have the ADM reactivated.

FIG. 30 shows various screens of a smartphone equipped with a Dx app in accordance with some embodiments. FIG. 30 interface 3001 shows accessing an active ADM. FIG. 30 interface 3002 shows options presented after “Access ADM” is selected.

FIG. 31 shows various screens of a smartphone equipped with a Dx app in accordance with some embodiments. FIG. 31 interface 3101 shows selection of a “Coach Me” function. FIG. 31 interface 3102 shows a patient disease management score and presentation of an option by which a patient can learn how to improve his or her patient disease management score. FIG. 31 interface 3103 shows suggestions provided to a patient who requests to learn how to improve his or her patient disease management score. FIG. 31 interface 3104 shows that a patient may be provided with various health promotion and monitoring apps via a Coach Me function.

FIG. 32 shows various screens of a smartphone equipped with a Dx app in accordance with some embodiments. A patient is offered the option to download various health promotion and monitoring apps.

FIG. 33 shows various screen of a smartphone equipped with a Dx app in accordance with some embodiments. FIG. 33 interface 3301 shows a home screen that includes an icon for a Dx app and icons for an Exercise app, Diet app, and Medications app located to the right of the Dx app. FIG. 33 interface 3302 shows a screen of an Exercise app that allows a patient to enter the intensity and duration of an exercise session by tapping or sliding their finger along an intensity scale and a duration scale. FIG. 33 interface 3303 shows a screen of a Diet app. A patient takes a photo of a plate of food using his or her smartphone. The app may extract information from the photo and may, for example, compute an estimated calorie count, classify the food items (e.g., as vegetable, fruit, carbohydrate, poultry, fish, meat, dairy, sweet, etc.). FIG. 33 interface 3304 shows a screen of a Medication app. A patient's medications are listed along with a corresponding image of the medication and the time at which it is supposed to be taken. The patient can indicate having taken the medication by clicking in the appropriate box. A check mark may then appear in the box.

FIG. 34 shows various screens of a smartphone equipped with a Dx app in accordance with some embodiments. FIG. 34 interface 3401 shows a home screen that includes an icon for a Dx app and an icon for a Body Monitoring app located to the right of the Dx app. FIG. 34 interface 3402 shows two body monitoring apps (weight and blood pressure) and an option by which a patient can add additional body monitoring apps. FIG. 34 interface 3403 shows that upon selecting the “other” option a patient is offered a chance to download an ECG app.

### DETAILED DESCRIPTION

Overview

In some aspects, a computer-implemented process of acquisition of health information by a business entity, the health information being useful for assembly of electronic medical records (EMRs), is presented.

In some aspects, a computer-implemented process of administering a business entity that has, as a business purpose, the acquisition of health information, e.g., health information sufficient to assemble a database of EMRs, is presented.

In accordance with some aspects, health information (data) regarding an individual may be electronically received from a contributor. A contributor may be a health care provider (HCP) of the individual. For purposes hereof, a collection (set) of health information regarding an individual may be referred to as a “health information dataset”. The health information dataset received from the contributor may be evaluated to determine whether the dataset fulfills a predetermined set of criteria that are required for assembly of a EMR (the “EMR criteria”). If the dataset fulfills the EMR criteria, the dataset may be deemed adequate. An EMR may be generated using the dataset and may be stored in a database (the “EMR database”). In some embodiments, a system, including the EMR database, may be at least in part owned or controlled or administered by a business entity. “At least in part owned or controlled by the business entity” may mean, with regard to the EMR database, that the business entity may at least exerts control over the format and/or use of the database. For example, the business entity may control the type of information included in the database and/or may control access to or use of the database by contributors and, e.g., other parties. The format of the database may be proprietary to the business entity. Ownership of the data itself may at least in part reside with a contributor, health care organization (HCO), and/or patient, e.g., in the sense that such individuals or organizations may be legally entitled to require removal of at least some of the data from the database or may be entitled to receive a copy of the data or use the data for their own purposes or may receive remuneration in exchange for certain uses of the data.

As with standard EMRs, assembling a database of EMRs may involve expenditure of time and effort on the part of health care providers, e.g., health care professionals such as physicians. For example, HCPs may need to spend time familiarizing themselves with a data input format and/or transmitting existing health information pertaining to their patients to the EMR system. In some implementations, an ongoing commitment may be required to maintain the quality of the EMRs by, for example, submitting new health information that becomes available. In some aspects, the business entity may compensate contributors based at least in part on the health information that the contributor submits. Such compensation (also referred to as “remuneration”, an “incentive” or a “payment”) may provide an incentive for contributors to submit health information adequate to meet certain standards specified by the business entity and, for example, to complete such tasks as are appropriate to maintain the quality of the data. Compensation may be provided based on any of a number of factors and in any of a variety of forms in various embodiments. For example, in certain embodiments, submission by a contributor of a sufficient number of adequate datasets to generate a selected number of EMRs or modules thereof, may entitle the contributor to receive an incentive. In some embodiments an incentive comprises a share in the business entity.

The EMR database may be used by any of a variety of individuals and/or entities. In some embodiments, HCPs who contribute health information may use the EMRs in the ordinary course of providing care for their patients. In some embodiments, patients may use the EMR database to, for example, review their health information. In some embodiments, the business entity may permit third parties to access the EMR database in exchange for a fee. Such third parties (sometimes referred to as “subscribers” herein) may use the database, for example, to perform medically related research or for any of a variety of other purposes. Some interesting potential uses of the database may include, for example, identifying previously unknown risk factors for diseases or adverse drug reactions, identifying unnecessary or counterproductive utilization of medical resources, identifying instances of failure to implement appropriate treatment or preventive measures, identifying or tracking outbreaks of infectious diseases or foodborne illnesses, initiating and tracking recalls of medications or medical devices where appropriate, tracking treatment outcomes attained by different health care organizations, etc. Retrospective and/or prospective studies may be performed.

A “user”, e.g., of the EMR database or of a system or apparatus comprising or accessing such database, may refer to any individual that transmits (submits) information for potential inclusion in the EMR database or that receives information retrieved from the EMR database (e.g., in response to a request by the user). At least some of such information may have been processed prior to being transmitted to the user, e.g., as described further below. Users may, for example, be contributors, subscribers, patients, patient representatives, government employees, or employees of the business entity, in various embodiments. A user may, in some embodiments, be an organization that, through its employees, contractors, or representatives, transmits or receives information to or from a database, apparatus, or system.

In some embodiments, an inventive system may comprise a database that contains user account data, e.g., contributor account data. Such account data may include, for example, data relating to EMRs to which the contributor contributed and/or incentives earned by the contributor. In some embodiments, the business entity may maintain or cause to be maintained (e.g., through another entity) a database that contains shareholder account data, as discussed further below. Such shareholder account data may include, for example, information identifying the business entity's shareholders and the number of shares owned by each shareholder.

As will be appreciated by one of ordinary skill in the art, the present invention or any one or more aspects thereof may be embodied, for example, as a system, apparatus, method or computer program product. Accordingly, the present invention or any one or more aspects thereof may take the form of hardware, software, or embodiments combining software and hardware aspects that may all generally be referred to herein as a “system”, which may comprise one or more “components”. Furthermore, the present invention or any one or more aspects thereof may take the form of a computer program product embodied in any tangible medium (e.g., a non-transitory storage medium) having computer usable program instructions embodied in the medium. Any combination of one or more computer usable or computer readable medium(s) may be utilized in various embodiments. The computer-usable or computer-readable medium may be, for example but not limited to, an electronic, magnetic, optical, electromagnetic, infrared, or semiconductor system, apparatus, device. Examples of a computer-readable medium include the following:, a hard disk, a random access memory (RAM), a read-only memory (ROM), an erasable programmable read-only memory (e.g., EPROM or Flash memory), a portable compact disc read-only memory (CDROM), a floppy disk, an optical storage device, or a magnetic storage device. A computer-usable or computer-readable medium may in some embodiments be paper or another suitable medium on which the program is printed or embodied, as the program may be electronically captured, for instance, via optical scanning of the paper or other medium (optionally employing optical character recognition), then compiled, interpreted, or otherwise processed in a suitable manner, if necessary, and then stored in a computer memory and/or executed by a computer processor. In the context of this document, a computer-usable or computer-readable medium may be any medium that may contain, store, communicate, propagate, or transport the program for use by or in connection with the instruction execution system, apparatus, or device. The computer-usable medium may include a propagated data signal with the computer-usable program code embodied therein. The computer usable program code may be transmitted using any appropriate medium, including but not limited to wireless, physical wires, wireline, optical fiber cable, etc.

For purposes hereof, the term “EMR system” may be used to refer to the one or more aspect(s) or feature(s) that receive health information from contributors, assemble EMRs therefrom, and/or perform any of a variety of other functions associated with the retrieval and/or processing of health information submitted to and/or stored in the EMR database. In many embodiments, the EMR system may interact with users (e.g., via a standard graphical user interface (GUI), analyze submitted health information, and/or assemble the health information into EMR database records. The EMR system may comprise multiple components. For example, one or more components may receive and/or transmit information to or from users. One or more components may analyze information received from users. One or more components may add information to the EMR database. One or more components may extract information from the EMR database, e.g., in response to a query from a user. One or more components may analyze extracted data and/or convert the data into an appropriate format for transmission to a user. One or more components may receive and/or transmit information between other component(s) of the EMR system or external to the EMR system. In some embodiments, the EMR system may include a clinical decision support system (CDSS) component. The CDSS may, for example, provide advice or suggestions to HCPs based at least in part on information entered into the EMR database and may perform any of a variety of other functions of such systems. Various other components that may be included in the EMR system are described below.

A user may transmit or receive health information via any type of electronic transmission in various embodiments. An electronic transmission may occur over a network, e.g., a computer network such as the Internet or a phone network. In some embodiments, in reference to exchange of information or data between a contributor (or other user) and the EMR system, the terms “transmit” and “submit” may be used interchangeably herein, as are related terms such as “transmitting” or “transmission”, “submitting” or “submission”, etc. Where reference is made to “entering” or “entry” of data into the EMR system, it should be understood that such data may be submitted to the EMR system unless otherwise indicated. Submission may occur in response to a request or action initiated by a user or in response to a request initiated by the EMR system. For example, at least some health information entered by a user may remain stored on a user's computer for a period of time prior to being transmitted to the EMR system. Such transmission may occur in response to a request initiated by the EMR system, which may occur automatically, e.g., at predetermined time intervals. In some embodiments at least some health information may remain stored on a computer or data storage system owned or controlled at least in part by a user (e.g., a HCP) or HCO but is made available to the EMR system for analysis and/or retrieval. For purposes hereof, such information may be considered to be submitted to the EMR system.

In some aspects the disclosure provides a computer program product sometimes referred to herein as an “application” for a portable electronic device. The application may allow contributors and/or shareholders to access at least some of their account data. An application that allows a user to access at least some of his or her account data may be referred to as an “account access application”. In some aspects the disclosure provides a computer readable medium having an account access application embodied in its memory. In some aspects the disclosure provides a portable electronic device having an account access application embodied in its memory.

In some aspects it is envisioned that the EMR system may interface with an application, e.g., an application for a portable electronic device, wherein said application allows users to interact with the EMR system using the portable electronic device. For example, in some embodiments HCPs may be able to enter or retrieve patient information, communicate with patients or other HCPs, or perform other activities described herein (e.g., data analysis activities) using a portable electronic device having such application embodied thereon. In some aspects the disclosure may provide a computer readable medium having such application embodied in its memory. In some aspects the disclosure may provide a portable electronic device having such application embodied in its memory.

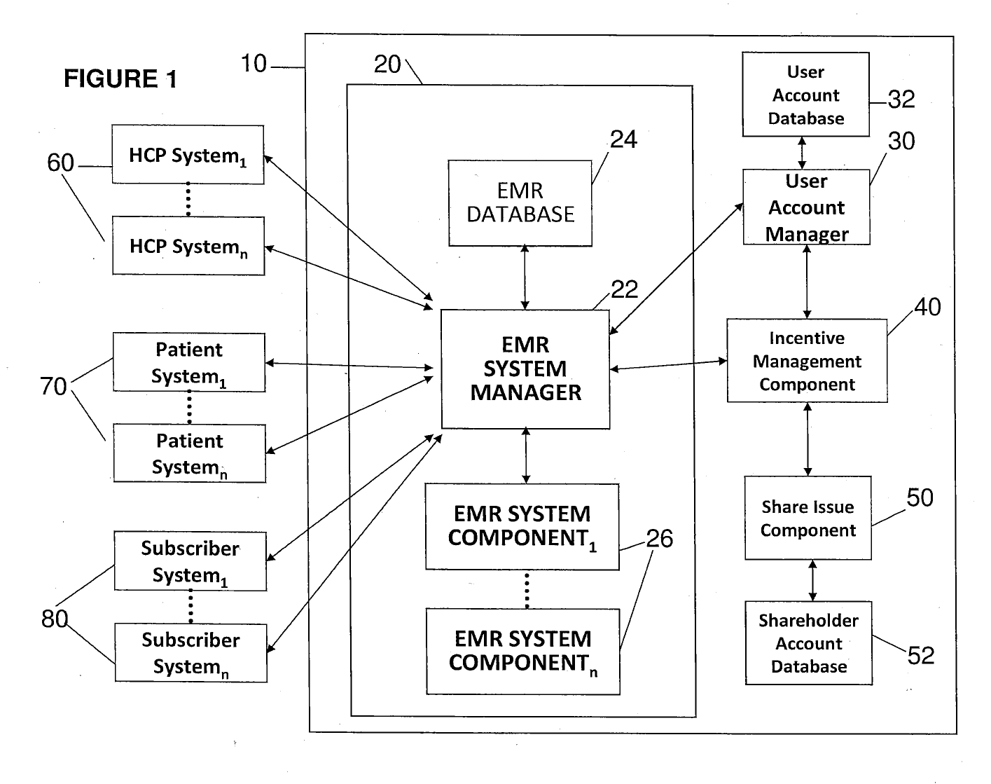

FIG. 1 shows a block diagram of an exemplary system 10 in accordance with some implementations. System 10 may include a EMR system 20 that handles (e.g., receives, analyzes, stores, transmits) health information. System 10 may include ancillary components (right) that may not be part of EMR system 20 (e.g., in that they may not directly handle health information) but that support activities of EMR system 10 and/or of the business entity. Interaction of system 10 with various categories of users in accordance with some embodiments is also depicted. For example, system 10 may interact at least with multiple health care providers. In many embodiments system 10 may interact with multiple patients and/or multiple subscribers. It will be understood that HCPs, patients, and subscribers may interact with system 10 using any of a variety of electronic systems labeled in FIG. 1 as systems 60 (for use by HCPs), 70 (for use by patients), and 80 (for use by subscribers), respectively that link to system 10 via one or more networks. Systems 60, 70, and 80 may comprise one or more computers, smart phones, or other suitable electronic devices. HCP systems 60, patient systems 70, and subscriber systems 80 are labeled 1 through n to indicate that numerous different HCPs, patients, and subscribers may interact with system 10. It should be understood that employees of the business entity may also interact with system 10 (not shown).

EMR system 20 may comprise EMR system manager 22, EMR database 24, and one or more additional EMR system components 26, labeled as EMR component₁-EMR componentₙ. The number of EMR system components may vary, and separate components may be defined as would be appreciated by one of ordinary skill in the art. EMR system manager 22 at least in part manages communication (interaction) between users and various components of the EMR system and, in some embodiments, at least in part manages interactions between various components of the EMR system. For example, EMR manager 22 may receive health information from an HCP, transfer health information to a EMR component 26 for analysis, receive a response from EMR component 26, and/or transmit the response to the user. EMR manager 22 may include or interface with a database management system (DBMS) for the EMR database 24, which database stores EMR data, as described further herein. EMR manager 22 may (via the DMBS) extract information from EMR database 24 in response to a request from a user or add data to EMR database 24 in response to a request from a user, e.g., after said data has been analyzed and approved by a EMR component 26. EMR manager 22 may perform one or more additional functions. For example, EMR manager 22 may process information received from users or from EMR components 26 prior to transmitting such information to EMR database 24. EMR manager 22 may perform various functions of the EMR system itself and/or may issue instructions to one or more EMR components 26 to perform any one or more functions of the EMR system described herein and/or may issue instructions to ancillary system components (described below).

EMR components 26 may include at least an analysis component that analyzes health information received from a HCP. Such analysis may include, for example, determining whether a health information dataset is adequate to meet EMR criteria, determining whether a conventional disease diagnosis proposed by a user meets conventional disease diagnosis criteria, etc. The analysis component may provide feedback based on the analysis, which feedback is transmitted to the user. EMR analysis component 26 may inform EMR manager 22 whether particular information is adequate to entitle a contributor to an incentive. If so, EMR manager 22 may inform user account manager 30 and/or incentive management component 40 accordingly. EMR components 26 may include a component that analyzes and, e.g., processes queries from users (e.g., HCPs, patients, subscribers). For example, the analysis component may convert a raw query (e.g., a natural language query) into a format acceptable to the DBMS. EMR components 26 may include a clinical decision support system component (CDSS) or an interface thereto, a computerized physician order entry component or an interface thereto, a billing component or an interface thereto, and/or a scheduling component or an interface thereto, etc. EMR components 26 may include one or more components that analyze health information entered into the EMR system and provide or trigger provision of an alert to a user upon entry of particular data, as described herein. EMR components 26 may include one or more components that interface with an application running on a portable electronic device, as described herein.

Ancillary components of system 10 may include, for example, a user account manager 30, an incentive management component 40, and/or a share issue component 50. User account manager 30 may perform any of a variety of functions connected with the establishment, maintenance, updating, etc., of user accounts. For example, user account manager 30 may perform functions such as receiving registration information from users, assigning user IDs or passwords, checking login information received from users, etc. User account manager 30 may maintain and/or interface with a user account database 32, which stores user account data. User account manager 30 may store user account information in user account database 32, extract information from user account database 32, and, e.g., analyze such information or transmit the information to other system component(s). User account manager 30 may be at least in part responsible for checking login credentials supplied by users against information stored in user account database 30 and instructing EMR manager 22 to provide, limit, or deny access to the EMR system or portions thereof based at least in part on the comparison. User account manager 30 may perform functions relating to subscriptions (e.g., billing subscribers, transmitting renewal reminders to subscribers, monitoring and controlling use of the EMR system by subscribers, etc.).

Incentive management component 40 may perform any one or more functions connected with the distribution of incentives to contributors, as described herein. For example, incentive management component 40 may determine the amount or nature of incentives due to a contributor, direct share issue component 50 to issue one or more shares to a contributor, may arrange for the dispatch of a check to a contributor, may arrange for a direct deposit of funds to a contributor's account at a financial institution, etc. In some embodiments, incentive management component 40 may perform such function(s) at least in part in response to instructions or information received from EMR manager 22 and/or user account manager 30.

Share issue component 50 may issue shares in the business entity, e.g., in response to a request from incentive management component 40. In some embodiments share issue component 50 may receive and/or respond to external requests (not shown), e.g., requests from within the business entity, directing the issuance of one or more shares. In some embodiments share account database 52 may receive and/or respond to external requests (not shown), e.g., from within the business entity. For example, share issue component 50 and/or share account database 52 may perform any conventional functions connected with the issuance, management, or tracking of shares in a business entity.

It should be understood that FIG. 1 shows only a subset of potential interactions that may occur between various components in certain embodiments. It should also be understood that at least some components of system 10 may communicate directly with one another and/or with users as well as, or in addition to, communicating via EMR manager 22, and that any communications may take place over a network or within or between one or more processors, as appropriate. For example, at least some requests for certain user account data or shareholder account data, and responses thereto, may be handled by a component of system 10 (not shown on FIG. 1) without involving EMR system 20. It should be understood that EMR manager 22 and any of EMR components 26 or ancillary components 30, 40, and/or 50 may be composed of multiple subcomponents and may include or access one or more databases in addition to those depicted.

It is noted that aspects or features comprising compensating contributors (e.g., health care providers) based at least in part on the submission of health information and/or based at least in part on the use of such contributed health information (after such de-identification as is appropriate) by third parties (e.g., subscribers), may be independent of any particular implementation of the system or components thereof

Business Entity

The business entity may be any of a variety of types of business entity in various embodiments. For example, the business entity may be a limited liability partnership (LLP), a limited liability company (LLC), a C corporation (C corp), or an S corporation (S corp), as those terms are understood in the United States. In various embodiments, the business entity may be organized under the laws of any state in the US (e.g., Delaware, New York, California, Kentucky) or may be organized under the laws of an ex-US jurisdiction. The business entity may be privately held or may be a public company (i.e., its shares may be publicly traded), in various embodiments. If publicly traded, the shares may be listed on one or more stock exchanges. It should be understood that the term “share” may be employed in regard to corporations. In embodiments in which the business entity is not a corporation, “share” as used herein may refer to an ownership interest in the business entity, which may include, for example, a share of the net profits of the business entity and the right to receive distributions of the business entity's assets, and may or may not include the right to vote and participate in management of the business entity. In some embodiments, the business entity may be a not-for-profit organization, also referred to as a nonprofit organization. It will be understood that if the business entity is a nonprofit organization, certain aspects or features pertaining to shares in the business entity may not apply. The business entity may in various embodiments have one or more subsidiaries that may, for example, be organized under the laws of different states or countries. In some embodiments, the business entity at least in part owns or controls a system of the present disclosure, e.g., an EMR system.

Contributors

In general, a contributor may be any individual who has health care information pertaining to one or more individuals and the right to submit the health information to the business entity. As noted above, a contributor may be a HCP of the individual to whom the health care information pertains, such that the individual may be a “patient” of the HCP. A patient may, and often does, have multiple HCPs. HCPs may include, for example, a patient's primary care physician, specialist medical practitioners such as cardiologists, dermatologists, or ophthalmologists who may provide care to a patient on a regular or as-needed basis, physician assistants, nurse practitioners, nurses, pharmacists, surgeons, dentists, or any other health care provider who has sufficient interaction with a patient to acquire health information adequate for assembly of, or appropriate for inclusion in, a EMR. In some embodiments a HCP may be member of an allied health profession, which term may refer to health professions distinct from medicine, surgery, dentistry, and nursing.

A contributor that first contributes health information regarding a patient that is used to assemble a EMR may be referred to as the “primary contributor” for that EMR. Contributors that subsequently submit health information regarding that patient may be referred to as “secondary contributors” for that EMR.

A contributor, e.g., a HCP, may be employed by and/or may be an at least partial owner of a health care organization (HCO) and/or a HCP may be affiliated with one or more HCOs. For example, a physician affiliated with a hospital may not be an employee of the hospital but may be entitled to admit patients to the hospital. “Health care organization” (HCO) may include any organization that provides health care to multiple persons. Such organizations include, e.g., hospitals, health clinics, health centers, skilled nursing facilities, and physicians' practices. A HCO may provide inpatient services, outpatient services, or both. A HCO may be a for-profit entity or a nonprofit entity. A HCO may have custody over and/or access to medical records of multiple patients. Certain types of outpatient health care may be delivered via organizations such as pharmacy clinics or temporary health clinics that offer services such as vaccinations, cholesterol screenings, and, in some instances, more extensive medical services. In some embodiments such organizations may be considered HCOs. A HCO may authorize multiple different individuals, e.g., employees, partners, or affiliates thereof to contribute health information to the EMR system. In some embodiments a HCP or HCO may be engaged mainly in primary care, secondary care, or tertiary care.

In some embodiments, a contributor may be a patient. In some embodiments, a contributor may be a patient's parent, legal guardian, health care proxy, or other representative. A representative may be any individual legally authorized (e.g., by the subject) to provide health information regarding the subject to the business entity. A person who contributes health information concerning himself or herself may be referred to herein as an “auto-contributor”. A person who contributes health information concerning a child, ward, or other individual for whom the person is acting as a representative may be referred to herein as a “proxy contributor”. In some embodiments an auto-contributor or proxy contributor may only be permitted to be a secondary contributor. Contributions by auto-contributors or proxy contributors may include, for example, symptom diaries, medication usage diaries, exercise diaries, food intake diaries, and/or results of self-administered monitoring tests such as weight, blood glucose tests, blood pressure checks, etc. In some embodiments it is envisioned that results of self-administered monitoring tests may be uploaded directly from a monitoring device into the EMR database. In some embodiments feedback is provided to a patient or HCP based at least in part on said results. For example, a patient or HCP may be advised to schedule a patient visit, modify a treatment, etc.

In some embodiments, contributors or other users may be offered an opportunity to elect to receive electronic updates (e.g., via email, text message, voicemail, or other electronically communicated form), said updates may contain information relevant, for example, to particular medical conditions, therapeutic agents, or other medically relevant topics. In some embodiments, updates may be customized based on HCP discipline, patient roster, or user preferences. The EMR system may store information pertaining to issuance of such updates, e.g., in account information.

In some embodiments, contributors or other users may or may be able to interact with each other, e.g., via or assisted by the EMR system. For example, contributors may be able to allow at least some other users or categories of users to contact them via, for example, email, text messaging, Internet forum, or Internet chat room, for any of a variety of purposes. In some embodiments email between patients and their HCPs and/or email between HCPs may be supported. In some embodiments such emails may be stored in the relevant patient's EMR. In some embodiments patients may allow themselves to be contacted by individuals or organizations seeking subjects for clinical trials, or vice versa. In some embodiments patients may allow themselves to be contacted by individuals or organizations engaged in or planning to engage in research relating to particular medical conditions from which the patient suffers, or vice versa. In some embodiments patients may interact with each other. Interaction may be anonymous, e.g., at the option of either interactor, in various embodiments. Further discussion of certain embodiments relating to patient interaction is provided below in the section entitled “Social Media”.

In some embodiments, at least some categories of users may be able to access information pertaining to HCOs or HCPs. For example, users may be able to view the number of patients having a particular diagnosis who are or have been under care of particular HCO or HCP within a predetermined time period, or to view HCO or HCP rankings, etc.

Health Information and Submission and Retrieval Thereof

In general, a contributor may submit health information to the EMR system and retrieve information from the EMR database in any of a variety of ways in various embodiments. It is envisioned that the EMR system may interact with a user via one or more computer-based documents (e.g., web pages, e.g., dynamic web pages). The user may navigate between different pages or portions thereof by clicking on “links” (which may be indicated using text of a different color or font), arrows, and other navigation methods typical of web page navigation. For example, users may log on to the EMR system using a computer and will be presented with a computer-based document (displayed on a screen) from which various options may be selected. The particular options available to the user could depend on the type of user (HCP, subscriber, etc.). For example, a HCP may be presented with options such as accessing existing EMRs of his or her patients, initiating creation of a new EMR, ordering a medication, or any of a variety of other functions. The options may be presented in a hierarchical manner.

In general, health information entry and submission, and use of database content may be facilitated by use of standard GUI elements (sometimes referred to as GUI widgets), such as buttons (e.g., boxes to check or fill in, radio buttons), lists (e.g., scroll-down or drop-down lists from which to select among various options), menus, etc. For example, in some embodiments, entry of health information by a contributor may be facilitated by providing the contributor on his or her computer with one or more document(s) (forms) that contain a template or other structured format in which the contributor is presented with various input fields to complete. At least some of the input fields may be presented as buttons or lists of options from which to select. Typical webform elements may be used. Various electronic data entry systems and input devices may be supported such as touch screens, light pens, keyboard, digital pen, stylus, scanners, cameras, etc. Handwriting recognition software may be employed. In some embodiments information may be entered verbally, e.g., in response to verbal prompts from the EMR system. For purposes hereof, a document having a preset input format may be referred to as a “template”. In some embodiments, a template may contain at least some fields that are to be completed by (a) making a selection from a set of predetermined options (whether presented visually, orally, or otherwise); (b) entering a numerical value; (c) answering questions where there may be a limited number of potential responses (such as yes/no questions). In some embodiments an option may be “none of the above” (indicating that none of the other presented options is appropriate), “unknown”, “not applicable”, “other”, or like terms. If “other” is selected, the contributor may in some embodiments be permitted to enter an item not included in the set of predetermined options, e.g., as free text. A template may contain at least some fields that request or permit entry of a specified type of data whose content may at least in part not be readily or conveniently entered by way of selecting from methods (a), (b), or (c). Examples of such data may be physician notes (e.g., progress notes) or images (e.g., images produced by diagnostic imaging devices, photographs, sketches, video or audio files, etc.). An appropriately labeled field allowing entry of free text may be provided, such as a field for entering physician notes. Appropriately labeled fields allowing the user to upload or provide a reference number or storage location for an image may be provided. The EMR system may generate a EMR by assembling the information and adding it to the EMR database, e.g., as a database record.

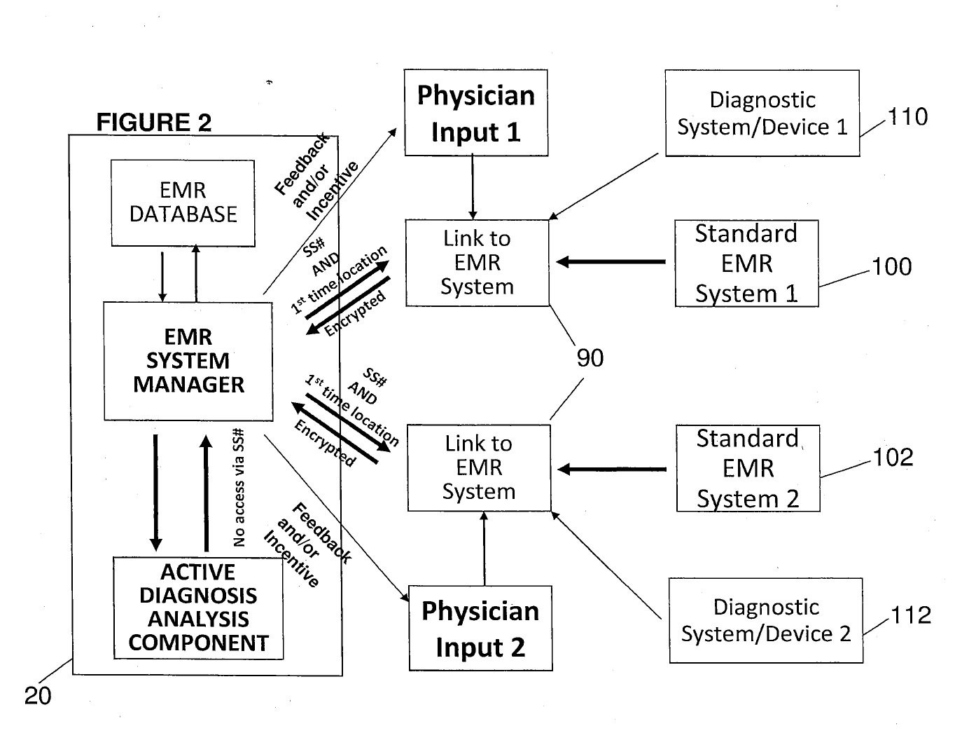

FIG. 2 shows certain details of exemplary interactions between EMR system 20 and certain types of contributors (physicians) in accordance with some embodiments. It will be understood that EMR system 20 may include additional components not shown on FIG. 2. EMR system 20 may interact with various systems 90 that communicates with (links to) system 20 via one or more networks. Systems 90 may each include one or more computers that may or may not be in communication one another but in some embodiments each such system may include at least one device that is capable of communicating with system 20. For example, system 90 may comprise, e.g., a computer located in a physician's office, a computer located in a facility that performs clinical chemistry tests, a computer that interfaces with or is part of a medical imaging system, or any standard EMR system or a system in which a standard EMR is stored, etc. For purposes hereof, the term “standard EMR system” or “existing EMR system” may refer to an existing (as of the date of the present invention and/or as of the date of the invention described in PCT/US2012/64125 (entitled “Systems and Methods for Assembling Electronic Medical Records” and filed Nov. 8, 2012)) EMR system, e.g., a computer program product for creation or maintenance of EMRs, that is commercially available or otherwise publicly known or used as of the date of the present invention and/or as of the date of the invention described in PCT/US2012/64125. In some embodiments a standard EMR system is an updated version of an existing standard EMR system, wherein the updated version does not comprise Active Diagnosis Module (ADM) templates or ADMs and is not equipped with functionality that makes possible the utilization ADM templates and/or ADMs and/or Experimental Therapies functions within or in connection with such EMR system, e.g., as described further below. The term “standard EMR system” as used herein thus encompasses the term “standard EMR system” as such term is used in PCT/US2012/64125. It will be understood that “standard EMR system” is not intended to refer to inventive EMR systems described in PCT/US2012/64125 and/or in US provisional patent application U.S. Ser. No.: 61/746,590 (entitled “Systems and Methods for Using Electronic Medical Records for Clinical Trials” and filed Dec. 28, 2012) and/or in US provisional patent application U.S. Ser. No. 61/763,890 (entitled “Systems and Methods for Using Electronic Medical Records for Clinical Trials” and filed Feb. 12, 2013). A “standard EMR” may be created in or using a standard EMR system. A standard EMR system may be, for example, listed on the Certification Commission for Health Information Technology website or another certification body. Commercial providers of certain standard EMR systems include, for example, Cerner, EPIC, and AthenaHealth. For purposes hereof, the term “EMR” or “EMR system” in the Summary, Brief Description of the Drawings, Drawings, Detailed Description, or Claims hereof should be understood to refer to an inventive EMR or inventive EMR system, respectively, unless indicated or evident from the context as referring to a standard EMR or standard EMR system or as evident from the context, respectively. Inventive EMRs and inventive EMR systems are disclosed herein or disclosed both herein and in PCT/US2012/64125 and/or in US provisional patent application U.S. Ser. No.: 61/746,590. In certain embodiments a standard EMR system may be any EMR system that does not comprise Active Diagnosis Module (ADM) templates or ADMs and is not equipped with functionality that makes possible the utilization ADM templates and/or ADMs and/or Experimental Therapies functions within or in connection with such EMR system, e.g., as described further below. It will be appreciated that in embodiments in which a standard EMR system is equipped with functionality that makes possible the utilization of Active Diagnosis Module (ADM) templates and/or ADMs and/or Experimental Therapies functions within or in connection with such standard EMR system, e.g., as described further below, the term “EMR system” may in certain embodiments refer to the resulting EMR system equipped with such functionality unless otherwise indicated or evident from the context.

When initially establishing or accessing a EMR for a patient, a physician may enter the patient's social security number (SS#). The SS# may be encrypted at least during transmission. The first entry of the patient's SS# by the physician may require that the patient be present at the same location as the physician (indicated as P^{t} time location on FIG. 2), e.g., as determined based on patient's mobile phone location. Subsequently, the physician could enter the patient's SS# without requiring that the patient be present. In some embodiments it is envisioned that the EMR system may provide a patient with a unique EMR ID distinct from the patients SS#, which unique ID could be used instead of a SS# solely, e.g., for EMR access purposes. In some embodiments, in addition to entering the patient's SS# or EMR ID, the physician may enter health information pertaining to the patient. As shown in FIG. 2, the EMR system may provide feedback and/or an incentive to the physician based on analysis of the health information, as described herein.

It is envisioned that, in some embodiments, at least some health information may be entered into EMR system 20 from one or more standard EMR systems, e.g., via system 90. Standard EMR systems are labeled 102 and 104 in FIG. 2. In some embodiments it is envisioned that the EMR system will interface with any of a variety of standard EMR systems. The EMR system may, for example, with authorization from a contributor, extract information pertaining to the contributor's patients from such standard EMRs and use the information to at least partially assemble EMRs for such patients as appropriate. Contributors may be requested to verify and/or supplement such imported health information. It should be understood that standard EMR systems may be running at least in part on a system 90 in a physician's practice or health care organization. Physician input may be requested or required in order to transmit information from system 90 into the EMR system and/or physician input may augment the information extracted from standard EMR systems. For example, information extracted from existing EMR system 102 may be used to populate at least some fields of a form, and the physician may input additional information to complete or correct the form. EMR system 20 may cause the information to be displayed for physician review and input.

It is envisioned that at least some health information may be entered into EMR system 20 from diagnostic systems or devices (labeled as 110 and 112 on FIG. 2). Physician input may be requested or required in order to select an active diagnosis module (described below) to which such information is to be added and/or to confirm that the physician has reviewed the health information. EMR system 20 may cause the information to be displayed for physician review and input.

In some embodiments, health information may be entered at least in part using dictation. The EMR system may include, make use of, or accept input from appropriate voice recognition software and/or speech recognition software. In some embodiments the EMR system may ask the HCP a series of questions, either orally or by presenting the questions on a computer screen (or combinations thereof). The EMR system may receive responses from the contributor, assemble the information into a EMR, and/or add the EMR to the EMR database.

In some embodiments a contributor need not physically enter all of the health information for a patient. For example, the contributor may delegate at least part of the task of entering at least some portion of the health information to an appropriately authorized individual operating under the contributor's direction. In some embodiments such information would not be eligible for incorporation into a EMR until the contributor acknowledges having reviewed the information. In some embodiments, policies may be set where the contributor permits incorporation from trusted entities or delegates directly into an EMR.

In some embodiments, the EMR system may analyze a health information dataset to ensure that it meets predefined criteria and may provide feedback to the contributor based on such analysis. For example, if a contributor fails to provide an item of information that is required according to the EMR criteria, the contributor may be informed that the health information dataset is not adequate and may be provided with suggestions regarding the additional information needed and/or the reason why the dataset was determined to be inadequate. The data may be checked for possible inconsistencies or other errors, e.g., laboratory values or drug doses outside the possible range or otherwise likely to be incorrect. The HCP may be requested to confirm such data and/or the data may be tagged in the database as being possibly erroneous. Health information added to an existing EMR may be checked in a similar way as it is entered. In some embodiments, more stringent criteria may be applied to current data than to data generated prior to creation of the EMR.

In some embodiments, the particular criteria that a health information dataset generally must meet in order to be deemed adequate to assemble a EMR may vary and may be determined by the business entity (or other entity that at least in part controls or administers the EMR system) within its discretion. In other embodiments, the criteria may be requirements or recommendations. In this regard, it should be understood that the term “EMR”, as used herein, may indicate that the health information contained therein, or at least a portion thereof, may have met predetermined criteria (such as completion of a set of fields of a template, e.g., a template that may be provided by the EMR system) but may not specify the criteria that must be met and may not require that the health information have any specific content. For example, at least some of the health information, may have been analyzed and determined to meet predetermined criteria. The criteria may be selected in any way of a variety of ways and may take into consideration any of a variety of different factors as appropriate for any one or more uses envisioned for the EMR or portion(s) thereof. In many embodiments it is envisioned that a EMR may be used by HCPs or HCOs in their ordinary activities and may replace wholly or at least in part the use of paper-based records or, in at least some embodiments, the use of standard electronic medical record systems. In some embodiments, it is envisioned that a EMR database may be structured in a way that facilitates the ability to perform useful research, e.g., medically related research, such as to collect information regarding outcomes or side effects associated with various treatments, medications, or combinations thereof or any of various types of analysis (which may be referred to as “meta-analysis”). The use of the EMR database to perform activities (e.g., medically related research) not directly pertaining to care for a particular patient may, in many embodiments, be subject to appropriate privacy and/or legal rules or considerations such as those of the Health Insurance Portability and Accountability Act (HIPAA), the Common Rule (45 CFR 46, Subparts A, B, C and D), etc. In some embodiments, such use may be subject to the privacy and/or other legal rules or considerations of a country or union in which a patient resides and/or in which a patient seeks health care and/or in which a HCP is registered to practice.

In some embodiments, the EMR system may facilitate the integration of health information held or generated at or by multiple different locations or individuals (e.g., at different HCOs or HCPs), which may exist or be generated in multiple diverse formats. The EMR system may convert information existing in diverse formats into a standard format that may, for example, be viewable on diverse computer hardware platforms or display devices, which may be supplied with appropriate software. A set of standard terms, e.g., to describe symptoms, physical exam findings, findings on diagnostic tests or images, diagnoses, treatments, etc., may be defined and used. A glossary may be provided so that users of the EMR system may look up the meaning of any term of whose meaning they are uncertain.

In general, health information suitable for inclusion in a EMR may comprise, for example, any of the following elements: medical history, surgical history, obstetric history, medications (sometimes abbreviated as Rx herein), allergies to medications, family history, social history, habits that potentially have an impact on health, immunization history, growth chart, developmental history, or any other health-related information. It will be appreciated that a medical record may contain at least several of the foregoing elements and that not all elements may be relevant, appropriate, available, or necessary for all or even most patients. For example, a growth chart may be relevant for a young child but not for a typical adult. In addition to health information, a medical record may contain potentially individually identifiable information such as the patient's name, address, phone number, birth date, social security number, etc. It is envisioned that a EMR may be presented to a user as one or more computer-based documents (e.g., web pages, e.g., dynamic web pages). The user may be able to navigate between different pages or portions thereof by clicking on links, arrows, icons, menu options, and/or other methods typical of web page navigation. The various elements of a EMR may be stored in different fields of the database record.

By way of example, in some embodiments, a EMR may include at least some of the elements listed below under the heading “Central EMR Database Format”. For example, a EMR may often include a Patient ID, at least some Patient Data, and at least one Active Diagnosis Module (described further below). It is noted that the term “Central” is used to indicate that the database records in the EMR system may have a common or uniform format and should not be interpreted as indicating that the EMR database must be a centralized database, although it may be in some embodiments. In some embodiments the EMR database may be a distributed database comprising multiple databases that may be uniform, similar, or heterogeneous in structure and may be stored in a single computer or multiple computers (or on single or multiple computer-readable media). Such computers or computer-readable media may be geographically located in the same place or different places and may be interconnected by a network in various embodiments. The EMR system components may in some embodiments include components for interfacing between multiple different databases, and, in some embodiments, providing data integration and/or presenting a uniform format to users.

### Exemplary Central EMR Database Format

1. Patient ID (e.g., encrypted SSN—retrievable by physician or other Health Care Provider (HCP) under specified circumstances, e.g., if during initial access the patient is present in the HCP's office)
2. Patient Data
   a. Demographic Information (e.g., date of birth, gender, etc.)
   b. Family History
   c. Diseases
   d. Surgeries
   e. Historical Diagnostic Tests
   f. Historical Rx
   g. Allergies to Rx
   h. Current Rx
3. ACTIVE DIAGNOSIS MODULE(S) (e.g., selected from scroll-down list or other selection means, e.g., arranged or based at least in part on HCP discipline)
   a. Existing ACTIVE DIAGNOSIS MODULE(S)
   b. New ACTIVE DIAGNOSIS MODULE(S)

Returning to the description of the Central EMR Database Format, in some embodiments, “Patient ID” may refer to an identifier that may identify a particular individual having a EMR stored in the EMR database. In some embodiments a patient ID may be a social security number. In some embodiments a patient ID may be provided by the contributor who submits the health information dataset used to assemble the EMR. In some embodiments a patient ID may be provided by the business entity following submission by a contributor of a health information dataset adequate to assemble a EMR for the patient. Exemplary “Patient Data” may generally include information of the type that may be found in a typical medical record, such as demographic information, family history, information regarding the patient's diseases and surgeries, diagnostic tests, treatments, allergies to medications, etc.

It will be appreciated that some of the information under Patient Data may not apply to a particular patient or may be unknown to the contributor. For example, a patient may not have any known allergies to medications, may not have any current medications, or may not have had any surgeries. In such cases, the relevant field of the EMR could be marked with a designation such as “none”, “unknown”, or “not applicable”. “Diseases” may include, e.g., data regarding at least those diseases of the patient that were diagnosed after creation of the EMR and may also include data regarding at least some diseases that were diagnosed before creation of the EMR, e.g., diseases that have been monitored or treated since the time that the EMR was created or that resolved prior to creation of the EMR. Data pertaining to a disease may include, for example, a diagnosis, symptoms experienced by the patient, physical exam findings, physician notes, treatment and/or follow-up plan, or any other disease-related information. “Surgeries” may include, e.g., information regarding surgeries (if any) that the patient has had since the EMR was created and may include information regarding at least some surgeries that the patient had before the EMR was created. “Historical Diagnostic Tests” and “Historical Rx” may refer to diagnostic tests (e.g., with results) and/or treatments that were performed or administered in the past (with respect to a time point at which the EMR is accessed). In some embodiments a EMR may include a “Past Medical/Surgical History” section, which may contain at least some of the patient's past medical/surgical history as of the date of the date of creation of the EMR. It should be understood that the information under “Patient Data” may be arranged in the EMR database and/or displayed to the user in any of a variety of ways, and the list below is not intended to require any particular structure or format. For example, diagnostic tests and treatments may be together with the particular disease or surgery to which they are relevant. Information may be arranged at least in part chronologically, e.g., by date of patient visit. Different formats may be used for outpatient visits versus hospitalizations. In some embodiments, the “Patient Data” section may comprise or may have at least some of the functionality of a standard EMR.

In some embodiments the EMR system may provide one or more medical history templates or physical exam templates, which may be specialized for a particular health care discipline. For example, a general physical exam template or a specialized physical exam template such as a neurological exam template or ophthalmological exam template may be provided. In some embodiments a HCP may select from among a set of such templates.

In some embodiments, a EMR may comprise or may be organized at least in part around a module referred to as an “active diagnosis module” (ADM), e.g., as shown below in some exemplary, non-limiting embodiments. In some embodiments of any aspect herein, an EMR database comprises, consists of, or consists essentially of an ADM database. In some embodiments of any aspect herein, one or more functions of an EMR system may be performed using ADMs.

In some embodiments, an ADM may correspond to a disease or a risk factor for a disease that has come to the attention of a HCP.

### Exemplary Active Diagnosis Module (e.g., Designed at Least in Part to Facilitate Future Research or Meta-Analysis)

1. Conventional Disease Diagnosis (e.g., selected from scroll-down list or other selection means)
2. Molecular Disease Diagnosis (if applicable, scroll-down list or other selection means may appear based on Conventional Disease Diagnosis)
3. Diagnostics
4. Rx

In some embodiments, an ADM may be designed to contain or reference at least a substantial portion of the health information that is directly relevant to a particular disease in a patient (at least to the extent that such health information has been gathered by or made available to HCPs who utilize the EMR system) so that it may be possible by reviewing the ADM and, if relevant, patient summary data (discussed below) to obtain a reasonably comprehensive understanding of the disease process in that patient and the diagnostic and therapeutic management thereof (at least starting from the date of creation of the ADM). In some embodiments, an ADM may, for example, include at least the following four elements: (1) a “conventional disease diagnosis”; (2) a “molecular disease diagnosis”; (3) diagnostic tests (“diagnostics”) performed that pertain to the disease and, in many embodiments, at least some results thereof; and (4) treatments prescribed or administered to the patient (abbreviated Rx). In some embodiments each ADM may be assigned a unique identifier. In some implementations, the identifier may be used to refer to the ADM in research studies, publications, reports, etc., thereby potentially facilitating verification of the study results or performance of follow-on studies.

In some aspects, an ADM is a disease-specific data module that aggregates a patient's medical data relevant to a particular disease. The ADM may be updated by or at, e.g., one or more HCPs or HCOs, as the patient receives medical care for the disease over time. A patient or patient's EMR may be associated with a single ADM or multiple ADMs. For example, a patient may have a Type II diabetes ADM and a carcinoma ADM. A patient's EMR may comprise a Type II diabetes ADM and a carcinoma ADM. In some embodiments an ADM aggregates all or substantially all of a patient's medical data relevant to a particular disease. Such disease-relevant information may comprise disease-specific information, which may be relevant specifically to the disease or to a set of diseases, and, in at least some embodiments, information that may be relevant to general health and/or to any of a wide variety of diseases such as demographic data, selected physical examination data such as weight, height, blood pressure at most recent patient visit, medical history, surgical history, family history, medications. In some embodiments an ADM is linked to a patient or EMR via an identifier, which may be encrypted. In some embodiments an ADM is or can be de-identified. In some embodiments an ADM is or can be linked to multiple EMRs of a patient. At least some such EMRs may in some embodiments have been created at different health care organizations and/or by different HCPs.

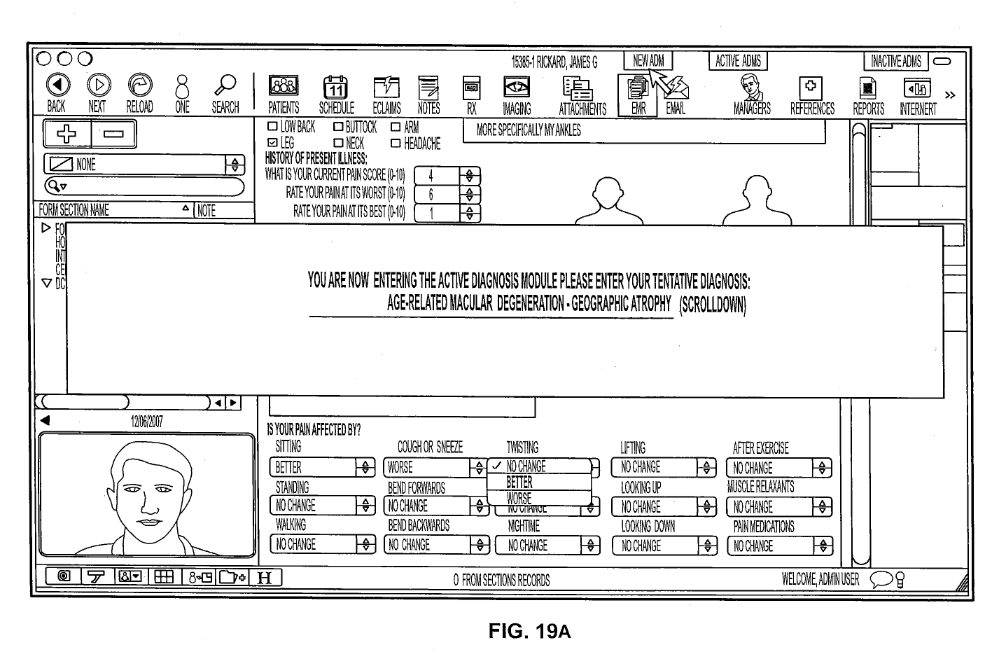

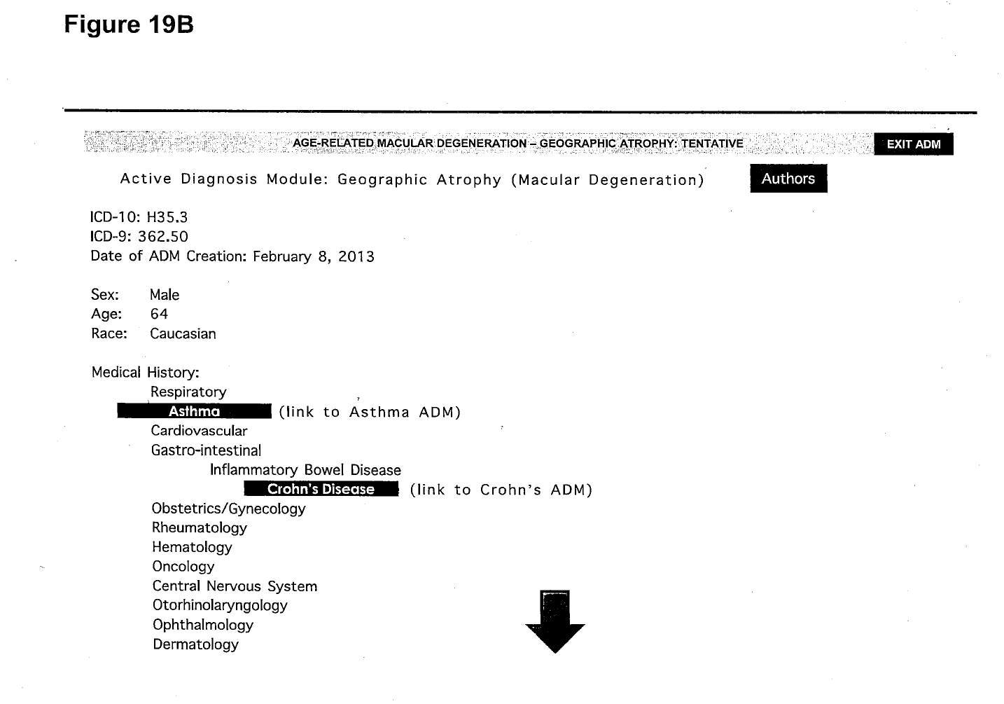

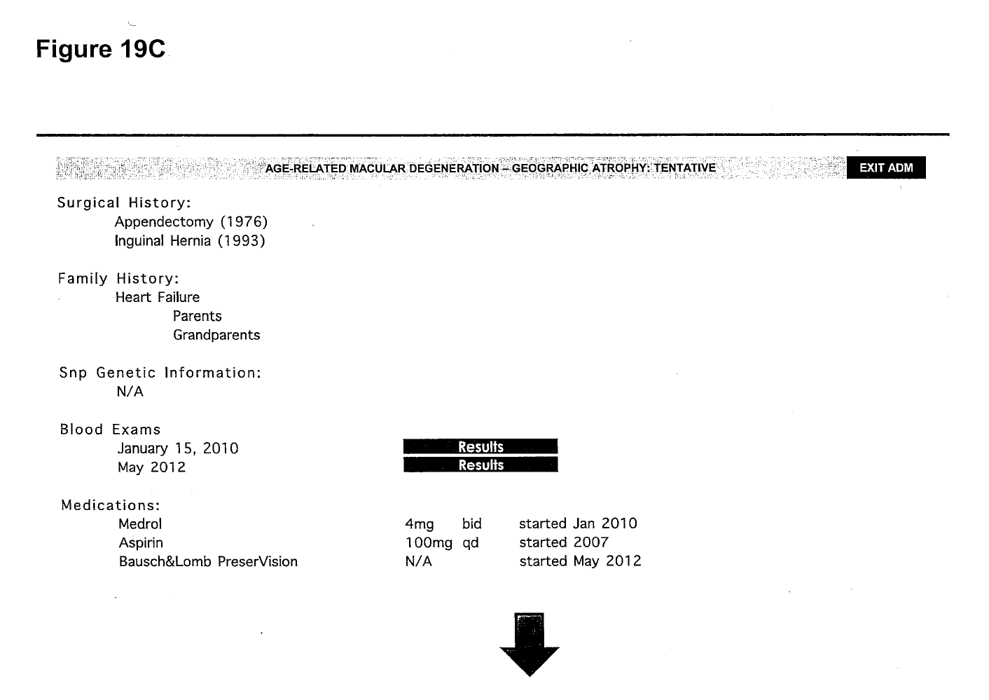

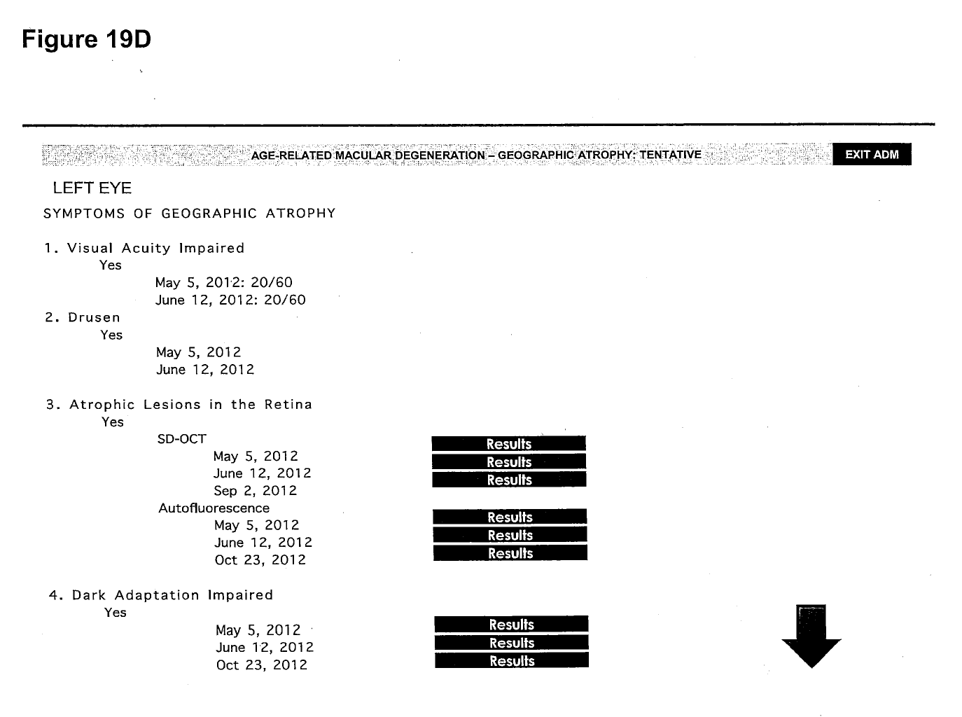

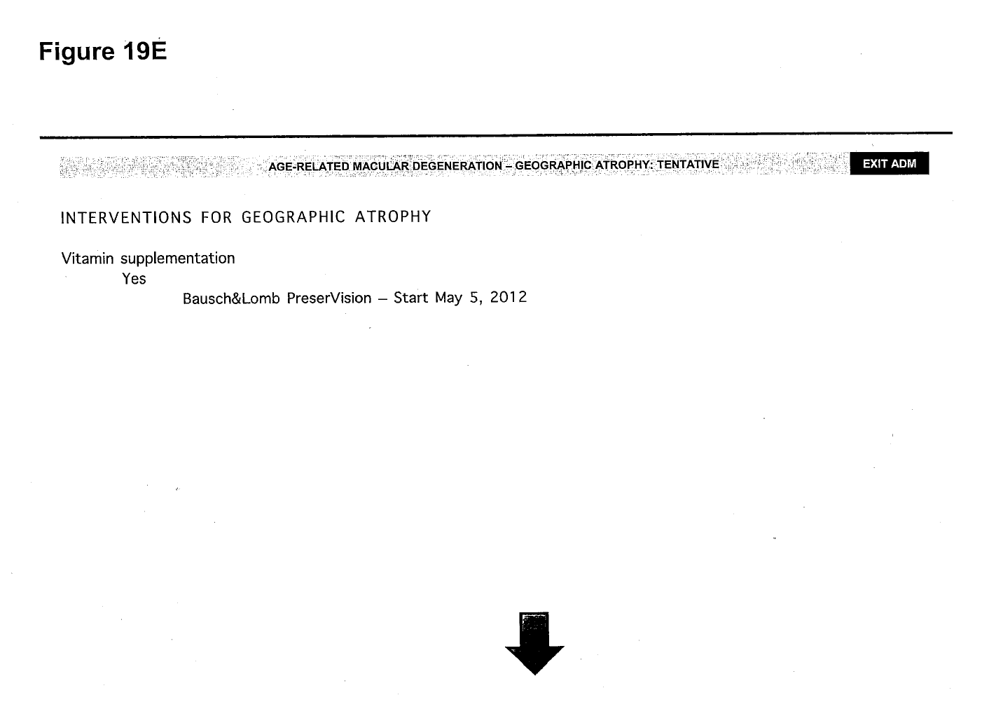

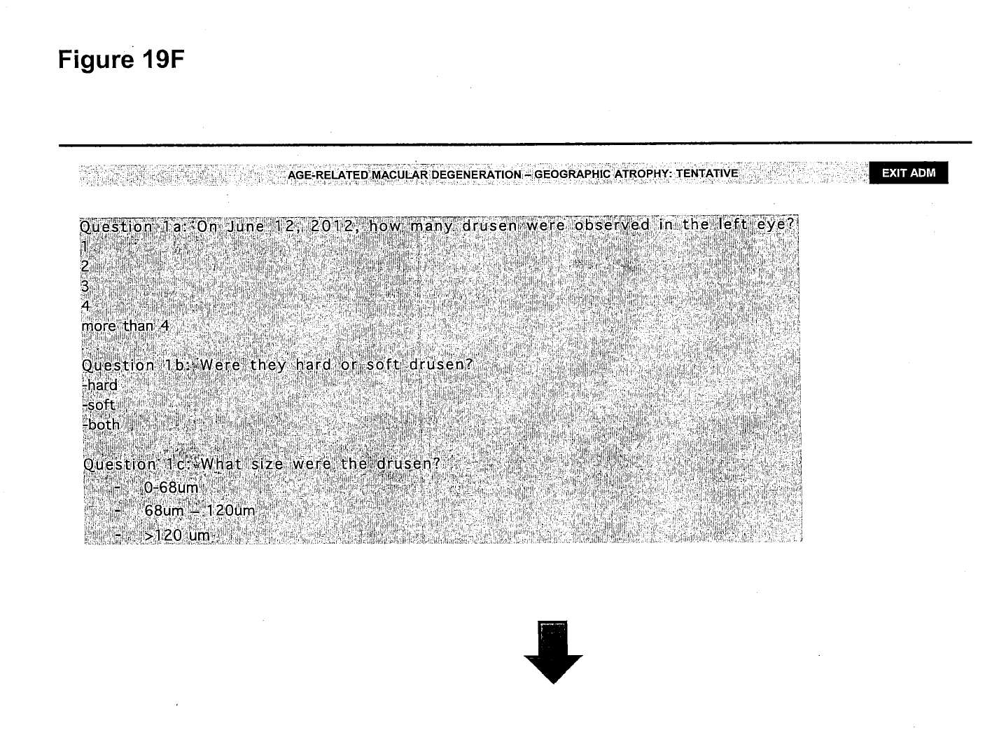

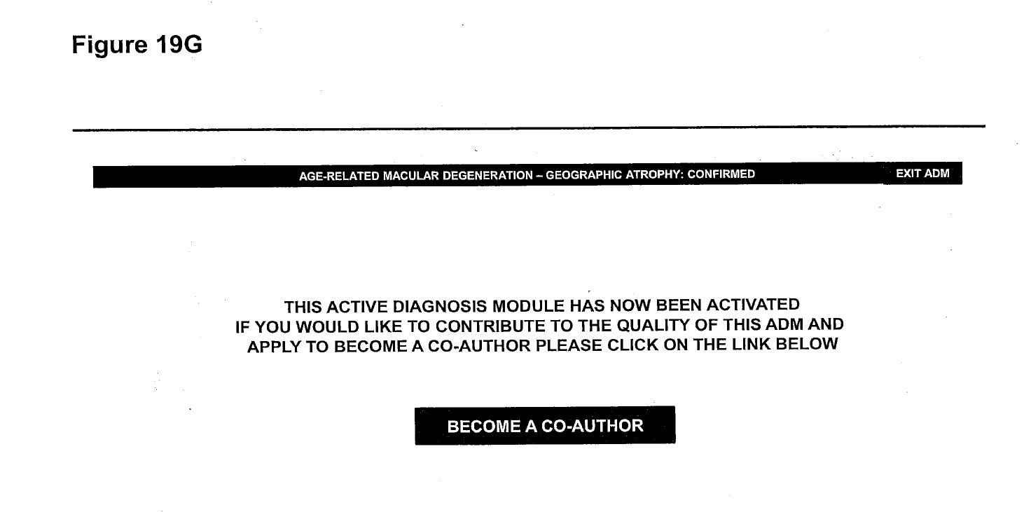

In some embodiments, once an ADM has been created using a particular EMR system, access is provided to other ADMs of the patient that may have been created using EMR systems at different HCOs, which may or may not utilize the same EMR system platform. In some embodiments an ADM comprises a link that permits a user (e.g., an HCP) to access other ADMs of that patient. For example, as shown in FIG. 19B, an ADM for a particular disease may comprise a Medical History section that comprises links to ADMs for other diseases that the patient has. Thus, an ADM or set of ADMs may comprise or provide access to aggregated disease-relevant information for a particular patient, including information relevant to many, most, or all diseases that the patient has been diagnosed as having. An ADM or set of ADMs may thus represent an electronic medical record containing the data relevant to a patient and that patient's diseases that is independent of any particular EMR format or EMR system and that separates certain core functions of the practice of medicine, namely diagnosis and treatment of disease, from other functions for which standard EMR systems and EMRs may be used, such as billing, scheduling, etc. In some embodiments an EMR system may comprise ADMs and computer-executable instructions for using ADMs in any one or more ways described herein.

The term “Existing Active Diagnosis Module” may refer to an Active Diagnosis Module that has already been created at a particular time that the EMR is accessed. “New Active Diagnosis Module” may refer to an Active Diagnosis Module that is being created or has been created during a current access session. In some embodiments, it is envisioned that a new ADM may be created by a HCP when or shortly after a patient health problem initially comes to the attention of the HCP, e.g., during or shortly after a patient visit during which the health problem is first discussed or detected. In some embodiments, a HCP may be presented with the option of creating an ADM by, for example, selecting an icon labeled “Create ADM”, “New ADM” (or similar term) or selecting such option from a list. In some embodiments, the HCP may then be presented with a template (“ADM template”) containing fields for entering the relevant information for elements (1) through (4), as available. If there are already existing ADMs for the patient, the HCP may be presented with the option of opening such ADMs. The HCP may then add information to the ADM, such as an entry for a patient visit or a newly received diagnostic test result.

Conventional Disease Diagnosis (element 1) may be selected from, e.g., a predetermined set of possible diagnoses, which may be presented in the form of one or more scroll-down lists, for example. “Disease” may be used herein to refer to any disease, disorder, syndrome, injury, or condition for which a person may seek or receive professional advice or treatment by a health care provider (or on whose behalf such advice or treatment may be sought), e.g., any disease, disorder, syndrome, injury, or condition that would be documented in a medical record. In certain embodiments, “disease” may refer to any diagnostic entity that has been assigned a code in the International Statistical Classification of Diseases and Related Health Problems, 10th Revision, 2007 (known as “ICD-10”), published by the World Health Organization, or any updated version or successor thereof. In some embodiments, the, e.g., predetermined set of possible diagnoses may be at least in part, selected from the diagnoses included in ICD-10 or any updated version or successor thereof. In some embodiments, the predetermined set of diagnoses may be, at least in part, selected from the diagnoses included in International Statistical Classification of Diseases and Related Health Problems, 10th Revision, Clinical Modification, 2011 (known as “ICD-10-CM”), developed by The National Center for Health Statistics (NCHS), the US Federal agency that is responsible for coordination of all official disease classification activities in the United States relating to the ICD and the use, interpretation, and/or periodic revision of the classification activities. In some embodiments, conventional disease diagnoses may be at least in part selected from diseases discussed in a standard medical or surgical textbook such as Goldman's Cecil Textbook of Medicine, Saunders, 23^{rd} or 24^{th} ed. (2007, 2012), Lango, D., et al., Harrison's Principles of Internal Medicine, McGraw Hill, 18^{th} ed. (2011), or McPhee, S., et al., Current Medical Diagnosis and Treatment, McGraw-Hill Medical; 51st edition (2011), or other members of the Current Diagnosis and Treatment series (Lange series) published by McGraw-Hill Medical or updated editions of such references as may be published from time to time. In some embodiments, if the set of conventional disease diagnoses includes diagnoses that differ from those listed in the ICD (e.g., the then current ICD version or any specified ICD version, which may be specified by the user), the EMR system may assign or assist the user to assign, an ICD diagnosis and code based on the conventional disease diagnosis.

In some embodiments, an HCP may often enter a conventional disease diagnosis when (or soon after) creating a new ADM. In some embodiments, a conventional disease diagnosis may initially be deemed “tentative” and may be marked as such in the EMR system. For example, the correct diagnosis may be unclear until results of appropriate diagnostic tests have been received. In some embodiments the HCP may be required to select a single tentative diagnosis in order to create an ADM. In some embodiments the HCP may select multiple alternative or co-existing tentative diagnoses. In some embodiments a differential diagnosis may be entered. In some embodiments, the HCP may be able to modify the tentative diagnosis at any time, e.g., as results of such tests are obtained. In some embodiments, once a HCP believes that an accurate diagnosis has or may have been reached, the HCP may update the status of the ADM to “definitive” (e.g., after changing the tentative diagnosis if appropriate). In some embodiments, the EMR system may only permit the status of the ADM to be changed to “definitive” if a set of predetermined criteria (“EMR system diagnostic criteria” or “EMR diagnostic criteria”) for the proposed definitive diagnosis have been met, based on data that have been entered into the EMR system. For example, the EMR system may check whether results of appropriate tests have been entered and, if so, whether such results are consistent with the proposed definitive diagnosis. If results of such tests have not been entered or are inconsistent or likely to be inconsistent with the proposed definitive diagnosis, the EMR system may not permit the tentative status to be updated to definitive and may inform the HCP accordingly, or may require an additional action on the part of the HCP to override the tentative status of the ADM. In some embodiments, a HCP may be required to enter a reason for overriding the tentative status. A diagnosis status may be indicated in any of a variety of ways in various embodiments, such as by using a field (e.g., a check box), color coding, icon shape, etc. In some embodiments the EMR system, e.g., at the request or option of the HCP, may provide additional feedback to the HCP following entry of a tentative or proposed definitive diagnosis or may offer the HCP the option of consulting the CDSS. For example, in some embodiments, after a HCP enters a tentative diagnosis, the EMR system may suggest one or more alternative tentative diagnoses and/or may suggest one or more diagnostics that may be useful to confirm or reject a tentative diagnosis or alternative tentative diagnosis or that may be useful to assist with treatment selection. In some embodiments, if the EMR system rejects a proposed definitive diagnosis, the EMR system may also indicate which of the EMR system diagnostic criteria have not been met. It should be understood that a definitive diagnosis may or may not actually be the correct diagnosis of a patient's disease. There may be instances in which a diagnostic test may provide an incorrect result and/or in which a patient has an unusual disease or combination of diseases and/or an atypical presentation. A definitive diagnosis may be changed if, for example, additional health information is gathered that suggests to the EMR system and/or to a patient's HCP, that a definitive diagnosis may be incorrect. Various means and/or criteria for changing a definitive diagnosis may be provided. Thus a definitive diagnosis refers to a diagnosis that has at least met predetermined criteria or, if such predetermined criteria have not been met, a tentative status has been overridden by specific HCP action. The term “definitive” diagnosis may be used interchangeably with “confirmed” or “established” diagnosis herein. It will be understood that in embodiments comprising or using ADMs, e.g., wherein diagnostic criteria are embodied within an ADM template and/or computer-executable instructions for determining whether data satisfies diagnostic criteria are associated with or operate on data in an ADM or ADM template, EMR diagnostic criteria may equally well be referred to as ADM diagnostic criteria.

In some embodiments, EMR diagnostic criteria may be based at least in part on recommended diagnostic guidelines published or approved by professional associations of various medical/surgical specialties or subspecialties, by expert panels or committees of HCPs in the relevant disease area, or by national or international organizations or government medical research institutes such as the National Institutes of Health (U.S.) or corresponding government entities in other countries, World Health Organization, the European Organisation for Research and Treatment of Cancer (EORTC), or by others, e.g., art-recognized organizations or bodies. It will be understood that EMR diagnostic criteria may be revised over time in at least some embodiments or diseases. Furthermore, certain diagnoses may be deleted from or added to the set of possible diagnoses. A diagnosis stored in the EMR database may be tagged with information indicating a version number for the diagnostic criteria that were applied at the time the diagnosis was entered. In some embodiments, if diagnostic criteria for a particular diagnosis are revised, the EMR system may check ADMs that have previously been assigned that diagnosis and may determine whether the diagnosis is still valid according to the revised diagnostic criteria. In some embodiments, if the diagnosis is invalid according to the revised criteria, the EMR system may tag the ADM accordingly and, in some embodiments, may attempt to assign a valid diagnosis to the ADM. In some embodiments, the newly assigned valid diagnosis may not replace the previously assigned diagnosis but rather may be provided as an additional element, e.g., of the ADM.

Molecular Disease Diagnosis (element 2) may include information regarding to certain biomolecules found in the patient (e.g., DNA, RNA, protein, etc.) that may be relevant to the disease and/or its treatment. Such information may have been obtained by analyzing, at the molecular level, a sample obtained from the patient. For example, a conventional disease diagnosis might be “lung cancer” or “lung adenocarcinoma” or “non-small cell lung cancer” (NSCLC). A molecular disease diagnosis of the same condition might be “non-small cell lung cancer positive for abnormal anaplastic lymphoma kinase (ALK) gene” (or simply “ALK-positive non-small cell lung cancer”, where “positive for abnormal anaplastic lymphoma kinase (ALK) gene” may indicate that the patient's tumor exhibits an abnormal ALK gene (e.g., as assessed using an FDA-approved test). Such patients may be candidates for particular treatments shown to be effective for treating patients with lung cancers that express ALK. In some embodiments, a molecular disease diagnosis may be of use to classify a conventional disease into one or more categories that differ in regard to prognosis or likelihood of responding favorably to a particular therapeutic agent or class of therapeutic agent. A molecular disease diagnosis may represent the result of analyzing a single biomolecule or multiple biomolecules, ranging from a small set up to hundreds or thousands.

A contributor may be presented with a list of potential molecular disease diagnoses that may be based at least in part on the identity of a tentative or definitive conventional disease diagnosis. The contributor may select from the list based, e.g., on results of one or more appropriate tests. In some embodiments such a list may be presented after a tentative diagnosis is entered. In some embodiments such a list may be presented after conventional disease diagnosis is changed to definitive. In some embodiments, if a molecular diagnosis is inconsistent with a proposed definitive diagnosis, the EMR system may present an error message and/or may not permit the status of the ADM to be updated to “definitive” or may require the HCP to override the tentative status of the ADM. It will be understood that molecular disease diagnosis may not be available or applicable for some diseases, in which case, in some embodiments, this element may be omitted from the ADM template. In some embodiments, whether to perform the diagnostic tests that may be needed to establish a molecular diagnosis may be within the discretion of the HCP. For example, if a molecular diagnosis would not alter the treatment, such tests may not be performed.

Diagnostics (element 3) may include diagnostic tests performed that relate to the disease and, in some embodiments, at least some diagnostic test results. For example, continuing with the example of NSCLC discussed above, the name of the test that was performed to demonstrate ALK positivity and results thereof may be included in element 3, as would names and, in some embodiments, results of other tests used to diagnose or monitor the disease. In some embodiments, diagnostics may be selected from a predetermined set, which may be provided by the EMR system and may be based at least in part on the tentative diagnosis or diagnoses. In some embodiments, diagnostics may include, e.g., laboratory tests (e.g., clinical chemistry), EKGs, procedures such as bronchoscopy, histopathologic tests on cell or tissue samples, diagnostic imaging studies, etc. In some embodiments, results may include, for example, “raw data” and/or reports describing, analyzing, or interpreting the data. For example, diagnostic images (e.g., X-rays) and histopathology slides, as well as reports interpreting such images/slides may be included. In some embodiments an ADM may comprise a link that provides access to an image, report, or other raw data. In some embodiments an ADM may comprise or provide access to information that may be useful or necessary for proper interpretation of results. For example, reference values such as normal or pathological ranges, values and/or cutoffs may be provided for tests for which such ranges, values and/or cutoffs may vary depending, e.g., on the particular version of the test or details of how the test was performed, etc. Examples of the type of molecular characteristics that may be assessed (e.g., to provide a molecular diagnosis) may include, e.g., DNA sequence or epigenetic modifications, RNA or protein level, or activity or post-translational modification of specific proteins. Any suitable method for analyzing the relevant biomolecule(s) may be used as molecular diagnostics. In some embodiments DNA may be analyzed to determine the presence or absence of particular mutations, polymorphisms, translocations, amplifications, modifications, or other aberrant characteristics associated with a disease. Exemplary techniques may include sequence analysis, hybridization-based analysis (e.g., microarray analysis), immunological techniques such as immunohistochemistry, ELISA assay, protein microarrays, mass spectrometry, etc.

In some embodiments, an ADM template may include one or more fields in which results of certain tests may be entered by a HCP by selecting from a predetermined set of options. For example, if an imaging study has been ordered, the ADM template may request entry of particular information regarding the resulting image, such as the presence, absence, or dimensions of lesion(s), so as to facilitate searching or analysis. In some embodiments, the EMR system may comprise appropriate analytical tools to extract, e.g., relevant searchable information from images or other test results. In some embodiments an ADM template may include one or more medical history templates or physical exam templates, which may be general or may be specialized for a particular health care discipline or disease. For example, a general physical exam template or a specialized physical exam template such as a neurological exam template or ophthalmological exam template may be provided. In some embodiments a HCP may select from among a set of such templates. An ADM or ADM template or associated computer-executable instructions may comprise any of a variety of quality control functions. For example, numerical data may be checked to ensure that its value is consistent with the units, not incompatible with life or with previously entered data, etc. Data that is implausible, incompatible with life, or inconsistent in light of previous data may be identified. In cases of potentially incorrect, implausible, or incompatible data, the system may request confirmation of the data and/or provide an indication that the data is likely incorrect, should be checked, and/or cannot be entered (which may in some embodiments be overridden by an appropriately authorized user, e.g., HCP). In some aspects, such data checking/quality assurance functions may improve data quality and/or reduce medical error rate.

In some embodiments the EMR system, e.g., via the EMR CDSS, may suggest diagnostic tests that may be useful to, e.g., establish a tentative diagnosis as definitive or to rule out a potential alternative diagnosis or to guide selection of appropriate therapy. It is noted that the use of “diagnostics” or “diagnostic tests” is not limited to determining the identity of a disease or determining whether a disease is or is not present. Diagnostic tests may be used after a diagnosis has been established, e.g., in order to monitor the disease and/or the effect(s) of treatment.

Treatment Information (element 4) may include information relating to treatments prescribed or performed to treat the disease. Such information may include, e.g., medication-related information (e.g., name of medication administered or prescribed, dosage unit, administration instructions such as frequency and timing of doses), description of surgery, physical therapy, or other procedures performed or prescribed, medical or surgical devices used (e.g., external devices, implantable devices, prostheses), etc. Treatment information may include data entered by pharmacies or other providers of pharmaceutical agents. Such information may include, e.g., drug lot number, date of prescription fulfillment, etc. In some embodiments, a medication may be any product or combination product listed in, e.g., the United States Pharmacopeia (USP) or the National Formulary (NF) (both published by The United States Pharmacopeial Convention, Rockville, Md.) or listed in The Anatomical Therapeutic Chemical (ATC) classification system (WHO Collaborating Centre for Drug Statistics Methodology (WHOCC) (Oslo, Norway) or having an assigned code in the US National Drug Code (NDC) numbering system (http://www.fda.gov/Drugs/InformationOnDrugs/ucm142438.htm) or an entry in the National Drug Data File Plus (First DataBank). In some embodiments the EMR CDSS may suggest therapies (e.g., medications) that may be useful to treat a disease, based on, e.g., the conventional disease diagnosis, molecular disease diagnosis, and/or results of diagnostics. In some embodiments, the EMR CDSS may take into consideration one or more patient data items, such as age, weight, co-existing diseases (e.g., as represented by ADMs in the EMR), etc. In some embodiments, the EMR CDSS may suggest alternate diagnostics or therapies that may be more suitable for the patient (e.g., having a better benefit-risk profile) or may be less costly without sacrificing quality of care. Such suggestions may, for example, be based at least in part on analysis of patient data, other ADMs for that patient, genetic information, etc. In some embodiments, the EMR system, via the use of ADMs, may encourage use of evidence-based approaches in health care. In some embodiments, diagnostic or therapeutic recommendations made by the EMR system may be based at least in part on diagnostic or therapeutic guidelines published or approved by, e.g., professional associations of various medical/surgical specialties or subspecialties, by expert panels or committees of HCPs in the relevant disease area, or by national or international organizations or government medical research institutes such as the National Institutes of Health (U.S.), the World Health Organization, the European Organisation for Research and Treatment of Cancer (EORTC), or by other art-recognized organizations or bodies. In some embodiments, the EMR system may provide research findings or guidelines that support its suggestions, or a link thereto.

In some embodiments, Diagnostics (II.3) and Rx (II.4) from Active Diagnosis Modules may be automatically added to the Patient Data (I.2) as diagnostics accumulate. In some embodiments items from 1.2 may also or alternately be imported to II.3 and II.4 but may require active transfer or approval by a patient's HCP.

In some embodiments, patient symptoms described to the HCP and/or the HCP's findings on physical examination may be included in Diagnostics. In some embodiments the ADM may include one or more elements for patient symptoms and/of physical examination findings, in addition to the above-mentioned four elements. As noted above, at least some such information may be entered by way of templates provided by the EMR system in some embodiments.

In some embodiments an ADM may include one or more fields for entering information pertaining to complications that may arise as a result of a disease or as a result of treatment. One of ordinary skill in the art would be aware of complications that may arise in patients with particular diseases. Certain complications may be the subject of an additional ADM for the complication. In some embodiments, an ADM for a complication of an existing disease (or of a treatment for a disease) may be a “sub-ADM” of an ADM for the existing disease or may be connected to it via a link or other connecting means such as arrows, menus, or a hierarchical arrangement.

In some aspects, an EMR may include a problem list for a patient. In some embodiments, a problem list may at least include diagnoses in patient's unresolved ADMs and may further include significant items from Patient Data. In some embodiments a problem list is generated or updated at least in part by the EMR system, and may be subject to modification by a patient's HCP.

In some embodiments an ADM template may include one or more elements in which the HCP may enter notes, thought processes, plans, etc., as text. Alternately, or additionally, such information may be entered in Patient Data in some embodiments. In some embodiments HCP notes, thought processes, and/or plans may be included in an EMR but not included in the ADM(s) that are part of or associated with that EMR.

In some aspects, an ADM template may interface with or may be integrated with a standard EMR system. A EMR in some embodiments may comprise a standard EMR for the patient, one or more ADMs in accordance with the present disclosure and, e.g., a patient summary. In some embodiments, a HCP may select one or more ADM templates from the EMR system, which ADM template may be incorporated into or interface with a standard EMR. The ADM template may interface with components of the EMR system such as the EMR manager, EMR analysis components, etc. The EMR database may thus at least in part comprise ADMs that may reside on HCP's or HCO's computers but that may be accessed by other HCPs or subscribers via the EMR system. A EMR database record may thus have different formats or may be a virtual database record that comprises a standard EMR (which may be created by any of diverse EMR systems) and one or more ADMs. The EMR system may thus allow HCPs or HCOs that have, e.g., invested in standard EMR systems and integrated them with other legacy health information systems or operations such as scheduling or billing to continue using such standard EMR systems if desired while adding ADMs and other functions of the EMR system and, e.g., transitioning completely to the central EMR database format over time. In some embodiments, the EMR system may provide multiple different versions of an ADM template, the different versions being adapted for integration into or interfacing with different standard EMR systems. In some embodiments a patient summary may be generated by the EMR system from information in the standard EMR. In some embodiments, the patient's HCP may review and, if appropriate, may correct and/or supplement the automatically generated patient summary. In some embodiments, the patient's HCP may enter information into a patient summary template provided by the EMR system to generate a patient summary. In some embodiments the EMR system may provide tools to extract or analyze data contained in standard EMRs as well as in ADMs. In some aspects, the EMR system may provide tools that support at least partial sharing of health information stored among multiple different standard EMR systems. In some embodiments, the EMR system may provide a uniform user interface, which may enable users (e.g., HCPs) to store and/or retrieve data from multiple heterogeneous standard EMR systems in addition to using and analyzing ADMs. In some embodiments the EMR system may fulfill or substitute for the functions of a health information exchange (HIE), e.g., a regional health information organizations (RHIO) in addition to providing users with the functionality of ADMs and, in some embodiments, the ability to search and analyze them. In some embodiments ADMs may be implemented in conjunction with or as part of a HIE, e.g., a RHIO. In some embodiments a database comprising ADMs may be implemented in conjunction with or as part of a HIE, e.g., a RHIO. In some embodiments, as described herein, ADMs, ADM templates, and, in some embodiments, computer-executable instructions for creating and/or using ADMs may be stored or executed remotely from locations (e.g., HCOs) at which patient data are generated or entered. In some embodiments such ADMs, ADM templates, and/or computer-executable instructions may be at least in part cloud-based, wherein access to such ADMs, ADM templates, and/or computer-executable instructions, is provided (e.g., as a service) over a network, e.g., the Internet or, e.g., a virtual private network. A cloud may be a public cloud, wherein cloud services are provided by, e.g., public cloud service providers that make such services available to the general public, such Amazon AWS, Microsoft, or Google, or may be a cloud that is not generally or broadly available to the public.

In some embodiments an EMR system that is not an ADM-equipped EMR system may be equipped with functionality that makes possible the utilization of ADM templates and/or ADMs within or in connection with such EMR system. In some embodiments, for example, a standard EMR system may be equipped with functionality that makes possible the utilization of ADM templates and/or ADMs within or in connection with such standard EMR system. In some embodiments, systems and methods of equipping a non-ADM equipped EMR system, e.g., a standard EMR system, with functionality that allows such EMR system to create and/or use ADM templates and/or ADMs are described herein. In some embodiments such functionality is provided via a component, e.g., a software component, which component may be referred to as an “ADM component”. In some aspects, a non-transitory computer-readable medium comprising an ADM component is disclosed herein. In some embodiments an ADM component may be provided to an HCP or to an HCO that has entered into an appropriate agreement with a business entity that at least in part owns, controls, makes, sells, or provides the ADM component. In some embodiments an ADM component may be provided to a member of an information technology (IT) department at a HCO, such as a system administrator. The HCO may provide access to the ADM component to a selected set of computers and/or HCPs.

An ADM component may be provided in any suitable way in various embodiments. For example, in some embodiments an individual visits a website and downloads from such website a plug-in, wherein the plug-in comprises or consists of an ADM component that provides such additional functionality. As will be appreciated, the term “plug-in” refers to a software component or set of software components that adds specific functionality (abilities) to another software application. In some embodiments an ADM component is a plug-in for a standard EMR system. In some aspects, a plug-in may extend the usability of a standard EMR system. The term “plug-in” is used interchangeably herein with “add-on” or “extension”. In some embodiments an ADM component, e.g., a plug-in, is designed to function specifically with a particular EMR system, e.g., a particular standard EMR system. In some embodiments an ADM component, e.g., a plug-in, is designed to function with any of multiple standard EMR systems. In some embodiments an individual may be required to enter appropriate identifying information and is then offered the option of downloading an ADM component. Identifying information may be, e.g., a license number, DEA number or other prescriber number, or a code. A code may be provided by, e.g., (i) a company that at least in part owns, controls, makes, sells, or provides an EMR system into which such component is to be installed, e.g., a company that at least in part owns, controls, makes, sells, or provides a standard EMR system, (ii) a business entity that at least in part owns, controls, makes, sells, or provides, the ADM component, (iii) a sponsor of a trial (e.g., as discussed further below), etc. In some embodiments the website is at least in part owned or controlled by a business entity that at least in part owns, controls, makes, sells, or provides an EMR system. In some embodiments the website is at least in part owned or controlled by a business entity that at least in part owns, controls, makes, sells, or provides the ADM component. In some embodiments an ADM component may be provided on a tangible computer-readable medium such as a CDROM. In some embodiments installation from a tangible computer-readable medium may require entry of identifying information or a code and/or may be limited to particular computers. In some embodiments an ADM component may be provided as part of an upgrade of a standard EMR system. In some embodiments an ADM component may be an option that may be furnished together with or after adoption of an EMR system lacking ADM functionality, e.g., standard EMR system, by a HCO or HCP. In some embodiments an ADM component may be provided for purposes of use in clinical trial enrollment and/or electronic data capture.

An EMR system that utilizes ADMs, e.g., an EMR system in which EMRs are organized at least in part around ADMs from the outset or a standard EMR system that comprises an ADM component, may be referred to as an “ADM-equipped EMR system”. In some embodiments an ADM-equipped EMR system, e.g., an EMR system comprising an ADM component, differs from a standard EMR system in one or more ways. In some embodiments, following installation of an ADM component into a standard EMR system, one or more new link(s) are displayed within EMRs of at least some patients of an HCP who uses the system. Such link(s), when active, may allow a user to access functions that allow creation and/or use of an ADM. For purposes hereof, a situation in which a user of an EMR system has access to functionality for creation and/or use of ADMs may be referred to as being in an “ADM environment”. Thus an ADM component may be a component that equips a standard EMR system with computer-executable instructions appropriate to establish an ADM environment and to manage and allow use of ADMs created using such an environment. In some embodiments, clicking on a link opens an ADM template or, after data has been entered into at least one ADM template, allows the user to select or open an existing ADM. The term “link” is used here in a general sense to refer to any element that allows navigation. In some embodiments a link may be in the form of, or contained within, an icon, tab, or other GUI element. In some embodiments clicking the link bring the user directly to an ADM template or existing ADM. In some embodiments clicking the link brings the user to an ADM template or ADM via one or more steps. For example, the user may be prompted to make a further selection after clicking the link. The ADM template or ADM may be used, in various embodiments, in any one or more ways or for any one or more purposes described herein.

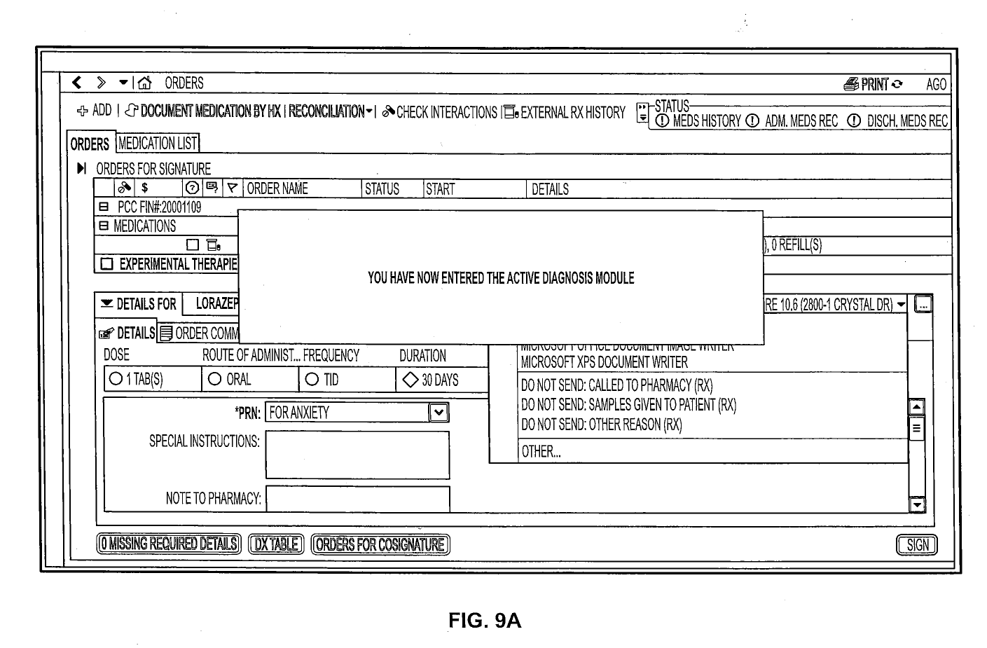

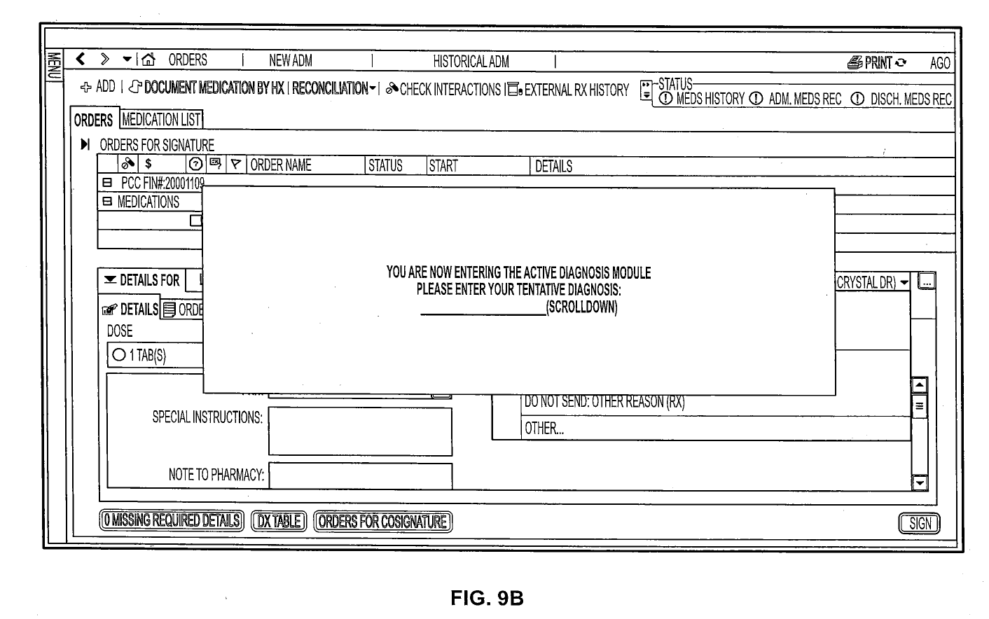

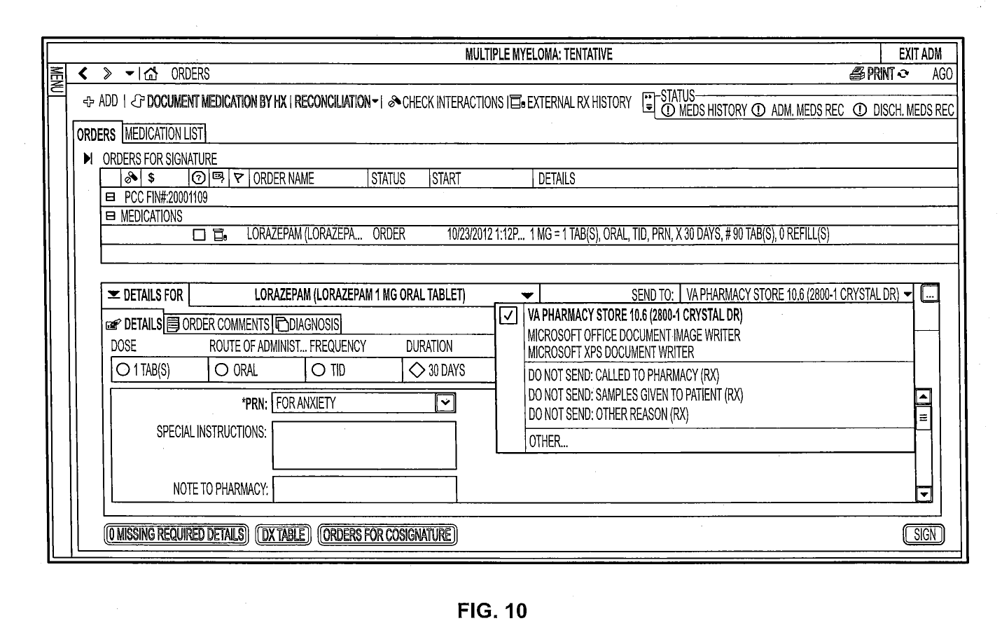

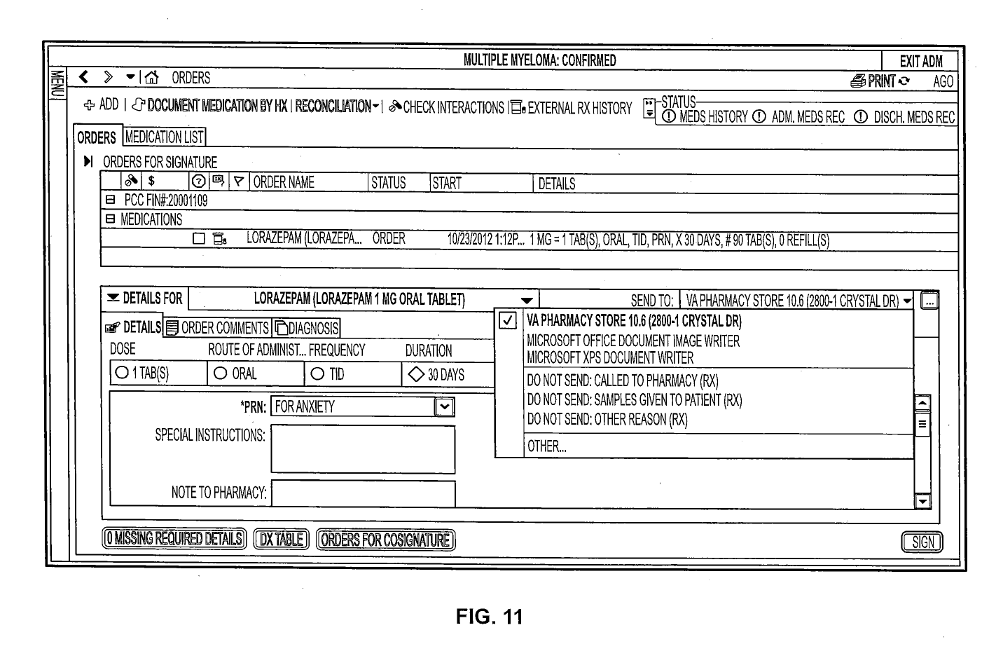

In some embodiments a prominent display element appears when the link is accessed, which indicates to the user that the ADM environment or an ADM template or ADM has been entered. The display element may remain visible as long as the user remains within an ADM environment, an ADM template or an ADM. In some embodiments, for example, a “ribbon” (e.g., a straight horizontal bar) appears at or near the top of the screen (or at or near another edge, e.g., a side or bottom) when the link is accessed, indicating that the Active Diagnosis Module environment or an ADM template or ADM has been entered. In some embodiments “near an edge of the screen” refers to a location within 10% of the total height or width of the display area. In some embodiments “near the top of the screen” refers to a location within 10% of the total height or width of the display area. The ribbon may have a distinct color as compared with the background color of the screen. In some embodiments the ribbon may be purple. FIGS. 9A and 9B shows screen shots illustrating exemplary appearance of a screen after experimental therapies functionality is accessed in accordance with certain embodiments. As shown, after such access, a ribbon at the top of the screen indicates to the user that they have entered the Active Diagnosis Module. A message is displayed stating, “You have now entered the Active Diagnosis Module”. In some embodiments a ribbon may appear at or near the bottom or a side of the screen instead of or in addition to at or near the top. Of course other display elements may be used instead of or in addition to a ribbon. In general such a display element may be readily visible yet not interfere with or intrude on the other contents displayed on the screen. In some embodiments the ADM environment may be entered as a new window on the screen. In some embodiments a message is displayed to indicate that the ADM environment has been entered. The message may state, e.g., “You have now entered the Active Diagnosis Module” or “You have now entered the Active Diagnosis Module Environment”. The user may proceed to use one or more ADM-associated functions. For example, the user may enter a tentative diagnosis and create an ADM for such diagnosis. The user may then enter additional data as specified by the ADM template. In some embodiments the ribbon (or other display element) may have a first color when a tentative diagnosis has been entered and a second (different) color when a diagnosis is confirmed as definitive. For example, a ribbon may be pink when a diagnosis is tentative and change to purple when confirmed. In some embodiments the ribbon may be labeled with the name of the diagnosis (e.g., “multiple myeloma”). In some embodiments the ribbon may be labeled with “tentative” or with “confirmed” or “definitive” depending on whether the diagnosis is tentative or has been confirmd. In some embodiments the ribbon may be labeled with both the name of the diagnosis and with “tentative” or with “confirmed” or “definitive” depending on whether the diagnosis is tentative or has been confirmed.

In some embodiments, by clicking on an appropriate area of the screen from within the ADM environment (e.g., from within an ADM) the ADM environment may be exited for a return to, e.g., the default EMR settings or the screen from which the ADM environment was entered. In some embodiments the ADM environment (or a screen within an ADM) may be exited by clicking the ribbon mentioned above. In some embodiments the ADM environment (or a screen within an ADM) may be exited by clicking an “X” in a corner, e.g., an upper corner (e.g., the upper right corner) of the screen. In some embodiments a link or icon labeled “exit ADM” or “return to EMR” or similar language is present within the ADM and can be used to return to a standard EMR screen.

FIG. 19A shows certain embodiments in which a patient's EMR contains links to New ADMs, Active ADMs, and Inactive ADMs. ADMs may be accessed or embedded into a physician/institution's EMR system via a plugin. FIGS. 19B-19F show that the ADM collects various data relevant to the tentative diagnosis. Such data may be obtained at least in part from the patient's EMR from which the ADM was created, from existing ADMs (which may be associated with the same EMR or with different EMRs that the patient may have at different HCOs). The ADM may structure the data and/or prompt the physician to complete the data relevant to the ADM (e.g., data that is not already present in the EMR) according to an interactive algorithm. Completion may occur over the course of multiple ADM access sessions. For example, the ADM may require input of information indicating whether the patient has atrophic lesions in the retina (FIG. 19D). Gathering such information may require performing one or more diagnostic tests, results of which may not be immediately available and may be entered during a subsequent session. It will be understood that the data listed in FIGS. 19B-19G are merely representative of data fields may be found in an ADM for Age-Related Diagnosis—Geographic Atrophy. For example, such an ADM may include fields for entering data for both right and left eyes, information regarding location of drusen, location and size of atrophic areas, presence or absence of evidence of neovascularization (which may suggest an additional diagnosis of neovascular macular degeneration). Once sufficient and appropriate data to meet diagnostic criteria for the tentative diagnosis are entered, the diagnosis changes to a confirmed diagnosis and the ADM becomes active (FIG. 19G). In some embodiments the final step prior to the status of the ADM switching to “active” is confirmation of the diagnosis by the HCP. In some embodiments such confirmation must be provided by a physician of the HCP in order for the ADM status to become “active”. In some embodiments upon activation of an ADM a notice inviting a user to contribute suggestions or comments, regarding the ADM is provided. The user may be invited to contribute suggestions or comments relating to design, format, content, or any aspect relating to quality of the ADM. Such suggestions or comments may be used in future revisions of the ADM template. In some instances such suggestions or comments may be of sufficient significance that the contributor thereof may become a co-author of the ADM.

In some embodiments an ADM component may provide some but not all functionality that may be associated with ADM templates or ADMs as described herein. For example, in some embodiments an ADM component may provide functionality that allows a user to view existing ADMs for a patient but not to modify such ADMs or create new ADMs. In some embodiments an ADM component may provide functionality that may allow a user to view and modify ADMs for a patient but not to create new ones. In some embodiments an ADM component may include functionality that allows an ADM template to import data from an existing standard EMR. Appropriate data fields of the ADM template may be populated with data from the existing standard EMR. In some embodiments data fields of an ADM are updated as data are entered into an existing standard EMR. In some embodiments updating occurs automatically. In some embodiments a user, e.g., a HCP, may initiate an update process. In some embodiments an ADM component comprises information that associates or maps one or more fields or sections of an ADM to one or more corresponding fields or sections of an EMR of a particular standard EMR system. The information may be held in any of a variety of different data structures, e.g., table, list, array, etc. Such corresponding fields or sections are intended to contain the same types of medically relevant data. For example, a medication list in an ADM would correspond to a medication list in a standard EMR. As another example, an ADM for a disorder involving the hematologic system may include fields for results of a diagnostic test, e.g., a complete blood count. Such fields would correspond to fields or section for such results in a standard EMR, if present. As another example, an ADM for a disorder involving the eyes may include fields for results of assessing visual acuity. Such fields would correspond to fields or section for such results in a standard EMR, if present. It will be understood that there may not be a one-to-one correspondence between ADM fields or sections and standard EMR fields or sections. For example, an ADM may have one or more fields that do not have a corresponding field or section in a standard EMR. If design of an ADM template or EMR format is altered, the ADM component may be altered to reflect an altered correspondence between fields or sections.

In some embodiments an ADM system, ADM template, or ADM component ensures that the data in an ADM and the EMR system(s) to which such ADM is linked is synchronized. For example, if a patient has an EMR created in a standard EMR system that has been equipped with ADM functionality, data entered into such EMR that is relevant to a particular ADM is promptly copied by the system into that ADM. Fields of the ADM may ordinarily be updated on an essentially continuous or very frequent basis as data are entered elsewhere in the EMR, e.g., during or after a patient visit. Similarly, if a patient has an EMR created in a standard EMR system that has been equipped with ADM functionality, data entered directly into an ADM, e.g., by a HCP, is promptly copied by the system into the appropriate section or field of the EMR, if any. The EMR may ordinarily be updated on an essentially continuous or very frequent basis as data are entered into an ADM, e.g., during or after a patient visit. In some embodiments an average time lag between entry of data into an EMR and its appearance in an ADM, or vice versa, is no more than 1, 2, 5, 10, 15, 20, 30, 40, 45, 50, or 60 seconds. In some embodiments an average time lag between entry of data into an EMR and its appearance in an ADM, or vice versa, is no more than 1, 2, 5, 10, 15, 20, 30, 40, 45, 50, or 60 minutes.

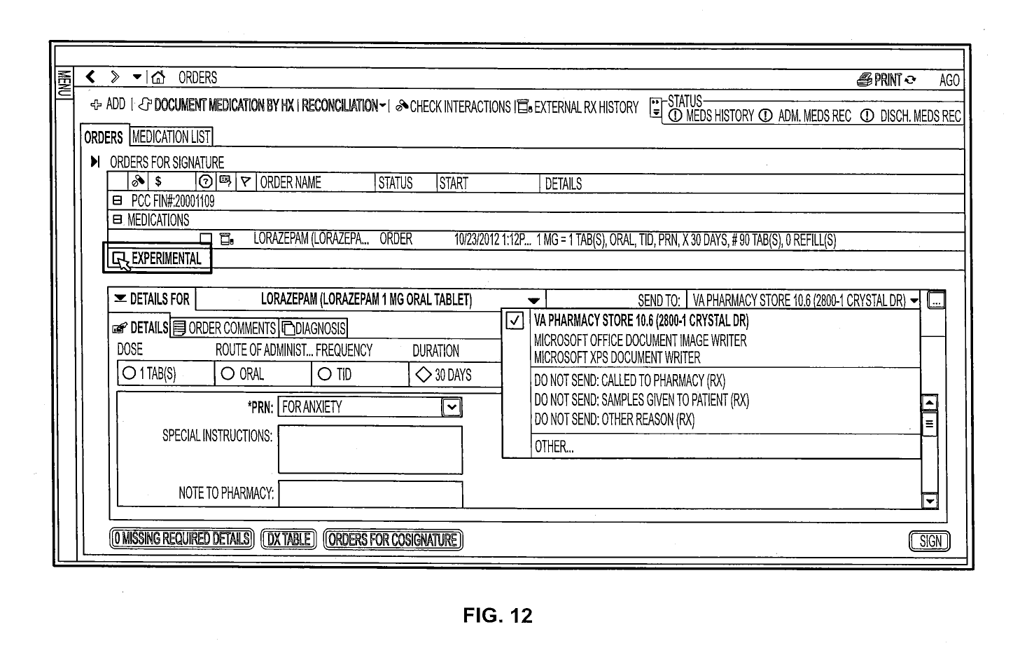

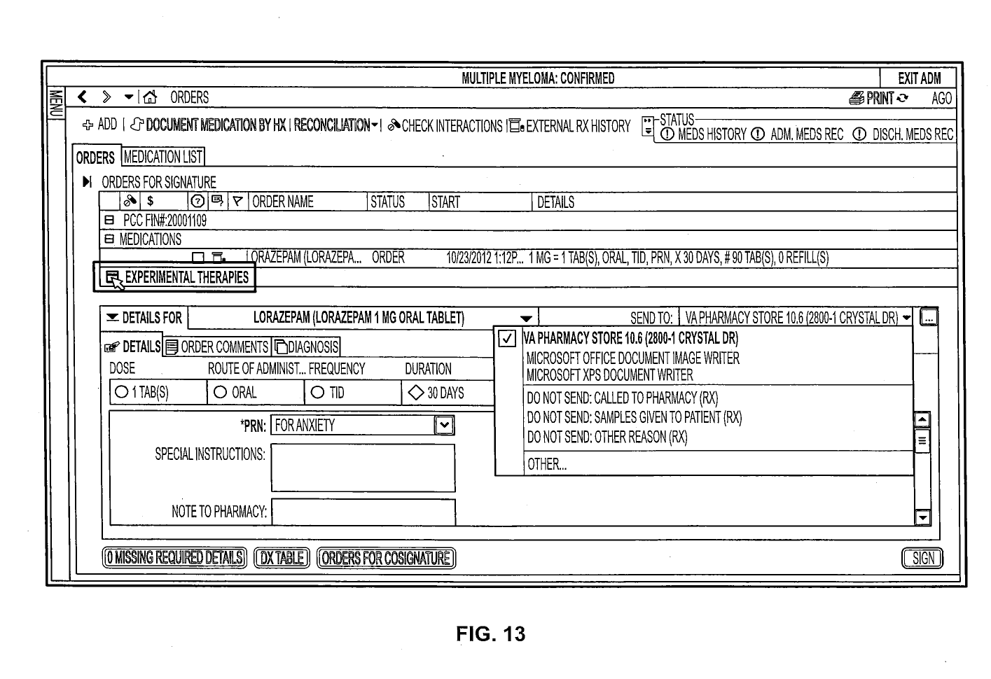

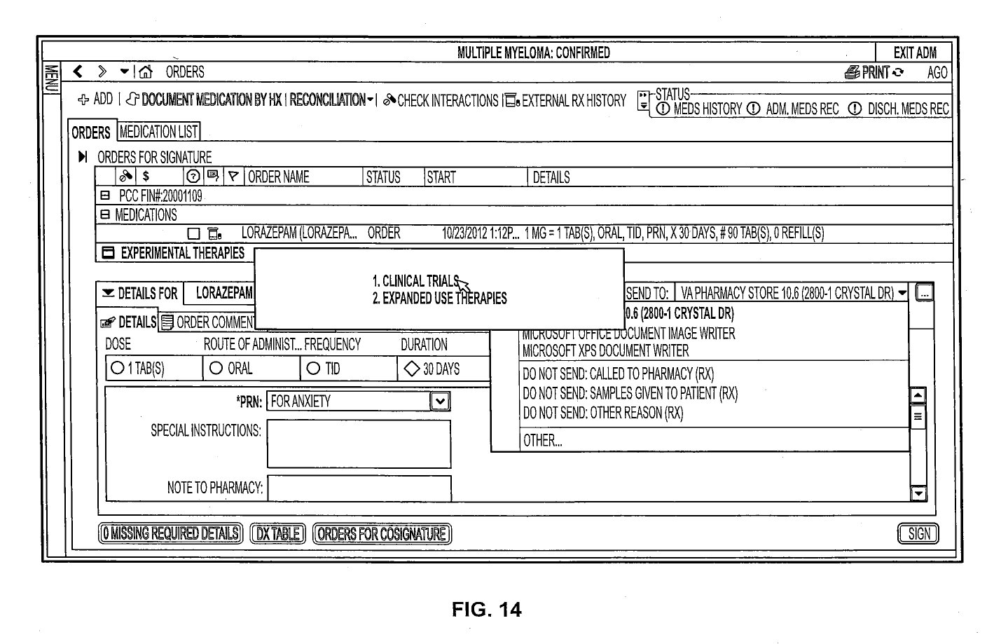

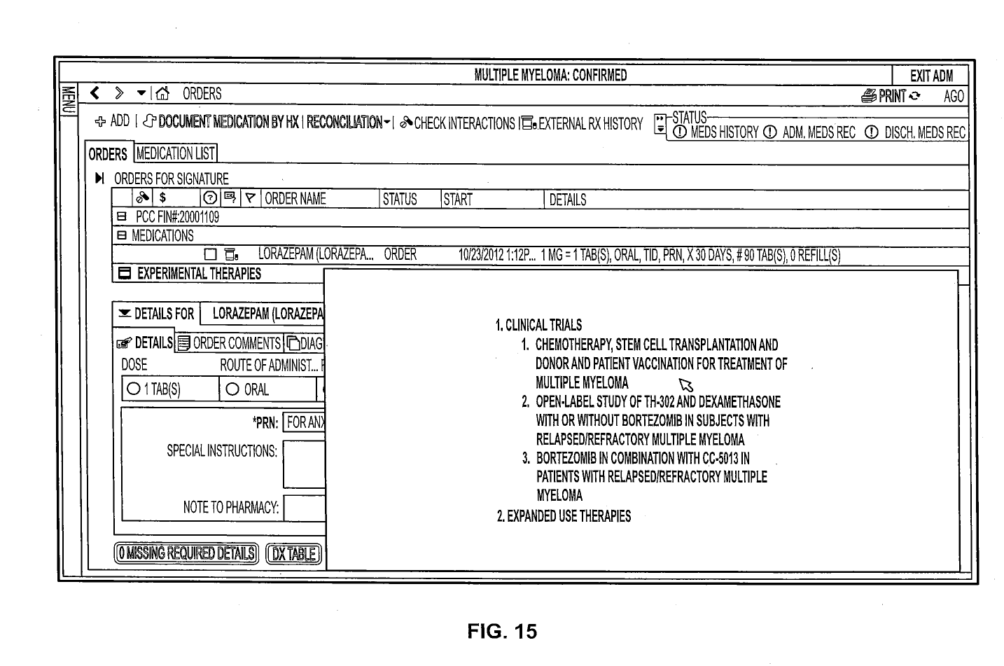

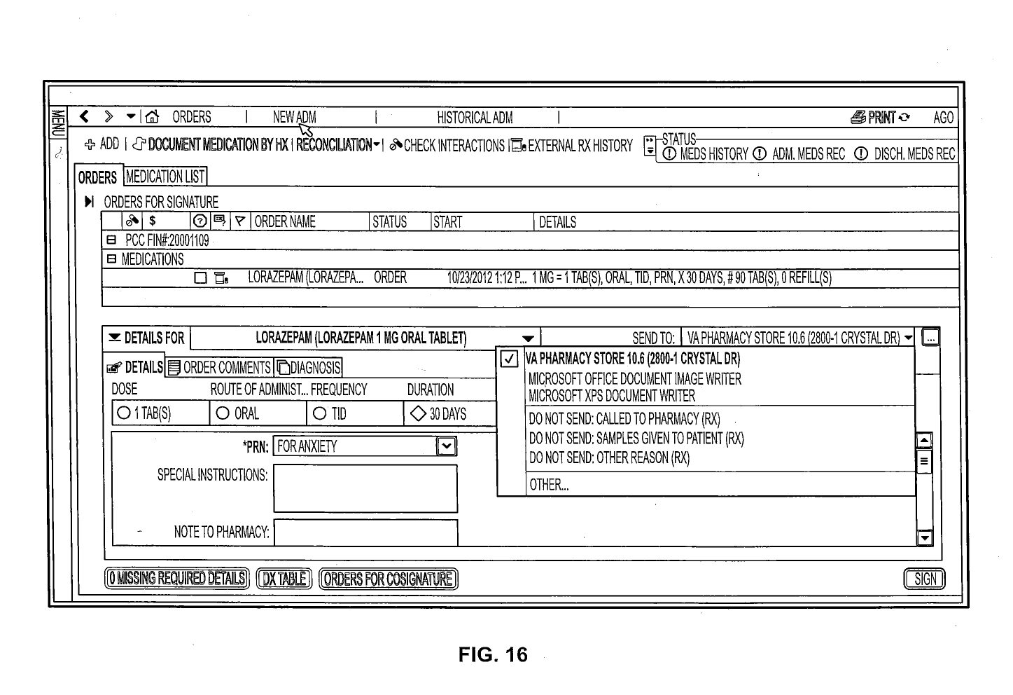

In some embodiments an ADM component may provide one or more functions that facilitate or make possible the use of ADMs for clinical trial purposes in the context of a standard EMR system. Use of ADMs and ADM-equipped EMRs for clinical trial purposes is also described further below. In some embodiments a component, e.g., an ADM component comprises, installs, or offers an option to install an “Experimental Therapies” component. An Experimental Therapies component may comprise computer-executable instructions to carry out one or more functions associated with experimental therapies. Such computer-executable instructions and/or functions may be provided in the form of a distinct module or component. In some embodiments an ADM component or Experimental Therapies component installs or offers an option to install an “Experimental Therapies” link into at least some EMRs of an EMR system. In some embodiments the “Experimental Therapies” link provides an entry point from a standard EMR into an environment that allows use of ADM templates and/or ADMs for clinical trial screening, electronic data capture, trial management, monitoring, and/or data analysis. A link may be in the form of an icon, menu options, tab, and/or any other element typically used for navigation in a graphical user interface, e.g., between screens or web pages. In some embodiments access to at least some “Experimental Therapies” functionality may be provided only from within the ADM environment. In some embodiments a link to Experimental Therapies may become available or accessible when a tentative diagnosis is confirmed. FIGS. 12 and 13 show screen shots illustrating exemplary means for accessing experimental therapies functions in an EMR system in accordance with certain embodiments. As shown in the figures, within an EMR, in the medication section, a link may be available to “Experimental Therapies”. When selected, the link may bring up a list of experimental therapies and/or clinical trials or expanded use therapies (which may be available under managed access programs) that may be available for a patient with the diagnosis that has been confirmed. In some embodiments a link to Experimental Therapies for a particular disease appears only in an ADM for that disease. In some embodiments a link to Experimental Therapies appears in an ADM for that disease and in an EMR of a patient that has or may have the disease. In some embodiments a link to Experimental Therapies appears in an EMR of a patient who may have a disease. A HCP may click on the Experimental Therapies link and may then be prompted to create an ADM for the disease.

In some aspects an ADM component suitable for use in connection with a standard EMR system may be generated with permission of or through collaboration with an entity that owns or controls or otherwise has a proprietary interest in the standard EMR system software, e.g., an entity that at least in part owns, controls, makes, sells, or provides the standard EMR system software or is authorized by such entity to disclose or modify such portion(s) of the standard EMR system, if any, that would facilitate proper interfacing or integration of the ADM component. In some aspects, ADMs may provide a common format that permits data sharing between diverse standard EMR systems, e.g., standard EMR systems that have been extended via installation of an ADM component.

In some aspects, the use of an ADM component to extend the functionality of a standard EMR system may facilitate transition to an EMR system that uses ADMs as its primary means of recordkeeping. In some aspects, the use of an ADM component to extend the functionality of a standard EMR system may leverage existing familiarity with such a standard EMR system that may already have been acquired by HCPs or other users. In some aspects, the use of an ADM component may provide means by which a standard EMR system may be used for clinical trial purposes as well as for ordinary patient care purposes. In some aspects, the use of an ADM component may provide means by which diverse standard EMR systems may be utilized for clinical trial purposes. In some aspects, the use of an ADM component may allow HCOs that have already invested in a standard EMR system to continue using such system while gaining access to additional functionality that may, for example, improve EMR quality, facilitate enrollment of patients in clinical trials, facilitate clinical trial data collection, facilitate interaction and/or data exchange with other EMR systems (e.g., standard EMR systems made by different vendors), etc.

In some embodiments the ability to paste information into an ADM or ADM template may be restricted or may be unavailable under at least some conditions. For example, it may be impossible to copy information that was entered during a previous patient visit (either into an ADM or elsewhere in an EMR) and paste such information into an ADM. In some embodiments at least some such copy and paste functionality may be enabled or disabled as appropriate for the circumstances or for the particular individual accessing the ADM. In some embodiments information entered via pasting may be tagged to indicate that it was entered by pasting.

In some embodiments, ADM templates, whether generic, discipline-specialized, disease-specialized, etc., in many embodiments may at least in part share a common predetermined format. As noted above, ADMs may at least have fields for conventional and (if applicable) molecular disease diagnosis, diagnostics, and treatments. They may in some embodiments differ at least in part with regard to fields for specific symptoms, signs, complications, etc. In some embodiments, different ADM templates may be designed so as to use the same design elements such as fonts, page layout, spacing, color, GUI elements, across different templates, etc., so as to provide a uniform user experience. ADM templates may include a logo (e.g., a graphic mark or emblem, which may be purely graphic (symbols/icons) or, e.g., composed at least in part of the name of the business entity or one or more suitable words, letters, and/or numbers) to promote rapid recognizability, e.g., across different computer systems or in the context of different EMR systems or as stand-alone elements. In some embodiments, by way of example, an ADM template may include the term “ADM”, e.g., as an unregistered or registered trademark (ADM™ or ADM®). Such term may be further specialized, e.g., by discipline or disease, such as ADM-Oncology, ADM-Neurology, etc. In some embodiments an EMR system and/or ADM template comprises one or more features adapted for use in outpatient care. In some embodiments an EMR system and/or ADM template comprises one or more features adapted for use in inpatient care. For example, one or more fields for tracking symptoms, signs, or performing tests or procedures or monitoring actions that may be performed in an outpatient or inpatient context, respectively, may be provided. In some aspects, an ADM template may include an option to toggle back and forth between outpatient and inpatient versions. In some embodiments an ADM, which may be a discipline-specific or disease-specific ADM, may be denoted as ADM-Inpatient or ADM-Outpatient.

In some embodiments an ADM may be characterized in that it comprises at least a tentative diagnosis and a definitive diagnosis. In some embodiments an ADM may be characterized in that it comprises a diagnosis status, wherein said diagnosis status may be tentative or definitive, and wherein said status may, e.g., be indicated to a user via a field, color or other indication means. In some embodiments both a tentative diagnosis and a and definitive diagnosis may be retained. In some embodiments a tentative diagnosis may, e.g., at the selection of a contributor that submitted it, be deleted or made unavailable for access (e.g., by at least some users of the EMR database) after a definitive diagnosis has been established. In some embodiments a field for entering a conventional diagnosis is provided, wherein an entered diagnosis is presumed to be tentative unless a contributor, e.g., an HCP, indicates otherwise. In some embodiments, a field is provided, wherein an option of tentative or proposed definitive may be selected for an entered diagnosis. In some embodiments an ADM is characterized in that it comprises a definitive diagnosis that has been confirmed by determining, based at least in part on entered results (e.g., results of at least one diagnostic test) that a predetermined set of diagnostic criteria have been met. In some embodiments, an indication is provided to a contributor, e.g., a HCP, when a predetermined set of criteria for confirming a tentative diagnosis as definitive have been met. A “set”, wherever such term appears herein, may contain a single member or multiple members in various embodiments. For example, a set of predetermined criteria may be one criterion or may comprise multiple criteria. In some embodiments an ADM template may be characterized in that it comprises a field for a tentative diagnosis and a field for a confirmed diagnosis and/or a field for indicating whether an entered diagnosis is tentative or proposed definitive, and/or a field for indicating that a diagnosis is definitive. In some embodiments an ADM is characterized in that it comprises a conventional diagnosis and a molecular diagnosis. In some embodiments an ADM template is characterized in that it comprises a field for a conventional diagnosis and a field for a molecular diagnosis.

In some embodiments an ADM template may have associated with it a set of predetermined options for selection of diagnostics or therapeutics, wherein the set of predetermined options may be based at least in part on conventional and/or molecular diagnostics. In some embodiments an ADM template may have associated with it a set of rules, a knowledge base and/or an inference engine, which may be at least a portion of an expert system. In some embodiments said rules, knowledge base, inference engine, and/or expert system may be at least in part embodied within an ADM template. In some embodiments said rules, knowledge base, inference engine, and/or expert system may be an EMR system component. In some embodiments said rules, knowledge base, inference engine, and/or expert system may be at least in part invoked in response to data entered into an ADM template. In some embodiments said rules, knowledge base, inference engine, and/or expert system provide a determination or recommendation based at least in part on data entered into an ADM template. For example, a determination may be a confirmation that a proposed definitive diagnosis meets a set of predetermined criteria.

In some embodiments an ADM template or interface to an ADM template comprises an expert system that ensures that (1) the ADM is populated with quality data and (2) the diagnosis is confirmed. In some embodiments ADMs and ADM templates are at least in part cloud-based and compatible with multiple different EMR systems. In some embodiments ADMs can be monitored and/or data-mined from one or more central locations, which may be remote from one or more sites at which data are entered. In some embodiments monitoring and/or data mining may occur in real-time. In some embodiments such data mining and/or monitoring may be performed in situations in which the patient is enrolled in a clinical trial or managed access program. In some embodiments such data mining and/or monitoring may be performed in situations in which the patient is not enrolled in a clinical trial or managed access program. In some embodiments sponsors and/or payors or other entities (e.g., providers of health-related products or services) may communicate to or with patients via their ADMs. Communication may be one-way in either direction (entity to patient or patient to entity) or two-way (entity can communicate to patient and patient can communicate to entity) in various embodiments. In some embodiments patients are provided an opportunity to choose to receive various types of communication or communication from various entities or types of entities. In some embodiments patients are provided an opportunity to choose not to receive various types of communication or communications from various entities or types of entities. In some embodiments patients may be able to alter their preferences with regard to receiving communications. In some embodiments communication is anonymous in that the entity does not have access to the identity of a patient with whom it communicates. An entity may have access to all or part of the information contained in a de-identified ADM. For example, the entity may have access only to the diagnosis or may have access to at least some demographic information (e.g., age, gender). In some embodiments the entity may have access to additional data such as e.g., medications, other ADM diagnoses.

In some embodiments, all or at least 90%, 95%, or 99% of the ADMs in a EMR or EMR database may be created at or shortly after the time at which the patient corresponding to the particular ADM initially seeks care for the relevant disease or the time at which symptom(s) or sign(s) of the disease first come to the attention of a patient's HCP. Such ADMs may be referred to as “type 1” ADMs. For example, an ADM may be created during the first patient visit during which symptoms or signs of the disease are discussed or detected or within the time period in which an HCP would ordinarily record information pertaining to such symptom(s) or sign(s) in a patient's medical record. If properly completed and updated, such an ADM may provide substantial information pertaining to the disease in that patient starting from the time that the disease first came to medical attention.

In some embodiments an ADM may pertain to a disease for which a diagnosis has already been established or for which the patient has already received at least one therapeutic intervention at the time the ADM is created. Such ADMs may be referred to as “type 2” ADMs. The comprehensiveness of a type 2 ADM in terms of the extent to which the type 2 ADM includes information gathered prior to the ADM's creation may vary. For example, a type 2 ADM may include a diagnosis and current treatment as of the creation date of the ADM but may not include information pertaining to previously performed diagnostics and/or previous treatments. Alternately, at least some information pertaining to previously performed diagnostics and/or previous treatments may be included in a type 2 ADM. Such information may, for example, be collected from existing health records (paper or electronic) and/or from the Patient Data section of the EMR. In some embodiments a type 2 ADM may contain less comprehensive health information pertaining to the disease than would ordinarily be the case for a type I ADM. For example, a HCP may not have access to health records held by previous HCPs of the patient, or it may be too burdensome to enter data that may not be directly relevant to the patient's current condition.

In some embodiments an ADM may be tagged with metadata indicating whether it is a type 1 or type 2 ADM. In some embodiments, type 1 ADMs may be preferred for certain purposes, e.g., for certain research or analysis purposes.

In some embodiments, an ADM may contain health information pertaining to a disease in a patient over a period of at least 3, 6, 9, or 12 months. In some embodiments, an ADM may contain health information pertaining to a disease in a patient over a period of at least 1, 2, 3, 4, or 5 years. In some embodiments, an ADM may contain health information pertaining to a disease in a patient over at least 3, 5, 10, or more patient visits, which visits may be separated in time by, e.g., intervals of at least 1 week or more, on average. In some embodiments, an ADM contains health information pertaining to a disease in a patient over a period of at least 1, 2, 3, 4, or 5 years. In some embodiments, a diagnosis, e.g., a tentative diagnosis, proposed definitive conventional diagnosis, and/or molecular diagnosis is entered by a HCP, e.g., a physician, during or after an outpatient visit by the patient. In some embodiments, a diagnosis, e.g., a tentative diagnosis, proposed definitive conventional diagnosis, and/or molecular diagnosis is entered by a HCP, e.g., a physician, during or after a visit by the HCP to a hospitalized patient.

In some embodiments, an EMR or ADM may comprise a field for entering information pertaining to patient satisfaction, e.g., patient satisfaction with care received from a HCP or HCO. In some embodiments entering information in such fields may be limited to a patient or patient representative(s). In some embodiments entering information in such fields may require verification by a patient or patient representative(s). In some embodiments patient satisfaction is determined based at least in part on a questionnaire or survey. In some embodiments, information pertaining to patient satisfaction may be at least in part accessible to at least some users or categories of users.

In at least some embodiments, an ADM does not contain information included solely for billing, reimbursement, or insurance claim purposes.

In some aspects, ADMs separate certain core functions of the practice of medicine, namely diagnosis and treatment of disease, from ancillary activities such as billing, hospital/clinic management, logistical support, resource management, interfacing with internal and external clinical/pathology/testing labs and services, scheduling, etc. HCPs, e.g., physicians may see patients in a variety of different health care organization settings, e.g., hospital, clinic, private practice and/or may work at multiple different HCOs over the course of their career. Different HCOs may perform at least some of the ancillary activities in different ways, which may, for example, be selected based at least in part on factors such as patient population, organizational capacities, preferences, or for historical reasons. HCOs may select an EMR system based at least in part on its usefulness or convenience for performing ancillary activities, for which different EMR systems may be more or less well suited than others, or based at least in part on cost, vendor support, or a host of other factors. In certain embodiments, although different HCOs may use different standard EMR systems, ADM components for use with such systems provide ADM templates with the same structure (e.g., same data fields, same menu options). Thus an ADM created in any such EMR system will contain the same data and be usable in the same way, regardless of the particular EMR system with which it interfaces. Without wishing to be bound by any theory, HCPs familiar with the ADM structure may find it easier to rapidly assimilate the details of a patient's disease(s) than would be the case if using a non ADM-equipped EMR system. Thus a person (e.g., an HCP) who has experience in using an ADM-equipped EMR system will already be familiar with the ADM structure when using a different ADM-equipped EMR system. In some embodiments stylistic features of an ADM (e.g., appearance, layout of the data screens, fonts, colors, choice of GUI elements, etc.) are the same across different EMR systems. In some embodiments stylistic features of an ADM (e.g., appearance, layout of the data screens, colors, choice of GUI elements, etc.) may differ at least in part across different EMR systems. For example, at least some such features may be selected to match those used by an EMR system with which they interface. In some aspects separating core functions from ancillary activities.

In some aspects, use of ADMs helps address the issue that important disease-relevant data may not be available in the EMR system of any single HCO and may not all be available to all of a patient's HCPs. Without wishing to be bound by any theory, in certain embodiments ADMs provide a way to address this limitation while allowing HCOs to avoid the cost and disruption associated with a change of EMR system or EMR provider or requiring full interoperability between different EMR systems. In some embodiments an HCO may select or continue to use an EMR system based at least in part on considerations other than its core medical record functions while using ADMs, e.g., via an ADM component, to provide and/or assure the quality of such medical record keeping functions. ADMs may allow a HCP to rapidly review and understand the time course and current status of a particular disease and the knowledge that at least certain predetermined diagnostic criteria for that disease have been met, regardless of where the data relevant to the disease originated. When a primary care physician (PCP) sees a patient who has visited a specialist for a particular disease, the PCP may, by viewing the patient's ADM for that disease, readily be able to determine if or the specialist performed tests (and review results of such tests) or changed the management of the disease. When a specialist for a particular disease sees a patient who may be under the care of one or more other HCPs, the specialist is, by viewing the ADMs for diseases within his or her specialty, able to focus on such disease(s).

As discussed herein, in some aspects, ADMs provide the capacity to differentiate between a diagnosis that may, for example, have been selected at least in part for billing and/or reimbursement purposes and a definitive diagnosis that has been arrived at after specified diagnostic criteria are met. In some aspects, this distinction enhances the usefulness of ADMs and ADM-equipped EMRs as sources of medical information, which may be used retrospectively or prospectively, e.g., for data-mining, trials, expanded access programs, repositioning, or other purposes. In some aspects, the distinction between tentative vs definitive diagnosis provided by ADMs enhances the usefulness of ADMs and ADM-equipped EMRs as sources of medical information, which may be used retrospectively or prospectively, e.g., for data-mining, trials, expanded access programs, repositioning, or other purposes.

It is noted that aspects or features may comprise creating a database comprising modules containing health information, e.g., active diagnosis modules, wherein such modules may have an at least partially predetermined format, may be searchable on e.g., disease diagnosis, diagnostics, and treatment, and may further be searchable on key symptoms, signs, complications, and/or outcomes, are independent of any particular implementation of the system or components thereof. In some embodiments: (a) a database may comprise at least 5,000; 10,000; 50,000; 100,000; 500,000; 1,000,000; 5,000,000; 10,000,000; 50,000,000; 100,000,000 or more such modules; (b) the health information may be de-identified; (c) the health information may be contributed at least in part by HCPs of the patient to whom it pertains; and/or (d) the database may be made available to subscribers, and may further be available for a fee. In some embodiments such modules may have any one or more features of ADMs, as described herein. Further, such modules containing health information may be created at least in part through providing templates and/or incentives to HCPs. Further, such modules may be made available to third parties, the use of such modules by third parties may be tracked, and, e.g., incentives may be provided to HCPs based at least in part on use of such modules by subscribers. It is also noted that in some embodiments that may include generating or providing (e.g., on a computer-readable medium and/or by transmission over a network such as the Internet) a collection that may include templates that may have an at least partially predetermined format, that may have fields for entering e.g., disease diagnosis, diagnostics, and treatment, and that further may have, e.g., fields for entering key symptoms, signs, complications, and/or outcomes, such embodiments, collection(s) and/or template(s) are independent of any particular implementation or use thereof. In some embodiments, a collection may comprise at least 2, 5, 10, or more such templates, e.g., at least some of which may be disease-specialized or discipline-specialized. In some embodiments, such templates may comprise means for providing feedback to a user who enters data into the fields; such feedback may in various embodiments include offering suggestions for completing the template and/or for disease diagnosis or management.

In some embodiments, at least some information may be time and date stamped upon receipt by the EMR system and/or upon addition to a EMR. For example, any additions or changes to a EMR may be time and date stamped and/or may include information identifying the contributor.

A EMR may contain health information relating to preventive health care (e.g., screening tests such as colonoscopies or cholesterol measurements; vaccinations, etc.), vital signs, or other data that may not necessarily be associated with a particular ADM. Health information pertaining to preventive care may be included in one or more preventive health care modules (PHCM). At least some such information may also or may instead be included in an ADM if appropriate. For example, a routine blood pressure check may be included in a PHCM, but if the patient has a diagnosis of hypertension or is discovered to have hypertension, the blood pressure may also or may instead be included in the ADM for hypertension. In some embodiments, an ADM may be an “active disease risk module” (ADRM), wherein the patient has not actually developed a disease but has one or more identified risk factors indicative of an increased risk of developing the disease and potentially warranting therapeutic intervention or more intensive monitoring than would otherwise be the case. For example, the patient could have a family history of the disease or a genotype or phenotypic characteristic associated with increased risk of developing the disease. The ADRM may, for example, include at least some of the same elements as an ADM pertaining to a disease. Numerous risk factors for diseases are well known to those of ordinary skill in the art. In some embodiments a risk factor may be associated with at least a 5%, 10%, 20%, 30%, 40%, 50%, 75%, 90%, or 100% risk of developing the disease, e.g., within a defined time period, such as 1-12 months, 1-2 years, 2-5 years, 5-10 years, or within the lifetime of the patient. In some embodiments a risk factor may be associated with at least a 5%, 10%, 20%, 30%, 40%, 50%, 75%, 90%, or 100% increase in risk of developing a disease, e.g., within a defined time period, such as 1-12 months, 1-2 years, 2-5 years, 5-10 years, or within the expected lifetime of the patient, as compared with the risk that a person would have in the absence of such risk factor. In some embodiments a risk factor may be associated with at least a 2-fold, 3-fold, 5-fold, 10-fold, or 20-fold increase in risk of developing a disease, e.g., within a defined time period, such as 1-12 months, 1-2 years, 2-5 years, 5-10 years, or within the expected lifetime of the patient, as compared with the risk that a person would have in the absence of such risk factor In some embodiments a risk factor may justify therapeutic intervention, e.g., in order to decrease the likelihood that a patient will develop a disease with which the risk factor is associated. For example, increased cholesterol may justify treatment with a cholesterol lowering agent. In some embodiments a risk factor may justify more intensive monitoring than would otherwise be the case, e.g., more frequent or extensive physical examinations or more frequent or extensive diagnostic testing.

A EMR may contain genetic information regarding the patient, which may include, for example, results of tests for the presence or absence of particular genetic variations, such as single nucleotide polymorphisms (SNPs), or, in some embodiments, partial or complete genome sequences. In some embodiments, genetic information may include the patient's genotype with regard to at least some polymorphisms or mutations that are associated with (correlated with) increased risk of developing a disease or condition or that are associated with e.g., outcome, severity, treatment response, or drug metabolism variation (e.g., polymorphisms in genes encoding cytochrome P-450 enzymes that are associated with increased or decreased metabolism of various drugs). In some embodiments, at least some such genetic information may be included in a relevant ADM or ADRM. For example, if the patient has a disease that is associated with particular haplotype(s), polymorphism(s), or mutation(s), the patient's genotype with respect to at least some such haplotype(s), polymorphism(s), or mutation(s) may be included in the ADM for that disease. In some embodiments, if the patient has particular haplotype(s), polymorphism(s), or mutation(s) that are associated with increased risk of a disease, the patient's genotype with respect to at least such haplotype(s), polymorphism(s), or mutation(s) may be included in an ADRM for that disease.

In some embodiments, the EMR interface may depict ADMs as icons. Selecting an icon for a particular ADM (e.g., by clicking on it) may take the user to a document that displays information pertaining to that ADM. The icons may be labeled with a tentative or definitive diagnosis. In some embodiments, different colors, shapes, and/or sizes may be used to mark the status of an ADM as tentative or definitive. For example, a tentative ADM may be pink. According to the example, when the diagnosis is deemed definitive, the color may be changed, e.g., to green. In some embodiments an HCP may designate an ADM as “resolved” or “recurrent” if appropriate for the particular disease or condition. Additional colors or symbols may be used to distinguish such ADMs and/or to distinguish ADMs for which the definitive diagnosis may be deemed invalid according to revised diagnostic criteria.

In some embodiments the status of a resolved ADM may be changed to “inactive”. In some embodiments the status of an ADM may be changed to “inactive” if the ADM has not been updated by addition of adequate information (e.g., as determined by the system) for a specified time period. For example, if no new information has been entered during the preceding 6, 12, or 24 months, the status of an ADM may be changed to inactive. The time period, amount and/or nature of information that needs to be entered in order for an ADM to retain “active” status may vary, e.g., depending on any of a variety of factors. For example, treatment of certain diseases may typically involve less or more frequent patient visits or monitoring than other diseases. In the case of ADMs for diseases that are typically monitored with patient visits at relatively long intervals, the ADM may remain active for longer than would an ADM for a disease that is typically monitored more frequently. In some embodiments ADMs that are inactive because they are resolved may be distinguished from ADMs that have become inactive for failure to be updated appropriately (which may be referred to as “lapsed” ADMs. In some embodiments a system sends one or more messages to lapsed ADMs or the patients to which they belong or such patients' HCPs in order to attempt to determine why the ADM has not been updated or marked as resolved.

In some embodiments a user who queries an ADM database (e.g., to extract or analyze data relating to a particular disease, therapy, etc.) may set a filter to ensure that only data from ADMs that are active and/or that have been updated within a specified time window or with a specified frequency and/or that are resolved are used. In some embodiments a user may define the filter, e.g., by selecting a time (e.g., 6 months) and/or frequency (e.g., at least once every 3 months on average).

In some embodiments a EMR or ADM may contain a patient summary. The patient summary may include, for example, (a) demographic information; (b) physiologically important measurements such as height, weight, blood pressure; (c) list of conventional disease diagnoses for current (unresolved) ADMs; (d) list of current therapeutics; (e) any hospitalizations within the preceding 6 months, etc. The patient summary may provide a user, e.g., a HCP or subscriber with a rapid overview of many or most significant aspects of a patient's current condition and may provide additional context that may be important for understanding or using an ADM. In some embodiments, a patient summary may be automatically generated by the EMR system and may be updated as new health information is entered.

In some embodiments, the EMR system may comprise a component that supports computerized physician order entry (CPOE). It is envisioned that in some embodiments a HCP may order a test or prescribe a treatment (e.g., a medication) from within an ADM (e.g., while viewing an ADM). For example, when an ADM element is displayed, the screen may include a menu option that permits the HCP to order a test or prescribe a treatment. In some embodiments, a HCP may order a test or prescribe a treatment from within a non-ADM element of a particular EMR and may be offered an option to designate one or more ADMs at the time of ordering the test or prescribing the treatment. In each case, the test name, test result, and treatment may be automatically become part of the appropriate ADM once entered. A prescription may be automatically transmitted to a pharmacy. “Pharmacy” as used herein may include e.g., traditional pharmacies, online pharmacies, and other medication suppliers able to fulfill a prescription. It is envisioned that in some embodiments, pharmacists or other pharmacy workers may access the EMR system and/or the EMR system may interact directly with existing pharmacy computer systems.

Any one or more of the EMR or ADM elements or portions thereof may, in some embodiments, be selected by a HCP from a predetermined set of options. For example, as noted above, entry of conventional disease diagnosis may be facilitated by providing an appropriate GUI element such as a scroll-down list, which may be organized at least in part based on discipline (e.g., physician specialty) or at least in part based on the ICD classification scheme. In some embodiments, entry of molecular disease diagnosis may be facilitated by providing a scroll-down list (or other suitable GUI element) which may include items determined based on the conventional disease diagnosis. For example, if the conventional disease diagnosis is non-small cell lung cancer, molecular disease diagnosis may include the epidermal growth factor receptor (EGFR) mutation status of the tumor (e.g., whether the tumor harbors an EGFR mutation and, if so, the identity thereof). It will be appreciated that a scroll-down list is but one of a variety of suitable formats by which a set of options may be presented to an HCP. In some embodiments, diagnoses are presented in a hierarchical manner. For example, a high level diagnosis might be “lung cancer”. According to this example, if the HCP selects this diagnosis, a set of more specific diagnoses within the general category of “lung cancer” may be made available for selection.

It is envisioned that the use of predetermined and standardized terms, options, and/pr formats may enable different HCPs who may use the EMR for a particular patient to more clearly understand the patient's health care history. It is also envisioned that the use of predetermined and standardized terms, options, and/or formats may facilitate a variety of other uses of the EMR database such as, for example, (i) searching for, analyzing, and/or extracting relevant health information from the database for any of a wide variety of research purposes; or (ii) identifying HCPs or HCOs experienced in caring for patients that have a particular disease or that have particular disease characteristics (e.g., unusual symptoms or signs). In many embodiments, the ADM will be searchable at least based on conventional disease diagnosis, molecular diagnosis, diagnostics, and treatments. For example, a user could extract or analyze all ADMs for patients diagnosed with NSCLC who were treated with a particular combination of chemotherapeutic agents.

In some embodiments, entry of test results (e.g., lab test results, images, etc.) may be performed at the site where such results are obtained, e.g., by appropriately authorized individuals. In some embodiments such results may not become part of the EMR until the contributor who ordered the test acknowledges having reviewed them. In some embodiments, individuals operating under the direction of a contributor or responsible for entering test results may be assigned an ID that allows them to perform a selected set of tasks relating to entering data but may have limited or no ability to view or modify previously entered data.

In some embodiments, when a contributor logs on to the EMR system, the contributor may be informed of any new information that has been entered for his or her patients, e.g., results of tests ordered by the contributor or entered at the contributor's direction, and that have not yet been reviewed by the contributor. The contributor may then review the information and may be offered an opportunity to acknowledge having done so, e.g., by checking a box. The information may then become part of the EMR and the relevant ADM(s). (If the information had been entered, e.g., by or under direction of a different HCP of the patient, the information may already be part of the EMR and relevant ADM.) In some embodiments the contributor may assign the information to an ADM. In some embodiments the information may be automatically assigned to the appropriate ADM and further may be subject to review and acknowledgement by a contributor.

It is envisioned that in some embodiments a contributor may order a test from within an ADM. For example, an ADM screen may include an option that permits the contributor to order a test. In some embodiments, a contributor may order a test from the main screen for a particular EMR and may be offered an option to designate one or more ADMs at the time of ordering the test. In both cases, the test result may automatically become part of the appropriate ADM once entered (subject to review and acknowledgement by the contributor). In some embodiments, screening tests may become part of the PHCM.

In some embodiments a contributor may receive or may elect to receive an alert (e.g., via email, text message, voicemail, fax, etc.) when information about that contributor's patients (or about specific patient(s) of the contributor) is entered. The system may thus facilitate timely conveyance of important health information to health care providers. Alerts may be prioritized by importance.

The EMR system may provide a contributor with reminders or suggestions to order particular diagnostic tests, fill or refill prescriptions, discontinue or consider discontinuing medications when no longer required or appropriate, etc.

In some embodiments a EMR or ADM may comprise one or more types of data that may facilitate medically relevant research but that may not be directly relevant to the care of the patient. Such data may include, for example, information regarding availability for use in research studies of biological samples obtained from the patient, answers to health-related questionnaires or surveys to which the patient has responded, etc.

In some embodiments, the EMR system may provide computer-based tools (which may be embodied in hardware, software, or a combination thereof) that permit HCPs to perform analyses of their patient population. For example, a tool may permit an HCP to retrieve the identity or number of patients in his or her patient population that have e.g., a particular disease diagnosis, are taking a particular medication, have received a particular screening test, etc. and, further may permit the HCP to track such information over time (e.g., in graphical or other display format). In some embodiments, a tool may permit a HCP to perform comparisons of his or her patient population with the overall population of patients having a particular disease. In some embodiments, a tool may permit a HCP to search for physicians who have within their patient population one or more patients who exhibit symptoms, signs, or other features similar to those of a particular patient of the HCP. The HCP may thereby identify physicians with particular expertise or experience who may be consulted for advice or to whom a patient may be referred. In some embodiments, a tool may permit a HCP to search for ADMs of patients who have received a particular treatment. Reviewing or analyzing such ADMs may assist the HCP in deciding whether such treatment may be appropriate for a particular patient.

In some embodiments the EMR system may comprise multiple different types of ADM templates. An ADM template may be a generic or universal template usable for any disease or may be a more specialized template. In some embodiments, the EMR system may comprise an ADM template designed for a particular disease, e.g., wherein the ADM template may be designed to be able to capture, in a searchable format, information pertaining to diagnostics and treatments relevant to the disease and, e.g., other features relevant to the disease and/or its treatment. In some embodiments, once a conventional disease diagnosis and, e.g., a molecular disease diagnosis, is established an ADM template designed for that disease may be used henceforth. Such an ADM template may be referred to as a “disease-specialized ADM template”. A disease-specialized ADM template may be adapted to capture information pertaining to e.g., a set of common or significant symptoms, signs (which may include diagnostic test results and/or physical exam findings), complications, or potential outcomes for the particular disease. Such common or significant symptoms, signs, complications, or potential outcomes may be referred to as “key” symptoms, signs, complications, or potential outcomes. In some embodiments, the designation of a set of key symptoms, signs, complications, or potential outcomes for a disease may be within the discretion of the individual or entity that implements or controls implementation of the EMR system or ADM template, e.g., the business entity. Designation of key symptoms, signs, complications, and potential outcomes may, for example, be based on sound medical judgment and current state of the art knowledge. In some embodiments, a key symptom, sign, complication, or outcome may be one whose presence, absence, and/or characteristics (e.g., severity) may affect management of the disease or may provide means to assess the effectiveness of disease management. For example, a key symptom, sign, complication, or outcome may be predictive or indicative of treatment efficacy or failure or side effect(s) or may be predictive or indicative of a possible need to modify disease management.

In some embodiments the EMR system may comprise disease-specialized ADM templates for each of multiple different diseases. It will be understood that the EMR system in at least some embodiments may not provide a disease-specialized ADM template for every disease. In some embodiments an ADM template may be, e.g., discipline-specialized but not disease-specialized. For example, the EMR system may provide an ADM template applicable for a range of diseases within the scope of a particular discipline. In some embodiments, the EMR system may include disease-specialized ADM templates for at least the 3, 5, or 10 most commonly diagnosed diseases in one or more disciplines. In some embodiments, the EMR system may include a disease-specialized ADM template for each of at least 10, 20, 30, 50, 100, or 200 diseases. In some embodiments an ADM template may be, e.g., discipline-specialized and disease-specialized.

In some embodiments, a disease-specialized ADM template may be applicable to multiple distinct diseases, which form a “disease group”. “Disease group” may refer to a group of diseases that are sufficiently similar such that the same ADM template may reasonably be used to capture information pertaining to at least diagnostics and treatments, and, e.g., key symptoms, signs, complications, and/or potential outcomes relevant to the disease while not requesting entry of significant amounts of information that is relevant to only a minority of diseases in the group. In some embodiments, the designation of a particular set of diseases as a disease group may be within the discretion of the individual or entity that implements or controls implementation of the EMR system or ADM template, e.g., the business entity and may, for example, be based on sound medical judgment and current state of the art knowledge.

In some embodiments, the EMR system may comprise more than one disease-specialized ADM template for a particular disease. In some embodiments the EMR system may comprise a “standard” ADM template for a disease and at least one additional ADM template. In some embodiments an additional ADM template may comprise a standard ADM template and further may comprise fields for one or more supplementary data elements to be entered. In some embodiments an additional ADM template may contain one or more fields included at least in part for particular research purposes. In some embodiments an additional ADM template may include at least some fields for data elements that pertain specifically to, e.g., a particular treatment, age group, or presence of a concomitant disease and may not be relevant to most patients having the disease.

In some aspects, the disclosure provides a method of collecting health information, the method may comprise providing an ADM template to a HCP and receiving information entered into the ADM template by or under direction of the HCP. In some aspects, the disclosure may provide a database stored on a computer-readable medium, the database comprising multiple ADMs. In some aspects, the disclosure may provide a computer program product comprising at least one ADM template. In some embodiments, the ADM template may be a disease-specialized ADM template adapted for collecting information pertaining at least to diagnostics and treatments for a disease and, e.g., to key symptoms, signs, complications, and/or potential outcomes relevant to the disease. In some embodiments the computer program product may comprise a collection of disease-specialized ADM templates, each applicable to a different disease. In some embodiments a collection of disease-specialized ADM templates may pertain to diseases corresponding to a particular health care discipline. In some aspects, the disclosure may provide a computer-readable medium having a computer program product of the disclosure stored thereon, wherein the computer program product comprises at least one ADM template. In some aspects, the disclosure may provide a computer-readable medium having a computer program product of the disclosure stored thereon, wherein the computer program product comprises a disease-specialized ADM template or a collection of disease-specialized ADM templates, wherein the ADM templates, e.g., pertain to a particular health care discipline. Many diseases may affect multiple organ systems and/or may be appropriately treated by HCPs who practice in any of various different specialties or subspecialties. ADMs for such diseases may be included in multiple discipline-specific sets of ADM templates. In some aspects, it may take on average no more than 1, 2, 5, 10, 15, or 20 minutes for a HCP to create and complete an ADM tempalate, e.g., a standard ADM template, for a particular disease. In some embodiments an average refers to average for a particular HCP (e.g., over the course of a period of at least a week, month, or year). In some embodiments an average refers to an average across multiple HCPs (e.g., at least 10 patients), e.g., over such time period. A HCP or HCPs may be randomly selected from the set of HCP that use a particular ADM template for a particular disease. Patients may be randomly selected from patients of a particular HCP or set of HCPs. In some embodiments a time may be measured after HCPs have become familiar with the ADM template, e.g., after they have completed at least 10 ADMs.

A health care “discipline” may refer to an area of practice in the art and science of health care. Broadly, disciplines may be classified as, e.g., medical (in which the main diagnostic and therapeutic activities are not major surgery) or surgical (in which surgery is a significant or main part of the diagnostic and/or therapeutic activities), by age range of patients (e.g., pediatrics), body system (where symptoms and diseases typically diagnosed and/or treated arise from or mainly affect a particular organ system or physiological system), as mainly diagnostic or mainly therapeutic, or based on techniques used (e.g., radiology). A discipline may be a specialty or subspecialty in which certification is offered by, e.g., a member board of the American Board of Medical Specialties (ABMS, http://www.abms.org), such as the American Board of Internal Medicine (http://www.abim.org/) or other ABMS member boards listed on the ABMS website (http://www.abms.org/About_ABMS/member_boards.aspx). A list is available at http://www.abms.org/Who_We_Help/Physicians/specialties.aspx. Exemplary specialties and subspecialties include, e.g., allergy and immunology; cardiovascular disease; dermatology; endocrinology, diabetes & metabolism; family medicine; gastroenterology; hematology; infectious disease; internal medicine; nephrology; neurology; obstetrics & gynecology; oncology (e.g., medical oncology); ophthalmology; otolaryngology; pediatrics; pulmonary disease; psychiatry; rheumatology; and urology. In some embodiments a discipline may be long term care, rehabilitation, or physical therapy. A specialty may embrace multiple specialties and/or subspecialties. For example, internal medicine encompasses multiple subspecialties. It will be understood that the scope of various specialties and subspecialties may overlap or change over time or not be precisely defined. However, specialties and subspecialties are well recognized by those of ordinary skill in the art, and they would know which diseases are commonly treated by HCPs practicing in a particular specialty or subspecialty. In some embodiments, multiple subspecialties or specialties may be aggregated into a single discipline for purposes of the EMR system. In some embodiments, a specialty or subspecialty may be divided into multiple (e.g., 2, 3, or more) disciplines for purposes of the EMR system.

In some embodiments, the EMR system may comprise a scheduling component. The scheduling component may, in various embodiments, include, e.g., any capability included in existing electronic or paper-based scheduling systems. The scheduling system may, for example, assist in the scheduling of, e.g., patient appointments (e.g., follow-up appointments, referrals, appointments for tests, etc.), scheduling of resources (e.g., examination rooms, diagnostic equipment, etc.).

In some embodiments, the EMR system may comprise a billing component. The billing component may, in various embodiments, include, e.g., any capability included in existing electronic or paper-based medical coding or billing system. In some embodiments, the EMR system may provide input to an existing medical billing system based on information entered into the EMR system.

In some embodiments, the EMR system may include, e.g., any capability included in any standard EMR system and/or practice management system.

The EMR system may include or may access any of a variety of collections of information in addition to patient health information incorporated into EMRs. For example, such information may include information pertaining to, e.g., diseases, diagnoses, diagnostics, medications (e.g., National Drug Data File Plus drug database), health related costs (e.g., of diagnostics and/or therapeutics), medical terms (e.g., glossaries, translations), medical coding systems, means for converting between different coding or terminology systems, etc. Such compilations may, in some embodiments, be in the form of tables in a database. In some embodiments, the EMR system may use such information in the course of analyzing health information submitted by contributors, assembling EMRs, and/or analyzing requests for information submitted by contributor or subscribers.

In some embodiments, data submitted to the EMR system may be tagged with metadata of any of a variety of types, which metadata may, for example, facilitate analysis of the data for research purposes. For example, a histopathologic test, biopsy, or surgery may be tagged with metadata indicating whether a related biological sample (e.g., a tissue sample) is available for research studies. An ADM may be tagged with metadata indicating whether the ADM was used in a published research study and, if so, a citation of the study or a link to the relevant study or an abstract thereof online, e.g., in Pubmed. In some embodiments an ADM template is published. The ADM template may be made available on Pubmed.

Accounts and Account Database

A user may register (or be registered) with the business entity before beginning to use the EMR system. For example, a contributor may register or may be registered before the contributor first submits health information. Contributor registration may entail providing information that includes at least the contributor's name and an address and, e.g., if applicable, any other information requested by the business entity (collectively “registration information”). For example, in some embodiments, HCPs, e.g., physicians, may be required to provide their license number, DEA number or other prescriber number, employer name or hospital affiliation (if applicable), mobile phone number, business address, and/or email address. In some embodiments, the HCP's credentials may be checked by the EMR system. For example, if the HCP indicates that he or she is employed by an HCO, the EMR system may determine whether that HCO has registered and, if so, whether the HCP is included among the list of HCPs provided by the HCO. In some embodiments, subscribers may be required to provide a name and billing address. In some embodiments, the EMR system may collect such information as the business entity may deem appropriate to, e.g., maintain proper shareholder lists and satisfy any relevant legal requirements. In some embodiments, the user may select an identifier (user ID) and, e.g., a password, for use in accessing the system. In other embodiments a user ID and/or password may be assigned by the business entity. A user ID and/or password may, for example, comprise numbers, letters, non-alphanumeric symbols, or a combination thereof. The EMR system may assign an internal identifier (internal ID) to the user as well, which internal ID may not be disclosed to the user. Information contributed by a contributor may be tagged with that contributor's user ID, internal ID, and/or password, allowing the source of the information to be traced.

In some embodiments, a user account for the contributor may be established by the EMR system that allows the contributor to submit health information to the EMR system and retrieve information therefrom. The particular access rights may vary depending at least in part on whether the contributor is an HCP, auto-contributor, or proxy contributor. In some embodiments, a user account may allow an auto-contributor to view his or her EMR and, e.g., to add certain types of health information to it. Similarly, in some embodiments, a proxy contributor may view and add to the EMR of an individual for whom he or she serves as proxy contributor. In some embodiments, a user account allows HCPs to access EMRs for his or her current patients and submit data to be added thereto. In some embodiments, patient authorization may be required prior to the initial access to the patient's EMR. In some embodiments an HCP may have access to only certain portions of the EMR of at least some of their patients. For example, in some embodiments not all HCPs may have access to all ADMs for a patient. In some embodiments a patient may be able to designate which HCPs are to be provided with access to an ADM, whether a particular HCP is to be provided with access to an ADM, or whether access to an ADM should be provided by default to all HCPs authorized to access the EMR. In some embodiments, ADMs may be assigned an access status at the time of their creation. For example, an ADM may be assigned an access status of “unrestricted” or “restricted”. ADMs with an access status of “unrestricted” may be automatically accessible by a patient's HCPs (after initial authorization), while ADMs with a status of “restricted” may be accessible only with patient authorization. For example, a patient may assign or request an HCP to assign a status of “restricted” to an ADM for a mental health disorder. In some embodiments, an HCP may select an access status for an ADM as part of the process of creating it. In some embodiments, at least some ADMs may be assigned a default access status of “unrestricted” at the time of creation. In some embodiments at least some ADMs may be assigned a default access status of “restricted” at the time of creation. In some embodiments access to one or more non-ADM elements of a EMR or to selected portions of an ADM may also or alternately be restricted.

In some aspects, an exemplary system may include a database containing information pertaining to the user accounts (“user account database”). The user account database may include, for example, at least the registration data, user ID, and access rights for each user.

In some aspects, an exemplary system may maintain a record for each contributor that contains data relating to the contributor's submissions and the incentives earned by the contributor. This data may be included in the user account database as part of the user account information or as a separate account. For purposes of description herein it will be assumed that such data may be maintained as part of the user account, but it should be understood that the data may be maintained as a separate account. In some embodiments, the account data may include, e.g., any of the following: (a) a record of at least some of the submissions from the contributor to the EMR system; (b) the number of EMRs or ADMs assembled from the contributor's submissions; (c) the number of EMRs or ADMs in which the contributor has an interest and may further include, the extent of such interest; (d) the number of times each EMR or ADM in which the contributor has an interest has been accessed by a subscriber; (e) the number of patients of the contributor for whom a EMR has been established; (f) the incentives that the contributor has earned, etc.

Incentives

The term “incentive” may be used herein to refer to any form of tangible or intangible good or service provided as compensation to a contributor by the business entity.

In some embodiments an incentive comprises a share in the business entity. For example, in some embodiments one or more shares would be issued by the business entity to the contributor or the contributor's designee upon submission of health information adequate to assemble a selected number of EMRs or ADMs. In some embodiments, after a EMR or ADM has been created, share(s) are issued based at least in part on the number of times the EMR or ADM is accessed by subscribers. By way of example, in some embodiments the primary contributor of a particular EMR or ADM would be entitled to receive one share each time that access to the EMR or ADM, respectively, has been accessed a specified number of times (e.g., 10, 20, or 50 times, etc.). As used herein, a contributor is said to have an “interest” in a particular EMR or ADM if the contributor is entitled to receive an incentive based at least in part on the contributor's submission of health information that is incorporated into the EMR or ADM or based at least in part on access of the EMR or ADM by a subscriber.

In some embodiments an incentive comprises a monetary incentive (also referred to herein as “money”), which may be provided as cash (currency), check, direct deposit to a contributor's account at a financial institution (optionally located in Switzerland), etc.

In some embodiments an incentive comprises a gift certificate that may be redeemed, for example, at any of one or more retailers, service providers, or other entities offering tangible or intangible items (goods and/or services). In some embodiments, an incentive may consist at least in part of one or more tangible or intangible items (s), such as medical supplies or equipment.

In some embodiments a contributor may receive an incentive in the form of “points” (which may also or alternately be termed virtual money) that the contributor may, for example, apply towards acquisition of selected tangible or intangible item(s), or exchange for money or shares in the business entity. In some embodiments, the contributor's account may keep track of the number of points earned by the contributor and their application by the contributor towards the acquisition of selected item(s) or their exchange for money or shares. In some embodiments, if a contributor elects to exchange points for a share, the share may be issued to the contributor or the contributor's designee, and the share database may be updated accordingly.

In some embodiments an incentive may comprise multiple different forms of incentive. For example, an incentive may include cash and one or more shares or points. In some embodiments an incentive may comprise the opportunity to offer experimental therapies to patients. In some embodiments an incentive may comprise the opportunity enroll patients in clinical trials and/or managed access programs or refer patients for enrollment in clinical trials and/or managed access programs, which opportunity may allow an HCP to offer experimental therapies to patients. In some embodiments an incentive may comprise the opportunity to have access to an EMR system that provides Experimental Therapies functions. In some embodiments an incentive may comprise the opportunity to have access to an Experimental Therapies component and/or ADM component.

A contributor may receive an incentive under any of a variety of circumstances in various embodiments. In some embodiments, receipt of an incentive may be based at least in part on submission of health information by the contributor and/or request(s) for use of such health information by a subscriber. For example, in some embodiments submission of health information that is, e.g., adequate to assemble a selected number of EMRs, may entitle the contributor to an incentive. In some embodiments submission of health information that is, e.g., adequate to assemble a selected number of tentative ADMs, may entitle the contributor to an incentive. In some embodiments submission of health information that is, e.g., adequate to assemble a selected number of definitive ADMs, may entitle the contributor to an incentive. In some embodiments submission of health information that contributes to an ADM may entitle the contributor to an incentive. In some embodiments, submission of health information that may allow a tentative diagnosis to be established as a definitive diagnosis according to the EMR system diagnostic criteria for that diagnosis may entitle the contributor to an incentive.

In some embodiments a contributor may receive an incentive following a request by a subscriber to access a EMR to which the contributor contributed, e.g., as a primary or secondary contributor. In some embodiments a contributor may receive an incentive following a request by a subscriber to access an ADM to which the contributor contributed, e.g., as a primary or secondary contributor. For example, if a subscriber requests access to ADMs having a definitive diagnosis of “rheumatoid arthritis”, contributors to such ADMs may receive an incentive. For example, if a subscriber requests access to ADMs having a definitive diagnosis of “rheumatoid arthritis”, and in which the patient was prescribed Enbrel® as a medication, contributors to such ADMs may receive an incentive. Thus, in some embodiments the incentive may be considered as a royalty to the contributor for use of the EMR or ADM. For example, the contributor may be entitled to a certain sum of money per access to a EMR or ADM to which the contributor contributed information, wherein the total amount to which the contributor is entitled (e.g., within a given month) depends at least in part on the number of requests for access to such EMRs or ADMs by subscribers and, e.g., at least in part on the subscription class of the subscriber(s) making such requests. For example, an incentive may be larger if the access was by a subscriber paying a larger fee for access versus a smaller fee (or no fee). In some embodiments the incentive may be a fraction of the revenue attributable to the EMR or ADM. For example, an incentive may be between 1%-99%, e.g., 5%-75%, e.g., 10%-50%, of the revenue (e.g., net revenue or gross revenue) attributable to the EMR or ADM (e.g., on a monthly, quarterly, or yearly basis) in various embodiments. In some embodiments, a contributor, e.g., an HCP may be guaranteed at least a minimum incentive for participation, regardless of whether the health information contributed is accessed by subscriber(s).

As noted above, in some embodiments an incentive may comprise or consist of at least one share in the business entity. For example, the contributor may receive one share as remuneration for submission of a health information dataset adequate to assemble one EMR or if an ADM contributed by the contributor is accessed by a subscriber. In some embodiments the number of shares received may depend at least in part on the share price. For example, the payment per EMR may be a number of shares worth a selected amount of money.

In some embodiments, if multiple contributors contribute to a EMR or ADM for a particular patient, the payment may be distributed in a variety of ways. In some embodiments the primary contributor of the EMR for that patient may receive the payment. In some embodiments, the contributor who contributed the ADM may receive the payment. In some embodiments the contributor who contributed the tentative diagnosis that is ultimately deemed definitive may receive the payment. In some embodiments the contributor who contributed the final item of data required to establish a tentative diagnosis as a definitive diagnosis may receive the payment. In some embodiments the contributor who contributed the definitive diagnosis may receive the payment. In some embodiments, only a patient's HCPs may contribute a tentative or potentially definitive diagnosis. In some embodiments, a contributor who is not the patient's HCP but who has access to the ADM may contribute a tentative or potentially definitive diagnosis that could be confirmed by the system as definitive. For example, a HCP who is not the patient's HCP or a subscriber who may or may not be a HCP may contribute a proposed definitive diagnosis in some embodiments.

In some embodiments an incentive may be divided among multiple contributors who have contributed to the definitive ADM. The formula for dividing an incentive may vary. For example, in some embodiments between 10% and 90% may be given to the primary contributor of the EMR that contains the ADM, and the balance may be distributed among secondary contributors (if any) who contributed data contained in the ADM. In some embodiments 10% may be given to the primary contributor of the EMR that contains the ADM, 50% may be given to the contributor of the ADM (who may or may not be the primary contributor of the EMR), and the balance may be distributed among secondary contributors who contributed data contained in the ADM (if any). In some embodiments, if the primary contributor contributed the ADM, all data contained therein, and the definitive diagnosis, then the entire incentive may be given to the primary contributor.

In some embodiments, an incentive may be distributed at least in part randomly. For example, any contributor to an ADM may have an equal chance of receiving an incentive attributable to a request of that ADM by a subscriber.

In some embodiments, an incentive distribution scheme may be disclosed to contributors. In some embodiments, an incentive distribution scheme may be at least in part not disclosed to contributors.

In certain embodiments ADM-equipped EMR systems may provide HCPs and/or HCOs with any one or more of the following, any of which may serve as incentives for HCPs to use ADMs or ADM-equipped EMR systems: rapid access to quality baseline EMRs to which their data are added, rapid access to peer networks for consultation and/or feedback, ratings based on objective measures such as outcomes and experience (e.g., number of patients whom a HCP or HCO has cared for who have a particular disease) that may supplement or replace subjective patient ratings or feedback forums, a database that can be used to perform intra-mural (within an organization) or extra-mural research (e.g., to gather information, analyze and/or compare different treatment options, e.g., in terms of marketed drugs, Expanded Access drugs, and their outcomes) and/or comparative analysis with other providers; increased access to Experimental Therapies (which may include, e.g., opportunity to serve as an investigator in a clinical trial, opportunity to provide a therapy to a patient under a Managed Access Program, opportunity to obtain a therapy that is a candidate for repositioning at a reduced or no cost, etc.). In certain embodiments use of ADMs may improve HCP (e.g., physician) perceptions of EMRs, at least in part by providing HCPs with ADM-based opportunities to, e.g., make experimental therapies available to their patients.

In some embodiments quality control associated with ADMs and its diagnostic support may limit liability exposure of HCPs and/or HCOs, e.g., in the case of alleged misdiagnosis or alleged inadequate or inappropriate treatment. In some embodiments use of an ADM and/or content of an ADM may be admissible in a court as evidence that a HCP or HCO adhered to a standard operating procedure and/or provided an appropriate standard of care. In some embodiments use of an ADM-equipped EMR system may result in lower malpractice insurance costs for a HCP or HCO than would be the case if such HCP or HCO did not use an ADM-equipped EMR system. In some embodiments an entity that at least in part owns, controls, makes, sells, or provides an ADM component, ADM-equipped EMR system, or ADM database may verify or certify that an HCP or HCO uses and/or appropriately maintains ADMs, e.g., for a specified proportion of patients.

It should be understood that the afore-mentioned incentives and incentive distribution schemes are exemplary, and many other incentives and incentive distribution methods could be used.

Different HCOs may have different policies regarding the distribution of incentives to their employees or affiliated HCPs. For example, in some embodiments, some HCOs may permit incentives, e.g., shares, to be issued to and owned by employees or affiliated HCPs. In some embodiments, some HCOs may require that incentives, e.g., shares, be issued to and owned by them. In some embodiments, some HCOs may require that incentives be issued to them for subsequent transfer to employees or affiliated HCPs. Some HCOs may restrict the type or amount of incentive that may be provided to their employees or affiliated HCPs. Various embodiments may accommodate these models for distribution and ownership of incentives and, e.g., other models as appropriate. In some embodiments, the type and/or amount of incentive for which a contributor may be eligible may be included in the account information for that user. It should be understood that incentive distribution schemes may change over time.

In some embodiments, different incentives may be provided for contribution to a type 1 ADM versus a type 2 ADM or for contribution to ADMs created using different ADM templates. For example, an ADM template containing numerous fields may merit a larger incentive than an ADM template that contains only a few fields.

In some embodiments an exemplary system may include a component that may be used to manage incentives, e.g., to keep track of the number and type of incentives earned by each contributor and, e.g., to analyze or provide information or report(s) relating to a contributor's remuneration, as requested, to arrange for distribution of incentives to contributors, etc. Data regarding the number and type of incentives may be stored in a database, e.g., in a user account database, a separate database, or both. The component may at least in part arrange for transfer of incentives to contributors. For example, the component may transmit instructions to a financial institution, retailer, or other entity in order to effect transfer of money, items, etc., to the contributor. The component may interface with the database and may, for example, update the database accordingly after arranging for transfer of an incentive.

As described further below, in some aspects the disclosure may provide an application that allows a contributor to request (via, e.g., a portable electronic device) information regarding the incentives earned by the contributor. In embodiments in which an incentive may be a share of the business entity, the component may keep track of the number and type of shares owned by each contributor and, e.g., may analyze or provide information or report(s) relating to the contributor's ownership interest as requested. The database may keep track of those shares of the business entity that are owned by non-contributors. In some aspects the disclosure may provide an application that allows a contributor to request (via, e.g., a portable electronic device) information regarding the number of shares owned by or earned by the contributor. Information or reports generated using information in the database may be used by the business entity in the course of doing business.

In some embodiments, the business entity may provide remuneration to patients based at least in part on access of their (ordinarily de-identified) health information (e.g., ADMs pertaining to them) by subscribers. The remuneration may take the form of incentives, as described herein, and may be managed by the business entity in the same way. The patient may or may not be a contributor. In some embodiments patients may have accounts and may, e.g., be provided with account data pertaining to use of their health information and/or remuneration, as described herein for incentives.

Subscriptions

In many embodiments the business entity may offer access to the EMR database to third parties, e.g., in exchange for a fee. Such third parties may be, for example, medical researchers, organizations such as pharmaceutical companies or insurance companies, government entities (e.g., Federal, state, and/or local government entities), or simply individuals interested in the content of the database. In some embodiments, individuals, organizations, or entities that are provided with access to the database, e.g., in exchange for a fee, may be referred to as “licensees” or “subscribers”. In some embodiments, the arrangement under which such access is granted or the set of access rights provided may be referred to as a “subscription” or “license”. There may be multiple classes of subscriptions that, for example, allow subscribers different access rights. For example, access rights may differ in terms of number of access sessions or queries permitted, the type of information that may be accessed, the type of analysis that may be performed, etc. In some embodiments, the different classes of subscriptions may have different fees and/or fee structures, which may depend at least in part on the extent and nature of the associated access rights. For example, a subscription may provide a single access session to the database in exchange for a one-time fee. In some embodiments a subscription allows the subscriber to access the database multiple times over a defined period of time, such as 1 month, 3 months, 1 year, etc. The number of access sessions and/or queries permitted within the defined time period may be limited or unlimited in various embodiments. In the case of organizations or other entities, a site license may be provided that allows multiple users to have access to the database. Each subscriber and/or user may select or may be assigned a user ID and, in at least some embodiments, a password. Some types of subscription may permit the licensee to print and/or download information while other types may only permit viewing. Some types of subscription may permit the licensee to access ADMs only. Some types of subscriptions may permit the licensee to access de-identified EMRs or portions thereof such as patient summary data, ADMs. For example, if a researcher is interested in identifying potential combination therapies for a particular disease, the researcher may want to obtain a complete list of the patient's medications in addition to the particular medications prescribed for the disease of interest. If a researcher is interested in studying an infectious disease such as tuberculosis, it may be relevant to know whether the patient is immunocompromised due to a co-existing condition or medication. In some embodiments, the fee for accessing different EMRs or ADMs may differ based at least in part on diagnosis, treatment or other elements of the EMR or ADM.

In some embodiments, the business entity may give subscriptions to at least some contributors (e.g., the contributor may receive a subscription without paying a fee). In some embodiments the business entity may give subscriptions to at least some HCPs who are not contributors (e.g., the HCP may receive a subscription without paying a fee). In some embodiments, the business entity may give subscriptions to at least some HCOs. In some embodiments, the business entity may give subscriptions to students (e.g., students studying a health care profession, such as medical students, nursing students, dentistry students, pharmacy students) and/or trainees in a health care profession (e.g., interns, residents, etc.). In some embodiments, subscriptions may be provided to members of HCP professional organizations, e.g., as a membership benefit. In some embodiments, third parties may purchase or otherwise sponsor subscriptions for others. In some embodiments the business entity may give subscriptions to nonprofit organizations that are engaged in research, e.g., medical research. In some embodiments the business entity may require at least some subscribers to publish, or to deposit in a publicly available repository, results of any studies performed using the EMR database. Such results may be required to be published or deposited within a reasonable time such as, for example, within no more than 12 months from obtaining a result or completing a study. It is envisioned that the value and utility of the EMR system, according to some embodiments, may increase over time in part as a result of the performance of such studies.

In some embodiments terms and/or conditions under which a subscription is provided are embodied at least in part in a subscription agreement. In some embodiments a subscription agreement may be accepted electronically by an entity or individual wishing to become a subscriber. In some embodiments terms of a subscription may include a requirement for payment (e.g., royalties) on any new products or new uses of existing products that are discovered or identified at least in part through use of the database. In some embodiments terms of a subscription may include a requirement for payment (e.g., royalties) on any new products or new uses of existing products that approved for marketing at least in part through use of the database. In some embodiments such payments are to be paid to an entity that owns or controls the database. In some embodiments at least some such payments may be distributed to individuals or entities that contributed data used in the identification, discovery, or approval of a new product or new use for an existing product. In some embodiments a subscription agreement comprises a license agreement that secures payments (e.g., royalties) on any new products or new uses of existing products that are discovered or identified or approved for marketing at least in part through use of the database. In some embodiments one or more template license agreements may be provided. In some embodiments a subscriber or potential subscriber may select among two or more such template license agreements.

In various embodiments users may be provided with access to the content of the EMR database up to the extent permitted by applicable law (including any regulations issued by government agencies pursuant to such laws), such as the U.S. Health Insurance Portability and Accountability Act of 1996 (“HIPAA”) Public Law 104-191, as amended from time to time. The extent of access that is permissible under applicable law may vary depending upon the user. For example, a patient's HCPs and the patient (and patient representatives, if any) may have access to the patient's complete EMR. Certain subscribers would be provided with access only to de-identified health information. In some embodiments, de-identification comprises removing or blocking access to protected health information (PHI) as defined in the regulations issued by the U.S. Department of Health and Human Services (HHS) under HIPAA, known as the Standards for Privacy of Individually Identifiable Health Information (“Privacy Rule”), as amended from time to time. In some embodiments, certain subscribers may be provided with access to at least some PHI if performing Research (as defined by the Privacy Rule) and if the requirements of the Privacy Rule have been satisfied. In some embodiments, laws and/or rules of countries or jurisdictions other than the U.S. may be applied in addition to or instead of those of the U.S., which may be selected based at least in part on where the patient resides, where the HCP practices, and/or where the business entity is incorporated or physically located.

An exemplary system may comprise one or more components that at least in part facilitates use of the EMR database by subscribers. For example, a component may provide subscribers with a GUI that facilitates query creation. Subscribers may, for example, be permitted to search the EMR database using various fields, Boolean operators, and/or natural language queries.

Subscribers may, in various embodiments, download ADMs or portions thereof onto their own computer systems for analysis independently of the EMR system (e.g., using their own proprietary tools) or may perform analysis using computer-based tools provided by the EMR system or may use a combination of such approaches.

In some embodiments, the EMR system may provide one or more computer-based tools that include facilities for data manipulation, calculation, graphical display, and/or statistical analysis of ADMs. A subscriber could perform any of a variety of types of analysis on ADMs in various embodiments. To provide a few examples, in some embodiments a subscriber may determine the frequency with which particular symptoms or signs are present in patients with particular disease(s), determine the frequency with which particular diagnostic tests or treatments are utilized in patients with particular disease(s), identify correlations between various symptoms, signs, complications, treatments, etc. For example, in some embodiments, a subscriber may determine the percentage of rheumatoid arthritis patients receiving Enbrel® (etanercept) versus Humira® (adalimumab) versus other medications or no medication, or may determine the distribution of patients receiving Enbrel or Humira by disease or may determine the percentage of patients receiving Enbrel or Humira who developed an infection from a particular pathogen within a given time period. In some embodiments, a tool may assist in data mining activities. In some embodiments, tata mining may encompass the automatic or semi-automatic analysis of large quantities of data to extract previously unknown interesting patterns such as groups of data records or structures in the data that are in some way “similar” (e.g., cluster analysis), unusual records (e.g., anomaly detection) and/or dependencies (e.g., association rule mining). In some embodiments the EMR system may provide tool(s) that facilitate assembling a more compact representation of a data set, such as visualization tools or report generation tools. In some embodiments, tools may be provided to extract data into standard available data analysis or statistical software programs, packages or environments such as SAS®, SPSS, Systat®, Minitab®, R, etc. or to analyze data using tools such as those provided in such software.

In some embodiments a business entity provides a service comprising organizing and/or analyzing drug and/or device distribution information and/or data analysis. In some embodiments such service(s) may be provided as part of a subscription. In some embodiments such service(s) are provided for a fee. Drug or device distribution information may comprise, e.g., number of units prescribed within a selected time period, locations to which drugs or devices are shipped or supplied, number of units prescribed or used by particular HCPs, number of units prescribed or used by HCPs affiliated with or employed by a particular HCO, etc. “Units” may be packages, unit dosage forms, or any other suitable means of quantifying a drug or device. Data analysis may comprise tracking changes in drug or device distribution or utilization over time, comprising drug or device distribution or utilization between different HCPs, HCOs, geographic regions, patient populations, etc.

It is envisioned that in some embodiments, the EMR system may empower HCPs to develop and explore health-related questions or hypotheses that may arise in the course of their practice activities. Such questions or hypotheses may be explored by analyzing ADMs from the EMR database. In some embodiments the EMR system may allow HCPs to submit the results of such analyses and makes the results available to other users and, e.g., to the public. In some aspects, tools that facilitate HCP interaction or collaboration to address health-related questions may be provided. In some embodiments, HCPs may submit suggestions via the EMR system for fields to be included in future ADM templates.

In some embodiments the EMR system may assist HCPs in remaining up to date with current health care-related knowledge, e.g., current diagnostic and/or therapeutic approaches. For example, in some embodiments the EMR system may provide information or links to information pertaining to at least some conventional disease diagnoses, molecular disease diagnoses, diagnostics, and/or therapeutics. Such information may include, for example, publications such as diagnosis or treatment guidelines, research articles, educational materials prepared by or on behalf of the business entity, etc. The EMR system may in some embodiments be useful as a tool to help students or HCPs learn or prepare for examinations including, but not limited to, board examinations or recertification examinations.

In some embodiments, the EMR system may be useful in preparing future revisions of diagnostic or therapeutic guidelines. Currently, such revisions may be based at least in part on information in published research studies (e.g., studies described in articles available in PubMed). Review and/or analysis of ADMs may supplement such information or may be used to address unanswered questions about a disease or therapy. In some embodiments, an ADM template may incorporate one or more fields designed to help an address a question or unresolved issue relating to disease diagnosis or treatment.

In some embodiments a subscriber (or other user) may elect to receive an alert (e.g., via email, text message, phone call, voicemail, fax, etc.) when particular information of interest to the subscriber is entered or when particular conditions of interest to the subscriber are met. For example, if a subscriber is interested in analyzing ADMs having a definitive diagnosis of rheumatoid arthritis and for which the patient was prescribed Enbrel, the EMR system may provide an alert to the subscriber when a predetermined number of such ADMs have been created. As another example, the EMR system may provide an alert to a government entity when a predetermined number of tentative diagnoses of “influenza” are entered within a particular geographic area during a particular time period. The system may thus, in some embodiments, facilitate timely conveyance of important health information to government authorities. In some embodiments, alerts may be prioritized by importance, e.g., as predetermined by the subscriber.

In some embodiments, an exemplary system may comprise one or more components that at least in part manages subscriptions. For example, in some embodiments, the component may keep track of access rights, use of the database by subscribers, payments due and received, etc. Such a component may be, e.g., part of a user account manager.

ADM-Assisted Clinical Trials and Managed Access Programs

In some embodiments, the EMR system may be used to facilitate the performance of clinical studies, e.g., clinical studies aimed at obtaining or maintaining regulatory approval or clearance or complying with legal requirements for marketing of medically related products such as pharmaceutical agents (also referred to as “drugs” herein, as defined, e.g., by the US Federal Food, Drug, and Cosmetic Act (FD&C Act)), diagnostic agents (for in vivo or ex vivo use) or diagnostic kits, or medical devices (e.g., implantable devices such as pacemakers, artificial joints, etc. or therapeutic or diagnostic equipment), or combination products (as defined in 21 CFR 3.2(e)) or medical or surgical procedures or other health related products or services (i.e., products or services that potentially affect health) such as food additives, color additives, personal care products such as toothpastes, dietary supplement products, dietary ingredients, pesticides, herbicides, etc., by government agencies responsible for overseeing such matters. In some embodiments the regulatory agency is the U.S. Food & Drug Administration (FDA) and the medically related or health related product may be a drug. The agency may be another regulatory agency such as a regulatory agency in another jurisdiction, such as Europe, Japan, China, India, Brazil, etc., and the medically related or health related product may be any medically related or health related product, e.g., any regulated medically related or health related product. All U.S. federal legislation (laws and related regulations) pertaining to regulation of medically related and/or health related products and processes included in the Code of Laws of the United States of America (variously abbreviated to Code of Laws of the United States, United States Code, U.S. Code, or U.S.C.) and/or included in the U.S. Code of Federal Regulations (C.F.R.), and all FDA Guidance documents, as amended or updated from time to time, are incorporated herein by reference. Without limiting the foregoing, U.S.C. Title 21 and C.F.R. Title 21, as amended or updated from time to time, are incorporated herein by reference. Such references may be consulted for relevant definitions or information but should not be considered as limiting the invention unless so indicated. A pharmaceutical company may be any company engaged in the development, production, and/or marketing of one or more pharmaceutical agents, diagnostic agents, diagnostic kits, medical or surgical devices, combination products (as defined in 21 CFR 3.2(e)). A drug may be a brand name drug, a generic drug, a prescription drug, or an over-the-counter (OTC) drug.

The term “clinical study” may be used interchangeably herein with “clinical trial” or “clinical investigation” and may be referred to simply as a “study” or “trial”. The term may be intended to encompass any biomedical or health-related research study in human beings (often referred to as “subjects”). A clinical study may follow an at least in part predefined protocol. A clinical study may be an interventional study or an observational study. Interventional studies may be those in which subjects are assigned to a treatment or other intervention, and one or more outcomes are assessed, e.g., to determine whether the intervention had an effect on the outcome. Observational studies may be those in which individuals are observed and at least one outcome is assessed (without having provided an intervention believed or known to potentially have an effect on such outcome(s)). The term “sponsor” may refer to an entity, organization, or individual who takes responsibility for and, may initiate a clinical investigation. The sponsor may be, for example, an individual or pharmaceutical company, governmental agency, academic institution, private organization, or other organization. A trial may have multiple sponsors and/or the sponsor may change during the course of the trial. It will also be understood that a sponsor may engage an organization such as a contract research organization, also referred to herein as a clinical research organization, (CRO) to fulfill at least some of its responsibilities for the trial or otherwise provide services relating to the trial (which services may include using the EMR database).

In some embodiments, the EMR system may be used in a clinical trial by using ADMs to identify or enroll subjects and/or to gather data pertaining to the trial. For purposes hereof, a clinical study in which an ADM is used, e.g., to identify or enroll subjects and/or to gather data pertaining to the study may be referred to as an “ADM-assisted study”. In some embodiments an ADM-equipped EMR system is used. In some embodiments an ADM-equipped EMR system which is a standard EMR system equipped with functionality that permits the utilization of ADMs is used, e.g., a standard EMR system comprising an ADM component (see description above) is used. ADMs and/or ADM-equipped EMR systems may be used in any of a variety of ways in connection with a clinical trial in various embodiments. For example, an ADM may be used to determine whether a subject meets predetermined inclusion criteria (subject eligibility) and/or to gather data pertaining to outcome. The term “outcome” encompasses any event, occurrence, measurement, etc., of interest in the context of a clinical study, e.g., any event, occurrence, or measurement that is relevant or possibly relevant to the effect of an entity being studied on a subject. An outcome may be an occurrence or change in a symptom, sign (e.g., physical exam finding, laboratory value or other test result), or disease (e.g., improvement or worsening). An outcome may be a predefined outcome (i.e., the outcome is defined as being of interest prior to the initiation of the study) or an outcome that is not necessarily predefined, such as an unexpected adverse event. An outcome may be a composite outcome derived from multiple individual outcomes or measurements. In some embodiments an outcome is an “endpoint”, which term generally refers to an outcome that is a target outcome of a trial (e.g., an outcome that the trial is intended to assess, e.g., an outcome that may be evaluated to determine whether a drug or device is effective or whose occurrence may mandate that a subject discontinue treatment with the entity under study).

A clinical trial endpoint may, in some embodiments, be a clinical endpoint or a surrogate endpoint. An endpoint may be designed or selected specifically for a particular clinical trial. Many typical clinical trial endpoints are known in the art. For example, in clinical trials of HMG-CoA reductase inhibitors, a common surrogate endpoint is serum cholesterol measurement. A relevant clinical endpoint for such agents may be major coronary heart disease events as myocardial infarction. As another example, in clinical trials of cancer therapies, common clinical endpoints include discovery of local recurrence, discovery of regional metastasis, discovery of distant metastasis, onset or change in symptoms (e.g., quality of life assessment), hospitalization, increase or decrease in pain medication requirement, onset of toxicity (e.g., dose-limiting toxicity), requirement of salvage chemotherapy, requirement of salvage surgery, requirement of salvage radiotherapy, death from any cause or death from cancer. Clinical trials in cancer may measure objective response rate, e.g., as defined using the Response Evaluation Criteria In Solid Tumors (RECIST) guideline (Therasse, P., et al., Journal of the National Cancer Institute, 92(3): 205-216 (2000) or revised RECIST guideline (version 1.1) (Eisenhauer, E. A., et al., Eur J Cancer. 45(2):228-47 (2009)) or other accepted guidelines, e.g., for hematological malignancies or brain tumors. For example, an outcome may be classified as a complete response, partial response, progressive disease, or stable disease. An endpoint may be agreed upon by a sponsor and the FDA prior to starting a trial.

The term “clinical trial data” may be used to refer to any item of health information or other information that is of interest or required for purposes of determining eligibility of a patient in a clinical trial and/or that is collected to fulfill one or more requirements of a clinical trial protocol. The process of determining whether a subject is eligible or potentially eligible to participate in a clinical trial may be referred to as “screening”. Information required to determine subject eligibility may be referred to as “screening data”. An item of screening data may or may not be part of health information that would ordinarily be collected for a patient. Health information that is required for purposes of fulfilling one or more requirements of a clinical trial protocol may include data that provides an indication of safety and/or efficacy of the therapy being studied in the trial, e.g., data of use in determining outcome or determining whether a trial meets one or more endpoints. Clinical trial data may include, in addition to data items specifically required by the protocol, additional health information that may be collected pertaining to a subject as a result of a subject visiting a HCP and/or visiting a site at which the trial is conducted, even if not specifically listed in the trial protocol. For example, clinical trial data may include unexpected symptoms experienced by a subject or unexpected signs manifested by a subject, which symptoms or signs may not be mentioned in the protocol (e.g., because they are unexpected). The term “trial-specific clinical trial data” may be used to refer to clinical trial data that is collected particularly for purposes of a clinical trial, e.g., such data would not ordinarily be collected as part of ordinary standard of care therapy for the subject. Trial-specific clinical trial data may be of the same general type as data that would ordinarily be collected as part of ordinary standard of care therapy for the subject, but may be collected according to a different schedule (e.g., more frequently) or in a different manner than would typically be the case if the patient was not enrolled in the trial. Trial-specific clinical trial data may be data that is not of the same general type as that which would ordinarily be collected as part of standard of care therapy for the patient. For example, if the trial involves administration of an experimental cancer vaccine to a subject, a test to determine whether the subject mounts an immune response to an antigen included in the vaccine may be performed. Such a test would not typically be part of the ordinary standard of care of the patient were the patient not participating in the trial. Results of the test would be considered trial-determined clinical trial data. Trial-specific clinical trial data may in some embodiments be specific to only a single trial or trials or may in some embodiments be of a type that is frequently or typically collected for clinical trials, e.g., trials in the particular disease being studied.

“Electronic data capture” (EDC) refers to the collection of clinical trial data in electronic format, typically for use in human clinical trials. EDC may be used in clinical trials at least in part instead of recording clinical trial data on paper forms (case report forms) that are sent to a sponsor (e.g., a pharmaceutical company) or CRO where the data are then entered into a database and analyzed. EDC may be performed for purposes of screening and/or for purposes of collecting clinical trial data pertaining to subjects enrolled in a trial. An “EDC system” is a system comprising computer-executable instructions that provide appropriate functionality for performing EDC. EDC systems allow study personnel, e.g., investigators and CRCs, to enter data at a site, typically into an electronic document referred to as an electronic case report form (eCRF). The entered data may then be electronically transmitted to a trial's sponsor or to a CRO managing certain aspects of the trial on behalf of a sponsor. A number of EDC systems are known in the art. For purposes hereof, the term “standard EDC system” may refer to an existing (as of the date of the present invention) EDC system, e.g., a computer program product for entry of data in the context of a clinical trial, that is commercially available or otherwise publicly known or used as of the date of the present invention. An EDC system may be installed on a computer located at a clinical trial site and used at the site for entry of data of interest in the context of the clinical trial. An EDC system may periodically transmit entered data to a sponsor or CRO e.g., using a suitable electronic transmission means. An EDC system may electronically receive queries from a sponsor or CRO to be addressed by investigator(s) or CRC(s) at the site. Alternately or additionally, an EDC system may comprise web-based software that can be accessed using a computer that runs a web browser, without the need for the computer to have special EDC software installed. The data may be transmitted to a sponsor or CRO via the Internet. Queries entered by a sponsor or CRO may be viewed at the site and responded to using such web-based software. A “site” may be, e.g., a hospital, medical center, clinic, practice, or any other HCO at which a trial is at least in part conducted, e.g., at which an investigator in the trial is employed or affiliated and/or treats patients in the trial.

In some aspects the disclosure encompasses the recognition that standard EDC systems may have a number of limitations. For example, existing EDC systems may be limited by their design to support specific clinical trials, do not offer solutions to support data management outside of formal clinical trials or to aggregate and meta-analyze data across multiple trials, and do not interact or integrate with standard EMR systems. Existing EDC systems may be imposed on clinical sites by trial sponsors or CROs. Single sites may have to use multiple EDC systems in parallel. In some embodiments ADM-equipped EMR systems, ADM-EDCs, or an ADM database provide solutions that may at least in part avoid or address one or more such limitations.

In some aspects an ADM-equipped EMR system, which may be an EMR system that is organized around ADMs as the primary means of data entry or a standard EMR system equipped with ADM functionality (e.g., via an ADM component), is used for EDC. Fields that specify entry of data appropriate to determine subject eligibility or outcome or any other clinical trial data of interest may be incorporated into an ADM template. Such data may, in various embodiments, be entered into an ADM as text, by selecting from among various options from a list, checking boxes, by voice, or using any other method for data entry described herein or known in the art. In some embodiments the standard ADM template provided by the EMR system for the particular disease of interest may be sufficient for purposes of a clinical trial. In some embodiments an ADM template may be designed specifically for purposes of a clinical trial. In some embodiments a standard ADM template for a disease of interest may be augmented to include additional fields (“trial-specific fields”) for purposes of using the ADM in a clinical trial. It will be understood that an ADM or trial-specific fields thereof may be modified during the course of a study under appropriate conditions. For example, if a possible adverse event emerges as a potential concern during the course of a trial, an ADM template may be modified to include a field that would require checking the subject (e.g., on a predetermined or recurring basis) for occurrence of such event or would require performing a test intended to identify subjects who may be prone to the occurrence of such event or who may be in the process of developing such event. In some embodiments, an ADM template or trial-specific fields thereof may be designed such that data gathered thereby may comply with a particular set of regulatory requirements and/or requirements selected by a sponsor and/or agreed on by a sponsor and the FDA.

In some embodiments the EMR system may check an ADM to determine whether a potential subject meets trial eligibility criteria and, in some embodiments, informs the HCP or a designee of the HCP accordingly. In some embodiments a subject may be considered to be enrolled in an ADM-assisted clinical trial after the subject's HCP has agreed to serve as an investigator and such agreement may be documented in the EMR system, the subject has met inclusion criteria for the study (if any) or at least does not meet exclusion criteria (if any), and an informed consent document for the subject has been entered into the EMR system. In some embodiments, at least one intervention being studied in the trial (e.g., administration of a drug being studied) must have occurred and have been documented by entering data in the appropriate ADM in order for the subject to be considered enrolled in the trial. In some embodiments, the number of subjects enrolled in a trial may be between 300 and 5,000 for, e.g., a Phase III trial. In some embodiments, the number of subjects enrolled in a trial may be between 5,000 and 10,000 for, e.g., a Phase III trial. In some embodiments the number of subjects enrolled is in the order of tens of thousands or hundreds of thousands of patients, e.g., 10,000-20,000; 20,000-50,000; 50,000-100,000; or more, in various embodiments.

In some embodiments the EMR system may check an ADM to ensure that the entered data meets specified criteria required for use of the ADM in a clinical trial. For example, the EMR system may check to ensure that all fields of an ADM pertinent to outcomes to be assessed in the trial are completed. In some embodiments, the EMR system may check to ensure that ADMs are updated at appropriate times. For example, if a trial protocol requires that a patient be evaluated at specified time intervals or over a specified time period, the EMR system may send an alert to the patient's HCP or a designee such as a clinical research coordinator if the data is not entered in a timely manner or is incomplete or may reject the ADM as inadequate for use in the study if the data are not entered within a predetermined time period or are incomplete. The EMR system may thus help enforce compliance with protocol requirements. In some embodiments, the EMR system may check an ADM to determine whether one or more endpoints for a clinical study is/are met. In some embodiments the EMR system may send an alert to a HCP, HCP designee, and/or to a sponsor or CRO if it determines, based on data entered into an ADM, that an outcome that requires subject withdrawal from a study has occurred. In some embodiments, enrollment in an ADM-assisted clinical trial may be considered to be completed once a predetermined number of subjects have been enrolled via the EMR system. In some embodiments, an ADM-assisted clinical trial may be considered to be completed once data adequate to determine whether an endpoint has been met (and, if applicable, any required follow-up data) has been entered into a specified (e.g., predetermined) number of ADMs.

In some embodiments, an ADM-assisted clinical trial may be conducted for purposes of generating data to be included in a New Drug Application (NDA), Biologic License Application (BLA), or Abbreviated New Drug Application (ANDA). In some embodiments it is envisioned that an ADM-assisted clinical trial may be conducted to generate data to be included in an application for approval of a so-called follow-on or biosimilar biologic drug, e.g., as specified in the framework established under Patient Protection and Affordable Care Act of 2010 (“PPACA”).

In some embodiments, an ADM-assisted clinical trial may be conducted at least in part for purposes of generating data to be included in a Pre-market Application (PMA) for a medical device, e.g., a class III medical device. In some embodiments, an ADM-assisted clinical trial may be conducted at least in part for purposes of generating data to be included in an application for 510(k) clearance for a medical device, such as class II medical device, e.g., to show that the device is “substantially equivalent” to a predicate device already on the market is required for class II devices.

It is noted that an ADM-assisted clinical study may be performed for any purpose in which it is legally permissible to perform a clinical study, including, but not limited to, for regulatory approval purposes. For example, a clinical study may be performed to evaluate the efficacy of a treatment, e.g., to support a contention that a treatment may be efficacious or may not be efficacious, e.g., for purposes of showing that the cost of the treatment should be covered by health insurance or (if the treatment is found not to be efficacious) need not be covered by health insurance, or to compare efficacy or side effects of different treatments, etc. In some embodiments, an ADM-assisted clinical trial may be performed to test a medically related product that has already been approved in at least one indication. In some embodiments, the trial may be performed to evaluate the product in an indication different from that for which it was approved or in a patient population having specified characteristics (e.g., patients within a specified age group (e.g., children), or having particular disease characteristics). For purposes of description herein a therapy that is to be studied or is being studied in a clinical trial, e.g., a Phase I, II, or III clinical trial (e.g., to assess safety and/or efficacy) may be referred to as an “experimental therapy”. In some embodiments an experimental therapy may comprise any agent intended for diagnosis, treatment, or prevention of a disease. In some embodiments an experimental therapy comprises a drug or device. In some embodiments a drug or device has not been approved for commercial use for treatment of humans. For example, a drug may be a new chemical entity. A drug, e.g., a new chemical entity, may comprise, e.g., a small molecule, protein, antibody, nucleic acid (e.g., short interfering RNA), cell, vaccine, etc. In some embodiments a drug or device has been approved for commercial use for treatment of humans but not specifically for the particular disease, indication, use, dose, administration route, patient population (e.g., age group), procedure, etc., in which it is studied in a trial, or not in a particular regimen or combination therapy in which it is studied in a trial. In some embodiments an experimental therapy comprises surgery and/or radiation. In some embodiments an experimental therapy comprises surgery and/or radiation and one or more drugs or devices. In some embodiments an experimental therapy comprises or consists of a therapy that is a candidate for repositioning. In some embodiments a function of an ADM, ADM template, or EMR system used for purposes of a clinical trial or other use of an experimental therapy may be referred to as an “experimental therapies function”. In some embodiments, functions relating to clinical trials may be provided as or included in an Experimental Therapies component, which may be provided as part of an ADM component.

In some embodiments, an ADM-assisted study may, as appropriate, be registered with ClinicalTrials.gov or other appropriate public clinical trial registry. The information provided for such registration and posted on the ClinicalTrials.gov website (or other registry) may indicate that the study is an ADM-assisted study.

In some embodiments, a HCP who has at least one patient participating in an ADM-assisted clinical trial (or who is authorized to have patients participate in the trial but has not yet done so, e.g., because the trial has not yet started or because no patients of the HCP have yet been enrolled) and who agrees to comply with sponsor-specified requirements (such as completing or ensuring completion of the appropriate ADM) may be referred to as an “investigator”. An investigator may be a HCP who administers a drug or under whose immediate direction a drug or medical device may be administered or dispensed or deployed, or, may be a HCP who performs a medical or surgical procedure or under whose immediate direction such procedure is performed. If research is conducted by a team of individuals at a research site, an investigator may be the leader of the team and typically has overall responsibility for the conduct of a trial at a site. It will be understood that an investigator may have one or more co-investigators or associate investigators. Such individuals may all be referred to as investigators. It will also be understood that in some instances an investigator may be a pathologist, radiologist, or other medical specialist who may not be directly responsible for administration or deployment of an intervention for patient treatment. An investigator who serves as the sole investigator for a trial at a site or who serves as the primary investigator with overall responsibility for conduct of the trial at a site may be referred to as a “principal investigator”. A trial conducted at more than one site (multi-site trial) may have an investigator who oversees multiple sites, in addition to an investigator at each site who is responsible for the conduct of the study at that site. Such an investigator may be considered a principal investigator for the trial as a whole. It will be understood that an investigator may be a sponsor of the trial in certain embodiments.

The term “study personnel” may be used to refer to individuals involved in conducting a trial. An investigator at a site, e.g., a principal investigator, may delegate various study-related tasks to other study personnel, typically other study personnel who are employed by the entity that serves as the site. In addition to at least one investigator, study personnel at a site may include one or more nurses, physicians in training and/or one or more clinical research coordinators (CRC, sometimes referred to as “study coordinator” or “clinical study coordinator”). In general, a CRC may be an individual who conducts investigator-delegated tasks related to a study at an individual study site. Tasks that may be performed at least in part by CRCs include, e.g., preparing documents for submission to an IRB, recruiting subjects, screening potential subjects for eligibility in a trial, administering the informed consent process, entering data into case report forms, etc. In some embodiments an EMR system may at least in part perform or assist with performance of one or more tasks that may otherwise be performed by study personnel, e.g., a CRC. Clinical research associates (CRA, sometimes referred to as a “monitor”) are individuals who, among other things, may monitor studies remotely and/or by traveling to research sites on behalf of entities, e.g., companies, that sponsor clinical trials, or for CROs that are engaged by a sponsor to perform certain trial-related activities. Among other things, CRAs may review original documents and records (whether paper or electronic) for errors or omissions and/or for consistency with the data that has been provided to the sponsor or CRO or that has been entered into a clinical trial database. Sponsor or CRO personnel located away from the site, e.g., at a sponsor or CRO location, may detect certain types of errors, inconsistencies, implausible data, or omissions. They may then send queries to the site, prompting review and possibly correction.

In some embodiments systems and methods are provided herein in which an ADM-equipped EMR system is used to facilitate one or more aspects of subject recruitment, subject enrollment, and/or data collection for a clinical trial. For example, in some embodiments an ADM-equipped EMR system is used to facilitate screening or enrollment of subjects in a clinical trial. In some embodiments an ADM-equipped EMR system is used to identify one or more clinical trials for which a patient is eligible or may be eligible. In some embodiments an ADM template is used to facilitate clinical trial data collection, e.g., before a subject is enrolled in a trial (e.g., collection of screening data), after a subject is enrolled in a trial, or both.

In some embodiments, e.g., certain embodiments in which an ADM-equipped EMR system is used, at least some clinical trial data may be extracted from a standard EMR, imported into an ADM template, and used to populate the appropriate fields of the ADM template. In some aspects, extracting data directly from a standard EMR and importing it into an ADM template may reduce or, in some embodiments, may entirely avoid, the need to enter clinical trial data into a separate data collection system such as a standard EDC system and/or into a separate data collection form such as a case report form. In some embodiments an investigator or other study personnel (e.g., CRC) fills out at least some remaining fields of the ADM (i.e., those fields not populated by data imported from the patient's standard EMR. In some embodiments enrollment may comprise or be followed by randomization, e.g., to receive a particular intervention or placebo or be in particular arm of a study.

In some aspects the disclosure provides systems and methods in which an ADM template or ADM-equipped EMR system may be used for purposes of determining whether a subject is eligible or potentially eligible to participate in a clinical trial (screening). For purposes of description an ADM template that is adapted for clinical trial eligibility determination purposes may be referred to as a “screening ADM template” or ADM-SC template. In some aspects the disclosure provides systems and methods for generating an ADM-SC template. An ADM-SC template may differ from an ordinary ADM template in that it may comprise one or more data fields that are relevant to determining whether a patients meets or does not meet one or more inclusion criteria or exclusion (ineligibility) criteria for one or more trials, wherein such data field is not present in a typical ADM template for the disease of interest. In some embodiments a patient may be deemed “potentially eligible” for a trial if the patient has been determined to have a disease for which an experimental therapy is to be tested or is being tested in a clinical trial. In some embodiments a patient may be deemed “potentially eligible” in situations in which one or more additional items remains to be entered, evaluated, confirmed, and/or received in order for a definitive or final determination of eligibility to be made. For example, in some embodiments one or more items of data (e.g., a small number such as no more than between 1-5 items or no more than 1%, 5%, or 10% of the total number of items required to evaluate eligibility) may need to be entered, evaluated, and/or confirmed.

In some embodiments screening data comprises one or more data items that need to be assessed to determine whether a patient meets (satisfies) or does not meet an inclusion criterion for a trial or meets or does not meet an exclusion criterion for a trial. A data item relevant to clinical trial eligibility may be, e.g., an item of demographic information, medical history information, physical examination information, medication information (e.g., names of previous and current medications, start and stop dates of medications), laboratory values, imaging study result, diagnostic study result, pathology result, molecular diagnostic information, etc. A screening data item may or may not be a data item that is also part of a standard ADM template for a particular diagnosis. For example, an ADM template for lung cancer may ordinarily include a field for molecular information indicating whether the lung tumor is ALK positive. Such a data item may often be an inclusion criterion for a clinical trial of an ALK inhibitor, i.e., the patient must have an ALK positive tumor in order to be eligible to participate in the trial. However, the question of whether a patient has a malabsorption syndrome or other gastrointestinal illness that could affect oral absorption of the drug to be tested may not be part of a standard ADM template for lung cancer but may need to be considered for purposes of determining eligibility in a trial in which presence of such a disorder is an exclusion criterion. As another example, a standard ADM template for a particular diagnosis may not include fields for at least some health information that would typically be found elsewhere in an EMR, e.g., in a general Patient Data section and/or Patient Summary, e.g., age, concomitant illnesses, medications prescribed for other diagnoses, etc. However, such health information may be needed as part of screening data. In some embodiments an ADM-SC template may contain fields for at least some such health information. In some embodiments such health information may be imported into an ADM-SC template from wherever it may reside within an associated EMR.

In some embodiments an ADM-SC template is adapted for screening a patient for eligibility in a particular clinical trial. For example, an ADM-SC template may comprise data fields that are sufficient, when completed, to allow determination of patient eligibility for a particular trial of interest. In some embodiments an ADM-SC template is adapted for screening a patient for eligibility in any of multiple clinical trials. For example, an ADM-SC template may comprise data fields that are sufficient, when completed, to allow determination of patient eligibility for each of multiple trials. For purposes hereof, a set of one or more clinical trials may be referred to as a “trial set”. In some embodiments a trial set consists of 2, 3, 4, 5, 10, 15, 20, or more trials. In some embodiments a trial set consists of one or more trials of therapies intended for subjects in need of treatment for a particular disease of interest. In some embodiments a trial set consists of one or more trials having a particular HCP as an investigator, e.g., as a principal investigator. In some embodiments a trial set consists of one or more trials available at a particular site, e.g., for therapies intended for treating a particular disease of interest. In some embodiments a trial is considered “available” if it is open for enrollment or is expected to be open for enrollment within a reasonably short time period, e.g., within the following 1, 2, 3, or 4 weeks. In some embodiments a trial set consists of one or more trials available within a particular geographic region. A geographic region may be defined in any reasonable way. In some embodiments a geographic region corresponds to a city, metropolitan area, county, state, province, or country. In some embodiments a geographic region corresponds to a reasonable distance, e.g., a reasonable driving distance, from a particular location, e.g., a distance that a patient could reasonably be expected to travel in order to participate in a clinical trial. In some embodiments a reasonable distance is up to 25, 50, 75, 100, 150, or 200 kilometers. In some embodiments a reasonable distance is up to 25, 50, 75, 100, 150, or 200 miles. In some embodiments a trial set consists of one or more trials available within a particular network, e.g., a network of health care organizations or investigators. In some embodiments a trial set consists of one or more trials sponsored by a particular sponsor or sponsors. In some embodiments a disease of interest is cancer, e.g., a cancer of a particular type or subtype. In some embodiments a cancer type affects a particular organ. In some embodiments a trial set consists of Phase III trials. In some embodiments a trial set may consist of a set of trials selected by a business entity, sponsor, investigator, health care organization, or network of health care organizations. In some embodiments a network is the National Comprehensive Cancer Network (http://www.nccn.org/index.asp). In some embodiments a network is a cooperative group, e.g., one of the cooperative groups that together comprise the US National Cancer Institute's (NCI's) National Clinical Trials Network, e.g., the Eastern Cooperative Oncology Group (http://www.ecog.org), European Organisation for Research and Treatment of Cancer (http://www.eortc.be/default.htm), Children's Oncology Group (http://www.childrensoncologygroup.org), SWOG (http://www.swog.org), etc. In some embodiments trials in a trial set conform to a uniform set of guidelines, regulations, or policies. For example, trials may conform with at least some or, in some embodiments, all policies of a cooperative group. In some embodiments a method comprises selecting a set of trials to be included in a trial set. In some embodiments a method comprises generating an ADM-SC template that comprises data fields appropriate to permit determination of patient eligibility in each trial in a trial set. In certain embodiments a trial set may consist of trials for a specified disease that are available within a specified network or at a specified site. For example, a trial set may consist of trials for treatment of glioblastoma in adults that are available at sites that are members of a particular cooperative group, e.g., members located in a particular geographic region.

An ADM-SC may be updated as trials are added to or removed from a trial set and/or if eligibility criteria change during the course of a trial. For example, when a trial for an ET intended to treat a particular disease becomes available at a site, the trial set for ETs for that disease at the site may be updated to include additional data items that would be necessary to determine patient eligibility for the new trial. When recruitment for a trial in a trial set is complete, the screening data specifically required to determine eligibility for that trial may be removed from the ADM-SC for that trial set. An ADM-SC may be updated as available trials change or on a regular basis, e.g., weekly, monthly, etc. In some embodiments the task of updating an ADM-SC may be performed by the site or network that uses the ADM-SC. If eligibility criteria change during the course of a trial (e.g., if a potential adverse effect warranting exclusion of certain potentially vulnerable patients is recognized), the ADM-SC may be updated appropriately. In some embodiments updating an ADM-SC may be performed by a business entity that at least in part owns, controls, makes, sells, or provides an ADM component or ADM-equipped EMR system. In some embodiments a sponsor, site, or network provides eligibility criteria to a business entity that at least in part owns, controls, makes, sells, or provides an ADM component or ADM-equipped EMR system. The business entity may, if appropriate, convert the criteria into data fields appropriate for inclusion in an ADM-SC. The data fields may be subjected to a quality assurance process. For example, data fields may be required to adhere to a uniform set of units, avoid ambiguous questions, etc. Methods of designing data collection instruments, e.g., best practices, known to those of ordinary skill in the art, may be employed. The business entity may provide the data fields to the sponsor and/or to sites or networks at which the trial is or may become available. The data fields may be used to update the appropriate ADM-SC(s). For example, the site or network may perform the task of updating the appropriate ADM-SC. In some embodiments the business entity may maintain control of the ADM-SCs and may, e.g., update them remotely. In some embodiments, sites or networks interact with the business entity to inform the business entity of which trials are available at the site or in the network. In some embodiments data fields in an ADM-SC may be tagged with metadata indicating the particular trial or trials for which completion of such data field is required to determine eligibility. Such tags may facilitate removal of the screening data from an ADM-SC when the corresponding trial is removed from a trial set. In some embodiments updating and/or managing ADM-SC templates is provided as a service, e.g., to sponsors, sites, networks, etc., in exchange for a fee. A business entity may maintain a database comprising ADM-SCs. The database may further comprise additional information, such as, the trials for which an ADM-SC is appropriate for screening, sites at which such trial is or will be or was available, and/or sponsor of such trial, etc.

In some embodiments at least some EMRs in an EMR system suitable for use in an ADM-assisted clinical trial may comprise an “Experimental Therapies” (ET) link, which link may be used to access ADM template(s), ADM(s), or ADM-associated functions useful for one or more clinical trial purposes. In some embodiments an ET link may bear an identifier such as “Experimental Therapies”, “Clinical Trials”, “Experimental Protocols”, “Clinical Studies”, “ET” or any other symbol, word or phrase, or combination thereof, that indicates its purpose, identity, or connection with clinical trial(s). In some embodiments an ET link may have one or more characteristics that distinguish(es) its appearance from that of other elements used in an EMR. For example, an ET link may have a distinctive color(s), shading, shape, size, border, and/or logo and/or may use a distinct font as compared with at least some links in EMRs of the EMR system in which it is used. In some embodiments one or more characteristics of an ET link are selected to match those of at least some elements used in an EMR system in which the ET link is used, e.g., so that the ET link appears consistent with other elements of the EMR rather than appearing out of place. For example, an ET link may have the same color, shading, shape, and/or size as at least some elements in the EMR. In some embodiments an ET link uses the same font as do at least some elements in the EMR. In some embodiments an ET link appears on each screen of an EMR. In some embodiments an ET link appears on only one screen or one some but not all screens of an EMR. In some embodiments an investigator, investigator designee, or site may select from various options as to where an ET link appears in an EMR. In some embodiments options may be selected when an ADM component is installed and/or may be selected or changed after installation of an ADM component, e.g., via an “Options” menu.

In some embodiments, clicking an ET link brings a user into an ADM environment or into a particular portion of an ADM environment in which ADM templates adapted for use in clinical trials are available and/or ADM-associated functions specifically useful for one or more clinical trial purposes are provided. For example, in some embodiments clicking an ET link opens an ADM template, which may be an ADM-SC template or a trial-specific ADM template or an ADM. In some embodiments, a display element such as a ribbon appears, e.g., at or near the top of the screen, when the link is accessed, indicating that the Active Diagnosis Module environment or an ADM template or ADM has been entered. The ribbon may have a distinct color as compared with the background color of the screen. In some embodiments the ribbon may be purple. Other display elements may be used to indicate that the ADM environment has been entered, e.g., as described above. In some embodiments the ADM environment may be entered as a new window on the screen. In some embodiments a message is displayed to indicate that the ADM environment has been entered. The message may state, e.g., “You have now entered the Active Diagnosis Module” or “You have now entered the Active Diagnosis Module Environment”. In some embodiments a message is displayed to indicate that a portion of an EMR or ADM environment relating to experimental therapies has been entered. The message may state, e.g., “You have now entered the Experimental Therapies Module” or “You have now entered the Experimental Therapies Environment”. For example, in an EMR system that uses ADMs to at least in part fulfill its ordinary (non trial-related) medical record keeping functions, it may be appropriate to indicate when trial-related functions or trial-related ADMs are available or being used. In an EMR system that uses ADMs only for purposes relating to clinical trials (e.g., subject enrollment, EDC), informing the user that the ADM environment or an ADM has been entered may be sufficient to inform the user that trial-related functions or trial-related ADMs are available.

In some embodiments visibility of the ET link and/or access to the trial-associated functions may be restricted to a selected group of individuals, physical locations, and/or devices (e.g., particular computers). For example, in some embodiments, the ET link is visible and the trial-associated functions are accessible only to individuals involved in the conduct of the trial who have a need to view and/or enter data relevant to the trial into the subject's ADM. Such individuals may include investigators, CRC(s) for the trial at the site (where applicable), and, in some embodiments, individuals who are not investigators in the trial but are involved in providing care to the subject as part of the trial. In some embodiments an ET link is visible to at least some individuals who access a subject's EMR but are not involved in the trial. However, the link and/or at least some of the trial-associated functions may be nonfunctional to such individuals. In such embodiments, for example, a HCP who treats the subject for a condition not related to the clinical trial and accesses the subject's EMR may see the ET link and thereby be made aware that the subject is participating in a clinical trial but would not be able to view or modify the trial-specific fields of the ADM. In some embodiments such a HCP would be able to view but not modify at least some fields of the ADM, e.g., the trial-specific fields of the ADM. In some embodiments, information entered into the subject's EMR outside the context of the trial (e.g., results of tests ordered by HCPs not involved in the trial, medications prescribed by HCPs not involved in the trial, etc.) is automatically captured and entered into the ADM if relevant. In some embodiments such information is not entered into a data field intended for capture of data required by the protocol. In some embodiments such information may be documented within a special section of the ADM. In some embodiments such information may, if appropriate, be documented as a protocol deviation. Which information is considered “relevant” may differ depending on the particular trial. In some embodiments, capturing such information may, for example, reduce the likelihood of protocol deviations or violations (e.g., taking of medications of the subject that are not allowed under the protocol); permit detection of adverse events (AEs that may be related to the study drug/device or unrelated AEs) in a more comprehensive and/or more timely manner than may otherwise be the case. Thus, in some embodiments, by capturing information generated outside the context of a trial, an EMR system may enhance the quality of the trial and/or may improve subject safety.

In some embodiments an ADM-equipped EMR may be used as follows: A patient who may benefit from experimental therapy visits a physician. The physician may or may not be an investigator. The physician recognizes that the patient may benefit from experimental therapy, contacts a clinical research coordinator (CRC), e.g., a CRC working at a site where a trial is available, and indicates to the CRC that the patient may be a candidate for experimental therapy. (It is sometimes assumed in the following description for purposes of convenience that the individual interacting with the EMR system is a CRC. However, it may be the physician or any appropriate individual authorized to view the patient's health information. It should also be understood that individuals such as parents, legal guardian, health care proxy, or other representatives of the patient may be involved in the process of selecting a clinical trial as well as or in addition to the patient.) The CRC may speak with the patient and explain which experimental therapies and/or which clinical trials may be available, e.g., at the site. In some embodiments the CRC is trained to be knowledgeable regarding multiple experimental therapies and/or clinical trials available at the site, e.g., multiple ETs for the patient's disease and/or trials of such ETs. The CRC accesses the patient's EMR in an ADM-equipped EMR system that contains ET functionality. As described above, a link is present in the EMR (e.g., in the “Medication” section) to “Experimental Therapies”. The CRC accesses the link (e.g., by clicking on it). An indicator and/or message that the ADM environment, ADM, or Experimental Therapies Module has been entered appears, e.g., as described herein. In some embodiments, the CRC enters a tentative diagnosis, e.g., into an ADM template. The tentative diagnosis will typically be the disease for which the physician recognized that experimental therapy may be appropriate and will typically be evident to the CRC, e.g., from interaction with the physician or patient or from the patient's existing EMR. In some embodiments at least some data already present in the patient's EMR are imported and used to populate the appropriate fields in the ADM. In some embodiments the CRC or physician or another HCP is required to confirm the accuracy of at least some imported data. The CRC may fill in at least some data items that are missing (e.g., data items that are not already populated based on the existing content of the patient's EMR). In some embodiments the required data include those data items required by the ADM to establish the tentative diagnosis as a definitive diagnosis, e.g., as described herein.

After a definitive diagnosis is established, the ADM may be modified to include additional fields for entry of screening data, i.e., it may become an ADM-SC for the particular diagnosis that was established as definitive. (Of course if the EMR system already uses ADMs as its primary means of maintaining the patient's health information, the EMR may already include an ADM with a definitive diagnosis of the disease for which ET is sought but may, in some embodiments, lack at least some screening data relevant specifically to clinical trial eligibility. In such instances, the CRC clicks the Experimental Therapies link (e.g., within the ADM relating to the disease for which experimental therapy is sought) and the ADM is modified to become an ADM-SC for that disease.)

In some embodiments at least some data is required to be entered into an ADM-SC based on requirements of one or more clinical trial protocols that are available in a trial set, e.g., the additional data comprises screening data for a trial set. In some embodiments the trial set consists of one or more trials available at the site at that time for the patient's disease. For example, if the patient is to be screened for potential enrollment in clinical trials for treatment of lung cancer, an ADM template suitable for a diagnosis of lung cancer would be used, augmented by additional data fields (if any) required to assess eligibility for at least one clinical trial in lung cancer that is being conducted at the site. The CRC may be prompted to enter at least some screening data that is missing in the ADM-SC. In some embodiments the system checks whether the ADM-SC is filled out satisfactorily.

In some embodiments, after entry of sufficient data to establish a definitive diagnosis, and, in some embodiments, the CRC may be presented with or permitted to open a list of experimental therapies and/or clinical trials for therapies relating to the definitive diagnosis. In some embodiments the experimental therapies and/or clinical trials are those within a particular clinical trial set, e.g., clinical trials available at the site for patients having the definitive diagnosis. In some embodiments the CRC selects a particular therapy or a particular trial. In some embodiments the CRC may select two or more therapies or trials. In some embodiments, if the CRC selects a particular therapy (or therapies), then following such selection the ADM is modified or updated to become an ADM-SC that contains fields for entering screening data appropriate for determining eligibility for those trials within a trial set (e.g., a trial set consisting of trials available at the site) that involve use of that particular therapy (or therapies). For example, if the patient has glioblastoma, a therapy may be temozolomide. There may be multiple trials involving use of temozolomide within a trial set, e.g., available at a site. The ADM-SC may contain fields for entry of screening data sufficient to determine eligibility of the patient for each of these trials. The CRC then enters the required screening data for those particular trials. In some embodiments, if the CRC selects a particular trial or trials (rather than a particular therapy or therapies), then following such selection the ADM is modified or updated to become an ADM-SC that contains fields for entering screening data appropriate for determining eligibility for that particular trial or those particular trials.

In some embodiments satisfactory completion of the ADM-SC requires entry of data at least sufficient to determine eligibility for all clinical trials in a trial set, e.g., all trials for ETs for the disease that are available at the site. The CRC may be prompted to continue entering data until the ADM is satisfactorily completed with all such data. Following entry of all such data a list of trials for which the patient is deemed eligible appears. The CRC may select a trial and, e.g., send a request to the sponsor that the patient be permitted to enroll in the trial. In some embodiments an iterative data collection and checking process may be used rather than requiring entry of all screening data for trials in a trial set. For example, a relatively small set of screening data may initially be required to be present in the ADM-SC. The screening data is checked against inclusion and/or exclusion criteria for the trial set. Based on this initial check, a list of trials for which the patient may be eligible is generated. Additional data items are then requested, but only or primarily those items that are actually relevant to inclusion and/or exclusion criteria for at least some trials that appeared on the initial list. The process continues in this manner, and the list is narrowed down to the trials for which the patient is likely to be eligible. In some embodiments the order in which the CRC is prompted to enter data is prioritized, beginning with general data items (which are likely to be available or readily ascertainable for many or most patients) and proceeding to specific data items that are relevant to only one or a small number of trials in the trial set. In this manner, collection and entry of data may be reduced as compared with an approach that would require entry of all data pertinent to trial eligibility.

In some embodiments the CRC may be prompted to enter at least some further data (in addition to the data required to establish a definitive diagnosis and to determine eligibility for at least one trial) in order to satisfactorily complete the ADM. In certain embodiments the data required to satisfactorily complete the ADM include at least some data that would ordinarily be required for an acceptable ADM for the particular disease of interest. This additional data may be required to be entered before the screening data is required or afterwards in various embodiments. In some embodiments an ADM that is filled out for screening purposes (ADM-SC) need not include all data that would ordinarily be required to be provided for an acceptable ADM for the disease of interest. For example, certain data that is unknown and/or not ascertainable, e.g., from the patient, physician, or existing contents of the patient's EMR, may be entered as “unknown” or “not available”.

In some embodiments, data that requires input or verification by a physician, e.g., an investigator, may be entered or verified separately, e.g., after entry of other data by the CRC or after an initial determination of trial eligibility. In some embodiments a physician, e.g., an investigator, may confirm at least some of the data prior to submission of a request to a sponsor. For example, certain screening data may require that a patient meets certain criteria according to the opinion of the investigator.

In some embodiments, completing the ADM-SC may require performing one or more tests on the patient or obtaining results of one or more studies on the patient. For example, a female patient may need to be documented as having had a negative pregnancy test. As another example, a clinical trial involving administration of a vaccine may require that the patient is documented as having a negative HIV test. In some embodiments a HCP or CRC is able to order one or more tests or studies, results of which are required as screening data, from within a ADM-SC. In some embodiments, results of tests or studies ordered from within an ADM-SC are entered automatically into the ADM after they become available. In some embodiments results of tests or studies ordered from within an ADM-SC may be entered or available only within the ADM-SC and not from elsewhere in the EMR.

In some embodiments, following at least partial completion of a ADM-SC, a list with available therapies and/or corresponding clinical trials for which the patient appears to be eligible is provided (e.g., as a list on the screen). In some embodiments, if the patient is deemed not eligible for a particular trial (or trials) an indication of the reason(s) that the patient was deemed not eligible may be provided. The relevant data items that resulted in a determination of ineligibility may be further evaluated (e.g., a test may be repeated for confirmation purposes) or considered by the CRC, physician, an investigator on the particular trial, or a sponsor of the trial. In some embodiments, e.g., if a patient is deemed ineligible for a particular trial, information is provided regarding a managed access program, e.g., an expanded access program, that uses the same experimental therapy and for which the patient may be eligible.

In some embodiments, e.g., after presentation of a list of one or more trials for which the patient is eligible or potentially eligible, the CRC clicks on (or otherwise selects) a trial of choice. Clicking on the link may bring up additional information regarding the trial. In some embodiments an option is provided whereby a request that the patient be permitted to enroll in the trial may be sent electronically to the sponsor. A decision as to which trial to select may be made by the patient, physician, or CRC in various embodiments. In some embodiments the selection is made through consultation, e.g., between the patient and physician, or in any manner appropriate to the circumstances.

In some embodiments the patient may meet with an investigator in a particular trial in which he or she desires to enroll. In some embodiments assent of an investigator in a particular trial in which the patient desires to enroll is required in order for such enrollment to proceed. Such assent may be indicated in a field of the ADM-SC. In some embodiments, an email, text message, or other electronic alert is sent to an investigator for a trial in which a subject has been deemed potentially eligible to participate. In some embodiments the investigator may access the EMR system, view the ADM-SC, and indicate (e.g., by checking a box) whether enrollment may proceed.

In some embodiments an investigator on the particular trial must make or approve a request to the sponsor prior to the request being submitted to the sponsor. In some embodiments a request to the sponsor may include or be accompanied by the ADM-SC or at least sufficient data from the ADM-SC for the sponsor to determine whether to permit the patient to enroll in the trial. In some embodiments the data is sent in a de-identified manner. In some embodiments the sponsor may access the ADM-SC electronically to review the data. After receiving the request and reviewing the data the sponsor responds and, e.g., approves or rejects enrollment of the patient in the trial, requests additional information, requests confirmation of one or more data items, etc. In certain embodiments it is envisioned that the sponsor may respond within one hour of the request being sent or, in the case of a request sent outside the working hours of the person responsible for responding, within one hour of the start of working hours of such person. In some embodiments the sponsor may respond within the ADM-SC. In some embodiments the sponsor may respond by email, text message, fax, or other means.

If the patient is accepted into the trial, enrollment may proceed, e.g., the patient may be asked to read and sign an informed consent form. In certain embodiments a CRC, physician, investigator, or sponsor personnel may provide an electronic signature in connection with entering or confirming a data item, request, response, etc.

In some embodiments, if a patient is not accepted into a particular trial (e.g., the first choice of the patient or physician), the CRC may submit requests for the patient to be permitted to enter other clinical trials, e.g., within the same trial set or a different trial set. Even if none of the experimental therapies can be offered to the patient, a well-completed ADM may nonetheless have been created in certain embodiments. In some embodiments the ADM may be used to identify the patient as a potential subject for future trials. With the consent of the patient, the data of the ADM may be used, in a de-identified fashion, to conduct research, e.g., as described herein. In some embodiments at least some of the screening data may be analyzed for one or more purposes other than determining eligibility. Such analysis may, for example, help identify criteria that contribute to low or lower than expected enrollment.

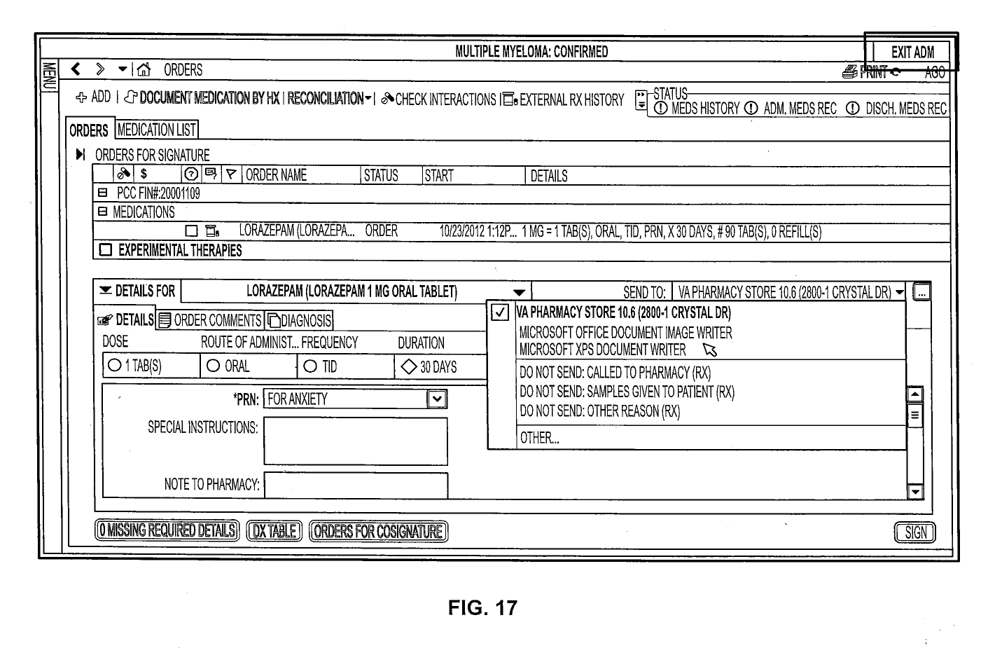

In some embodiments, by clicking on an appropriate area of the screen, e.g., the ribbon described above, the ADM-SC may be exited for a return to, e.g., the default EMR settings, home screen of the EMR or the screen from which the ADM environment was entered. In some embodiments the ADM-SC (or a screen within the ADM-SC) may be exited by clicking an “X” in a corner, e.g., an upper corner (e.g., the upper right corner) of the screen. In some embodiments a link or icon labeled “exit” or “return to EMR” or similar language is present within the ADM and can be used to return to the default settings of the EMR, home screen of the EMR or to the screen from which the ADM environment was entered (see, e.g., FIG. 17).

In some embodiments, once a subject is enrolled in a trial, the ADM is used for electronic data capture purposes during the trial. In some embodiments an ADM template, e.g., an ADM-SC template, is modified or updated to include fields for data to be collected for purposes of the trial, e.g., the ADM-SC is changed to a trial-specific ADM for the particular trial in which the patient enrolls. An ADM used or adapted for purposes of clinical trial data collection may be referred to as an ADM-EDC. In some embodiments an ADM-EDC contains one or more data fields specifically included for purposes of clinical trial data collection. Such a data field may be for data that would not ordinarily be collected as part of normal standard of care treatment of the subject. In some embodiments the sponsor of a trial and/or a CRO for the trial is able to access and view ADM-EDC(s). The sponsor and/or CRO may thereby be able to at least in part monitor the trial remotely. In some embodiments a data safety monitoring board (DSMB) and/or regulatory agency, e.g., the FDA, is able to access and view ADM-EDC(s). The regulatory agency may thereby be able to at least in part regulate or evaluate the trial or evaluate or address potential subject safety concerns remotely. The DSMB may thereby be able to at least in part evaluate or address potential subject safety concerns remotely. In some aspects, an EMR system permits a sponsor or CRO to employ centralized subject selection and/or monitoring. A sponsor, CRO, DSMB, or regulatory agency may, in some embodiments, access and, in some embodiments may analyze, data collected from multiple sites and/or in multiple trials, through a single portal. In some embodiments, for example, a sponsor may log on to a website or other portal that provides access to data collected in any of multiple trials sponsored by that sponsor, wherein such trials have been or are being conducted through use of one or more ADM-equipped EMR systems. In some embodiments, for example, a CRO may log on to a website or other portal that provides access to data collected in any of multiple trials for which such CRO is providing services, wherein such trials have been or are being conducted through use of one or more ADM-enabled EMR systems. In some embodiments, for example, a regulatory agency or DSMB may log on to a website or other portal that provides access to data collected in any of multiple trials over which such regulatory agency has jurisdiction or for which such DSMB has responsibility. In some embodiments an ADM template, e.g., an ADM-SC or ADM-EDC template, may be updated during the course of a trial, e.g., if the protocol is amended during the trial, e.g., in order to reflect such amendment(s). In some embodiments an ADM-equipped EMR system may be used in an adaptive trial, e.g., a trial in which data gathered as the trial progresses may be used to alter the design of the trial. For example, a treatment arm that is ineffective may be dropped; a trial size may be increased; dose levels may be altered, etc., based at least in part on data gathered during the trial. In some embodiments an ADM-equipped EMR system facilitates adaptive trial designs and/or rapid proof-of-concept trials. In some embodiments an ADM template, e.g., an ADM-SC or ADM-EDC template, may be modified if a trial design is changed during the trial, e.g., in order to reflect such change(s).

In some embodiments an ADM-EDC may import data from elsewhere in an EMR, e.g., as such data is entered or subsequently. In some embodiments an ADM-EDC may interface directly with a site's usual computer system for providing test results, so that such test results are or can be imported directly into the ADM-EDC when available.

In some embodiments an ADM-EDC may distinguish or allow distinction between those subject visits, tests, and/or procedures that would ordinarily be part of the normal standard of care for a patient having the disease for which the experimental therapy is being tested in the trial and those that occur or are ordered specifically to comply with requirements of the clinical trial protocol. In some embodiments an ADM-EDC may distinguish or allow distinction between those subject visits, tests, and/or procedures that would ordinarily be part of the normal or typical standard of care for the patient and those that occur or are ordered specifically to comply with requirements of the clinical trial protocol. In some embodiments such distinguishing may facilitate appropriately allocating the cost associated with such subject visit, test, procedure, etc., between the sponsor and an appropriate payor or the subject. For example, the sponsor may be responsible only for payment for those visits, tests, and/or procedures that are specifically required to comply with the protocol. In some embodiments the sponsor may not be responsible for payment for at least some visits, tests, and/or procedures that are part of the ordinary standard of care for patients having the disease for which the experimental therapy is being tested in the trial. In some embodiments the sponsor may not be responsible for payment for at least some visits, tests, and/or procedures that are performed for purposes of monitoring or treating conditions that the subject is documented to have already had prior to entering the trial. In some embodiments the sponsor may not be responsible for payment for at least some visits, tests, and/or procedures that are performed for purposes of diagnosing and/or for purposes of treating conditions that are determined to be unrelated to the experimental therapy or trial. In some embodiments a normal or typical standard of care for patients with a particular disease may be determined at least in part by analyzing EMRs of patients the disease who are not receiving experimental therapy. In some embodiments such EMRs comprise standard EMRs. In some embodiments a normal or typical standard of care for patients with a particular disease may be determined at least in part by analyzing ADMs of patients with the disease who are not receiving experimental therapy. In some embodiments a standard of care may be a national standard. In some embodiments a standard of care may be determined within a geographic region, network, and/or within one or more health care organization(s). In some embodiments a standard of care may be determined based at least in part on guidelines promulgated by professional associations of various medical/surgical specialties or subspecialties, by expert panels or committees of HCPs in the relevant disease area, or by national or international organizations or government medical research institutes, or other bodies. In some embodiments the EMR system may provide a document that sets forth requirements to which a HCP must agree in order for the HCP to be an investigator in an ADM-assisted study and/or checks to ensure that such a document maybe appropriately completed and entered into the EMR system before permitting a HCP to serve as an investigator in an ADM-assisted trial.

In some embodiments the EMR system may provide informed consent documents and/or checks to ensure that such a document may be appropriately completed and entered into the EMR system before permitting a subject to be enrolled in an ADM-assisted trial.

In some embodiments the EMR system may send reminders to subjects regarding upcoming scheduled visits or otherwise assists in scheduling patient visits or follow-up.

In some embodiments access to an ADM being used in an ADM-assisted trial, or access to at least some trial-specific fields may be restricted, as compared, for example, with accessibility of the subject's other ADMs and/or as compared with one or more other portions of a subject's EMR. In other words, only a subset of users of the EMR database may be able to access such ADMs or trial-specific fields. In some embodiments, at least during the course of an ADM-assisted clinical trial, access to the ADMs or trial-specific additional fields may be limited to the subject's HCP who is serving as an investigator or their designees (or, e.g., may also be provided to at least some of the subject's other HCPs, if any) and/or to the sponsor or individuals or entities authorized by the sponsor (e.g., a CRO). Without limiting the foregoing, in some embodiments, ADMs being used in a clinical study or trial-specific fields thereof, may not be accessed by subscribers to the EMR database (other than the sponsor or sponsor-authorized subscribers). In some embodiments, the complete ADMs may be made available to subscribers after a specified time period (e.g., between 3 months and 5 years) has elapsed following termination or completion of the trial or when results of the trial are published.

In some embodiments an EMR system, e.g., an ADM-equipped EMR system that uses ADM-EDC, provides real-time checking during a subject visit to a site as clinical trial data is entered. An EMR system may prompt an investigator to enter required data, order required tests, etc., in order to properly complete the ADM-EDC.

In some aspects, the use of an ADM-equipped EMR system for EDC may improve certain aspects of collecting clinical trial information relative to use of a standard EDC system. For example, the use of ADM-EDC may reduce the amount of data that may need to be entered (e.g., typed, scanned, etc.) more than once, may reduce the average amount of time spent on data entry on the part of trial personnel at a site, may reduce or eliminate a time lag between acquisition of data and entry of such data into a CRF or clinical trial database, may reduce the number of errors, inconsistencies, and/or omissions, and/or may reduce the number of queries on the part of the sponsor or CRO, may reduce the average amount of time spent on monitoring by sponsor or CRO personnel, etc.

In some embodiments use of an ADM-equipped EMR system, e.g., for EDC, may reduce the likelihood that a site or trial will fail to comply with one or more protocol requirements, IRB requirements, requirements of a sponsor, or regulatory requirements or guidelines, e.g., one or more requirements or guidelines relating at least in part to data collection, e.g., requirements or guidelines relating to accuracy, completeness, or timeliness of data collection, timeliness of reporting adverse events, etc, as compared with use of a standard EDC system. In some embodiments use of an ADM-equipped EMR system may reduce differences in data collection practices between different sites of a multi-site trial.

In some embodiments guidelines comprise Good Clinical Practice (GCP) Guidelines. Good Clinical Practice (GCP) is an international quality standard for clinical trials involving human subjects that is provided by International Conference on Harmonisation (ICH). ICH is an international body that brings together regulatory authorities of Europe, Japan and the United States and representatives from the pharmaceutical industry in the three regions to discuss scientific and technical aspects of pharmaceutical product registration. In some embodiments regulations or guidelines comprise government regulations or guidelines that are at least in part based on or at least in part incorporate GCP guidelines.

In some embodiments an EMR system that uses ADM-EDC may send or facilitate sending certain clinical trial data to a quality assurance center or data repository or may indicate whether certain clinical trial data has been reviewed by a quality assurance center and/or determined to meet appropriate quality standards. In some embodiments an EMR system may require that certain clinical trial data is entered in a format suitable for evaluation or acceptance by a quality assurance center or data repository. In some embodiments an EMR system may require that certain clinical trial data has been reviewed by a quality assurance center and determined to meet appropriate quality standards in order for such data to be accepted by an ADM template. A quality assurance center may be, e.g., the Quality Assurance Review Center (QARC) (http://www.qarc.org/). In some embodiments a quality assurance center or data repository is mandated or approved by a government entity (e.g., the FDA, NIH, or an NIH institute such as the NCI), cooperative group, or network. In some embodiments an EMR system may require that certain clinical trial data has been gathered using an instrument or device (e.g., an imaging device) that is certified or otherwise determined to have been properly calibrated and/or to be functioning appropriately. In some embodiments an EMR system may require that certain experimental therapy being studied in a clinical trial data has been administered using an instrument or device that is certified or otherwise determined to have been properly calibrated and/or to be functioning appropriately. An ADM may include fields indicating that a data item or an ADM has or has not been submitted to or approved by a quality assurance center or submitted to or accepted by a data repository.

In some embodiments a system, e.g., a system useful for clinical trial purposes, comprises an EMR system, one or more ADM templates (which may be provided via an ADM component), and an EDC system. In some embodiments such a system comprises an EMR system, an ADM component, and an EDC system. In some embodiments an EMR system comprising one or more ADM templates, e.g., provided via an ADM component, interfaces with an EDC system. In some embodiments an ADM template or ADM component interfaces both with an EMR system (e.g., a standard EMR system equipped with an ADM plug-in) and with an EDC system. Thus an EDC system may be an ADM-interfaced EDC system in certain embodiments. In some embodiments an interface between an ADM template or ADM component and an EDC system is provided via a plug-in to the EDC system. In some embodiments at least some trial-relevant data may be entered directly into an ADM (e.g., by a CRC). In some embodiments at least some trial-relevant data may be extracted by the system from a patient's standard EMR and used to populate the appropriate fields in an ADM. If the patient is or becomes a subject in a clinical trial or, in some embodiments, if the patient is screened as a potential subject in a clinical trial, at least some of the data may be copied by the system (e.g., by the ADM or ADM component) from the ADM into a record for that individual in an EDC system. In some embodiments the ADM is or is replaced by an ADM-EDC. In some instances the data is sufficient to fulfill the data requirements of the EDC system for the trial. In some instances the data may not be sufficient to fulfill the data requirements of the EDC system for the trial. If additional data is required, the data may be obtained, e.g., from the individual's EMR, by a CRC or other study personnel and entered into the EDC system. In some embodiments the use of ADMs may reduce but not eliminate the need for data entry into an EDC system by study personnel. The EDC system may be used as usual by sponsors, CROs, monitors, or others, without a need for such users to become familiar with the ADM format. In some embodiments a sponsor or CRO may utilize legacy or proprietary EDC software and/or may utilize legacy or proprietary software in conjunction with an EDC system. For example, such software may extract data from the EDC system and enter it into a database for the trial. In certain embodiments a system that comprises an EMR system, ADM templates (e.g., an ADM component), and an EDC system allows sponsors, CROs, etc., to benefit from reduced data entry requirements and/or reduced number of human errors through use of ADMs, while retaining the option to use EDC systems and/or associated software. In certain embodiments an EDC system may be used in parallel with ADM-EDC for data collection, e.g., in a clinical trial. For example, ADM-EDCs may be used as described herein, and a conventional EDC system not interfaced with EMRs may also be used. Using two systems for clinical trial data collection (a system employing ADM-EDC and a “stand-alone” EDC system) may improve overall accuracy and/or completeness of data entry than would be the case if the EDC system alone was used. Performance of the two systems may be compared.

In some embodiments trial-specific ADMs, ADM-SCs, and/or ADM-EDCs are compatible with an ordinary, non-trial related ADM for the particular disease. Additional fields or functions, if any, required for screening and/or trial-related purposes may be provided as extensions or versions of an ordinary, non-trial related ADM template. An ADM template for a disease may be designed to accommodate such fields or functions.

In some embodiments, it is envisioned that the EMR system may be used to facilitate clinical studies that may at least in part replace the currently mandated randomized controlled Phase III trial(s) required for regulatory approval of drugs in the U.S. and various other jurisdictions. For example, in some embodiments, following standard Phase I and Phase II trials a drug may be made available (e.g., by a sponsor) for use by physicians under a so-called “white label”. The white label would, in various embodiments, permit the drug to be administered to patients who meet certain criteria (“inclusion criteria”) or do not meet certain criteria (“exclusion criteria”) or at the physician's discretion. Inclusion or exclusion criteria may be agreed upon by the sponsor and the FDA. The sponsor and the FDA may agree on certain endpoint(s) that must be met in a white label trial in order for the drug to receive approval for marketing. Data that may be used to determine whether inclusion criteria and/or endpoints are met may be incorporating fields specifying entry of such data into an ADM template.

In some embodiments, at least one of two Phase III studies required for regulatory approval may be a white label, ADM-assisted study. In some embodiments, a standard Phase III trial (which may or may not be ADM-assisted) may overlap in time or may proceed substantially concurrently with a white label, ADM-assisted study. In some embodiments, a white label, ADM-assisted study may take place after a standard Phase III trial, e.g., a standard Phase III trial that demonstrates efficacy.

In some embodiments, a white label drug is made available to any HCP who agrees to use the EMR system and, in at least embodiments, agrees to use a designated ADM for patients to whom the drug may be administered. In some embodiments, a white label drug may be made available to only a subset of HCPs (provided such HCPs agree to use the EMR system and, in at least some embodiments, agrees to use a designated ADM for patients to whom the drug is administered). For example, a sponsor may desire to limit a study to HCPs who have at least a minimum number of patients with a disease of interest. In some embodiments, only a subset of patients who receive a white label drug may be evaluated for purposes of determining whether an endpoint is met in a clinical trial. For example, the criteria for permitting a white label drug to be used in a patient may be different to the inclusion criteria for a clinical trial. In some embodiments, safety-related data may be gathered via an ADM for all patients who receive the drug, but only a subset of patients (i.e., those who meet specified inclusion criteria) may be assessed for purposes of determining whether an endpoint is met. It may thus be possible to gather safety-related data in a large number of patients, which data may be considered along with efficacy data gathered in a smaller number of patients, for evaluating a drug for approval. In some embodiments, a study may be randomized. In some embodiments, a study may not be randomized. In some embodiments a study is at least in part blinded. In some embodiments a study may be at least in part not blinded (e.g., the HCP and, e.g., the patient, may be aware of which of various drugs is used). In some embodiments, a control group may be selected from among patients who have been diagnosed with the disease of interest but who are not receiving the drug being studied. The sponsor may select ADMs having appropriate characteristics for a control group from among the set of ADMs for the relevant disease in the EMR database. For example, in a clinical trial of a new cholesterol-lowering agent, a sponsor may select ADMs for patients who are diagnosed with elevated cholesterol levels within a particular time period and who receive typical standard of care therapy for use as a control group. In some embodiments, a matched control patient (i.e., a subject having characteristics comparable to those of a subject who is treated with a study drug but who receives a different treatment (e.g., a standard of care therapy, or any particular specified therapy) may be selected. In some embodiments, ADMs to be used as a control group may be selected randomly (e.g., from among ADMs that meet a predetermined set of criteria).

In some embodiments the EMR system may facilitate recruitment of HCPs for service as investigators and/or facilitates recruitment of subjects for participation in an ADM-assisted clinical study. For example, in some embodiments, if a HCP enters a tentative or definitive disease diagnosis into the EMR system or into an ADM, and a drug is available for the disease under a white label, the HCP may receive an alert informing him or her of the availability of the drug. In some embodiments the EMR system may periodically inform HCPs, e.g., physicians, of the availability of white label drugs. Such information may be provided at least in part based on HCP specialty. For example, oncologists may receive alerts regarding white label drugs for cancer treatment; ophthalmologists may receive alerts regarding white label drugs for macular degeneration or glaucoma; neurologists may receive alerts regarding white label drugs for multiple sclerosis, etc. In some embodiments a link to a web page for the study on ClinicalTrials.gov or other public clinical trial registry is provided. The information posted for the trial on the ClinicalTrials.gov website (or other registry) may indicate that the study is a white label study. In some embodiments, if a patient receives a tentative or definitive diagnosis of a disease for which a drug may be available under a white label, and such diagnosis may be entered into the EMR system or into an ADM, the patient may receive an alert informing him or her of the availability of the drug.

In some embodiments, any one or more of the functions or features described above relating to use of an EMR system, e.g., an ADM-equipped EMR system, in clinical trials may be employed in white label trial(s), and vice versa. In some embodiments a link specific for white label trials is provided, which is labeled in a manner to indicate its association with white label trials. For example, the link may be labeled “White Label Trials” or with any other suitable label. In some embodiments after clicking on an “Experimental Therapies” link a user may select a “Clinical Trial” link or a “White Label Trial” link. In some embodiments an ADM-SC template is designed to facilitate screening for or enrollment in a white label trial.

In some embodiments an ADM or ADM template, which may be a standard ADM or ADM template, ADM-SC, or ADM-EDC, may interface with, e.g., accept or transmit data from or through, an interactive voice response system (IVRS). Such system may comprise any system by which a computer may interact with humans through the use of voice, DTMF tones input via keypad, or other forms of phone-mediated and/or voice-mediated communication. Such systems may include, e.g., voice recognition, transcription, prerecorded messages or prompts, dynamically selected or generated voice messages, prompts, or responses, etc. In some embodiments ADM, ADM-SC, or ADM-EDC may at least in part perform or provide one or more functions of an IVRS.

In some embodiments a EMR system described herein may be used to facilitate one or more aspects of a managed access program. The term “managed access program” (MAP) refers to a program under which a drug or other medical product such as a medical device is made available, under a particular regulatory regime, to one or more patients with unmet medical needs in situations in which such patient(s) are not able to obtain the drug or medical product by participating in a clinical trial or through ordinary commercial routes, e.g., by prescription or over-the-counter. MAPs may be referred to by a variety of terms including, e.g., expanded access program, early access, compassionate use, named patient, pre-approval access, unlicensed use, or off-label use (in jurisdictions where off-label prescription is prohibited), and like terms. For the sake of brevity, for descriptive purposes herein it will generally be assumed that a MAP relates to a drug. However, the disclosure provides analogous embodiments in which a EMR system may be used to facilitate a MAP under which a device or other medical product that may not include a drug is provided. In some embodiments a EMR system may (i) determine whether a patient meets criteria for participating in a clinical trial or MAP; (ii) analyze an ADM or EMR to determine whether a patient is eligible or potentially eligible for a clinical trial or MAP; (iii) generate or provide, optionally in response to a request from a user, a list of clinical trials or MAPs for which a patient is eligible or potentially eligible; (iv) complete or assist a HCP to complete at least a portion of a form to request that a patient be permitted to participate in a clinical trial or MAP; (v) transmit, to a sponsor or regulatory agency, a request that a patient be permitted to participate in a clinical trial or MAP; (vi) receive, from a sponsor or regulatory agency, an indication whether the patient is permitted to participate in a clinical trial or MAP; (vii) provide an ADM template designated for a clinical trial or MAP; (viii) comply or assist an investigator or sponsor to comply with a law or regulation pertaining to a clinical trial or MAP; (ix) collect or analyze data pertaining to a patient participating in a clinical trial or MAP; and/or (x) transmit data generated in a clinical trial or MAP to a sponsor or regulatory agency.

In various embodiments a MAP may encompass, for example, providing a drug that: (1) is still in clinical development but has not been approved in any jurisdiction; (2) is not in clinical development and has never be approved, but still has or may have medicinal value for one or more patients; (3) is approved in at least one jurisdiction but is not approved in the jurisdiction in which a patient resides or receives health care or in which a patient's HCP is authorized to prescribe drugs; (4) has been discontinued in a particular market that includes the jurisdiction in which a patient resides or receives medical care or in which a patient's HCP is authorized to prescribe drugs; (5) has been discontinued globally; (6) is approved but not marketed; (7) is related to an approved drug, but is not approved for marketing and there is a shortage of the approved drug or the approved drug is unavailable due to failure to meet the conditions of the approved application; (8) is approved but whose availability is limited because the drug is subject to a risk evaluation and mitigation strategy; (9) is approved but the use for a particular patient would be “off-label” and the jurisdiction in which the patient resides or receives medical care or in which the patient's HCP is authorized to prescribe drugs prohibits off-label prescription.

MAPs exist in a number of jurisdictions. Laws and regulations governing the various types of MAPs available in different jurisdictions are known in the art. In the United States, FDA regulations authorize MAPs, typically referred to as expanded access programs, which allow patients access to investigational drugs under specified conditions. In some embodiments a MAP is an expanded access program. FDA regulations governing expanded access programs are set forth in 21 CFR § 312 (or amended versions or successors thereof as applicable). See, e.g., the final rule entitled “Expanded Access to Investigational Drugs for Treatment Use”, effective Oct. 1, 2009, published (along with Supplementary Information) in the Federal Register/Vol. 74, No. 155/Thursday, Aug. 13, 2009, pp. 40900-40945). In some embodiments a MAP comprises a single-patient IND, e.g., as described in FDA regulations set forth in 21 CFR § 312.310 (or amended versions or successors thereof as applicable). A single-patient IND refers to a request to the FDA that an individual patient be allowed access to a drug.

In some embodiment a MAP is for an intermediate-size patient population, e.g., as described in FDA regulations set forth in 21 CFR § 312.315 (or amended versions or successors thereof as applicable). A MAP under which a drug may be provided to an intermediate size patient population may be implemented when the FDA receives a significant number of requests (e.g., about 10 to about 100 in some embodiments) for single-patient access to an investigational drug for the same use. The FDA may ask the trial sponsor (e.g., the entity that is developing the drug for marketing) to consolidate these requests. In some embodiments the number of patients in an intermediate-size patient population is between 10 and 100, between 100 and 200, between 200 and 500, or between 500 and 1,000.

In some embodiments a MAP comprises a treatment IND or treatment protocol, e.g., as described in § 312.320 (or amended versions or successors thereof as applicable). In some embodiments a treatment IND or treatment protocol permits access for a large number of patients, e.g., at least 100 patients. In some embodiments at least 100 patients may be treated under a treatment IND or treatment protocol, e.g., between 100 and 1,000 patients, between 1,000 and 10,000 patients, or more, e.g., up to about 100,000 patients, or more. In some embodiments at least some such patients would not otherwise qualify for clinical trials of such drug.

In some embodiments a MAP is listed on ClinicalTrials.gov. In some embodiments a MAP is not listed on ClinicalTrials.gov.

In some embodiments a MAP is implemented in one or more countries outside the US. For example, in some embodiments a MAP is implemented in the European Union (EU), e.g., in one or more countries that are members of the EU. In the EU, MAPs are often referred to as named patient compassionate use program (NP-CUP) or cohort compassionate use programs (Coh-CUP). A NP-CUP is typically initiated by a physician for an individual patient. A Coh-CUP is typically a defined program initiated by a pharmaceutical company to allow access for a group of patients to an unauthorized drug provided by such company. In some embodiments a MAP in the EU allows access to an investigational drug. In some embodiments a MAP in the EU allows access to a drug that is not approved in a patient's country of residence but is approved in one or more other countries. In some instances such a program may permit a patient to use a drug during the time period between centralized European Medicines Agency (EMA) approval and the launch of such drug in the patient's country of residence. In some embodiments a drug is provided to one or more patients with a chronically or seriously debilitating disease, or a life threatening disease, and who cannot be treated satisfactorily by an authorized medicinal product in the EU or in the patient's country of residence. In some embodiments a patient who cannot be treated satisfactorily refers to a patient left without treatment options or a patient whose disease does not respond to or relapses to available treatments, or for whom available treatments are contraindicated or inadequate.

In some embodiments a MAP is implemented in Canada. In Canada a MAP may be referred to as a “special access program”. In some embodiments a special access program provides access to non-marketed drugs to practitioners treating patients with serious illnesses when conventional therapies have failed, are unsuitable, or are unavailable. In some embodiments a MAP is implemented in Australia. Australia provides patients access to experimental drugs via MAPs under the “special access scheme”. In some embodiments a MAP is implemented in Japan. For example, Japan's “named patient access” scheme makes drugs available if they are approved in the exporting nation.

In some embodiments a MAP allows for access to a drug that is in an early stage of development, e.g., following completion of at least one Phase I trial of the drug but prior to initiation of a Phase II trial. In some embodiments a MAP allows for access to a drug following initiation of a Phase II trial of the drug but prior to initiation of a Phase III trial. In some embodiments a MAP allows for access to a drug for which at least one Phase II trial has been completed. In some embodiments a MAP allows for access to a drug for which at least one Phase II trial has been completed and from which compelling data regarding efficacy of the drug are available. In some embodiments a MAP allows for access to a drug for which sufficient data regarding safety and efficacy to support initiation of a Phase III trial has not been acquired. In some embodiments a MAP allows for access to a drug for which sufficient data regarding safety and efficacy has been acquired to support initiation of a Phase III trial. In some embodiments a MAP allows for access to a drug in which a Phase HI trial is ongoing. In some embodiments a MAP allows for access to a drug in which at least one Phase III trial has been completed. In some embodiments a database for a completed trial has been locked. In some embodiments data from a completed trial has been analyzed. In some embodiments it has been determined that a trial met at least one endpoint.

In some embodiments a MAP allows access to a drug by patient(s) with a severe and/or life-threatening illness. In some embodiments a severe disease is a disease associated with morbidity that has substantial impact on day-to-day functioning. In some embodiments an immediately life-threatening illness is a stage of disease in which there is reasonable likelihood that death will occur within a matter of months or in which premature death is likely without early treatment. In some embodiments the disease is one for which commercially available treatment options are limited or entirely lacking. In some embodiments the primary purpose of a MAP is to diagnose, monitor, or treat a patient's disease or condition, not to generate scientific data intended to characterize the drug.

In some embodiments a patient has failed to respond to one or more commercially available treatments. In some embodiments a patient cannot participate in a clinical trial of the drug to be provided under a MAP. In some embodiments a patient cannot participate in a clinical trial of any drug open to individuals with the patient's disease. In some embodiments a patient cannot participate in a clinical trial because, e.g., the patient fails to meet criteria for entering or remaining in a clinical trial. In some embodiments a patient is unable to participate in a clinical trial at least in part because the patient resides at a location that is so remote from a site at which the trial is being conducted as to make it unreasonable for the patient to participate in the trial.

In some embodiments a EMR system provides information regarding MAPs, e.g., single-patient, named patient, or other individual MAPs, intermediate-size population MAPs, treatment INDs or treatment protocols, etc., that are in place or have been in place to permit patients to have access to a particular drug. Such information may include, for example, the number of patients who have been treated or are being treated under one or more MAPs, outcomes of such treatment, etc. In some embodiments a EMR system provides information regarding one or more drugs available under a MAP. For example, the EMR system may provide a monograph on a drug, a summary of clinical trials conducted on the drug, a link to publications on the drug, etc. In some embodiments a EMR system provides information to inform HCPs regarding the availability of and/or requirements of a MAP.

In some embodiments a EMR system provides one or more forms to be filled out by, e.g., a HCP or patient to facilitate participation of a patient in a MAP. In some embodiments a form is to be filled out, e.g., by a HCP, in order to comply with regulatory requirements, to request a drug from an entity, e.g., a pharmaceutical company, that supplies the drug, or to obtain an import permit. In some embodiments a EMR system provides one or more form(s) selected at least in part based on applicable regulatory requirements, e.g., regulatory requirements governing MAPs in a jurisdiction in which an HCP is licensed to practice medicine. In some embodiments a form is an informed consent form. In some embodiments a EMR system provides assistance to an HCP in preparing an expanded access submission to, e.g., the FDA or a sponsor. In some embodiments a EMR system may provide a form for an expanded access submission. A form may contain fields to be completed by a HCP or sponsor. In some embodiments information may be entered into one or more of the fields automatically by a EMR system based on information available in the patient's EMR. For example, demographic information, diagnosis, results of one or more diagnostic tests, treatments that the patient has received or is receiving may be entered. In some embodiments a HCP may be required to confirm agreement with at least some such information. In some embodiments a HCP may select at least some such information from a menu. In some embodiments a EMR system may suggest diagnostic tests to be performed to determine whether a patient is eligible for a MAP. In some embodiments one or more such tests may be required by a sponsor or by the FDA as a condition of granting access to a drug. In some embodiments information may be entered into one or more of the fields automatically by a EMR system based at least in part on information available in existing MAPs for that drug and/or based at least in part on information provided by an entity that would provide the drug under the MAP.

In some embodiments an ADM template includes fields sufficient to permit a determination of whether a patient meets criteria for participating in a clinical trial or MAP. In some embodiments the fields are sufficient to allow a determination that the potential patient benefit justifies the potential risks of the treatment use and those potential risks are not unreasonable in the context of the disease or condition to be treated.

In some embodiments a EMR system database stores a list of drugs that have been or are being made available under one or more clinical trials and/or MAPs. In some embodiments a EMR system stores a list of clinical trials and/or MAPs under which a drug has been made available or is currently available. In some embodiments a EMR system provides means by which a sponsor is able to submit information regarding clinical trials and/or MAPs being sponsored by such sponsor. In some embodiments a EMR system may provide information about such clinical trials and/or MAPs to users of the EMR system, e.g., HCPs or patients. In some embodiments a EMR system comprises information acquired from publicly available sources such as ClinicalTrials.gov or other websites (e.g., pharmaceutical company websites, hospital or clinic websites) or journal articles.

In some embodiments a EMR system generates or is capable of generating a list of drugs that a patient may be eligible to receive under a clinical trial and/or MAP. In some embodiments a EMR system generates or is capable of generating a list of clinical trials and/or MAPs for which a patient may be eligible. Such list(s) may be generated, for example, after a tentative diagnosis is entered or after a definitive diagnosis is entered or confirmed. In some embodiments a EMR system allows a user to enter or select a diagnosis (e.g., by clicking on it) and provides a list of drugs available under one or more MAPs for that diagnosis. In some embodiments, e.g., upon request by a user (e.g., a patient's HCP or a patient), a EMR system analyzes data in a patient's EMR and generates a list of drugs that a patient may be eligible to receive under a MAP and/or generates a list of MAPs for which the patient may be eligible. In some embodiments at least some of the data analyzed is an ADM. In some embodiments a list is generated automatically and, in some embodiments, may be provided upon request by a user. In some embodiments users may access a list of MAPs available for a particular disease or drug. In some embodiments a list may be revised based on information entered into an ADM.

In some embodiments an EMR or ADM comprises an icon or link that when selected may take the user to a document that displays a list of MAPs for which a patient may be eligible and/or that comprises other information pertaining to one or more such MAPs. In some embodiments a user may select a MAP from a list, e.g., by clicking on it. In some embodiments selecting a MAP takes the user (directly or via one or more further links or documents) to a document that provides information regarding the MAP or to a form to be completed to request permission, e.g., from a sponsor or regulatory agency, for a patient to participate in a MAP. In some embodiments a HCP submits a request via an EMR system. In some embodiments a sponsor receives a request submitted via an EMR system and supplies a drug to be used in a MAP. In some embodiments, a HCP who has at least one patient participating in an ADM-assisted MAP may be referred to as an “investigator”. In some embodiments an investigator agrees to comply with sponsor-specified requirements (such as completing or ensuring completion of the appropriate ADM).

In some embodiments an ADM template is specialized for a particular MAP. An ADM template specialized for a particular MAP may be referred to as a MAP-specific ADM template. A MAP in which an ADM is used, e.g., to enter patients and/or to gather data pertaining to the MAP may be referred to as an “ADM-assisted MAP”. In some embodiments a MAP-specific template may be used for a patient being treated in an ADM-assisted MAP. A MAP-specific ADM template may include one or more fields designed to gather information regarding efficacy and/or potential side effects regarding the drug provided under the MAP and/or to facilitate satisfying one or more regulatory requirements associated with the MAP. In some embodiments a MAP-specific ADM template may be developed at least in part by or in cooperation with a sponsor of the MAP. A MAP-specific template may comprise one or more fields designed to gather safety and/or efficacy data regarding the drug or to enable detection of potential adverse events or contraindications that may, for example, indicate that treatment of a particular patient with the drug should be stopped. In some embodiments a EMR system may check an ADM to ensure that the entered data meets specified criteria required for use of the ADM in a MAP. For example, the EMR system may check to ensure that all fields of an ADM required by a sponsor are completed. In some embodiments, the EMR system may check to ensure that ADMs are updated at appropriate times. For example, if a MAP protocol requires that a patient be evaluated at specified time intervals or over a specified time period, the EMR system may send an alert to the patient's HCP if the data is not entered in a timely manner or is incomplete or may reject the ADM as inadequate if the data are not entered within a predetermined time period or are incomplete. The EMR system may thus help enforce compliance with MAP protocol requirements.

In some embodiments, a drug is made available under a MAP to a HCP who agrees to use the EMR system for a patient to be treated with the drug under the MAP. In some embodiments the HCP agrees to use a MAP-specific ADM for patient(s) to be treated with the drug under the MAP. In some embodiments an HCP is required to complete and/or update the ADM template satisfactorily in order for initial and/or ongoing participation of the patient to be permitted.

In some embodiments, a drug may be made available under a MAP to any HCP who agrees to use the EMR system and agrees to use a MAP-specific ADM template for a patient to whom the drug may be administered. In some embodiments, a drug may be made available under a MAP to only a subset of HCPs (provided such HCPs agree to use the EMR system and, in at least some embodiments, agree to use a MAP-specific ADM for patients to whom the drug is administered). For example, a sponsor may desire to limit participation in a MAP to HCPs who have at least a minimum number of patients with a disease of interest.

In some embodiments a EMR system reduces the administrative burden associated with implementing a MAP or complying with one or more MAP requirements. In some embodiments a EMR system reduces the time required to enroll a patient in a MAP. In some embodiments a EMR system reduces the time from submission of a request for a patient to participate in a MAP and receipt of a decision as to whether the patient is permitted to participate in the MAP. In some embodiments a EMR system reduces the time from submission of a request for a patient to participate in a MAP and receipt of the drug. In some embodiments a EMR system reduces the time required on the part of a HCP to comply with requirements of a MAP.

In some embodiments a EMR system facilitates communication between a first HCP who is considering seeking approval to initiate a single-patient MAP to treat a patient with a particular drug or is considering seeking approval to treat a patient under an ongoing MAP and one or more second HCPs who have treated or are treating patient(s) with the same drug, e.g., under a MAP. In some embodiments a EMR system provides means by which HCPs may share their experiences regarding use of the drug or regarding participation in a MAP.

In some embodiments a EMR system assists an investigator or sponsor in complying with one or more regulatory requirements associated with a MAP, such as adverse event reporting (e.g., reporting to an appropriate government agency such as the FDA and/or to the entity providing the drug), maintaining accurate case histories, maintaining accurate drug disposition records, retaining records in a manner consistent with the requirements of § 312.62, and/or complying with one or more other investigator responsibilities under subpart D that may apply. In some embodiments a sponsor may be an individual or entity that submits an expanded access IND or protocol. An investigator may be an investigator-sponsor.

In some embodiments a EMR system facilitates approval of a MAP by an IRB or facilitates approval by an IRB of a patient's participation in a MAP. For example, a EMR system may provide information that identifies one or more IRBs that has previously approved participation of a patient in a particular MAP or that identifies one or more IRBs that has previously approved a protocol for a clinical trial of the drug to be provided under a MAP.

In some embodiments a EMR system facilitates gathering safety and/or efficacy data for patients participating in a MAP. In some embodiments a EMR system facilitates the acquisition of useful safety and/or efficacy data from a MAP. In some embodiments it is envisioned that a EMR system may, e.g., through use of ADM templates, which may be MAP-specific ADM templates, enhance acquisition of useful safety and/or efficacy data from a MAP. For example, an EMR system or ADM template may require entry of particular information for proper completion or updating, may provide feedback to a HCP if appropriate information is not entered in a timely manner, etc. In some embodiments use of a MAP-specific ADM template may make it possible to draw meaningful conclusions from a MAP or from multiple single-patient MAPs by, e.g., ensuring collection of a defined sets of data by different HCPs who may be responsible for treating different patients receiving the drug. In some embodiments it is envisioned that a EMR system may increase the safety and/or effectiveness of MAP programs for patients by, e.g., detecting patient data that may indicate a potential adverse event or providing timely updates to HCPs regarding safety or potential adverse events associated with the drug.

In some embodiments safety and/or efficacy data from a MAP may be used, e.g., by a sponsor. In some embodiments safety and/or efficacy data from a MAP may be used to, e.g., support a marketing application to a regulatory authority, e.g., a new drug application (NDA) or biologic license application (BLA), a marketing authorization application (MAA), or equivalent in a jurisdiction of interest. In some embodiments, safety-related data may be gathered via an ADM for all patients who receive the drug, but only a subset of patients (i.e., those who meet specified criteria) may be assessed for purposes of evaluating efficacy in a particular indication. It may thus be possible to gather safety-related data in a large number of patients, which data may be considered along with efficacy data gathered in a smaller number of patients, for evaluating a drug. Although a MAP may typically not include a control group as part of a protocol, in some embodiments, a control group for comparison purposes may be selected from among patients who have been diagnosed with the disease of interest but not receiving the drug. ADMs having appropriate characteristics for a control group may be selected from among the set of ADMs for the relevant disease in the EMR database. In some embodiments, a matched control patient (i.e., a subject having characteristics comparable to those of a subject who is treated with a drug under a MAP but who receives a different treatment (e.g., a standard of care therapy, or any particular specified therapy) may be selected. In some embodiments, ADMs to be used as a control group may be selected randomly (e.g., from among ADMs that meet a predetermined set of criteria). In some embodiments data obtained from a MAP may be compared with data obtained from a Phase II or Phase III clinical trial. In some embodiments data from a MAP may be submitted to a regulatory agency together with data from a Phase II or Phase III trial. In some embodiments a MAP-specific ADM may comprise one or more fields designed to collect data that may be combined with or may supplement data obtained from a controlled clinical trial.

In some embodiments, if a tentative or definitive disease diagnosis for a patient is entered into a EMR system or into an ADM template, and a drug is available for the disease under a MAP, one or more of the patient's HCPs may receive an alert informing him or her of the availability of the drug. In some embodiments a EMR system may periodically inform HCPs, e.g., physicians, of the availability of MAPs. Such information may be provided at least in part based on HCP specialty. For example, oncologists may receive alerts regarding drugs for cancer treatment; infectious disease specialists may receive alerts regarding drugs for infectious diseases, etc. In some embodiments a link to a web page for the MAP on ClinicalTrials.gov or other public clinical trial registry is provided. Information posted for the MAP on the ClinicalTrials.gov website (or other registry) may indicate that the drug is available under a MAP. In some embodiments, if a patient receives a tentative or definitive diagnosis of a disease for which a drug may be available under a MAP and such diagnosis is entered into a EMR system or into an ADM template, the patient may receive an alert informing him or her of the availability of the drug under a MAP.

In some embodiments, HCPs who agree to be investigators in a MAP but subsequently fail to reasonably comply with the applicable requirements may be identified in the EMR database and/or may be precluded (for at least a period of time, or indefinitely) from future service as investigators in ADM-assisted MAPs.

In some embodiments, any one or more of the functions or features described above relating to use of an EMR system in the context of clinical trials and/or relating to use of ADMs in the context of clinical trials may be employed in the context of MAP(s), and vice versa. In some embodiments appropriate functionality may be appropriate specifically for use in the context of MAP(s). For example, in some embodiments functionality designed with regard to satisfying regulatory requirements applicable to MAPs is implemented. In some embodiments a link specific for MAPs is provided in EMRs of an EMR system, which is labeled in a manner to indicate its association with MAPs. For example, the link may be labeled “Managed Access Programs”, “Expanded Access Programs” or with any other suitable label. In some embodiments clicking the MAP link brings the user to an ADM-SC template designed to facilitate MAP eligibility determination and/or enrollment of a patient in an appropriate MAP. In some embodiments after clicking on an “Experimental Therapies” link a user may select a “Managed Access Program” link. In some embodiments a user may select either a “Clinical Trial” link or a “MAP” link. In some embodiments a link specific for MAPs and appropriate functionality to allow use of ADMs in a MAP is provided as part of an ADM component. The term “MAP-specific data” may be used to refer to data that is collected particularly for purposes of a MAP, e.g., such data would not ordinarily be collected as part of ordinary standard of care therapy for the subject. In some embodiments a template comprises at least one field for entry of MAP-specific data.

In some embodiments, the opportunity to prescribe a white label drug and/or to be an investigator in a clinical trial of a white label drug serves as an incentive for a HCP to use a EMR system and/or to use a designated ADM for a patient who receives the white label drug. In some embodiments, the opportunity to provide a drug under a MAP and/or to be an investigator in a MAP serves as an incentive for a HCP to use a EMR system and/or to use a designated ADM for a patient who receives the drug under a MAP.

In some embodiments, HCPs who agree to be investigators in an ADM-assisted study but subsequently fail to reasonably comply with the applicable requirements, may be identified in the EMR database and/or may be precluded (for at least a period of time, or indefinitely) from future service as investigators in ADM-assisted trials. In some embodiments, HCPs who agree to be investigators in an ADM-assisted managed access program but subsequently fail to reasonably comply with the applicable requirements, may be identified in the EMR database and/or may be precluded (for at least a period of time, or indefinitely) from future service as investigators in ADM-assisted managed access programs.

In some embodiments, it is envisioned that the EMR system may be used to facilitate Phase IV (post-approval) studies. Such trials may sometimes be required by the FDA as a condition to the approval. Failure to conduct such trials or failure of the drug to show adequate efficacy and safety in such studies may lead to revocation of the approval. In some embodiments, subjects who participated in a Phase III trial (which may or may not be an ADM-assisted Phase III trial) are followed after completion of the Phase III trial in an ADM-assisted clinical study. ADMs appropriate for a Phase IV study may be included in the EMR system.

In some embodiments an EMR system may be used in connection with or to facilitate off-label use and/or repositioning. “Repositioning” (sometimes termed “repurposing”) encompasses the application of existing drugs to new indications (e.g., new diseases). In various embodiments a drug that is a candidate for repositioning may be a drug that is approved and on the market, approved but not on the market, or not approved, in a particular jurisdiction of interest (e.g., the US). In some embodiments a drug that is a candidate for repositioning may be a drug that (i) failed to show efficacy in Phase II or Phase III clinical trials (and typically did not have significant safety issues in such trials); (ii) stalled in development for commercial reasons; (iii) passed the point of patent expiration for the compound and/or for use in the particular indication(s) for which it is approved. In some embodiments a drug that is a candidate for repositioning is approved for use in one or more diseases or patient populations and is on the market but may be useful in one or more different diseases or populations (that are not included as approved in its approved label). In some embodiments a company that makes, distributes, supplies, holds rights to, or profits from a drug and/or use of a drug in one or more indication(s) may seek to assess the use of the drug in one or more different indications. In some embodiments such a company may seek to obtain clinical evidence supporting the use of the drug in one or more different indications.

In some embodiments drugs that are candidates for repositioning may be listed in an Experimental Therapies section. In some embodiments approved drugs that are candidates for repositioning may be listed in a section called “Off-label Therapies” (or equivalent terminology) in addition to, or instead of, in the Experimental Therapies section. In some embodiments a drug that is a candidate for repositioning may be made available at a reduced cost or no cost, e.g., to a patient whose HCP selects the drug for the patient via an ADM or who uses or agrees to use an ADM to follow the patient.

In some embodiments candidates for off-label use and/or repositioning that are listed in an Experimental Therapies or Off-Label Therapies section may be selected by or with advice of an expert panel, committee, or organization (e.g., a professional association) that is not under control of a company that is seeking to reposition a drug or would profit from off-label use. The panel, committee, or organization may review available preclinical and/or clinical information and conclude that the drug should be listed as an Experimental Therapy or Off-Label Therapy for use in the disease, e.g., that a reasonable physician would agree with using the drug to treat a patient with the disease in question. In some embodiments information regarding available evidence supporting the use is made available via an Experimental Therapies or Off-Label Therapies section of an ADM. Such information may include a rating or assessment of the strength of the evidence supporting the use in a particular disease or patient population. Such evidence may comprise, e.g., in silico data (e.g. from computer-based structure screening), in vitro data (e.g., from cell-free or cell-based assays), preclinical data (e.g., gathered from non-human animals that serve as models of the disease), and/or clinical data. Clinical data may have been gathered from prior trials of the therapy and/or, in the case of therapies that have been or are currently marketed in at least one jurisdiction, from reports of use of the therapy in treating patients (e.g., off-label use or approved use in a jurisdiction where the therapy is approved for use in the indication).

In some embodiments, a HCP and/or HCO may receive an incentive for serving as an investigator or site, respectively, for an ADM-assisted clinical study. Such incentives may be provided, for example, on a per-ADM or per-subject basis. In some embodiments, an incentive may differ in type or amount from that which may be provided to the HCP or HCO as consideration for contributing the ADM. For example, a larger incentive may be provided. In some embodiments an incentive may be calculated to be approximately equivalent to that which the HCP would receive had the HCP not chosen to serve as an investigator, such that the HCP does not receive a direct financial benefit from serving as an investigator. In some embodiments an incentive may not be provided to HCPs or HCOs in instances where an ADM is used for a clinical study. In some embodiments, reimbursement of the HCP and/or HCO may be provided in order to cover the costs of extra labor, tests, or other expenses incurred by the HCP or HCO as a result of HCP's service as an investigator in the clinical study. In some embodiments, subjects may be compensated (e.g., with money) for their participation in a study. In some embodiments, the EMR system at least in part manages reimbursement or compensation.

In some embodiments, the business entity may charge sponsors a fee in exchange for the opportunity to conduct an ADM-assisted trial. In some embodiments, the business entity may provide assistance to sponsors with the design, implementation, and/or data analysis of trial-related ADMs. In some embodiments the business entity may offer assistance to sponsors regarding assembly of data from an ADM-assisted study into a format appropriate for submission to the FDA. In some embodiments any of such services or assistance may be provided for a fee. In some embodiments a sponsor pays for use of an EMR system for purposes of a clinical trial or managed access program. For example, payment may be based at least in part on the number of subjects that are screened or enrolled in a trial or MAP through use of an EMR system and/or based at least in part on the number of subjects for which at least some clinical trial data or data required by a MAP is electronically captured through use of an EMR system. In some embodiments such payment is made at least in part to a business entity that at least in part owns, controls, makes, sells, or provides an EMR system or ADM component.

In some embodiments, ADMs may be made available to the FDA, to an internal review board (IRB), or to a data safety monitoring board (DSBM) via the EMR system. For example, FDA employees or IRB or DSMB members may be users of the EMR system. A sponsor may provide the FDA, IRB, or DSMB with a list of the ID numbers of the appropriate ADMs of subjects in the trial. In some embodiments, the ADM may include the identity of the treatment or other intervention being studied or indicates that the subject serves as a control. In some embodiments, e.g., for a blinded study, such information may not be included in the ADM (in which case it may be provided separately to the FDA) or such information may be included in a field that is not made accessible to the HCP. For example, the sponsor may enter the appropriate data into such field.

In various embodiments a disease, e.g., a disease for which a drug is tested or used in a clinical trial or MAP or for which an ADM template is provided, may be any disease. In some embodiments a disease is cancer. In some embodiments a cancer is a carcinoma. In some embodiments a cancer is a sarcoma. In some embodiments a cancer is a cancer of the adrenal gland, biliary tract, bladder, bone, breast, brain, cervix, colon, endometrium, esophagus, head or neck, kidney, liver, lung, oral cavity, ovary, pancreas, prostate, rectum, skin, testis, thyroid, or uterus. In some embodiments a cancer is a hematologic cancer, e.g., a leukemia, lymphoma, multiple myeloma, or myeloproliferative neoplasm. In some embodiments a cancer is metastatic. In some embodiments a cancer is resistant to one or more drugs ordinarily used to treat cancers of that type.

In some embodiments a disease is an infectious disease. In some embodiments an infectious disease is any disease caused by a virus, bacterium, fungus, or parasite. In some embodiments a virus, bacterium, fungus, or parasite is resistant to one or more drugs that are ordinarily used to treat patients infected by the virus, bacterium, fungus, or parasite, e.g., the virus, bacterium, fungus, or parasite has acquired drug resistance. In some embodiments the virus, bacterium, fungus, or parasite is multi-drug resistant. In some embodiments an infectious disease is HIV infection, a HIV-related condition, or AIDS. In some embodiments an infectious disease is an opportunistic infection.

In some embodiments a disease is an orphan disease. In some embodiments a disease is a rare disease. In some embodiments a rare disease is a disease that affects less than 200,000 persons in the United States, or less than about 1 in 1,500 people. In some embodiments a rare disease is a disease that affects less than about 1 in 2,000, less than about 1 in 2,500, less than about 1 in 3,000, less than about 1 in 4,000, or less than about 1 in 5,000 people. In some embodiments an orphan disease or rare disease is defined as set forth in a law or regulation such as the US Orphan Drug Act of 1983, US Rare Disease Act of 2002, or relevant law or regulation in an ex-US jurisdiction.

In some embodiments a disease is a genetic disease. In some embodiments a genetic disease is an inherited disease. In some embodiments a genetic disease is a single gene disorder, i.e., the disorder is the result of a single mutated gene. A single gene disorder may have an autosomal dominant, autosomal recessive, X-linked dominant, X-linked recessive, Y-linked, or maternal (mitochondrial) inheritance pattern. In some embodiments a genetic disease is cystic fibrosis. In some embodiments a disease is a metabolic disease, e.g., an inborn error of metabolism. In some embodiments a metabolic disease comprises an enzyme deficiency. In some embodiments a disease is a neurodegenerative disease, e.g., Huntington's disease or amyotrophic lateral sclerosis.

It is noted that while it is envisioned that the EMR system and ADMs may be implemented and used mainly in the context of human health care, the disclosure may encompass embodiments relating to veterinary medicine. For example, a EMR system and/or ADMs may be established for veterinary medicine and/or diseases that affect animals, or an ADM-assisted clinical trial may be conducted in animal subjects. Such animals may be mammals or avians or fish. Animals may be, for example, cows, horses, pigs, goats, sheep, chickens, turkeys, or other animals used commercially as sources of food for humans; companion animals such as dogs or cats, or any animal for which medications are regulated. Medications intended for use in animals are currently submitted to the FDA's Center for Veterinary Medicine (CVM) in a New Animal Drug Application (NADA). These medications are also specifically evaluated for their use in food animals and their possible effect on the food from animals treated with the drug. It should be noted that medications may be used in animals for purposes of treating disease or at least in part for other purposes such as promoting growth, milk production, etc. In some embodiments, an ADM-assisted clinical trial may be conducted at least in part for purposes of generating data to be included in a NADA. In some embodiments, such trial may be conducted using a white label drug.

It is also noted that the use of the EMR system for enrolling subjects and/or gathering data for clinical trial purposes may not be limited to ADM-assisted clinical trials.

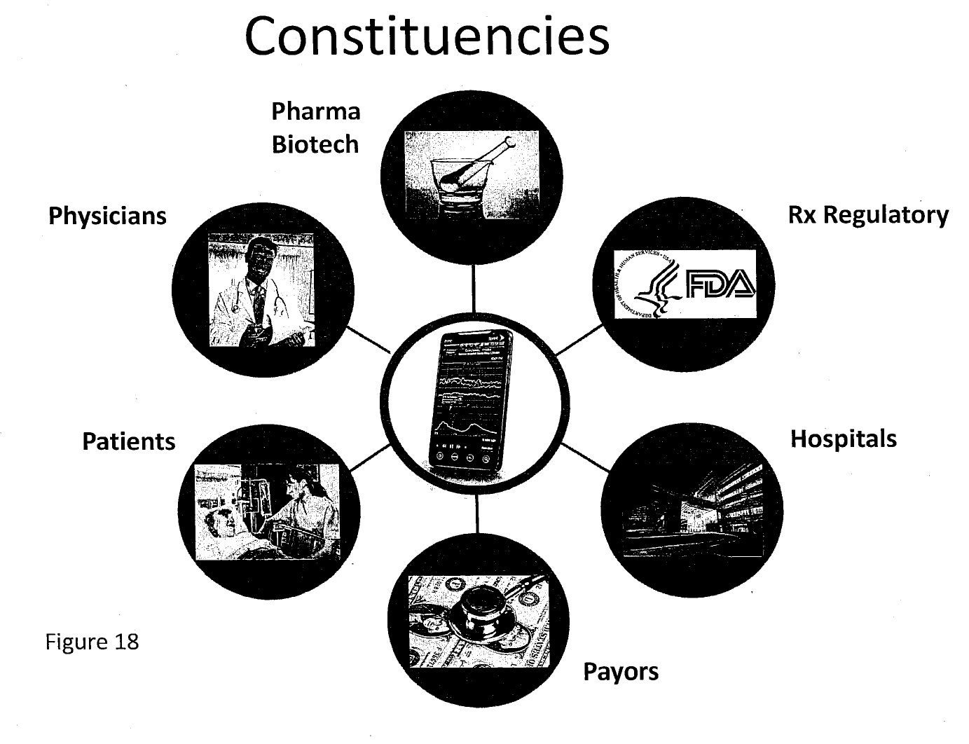

In certain embodiments a business entity that at least in part owns, controls, makes, sells, or provides an EMR system, e.g., an ADM-equipped EMR system, comprising experimental therapies functionality and/or that at least in part owns, controls, makes, sells, or provides an ADM component comprising experimental therapies functionality may interact with any of a variety of individuals or entities that have an interest in experimental therapies, clinical trials, and or MAPs. Such entities and individuals may collectively be referred to as “constituencies” or “constituents”. Constituencies may include, e.g., government entities (e.g., regulatory agencies such as the FDA), HCPs (e.g., individual HCPs, HCP organizations, societies, associations), HCOs (e.g., medical centers, hospitals, clinics), pharmaceutical/device manufacturers (e.g., which may be sponsors of a trial), patients (and/or caregivers, payors (e.g., insurance companies, Medicare, Medicaid and/or other government programs that may pay for health care products or services), or medical/scientific researchers. In certain embodiments an EMR system and/or ADM component or business entity may interact with any one or more such constituents, e.g., as represented schematically in FIG. 18 (wherein the EMR system, ADM component, or business entity is represented in the center of the figure as a portable electronic device). For example, the EMR system and/or ADM component may permit viewing, entering, modifying, or analyzing data, may receive queries, may transmit responses etc. In some embodiments the business entity may provide any of a variety of services to any such constituencies, e.g., as described herein.

In certain embodiments ADM-equipped EMR systems or an ADM database may provide patients with any one or more of the following: access to intelligible health care records that allow improved understanding of their medical condition; EMRs and/or ADMs that function as health assistants or coaches, by, e.g., providing patients with direct input on habits, checkups, etc.; a single central EMR or one or more ADMs that is/are or can be made available to all of the patient's HCPs and can accompany them to new HCPs; EMRs that improve the quality of the health care that they receive.

In certain embodiments ADM-equipped EMR systems or an ADM database may provide pharmaceutical companies, or other entities that may be sponsors of a trial or manufacturers or suppliers of a therapy or diagnostic tool (e.g., diagnostic equipment, diagnostic test), with any one or more of the following: more efficient and/or centralized subject screening and enrollment for clinical trials; centralized trial monitoring; organized collection of data from expanded access programs; rapid (e.g., real-time) and/or expanded evaluation of safety and/or efficacy of new drugs; data useful to support or identify opportunities repositioning of previously approved, previously marketed, and/or currently marketed drugs, data useful to support or identify opportunities for label refinement and personalized medicine; an alternative to controlled phase III clinical trials.

In some embodiments a sponsor or CRO can screen ADMs and can send a message to all ADMs for a particular diagnosis that meet exclusion/inclusion criteria for a particular trial. In some embodiments sponsors, investigators, or research coordinators can directly communicate with patients via their ADMs to notify them of upcoming or ongoing trials for which the data in the ADM indicate that the patient may be eligible. In some embodiments patients may be provided with information about the trial, instructions or directions regarding how to contact a clinical trial site, investigator, or research coordinator. In some embodiments patients may be invited to indicate whether they may be interested in participating in a trial for which they are potentially eligible based on their ADM. In some embodiments a sponsor may be able to project enrollment based on such responses. Such information may be useful in, e.g., selecting sites, projecting enrollment, etc. In some embodiments ADMs facilitate compliance with (adherence to) a therapeutic regimen or protocol. Compliance may comprise, e.g., taking a medication according to an appropriate schedule (e.g., daily, twice daily), avoiding certain foods or medications, etc. In some embodiments patients may be able to access their ADMs and directly report when they take a drug and/or report potential adverse events to the sponsor, CRO, or investigator. In some embodiments patients may be able to directly communicate with the sponsor, CRO, or investigator, e.g., via their ADM to express concerns or ask questions. In some embodiments patients may receive responses, e.g., via their ADM, from a sponsor, CRO, or investigator. Without wishing to be bound by any theory, such communication may reduce subject dropout and/or help identify issues that may lead to better design of future trials.

In some embodiments, ADM-equipped EMR systems or an ADM database may reduce the cost associated with a trial in any of a number of ways, e.g., by increasing efficiency of subject screening and enrollment, reducing costs of data entry and monitoring, reducing delays due to data collection issues (e.g., missing data that needs to be sought). In some embodiments, costs may be reduced because, through use of ADM-equipped EMR systems or an ADM database, one or more diagnostic tests, procedures, or patient visits to confirm the diagnosis, which may be covered by a regular payor (e.g., insurance company, government program), can be utilized for purposes of the trial protocol. In some embodiments, at least some diagnostic tests, procedures, or patient visits that would ordinarily take place for a patient having a particular disease, which may be covered by a regular payor as part of an ordinary standard of care for patients with that disease, can be utilized for purposes of the trial protocol.

In certain embodiments ADM-equipped EMR systems or an ADM database may provide makers or providers of diagnostic equipment (e.g., medical imaging devices), diagnostic tests, diagnostic testing services, with access to data relating to such equipment or tests. Such data may be used, e.g., to better understand how such equipment or tests are used in clinical practice or to identify opportunities or needs for improved or new equipment or services.

In certain embodiments ADM-equipped EMR systems or an ADM database may provide the medical research community with access to data that will permit the development of new insights into disease mechanisms, biomarkers, and patient populations.

In certain embodiments ADM-equipped EMR systems or an ADM database may provide health care organizations, e.g., hospitals, physician practices) with any one or more of the following benefits: streamlined healthcare delivery process, which may significantly reduce overhead; improved quality and/or improved patient outcomes (e.g., reduced readmissions for the same disease episode), which may lead to higher reimbursement under performance-based payment schemes (sometimes termed “pay-for-performance” or “outcomes-based” payment schemes); increased validity of performance-based reimbursement; improved ability to comply with accreditation standards, laws, and/or regulatory requirements, guidelines, or mandates; a database that can be mined to gather information useful, e.g., in making institutional decisions or policies.

In certain embodiments ADM-equipped EMR systems or an ADM database may provide payors with any one or more of the following benefits: increased feasibility and validity of performance-based reimbursement; reduced cost from inefficient or inappropriate prescriptions, tests, and procedures; reduced rate of medical errors through, e.g., more accurate diagnosis, rapid feedback to HCPs, checking the appropriateness of therapies, communicating to patients to, e.g., monitor or encourage compliance with therapy or recommendations, monitor symptoms or changes in a patient's condition or activities, or provide health information. In some embodiments, monitoring symptoms or changes in a patient's condition may permit timely intervention by a HCP or HCO. For example, a patient or a relative or caregiver may be contacted if monitoring indicates a worsening of symptoms or condition, and encouraged to visit a HCP or HCO. Such intervention may help reduce or avoid an exacerbation of an illness, reduce the likelihood of a hospital admission or readmission, etc.

In some aspects a system provides a patient-centric health hub. In some embodiments ADM-equipped EMRs or ADMs contribute to, facilitate, or form the basis of a patient-centric health hub. Such a patient-centric health hub may have functions that are focused on the patient as a consumer of health care products and services and/or as an active participant in their own health care. Patients may be notified rapidly of the latest information related to their diseases, their potential eligibility to participate in clinical trials, Managed Access programs, or receive repositioned therapies or therapies that are candidates for repositioning. In some embodiments ADMs facilitate health information integration, e.g., patients can access and understand their diseases by access to their ADMs, can co-manage their diseases, have rapid access to physicians/sponsors, and/or add health monitoring information and information on daily living to their ADMs. In some embodiments ADMs may communicate with patients and may inform them regarding any of a wide variety of matters, ranging, e.g., from physician visits to good habits, to health-related data or publications (e.g., in scientific journals, newspapers, websites, etc.), advocacy groups, support groups, products or services related to their disease, events related to their disease (e.g., fund-raising events, lectures), non-profit organizations (e.g., foundations) that support research into their disease, etc.

In some embodiments a patient-centric health hub includes one or more functions related to health promotion or wellness/wellbeing, which may not necessarily be related to a particular disease. For example, such functions may include exercise monitoring, diet monitoring, stress reduction, or other features that may contribute to overall wellness/wellbeing and/or may reduce the likelihood of developing a disease or reduce its severity or rate of progression. Health promotion refers to “the process of enabling people to increase control over, and to improve their health”. Health promotion may include any of a variety of activities such as health fairs, health education, medical screenings, health coaching, weight management programs, wellness newsletters, fitness programs and/or facilities, and educational programs relating to health.

Social Media

In some embodiments a system may provide various social media functions, which in some embodiments relate at least in part to ADMs. “Social media” refers to the various means of interactions among people in which they create, share, and exchange information and/or ideas in virtual communities and networks. Social media technologies encompass, e.g., Internet forums, weblogs, social blogs, microblogging, wikis, social networks, among others. In some embodiments a social media function may permit individuals having ADMs for the same disease to communicate with each other. Such communication may be, e.g., via email, text message, phone, or through other social media technologies. An ADM-related social network may comprise members who belong to one or more social networks such as Facebook, Twitter, LinkedIn, MySpace, etc., that was not established for purposes relating to a particular disease. In some embodiments an ADM-related social network is a subset of such a social network, e.g., an interest group within a larger social network. In some embodiments an ADM-related social network is an aggregate of such subsets. In some embodiments an ADM-related social network may comprise a network created at least in part for one or more purposes relating to a particular disease. Such purposes include, e.g., facilitating interactions among people having a particular disease, facilitating interactions among relatives, friends, and/or caregivers of people having a particular disease among themselves and/or with people who have the disease, etc. A caregiver may be any individual who assists a patient with one or more activities of daily living and/or with any of a variety of medical care needs (e.g., medication administration, dressing changes) outside the setting of a health care organization, typically in the patient's dwelling, and typically on a frequent (e.g., daily) basis. A caregiver may be a family member, friend, volunteer, or paid service provider. In some embodiments an ADM-related social network is open exclusively to persons having a particular disease, e.g., as evidenced by their having an ADM, e.g., an active ADM, for that disease. In some embodiments an ADM-related social network may be open to relatives, friends, and/or caregivers of people having the disease. In some embodiments an ADM-related social network is open to designees or invitees of people who are already members. It will be appreciated that “open to” in this context means that an individual is permitted to join a social network.

In some embodiments an entity, e.g., a business entity, that at least in part that at least in part owns, controls, makes, sells, or provides an ADM component and/or an ADM database establishes or arranges for establishment and/or maintenance of an ADM-related social network. In some embodiments an entity, e.g., a business entity, that at least in part that at least in part owns, controls, makes, sells, or provides an ADM component or ADM database, also at least in part owns or controls a website and/or computer program product that provides ADM-related social network function(s). In some embodiments an entity, e.g., a business entity, that at least in part that at least in part owns, controls, makes, sells, or provides an ADM component or ADM database, controls membership in an ADM-related social network. The entity may receive requests for membership from people seeking to join the social network and may grant or deny such requests. In some embodiments such a person is asked to provide identifying information, and the ADM database is checked to determine whether a person requesting to be allowed to join a particular ADM-related social network has an ADM for that disease or is a designee or invitee of a person who has an ADM for that disease. In some embodiments the individual is permitted to join the ADM-related social network for that disease if he or she has an ADM for that disease or, in some embodiments, is a designee or invitee of a person who has an ADM for that disease.

In some embodiments advertisements for products and/or services relevant to the disease corresponding to an ADM are displayed or sent to members of an ADM-related social network for that disease. In some embodiments an entity, e.g., a business entity, that at least in part that at least in part owns, controls, makes, sells, or provides an ADM component or ADM database, also at least in part owns or controls a website and/or computer program product that provides ADM-related social network function(s) may charge a fee to entities in exchange for placing such advertisements. In some embodiments members of an ADM-related social network may be asked or provided an opportunity to rate and/or review products and/or services relevant to the disease and/or providers of such products and/or services. Products relevant to the disease may include, e.g., monitoring devices, prosthetic devices, mobility aids, certain foods (e.g., foods that are gluten-free or free of one or more allergens), over-the-counter or prescription medications, etc. In some embodiments questionnaires, rating scales, or instructions are provided to help enhance the usefulness and objectivity of the ratings or reviews. In some embodiments products and/or services may be relevant to wellness/wellbeing in addition to or instead of being relevant to a particular disease or diseases. Such products and/or services may include, e.g., exercise/fitness equipment, gym memberships, exercise/fitness programs or classes, wellness coaches, personal trainers, etc.

In some embodiments members of an ADM-related social network are able to locate one another using, e.g., GPS or other location-based tools. In some embodiments, via location-based software patients can determine whether other patients with ADMs for the same disease(s) live in the same area or, in some embodiments, are present in the same room.

In some embodiments ADM-related social media functions are provided as part of a patient-centric health hub.

Shares

In some embodiments the business entity may offer shares of a single class. In some embodiments the business entity may offer multiple share classes, including, in some embodiments, at least one class of shares reserved for contributors. Other classes may be made available to investors in the business entity, who may or may not also be contributors. Investors may, for example, be members of the general public, government entities, accredited investors (as defined in Rule 501 of Regulation D of the U.S. Securities and Exchange Commission and as amended by the Dodd-Frank Wall Street Reform and Consumer Protection Act, and as otherwise amended from time to time), etc. For example, investors may be natural persons, corporations, venture capital firms, etc. In some embodiments multiple classes of shares may be reserved for contributors, including at least one class reserved for HCPs (e.g., physicians) and/or HCOs and at least one class reserved for auto-contributors. In some embodiments, share ownership may be managed such that the business entity remains at least 50% owned or controlled by contributors. In some embodiments, share ownership may be managed such that the business entity remains at least 50% owned or controlled by contributors who are HCPs. In some embodiments, share ownership may be managed such that the business entity remains at least 50% owned or controlled by contributors who are physicians. In some embodiments, share ownership may be managed such that the business entity remains at least 50% owned or controlled by physicians, who may or may not be contributors. In some embodiments, at least 50% owned or controlled may be more than 50% owned or controlled e.g., at least 50.1% owned or controlled.

In some embodiments, shares of at least one share class provides the shareholder with a subscription to the database. Varying levels of subscription privileges may be provided, e.g., as described above.

In some embodiments, the business entity may have a share account database that keeps track of the ownership of the business entity's shares. In some embodiments, each shareholder has an account in the share account database. In some embodiments, the component comprising the share account database may, via appropriate computer instructions, communicate with the component comprising the user account database. For example, the component that comprises the user account database may inform the component that comprises the share account database when a contributor earns a share. In some embodiments, the component that comprises the share account database may issue the share and may update the share account database accordingly. It should be understood that the share account database need not be separate from the user account database (discussed above) in some embodiments. For example, in some embodiments, a single account database format with appropriate fields could serve as a user account database and a shareholder account database. In some embodiments, shareholders may automatically receive user accounts along with their share ownership. In some embodiments (if the business entity is privately held) share ownership may be restricted to contributors and, e.g., at least some other types of users.

Security, Data Integrity, and Legal Compliance

In some aspects, the disclosure provides a system for assembling EMRs wherein the system comprises security features that guard against the fraudulent or unauthorized submission of health information, help protect the integrity of the health information submitted, and help limit the rights to access, contribute, or change information to those persons with credentials that entitle them to do so. Security and data integrity may be addressed in any of a variety of ways. It is contemplated that any methods known in the art for identity and access management may be used or adapted for purposes of the disclosure. In some aspects, the disclosure provides security-enhancing methods particularly suited for various embodiments. It should be understood that these aspects and features of the disclosure may find use in other contexts as well. As noted above, one or more passwords may be selected by or assigned to a user and may be used for access control. Passwords may be required to be “strong” passwords and/or may be required to be changed on a regular or irregular basis. Access from a particular computer, device, or Internet address may be automatically disabled after a predetermined number of incorrect password entries. Alternatively or additionally, smartcards (which may contain an embedded computer chip or magnetically stored identifying information), digital certificates (optionally encrypted in a smart card), and/or biometric identification may be used to control access.

In some embodiments, an EMR system maintains a list of an HCP's patients, which may be referred to as an HCP list or roster, e.g., a physician list or roster. An HCP may be permitted to access EMRs of patients on his or her roster but may not be permitted to access EMRs of patients not on his or her roster (except to the extent such access may be permitted to the HCP as a subscriber, e.g., in de-identified form). An HCP may be permitted to modify (e.g., change or add information to) EMRs of patients on his or her roster but may not be permitted to modify EMRs of patients not on his or her roster. An HCP may be permitted to modify ADMs of patients on his or her roster but may not be permitted to modify ADMs of patients not on his or her roster. In some embodiments, an EMR system of the disclosure may maintain a list of the HCPs who are authorized to access and/or modify a patient's EMR and/or to modify a patient's ADMs. In some embodiments a location-based identification system may be used wherein, for example, a EMR may only be accessed from a particular computer if an electronic device belonging to an individual authorized to do so is located within a specified distance of the computer. The electronic device may include a suitable positioning system. The positioning system may include any suitable system such as, for example, a global positioning system (“GPS”) receiver for, e.g., accessing a GPS application function call that returns the geographic coordinates (i.e., the geographic location) of the electronic device. In some embodiments the positioning system may utilize any suitable trilateration or triangulation technique to determine the geographic coordinates of the electronic device. In some embodiments localization may occur either via multilateration (e.g., trilateration) of radio signals between radio towers of the network and the phone. In some embodiments, the positioning system may determine various measurements (e.g., signal-to-noise ratio or signal strength measurements) of a network signal (e.g., a cellular telephone network signal, a wireless network access point or “hot spot”, or any other suitable network signal) associated with the electronic device to determine its location. While it is envisioned that mobile phones may often be used for localization, it should be understood that other personal, localizable electronic devices could be used for the same purpose. In some embodiments, the business entity issues such electronic devices to a patient or patient representative. The patient or patient representative may take the electronic device with him or her when visiting a HCP.

In some embodiments, patient consent may be required in order for a contributor to initiate establishment of a EMR for that patient or to initially gain access to the patient's EMR. In some embodiments, patient consent could be verified, for example, using a password-based approach, wherein both the contributor and the patient (or the patient's agent) need to enter a password into the same electronic device, and/or using a location-based approach, wherein the patient (or the patient's agent) and the contributor need to be co-localized (as determined, for example, by mobile phone tracking). In some embodiments, a patient (or their agent) could authorize different levels of access. For example, a patient may want a particular HCP to be able to update that patient's EMR with new information but not to be able to view the entire EMR. In some embodiments a feature is provided that may, e.g., if selected by the patient, permit overriding the requirement for patient consent. For example, patient consent may be overridden in case of an emergency, such as the patient being admitted unconscious to a hospital after an accident. In some embodiments a signature capture pad may be used in various aspects herein, e.g., to document informed consent for diagnostic tests, treatments, etc. and/or for capturing HCP signature.

In many embodiments, health information may be encrypted at least while being transferred over a network. In some embodiments at least some health information is stored in encrypted form. In some embodiments, an encryption standard that has been adopted by the U.S. Federal government or mandated by U.S. federal law (e.g., under the Health Information Technology for Economic and Clinical Health (HITECH) Act (part of the 2009 American Recovery and Reinvestment Act) as it may be amended or updated from time to time may be used. For example, the encryption standard known as Advanced Encryption Standard (AES) or its successors may be used.

In some embodiments, voice recognition technology may be used to facilitate security and/or integrity of the health information. For example, in some embodiments, only a user whose voice may be recognized by the EMR system as belonging to an individual authorized to modify a EMR or ADM may be able to do so. Multilanguage support and/or translation capability may be provided in some embodiments.

In some embodiments, the EMR system may comply with and/or facilitate HCP and/or HCO compliance with legal requirements such as those of the HITECH Act and/or HIPAA. In some embodiments, the EMR system qualifies as an EMR system whose adoption and demonstration of meaningful use may entitle HCPs and/or HCOs (e.g., eligible professionals (EPs), eligible hospitals, and critical access hospitals (CAHs) to incentive payments available under U.S. federal laws such as the HITECH Act and regulations issued pursuant thereto (see 42 CFR Parts 412, 413, 422 et al. Medicare and Medicaid Programs; Electronic Health Record Incentive Program; Final Rule, published in the Federal Register on Jul. 28, 2010, Vol. 75, No. 144; http://edocket.access.gpo.gov/2010/pdf/2010-17207.pdf). In some embodiments the EMR system may satisfy standards, implementation specifications, and/or certification criteria set forth in 45 CFR Part 170; Health Information Technology: Initial Set of Standards, Implementation Specifications, and Certification Criteria for Electronic Health Record Technology; Final Rule, published in the Federal Register on Jul. 28, 2010, Vol. 75, No. 144; http://edocket.access.gpo.gov/2010/pdf/2010-17210.pdf), and/or any subsequent standards issued pursuant to the HITECH Act. In some embodiments, the EMR system or at least some components or functions thereof, comply with applicable HL7 standards version 2.x or version 3, if any, and/or are adapted to interface with other systems that comply with such standards. In some embodiments the EMR system is certified by an organization recognized by the Office of the National Coordinator for Health Information Technology (ONC) as an Authorized Testing and Certification Body (ONC-ATCB) In some embodiments the EMR system is certified by the Certification Commission for Health Information Technology (CCHIT®) http://cchit.org and/or another certification body.

In some embodiments, the EMR database or at least a portion thereof, is accessible via a virtual private network (VPN). In some embodiments a VPN is a mobile VPN.

Various Embodiments

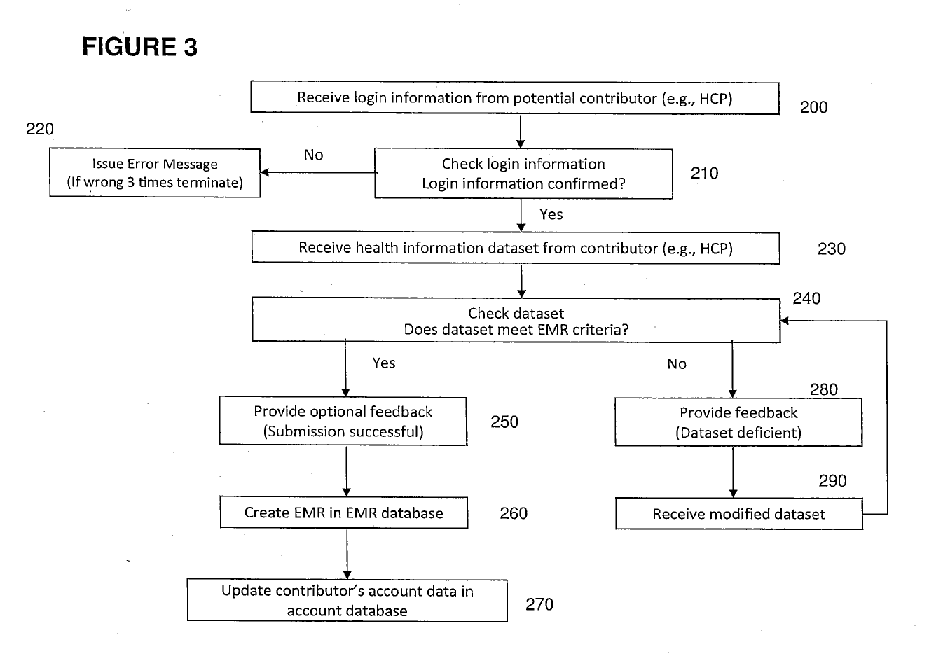

FIG. 3 shows a flow chart of a process in accordance with some embodiments. In step 200 a system receives login information from a potential contributor (e.g., a HCP). In step 210, the system checks the information to determine whether it corresponds with information in a user database. For example, the system may check a username, password, HCP identifier, biometric information, etc., against information stored in a database. If the information does not match, login may be refused (step 220). The system may terminate login attempts after a predetermined number of failed attempts. If login is successful, the system may receive health information (step 230). The system may check the health information to determine whether it satisfies a predetermined set of criteria, e.g., a set of EMR criteria (step 240). If said criteria are met, the health information may added to the database, e.g., as a created EMR (step 260), optionally after providing feedback indicating that submission was successful (step 250). A user account database may be updated upon successful submission (step 270). If health information to is determined not to satisfy a predetermined set of criteria, e.g., a set of EMR criteria, feedback may be provided accordingly (step 280). Such feedback may identify the deficiency and/or provide suggested remedy. A modified dataset may be received (step 290), which may then be checked (step 240). The process may continue, e.g., until a dataset is accepted. A modified dataset may comprise, for example, additional data, or at least some different data, e.g., as compared with a previously submitted version, in various embodiments.

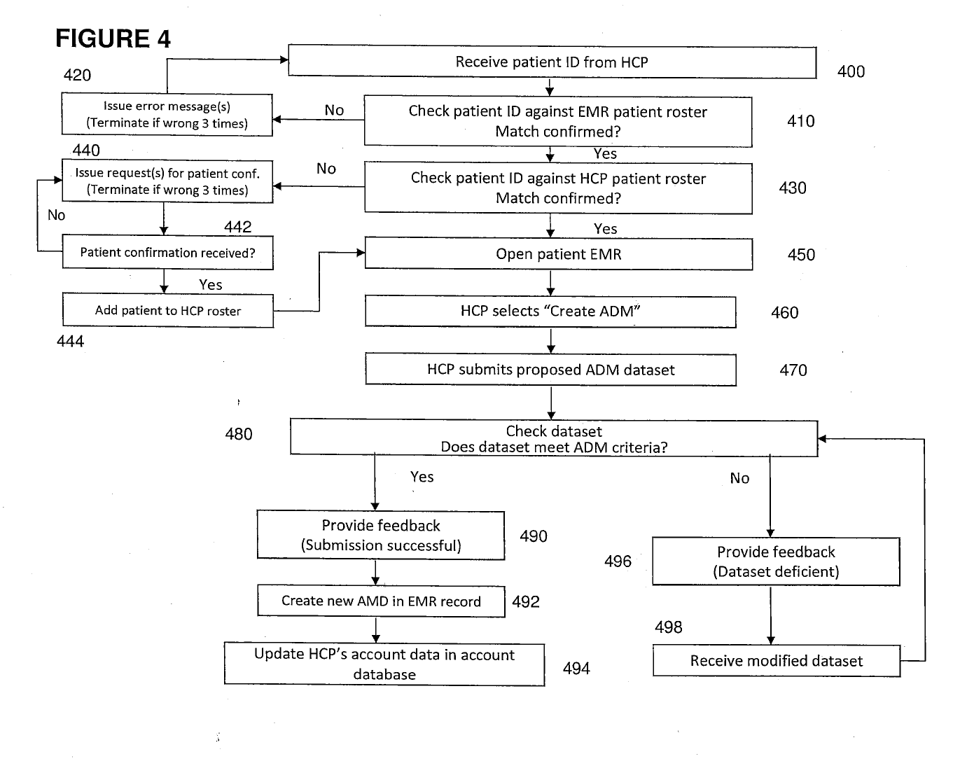

FIG. 4 shows a flow chart of a process in accordance with some embodiments. It is assumed that a HCP has logged on to (gained access to) the system. The HCP submits a patient ID, which is received by the system (step 400). The system checks the patient ID against a roster of patients that have EMRs stored in the database (step 410). If there is no matching patient ID in the patient roster, an error message may be issued (step 420). The system may terminate patient ID submission attempts after a predetermined number of failed attempts. If successful, the system may checks the patient ID against a HCP roster (step 430). If a match is not found (patient is not on HCP roster), the system may request patient confirmation (step 440). The system may check whether patient confirmation has been received (step 442) and may issue repeated requests therefor, which may terminate after a predetermined number of requests or failed submissions (e.g., 3). If patient confirmation is received, the system may add the patient to the HCP's roster (step 444). These steps may permit a HCP whom a patient visits for the first time (e.g., a referral) to gain access to the patient's EMR. If a match is found (patient is on HCP roster) or patient has been added to HCP roster, e.g., in step 444, the patient's EMR may be opened (step 450). In step 460, a HCP (e.g., after determining that a patient has or may have a disease) selects a “create ADM” option. In step 470, the HCP submits a dataset. Optionally the data is entered in an ADM template. In step 480, the submitted dataset is checked to determine whether it meets predetermined criteria. If such criteria are met, feedback is optionally provided accordingly to the HCP (step 490), and the dataset is added to the patient's EMR as a new ADM (step 492). The HCP's account data may be updated to reflect addition of the ADM (step 494). If such criteria are met, feedback is optionally provided accordingly to the HCP (step 496). The system may then receive a modified dataset or additional data (step 498), which may be checked for compliance with ADM criteria (step 480).

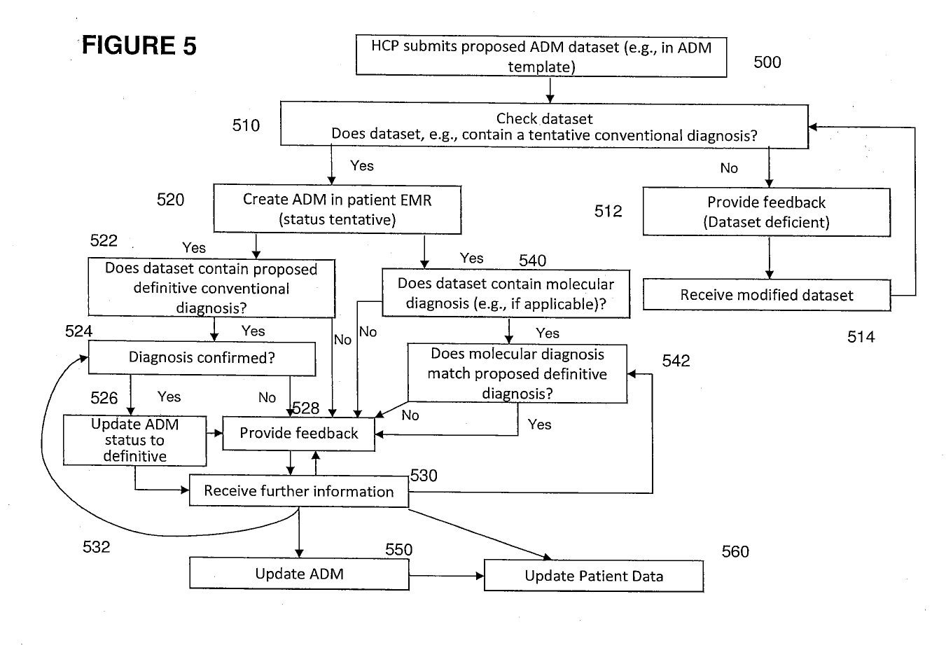

FIG. 5 shows a flow chart of a process in accordance with some embodiments. It is assumed that a HCP has logged on to (gained access to) the system and a patient's EMR. In step 500, the HCP submits a proposed ADM dataset, which may be submitted at least in part by entering information into an ADM template. In step 510, the system checks the submitted information. For example, the dataset may be checked to determine whether it contains at least a tentative conventional disease diagnosis. If predetermined criteria are met, an ADM may be created, which may be assigned a status of “tentative” (step 520). If predetermined criteria are not met, feedback may be provided accordingly (step 512). A modified dataset may then be received (step 514) and checked (step 510) which process may continue until a dataset is accepted. A dataset containing a tentative diagnosis may be checked to determine whether it contains a proposed definitive conventional diagnosis (step 522). If so, the proposed definitive conventional diagnosis may be checked to determine whether it should be confirmed (step 524). For example, the system may check whether appropriate diagnostic tests have been performed and/or may analyze results thereof that have been entered (e.g., entered in step 500). If the proposed definitive diagnosis is confirmed, the status of the ADM may be updated to “definitive” (step 526). Feedback may accordingly be provided to the HCP, e.g., indicating that the diagnosis is confirmed (step 528). If the proposed definitive diagnosis is not confirmed (e.g., if appropriate diagnostic tests have not been performed, and/or if results thereof are not consistent with the diagnosis or suggest an alternative diagnosis), feedback may be provided (step 528). Further health information (e.g., diagnostic test results) may be received (step 530), optionally at least in part in response to feedback provided in step (528). A process of additional feedback and submission of health information may occur. It should be understood that the process may occur during a single session or over multiple sessions, and that health information may be submitted from a variety of sources. Additional information may also or alternately be received in step 530 after a diagnosis is confirmed. At any point the system may check whether information sufficient to confirm a proposed definitive diagnosis has been received. Thus, step 532 represents a loop back to step 524, which may occur multiple times. Further information entered may be added to an ADM (step 550) and/or to Patient Data (step 560). Such information may be subject to checking by the system prior to addition. Returning to a created ADM (step 520), the system may check whether the dataset contains a molecular diagnosis (step 540). If the dataset does not contain a molecular diagnosis, feedback may be provided, such as a suggestion to perform a diagnostic test appropriate to obtain a molecular diagnosis (step 528). If a molecular diagnosis either matches (e.g., is consistent with) or fails to match (e.g., is inconsistent with) a proposed definitive diagnosis, appropriate feedback may be provided (step 528). Additional information may be received, and another determination of whether a molecular diagnosis matches a proposed definitive diagnosis may be performed (step 540). Steps of FIG. 5 may be repeated multiple times, e.g., for different diseases of a patient.

Feedback, e.g., represented in step 528 (or any other step comprising providing feedback or as described herein) may comprise any of multiple different types or content of feedback depending at least in part on, e.g., preceding step(s) of the process or information previously submitted or received.

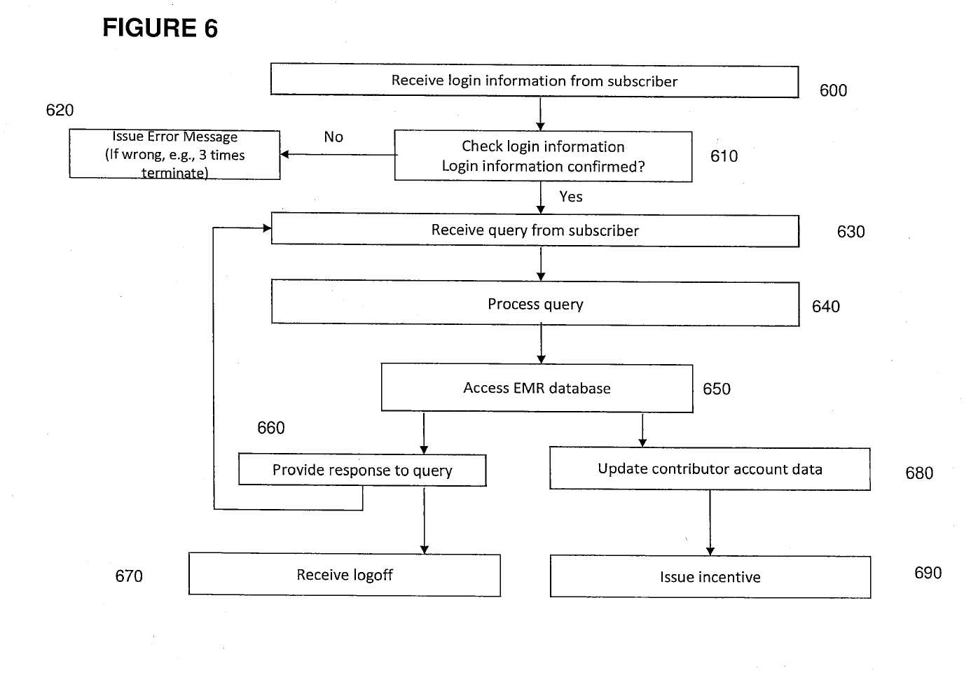

FIG. 6 shows a flow chart of a process in accordance with some embodiments. Login information is received from a subscriber (or possible subscriber) (step 600). Login information is checked against a database that stores a list of subscribers and login information thereof (step 610). If login information does not match, an error message may be issued (step 620). The system may terminate login attempts after a predetermined number of failed attempts (e.g., three). If login is successful, the system may receive a query from the subscriber (step 630). The system may process the query (step 640). Processing may include any of a wide variety of types of processing steps, which may depend, e.g., at least in part on the nature or complexity of the query, the data required to address the query, etc. For example, a query may comprise a request to retrieve an ADM having a particular identifying number, or may comprise a more complex query that may entail the system, for example, accessing multiple ADMs which may be identified by searching on one or more input terms, extracting one or more specified data elements therefrom, manipulating said data elements, e.g., combining or analyzing said data elements, performing one or more mathematical computations (e.g., generating a statistic such as a mean, standard deviation, confidence interval, p-value), performing one or more statistical tests, generating a requested display format or report, outcome analysis, etc. One of ordinary skill in the art would be aware of numerous types of queries or analysis that may be performed on health information. In step 650, the database is accessed, e.g., to identify and/or retrieve data to respond to the query. Step 650 may include one or more steps of manipulating or analyzing data retrieved from the database. A response to the query is returned in step 660. A subscriber may log off (step 670) or may submit an additional query which may be received by the system (step 680). An additional query may, for example, be based at least in part on results of a previous query. In step 680, contributor account data is updated, based at least in part on data accessed in step 650. For example, if an ADM to which a subscriber contributed was accessed, the contributor's account data may be updated. In step 690, an incentive is issued to a contributor (e.g., at some later time point), the amount or timing of which may depend at least in part on content of the contributor's account information.

Processes as shown in FIGS. 3, 4, 5, and 6, and descriptions thereof, are exemplary and should not be construed as limiting. Any one or more steps may be omitted, added, or modified in various embodiments. A step may comprise multiple sub-steps, which may not be depicted, or multiple steps may be combined. Certain steps may occur concurrently, in parallel, or in a different order than depicted in some embodiments. For example, updating a databases and providing feedback may occur substantially at the same time, or in either order, in various embodiments. It should be understood that any one or more steps may be implemented in hardware, software, or a combination thereof. It should be understood that any step(s) may incorporate or be augmented based on any applicable description herein. It should be understood that any one or more steps, or substep(s) thereof, may be executed on one or more processors, which may be part of one or more computers or networks.

Feedback may comprise, e.g., an indication that a dataset was accepted or rejected, a reason why a dataset was accepted or rejected (e.g., an indication of a deficiency in said dataset or a failure to meet a predetermined criteria), a suggestion, a set of options, or modifying an ADM template, in various embodiments. A suggestion may comprise, for an example, a suggested diagnostic test to confirm a diagnosis, an alternative diagnostic test, a suggested treatment, an alternative treatment, etc.

In some aspects, a database comprising EMRs is provided, said database being usable by HCPs for health care purposes and usable in addition for at least one non-health care purpose (e.g., a research purpose). In some aspects, health information is provided at least in part in modules comprising de-identified health information. In some embodiments the EMRs comprise or consist essentially of ADMs. A HCP may access such module(s), e.g., in the context of providing health care to a patient. An individual who is not an HCP of the patient may access or retrieve data from said module(s), e.g., for research purpose(s), clinical trial purposes, regulatory purposes, or because the individual is the patient or a designee of the patient.

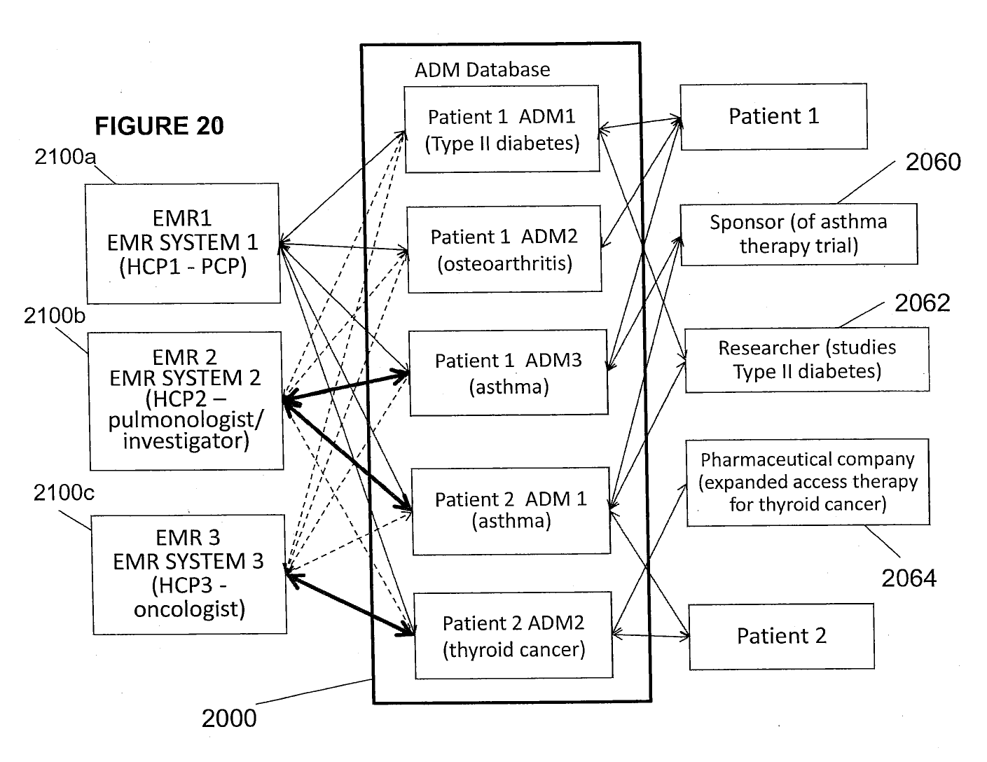

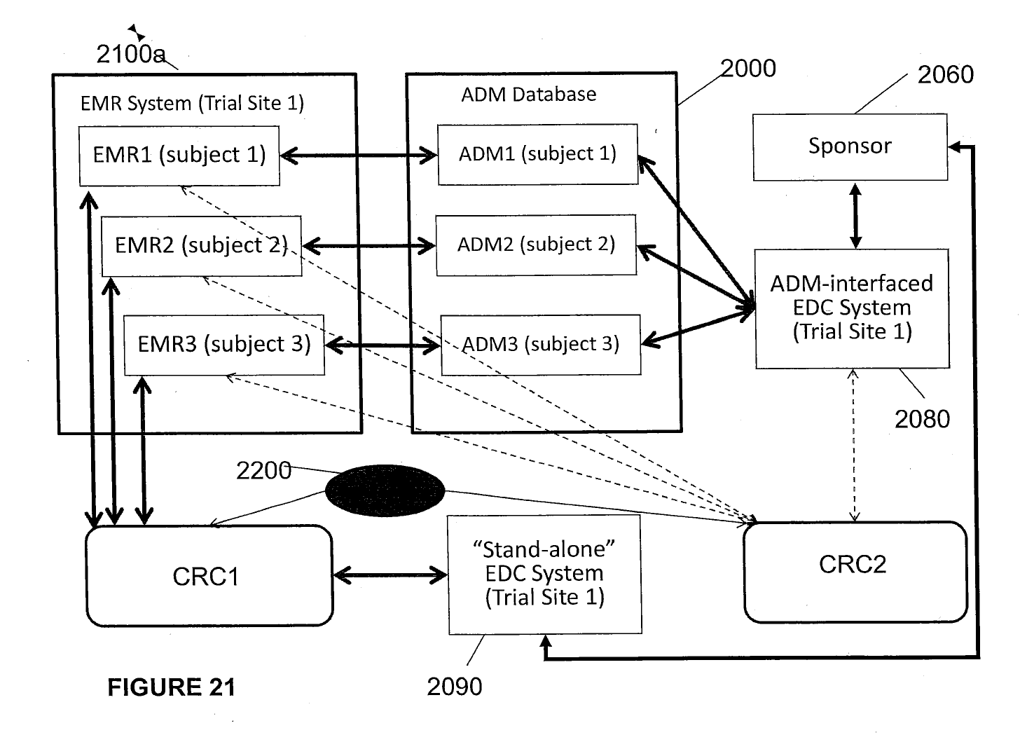

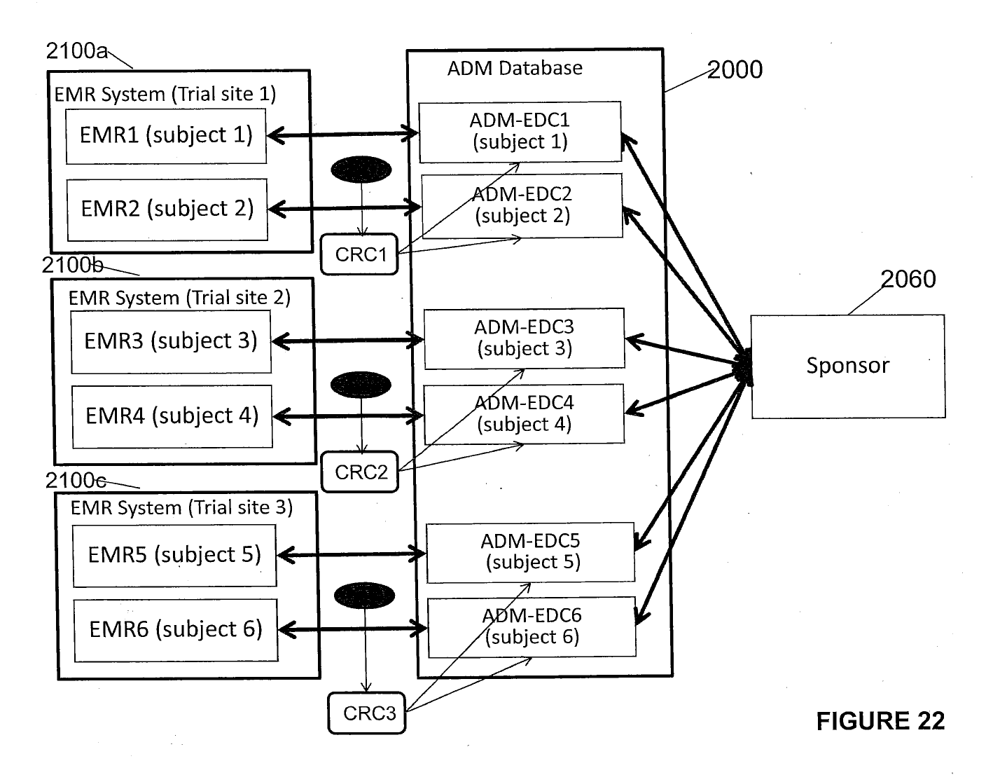

FIG. 20 is a schematic diagram showing various interactions between ADMs for two patients and various ADM-equipped EMR systems at different health care organizations (HCOs) that have EMRs for those patients in accordance with some embodiments (left side of diagram). The appearance of the arrows in FIG. 20 (and in FIGS. 21 and 22) is used as an indicator of the amount (extent) of the interactions represented. Thick arrows represent more extensive interactions than do thin arrows and arrows with dashed lines. Arrows are often represented as two-headed for convenience and without implying anything about the nature of the interactions. It will be understood that the nature of the interactions represented by the arrows may vary. Interactions may or may not be bidirectional, may or may not be electronic, may include, e.g., reviewing data, entering data, copying data from one record (e.g., an electronic record), file (e.g., an electronic file), device, or storage medium (e.g., non-transitory computer-readable storage medium) to another, making queries, receiving responses. For example, in general, a HCP would be able to review and/or query an ADM and/or the ADM database and would also be able to enter data into ADMs of his or her patients (directly and/or via their linked EMRs). However, a medical researcher may be able to query an ADM and/or the ADM database and receive a response but would not be able to enter data or modify the ADM data (unless the medical researcher is a HCP with patients having ADMs in the ADM database). The various different types of interactions are not differentiated in the figure. It should be understood that arrows may represent making queries or requests, reviewing data, extracting data, entering data, copying data, etc., as appropriate to the particular entities or individuals involved.

ADM database 2000 holds ADMs for Patient 1 and Patient 2. Patient 1 has confirmed diagnoses of Type II diabetes (Patientl ADM1), osteoarthritis (Patient1 ADM2), and asthma (Patient1 ADM3). Patient 2 has confirmed diagnoses of asthma (Patient2 ADM1) and thyroid cancer (Patient2 ADM2). Both patients have the same primary care provider (PCP), indicated as HCP1. HCP1 practices at a first HCO (e.g., a physician practice in the community where Patient 1 and Patient 2 live), which uses ADM-equipped EMR system 1 (2100a) to maintain EMRs for its patients. Thus EMR system 1 contains EMRs for Patient 1 and Patient 2. For the sake of clarity, the EMRs themselves are not shown in the figure. Medical data is entered into these EMRs during or after patient visits to HCP1 by Patient 1 and Patient2. The data may be entered by PCP1 and/or by other care providers working at HCO1 such as nurses, may be entered from a clinical lab report or information system, etc. The data are transmitted to the appropriate ADMs in ADM database 2000 upon entry into EMR system 1. For example, if PCP1 orders a blood glucose measurement for Patient 1, the result is transmitted to ADM1 for Patient 1 (the Type II diabetes ADM), which contains a field intended for blood glucose measurements, a standard part of care of a patient with Type II diabetes. Similarly, if HCP1 measures a range of movement of Patient 1's knees, the data is entered into Patient 1's EMR and is transmitted to Patient 2's ADM2 (osteoarthritis). HCP1 oversees the overall care of Patients 1 and 2 and generates and reviews data relevant to each of their diseases. Thus, EMR System 1 interacts with all ADMs of Patient 1 and Patient 2. (It will be understood that the amount of such interaction may vary and may differ for different diseases, patients, HCPs, etc.)

Patients 1 and 2 may receive medical care from a variety of specialists in addition to their PCP. For example, Patients 1 and 2 may receive care for their asthma from a pulmonologist, indicated as HCP2. HCP2 may work in a different physician practice (HCO2), which uses ADM-equipped EMR system 2 (2100b) to maintain EMRs for its patients. EMR System 2 may be a different EMR system than EMR System 1 (e.g., from a different EMR system vendor) and use a different ADM component to interface with the ADM database. HCP2 is also an investigator in a clinical trial of a new therapy for asthma, and Patients 1 and 2 are subjects in the trial. The asthma ADMs may therefore contain certain fields that are specifically included for purposes of the clinical trial and/or may be ADM-EDCs. Patient 1 and Patient 2 each have an EMR in EMR System 2. Medical data is entered into these EMRs during or after patient visits to HCP2 by Patient 1 and Patient2. The data may be entered by PCP2 and/or by other care providers working at HCO2 such as nurses, may be entered from a clinical lab report or information system, etc. The data are transmitted to the appropriate ADMs in ADM database 2000 upon entry into EMR system 2. HCP2 is concerned mainly with asthma when seeing Patient 1 and Patient 2; thus most of the data generated as a result of visits by Patients 1 and 2 pertains to asthma. HCP2 may be interested in reviewing data that may have been entered at HCO1 after visits by Patient 1 and Patient 2 to their PCP (HCP1). HCP2 can review this data in the asthma ADMs, which are accessible from within Patient 1 and Patient 2's EMRs in EMR System 2. If desired, HCP2 can import this data into the EMRs in EMR System 2. Such importing may be selected (e.g., by HCP2 or HCO2) to occur automatically or upon request. In certain embodiments, at least some data that are entered into the asthma ADMs specifically for clinical trial purposes are not accessible to other EMR systems. When HCP2 enters data pertaining to asthma into EMR System 2, this data is transmitted to the appropriate asthma ADMs in ADM Database 2000. Thus there is extensive interaction between EMR System 2 and the asthma ADMs in ADM database 2000, as indicated by the use of thick arrows. HCP2 may, if desired, review data relating to other illnesses in the appropriate ADMs and data pertaining to such diseases can also be entered via EMR System 2. However, in general, HCP2 may typically be expected to devote less attention to non-pulmonary diseases such as osteoarthritis or Type II diabetes than to pulmonary diseases such as asthma; thus there may be less interaction between EMR System 2 and the non-asthma ADMs than between EMR System 2 and the asthma ADMs. By accessing the asthma ADMs, HCP1 is able to review data relating to asthma that was entered by HCP2, even though such data was entered at a different HCO that uses a different EMR System. Such data may also be copied into the EMRs in EMR System 1 (and/or EMR System 3) either automatically or on request in certain embodiments.

Patient 2 has a confirmed diagnosis of thyroid cancer (in addition to asthma) and is under the care of an oncologist (HCP3) for this disease. HCP3 may practice at a hospital (HCP3) with special expertise in treating cancer patients. HCO3 may use ADM-equipped EMR System 3 (2100c), which may be different from those used by HCO1 and HCO2 (e.g., from a different vendor). Since HCP3 is typically mainly concerned with treating Patient 2's thyroid cancer, EMR System 3 is shown as interacting mainly with Patient 2's thyroid cancer ADM (Patient2 ADM2), as indicated by the thick arrow. HCP3 may at times be interested in reviewing the status of Patient 2's asthma and can do so by accessing Patient 2 ADM1. Data in Patient2 ADM1 may also be copied into the Patient 2's EMR in EMR System 3 either automatically or on request in certain embodiments. HCP1 can follow the treatment of Patient 2 by HCP3 for thyroid cancer by accessing Patient 2 ADM2. Data in Patient2 ADM2 may also be copied into Patient 2's EMR in EMR Systems 1 and 2 either automatically or on request in certain embodiments.

In certain embodiments Patients 1 and 2 can interact with their ADMs, as indicated on the left side of FIG. 20. They may, for example, enter symptom data, review their medications and lab results, receive reminders about appointments, receive updates about clinical trials relating to their diseases, etc.

As mentioned above, Patients 1 and 2 in FIG. 20 are subjects in a clinical trial of a new therapy for asthma, which trial is sponsored by a pharmaceutical company that seeks to obtain approval to market the therapy. Sponsor 2060 is able to interact with the asthma ADMs in any of a variety of ways in various embodiments. For example, the ADMs may be accessed remotely by Sponsor 2060 and reviewed or data may be extracted therefrom. Data may be transmitted in real-time from the ADM database to a database at Sponsor 2060's location. Prior to enrolling Patients 1 and 2 in the trial, Sponsor 2060 may have reviewed their asthma ADMs and determined that they were potentially eligible for the trial. Sponsor 2060 may have contacted Patients 1 and 2 via their ADMs to inform them of the upcoming trial. Sponsor 2060 may have invited HCP2 to be an investigator in the trial based at least in part on reviewing asthma ADMs of HCP2's patients and determining that HCP2 has multiple potentially eligible patients. As discussed herein, ADM data may be de-identified, thus Sponsor 2060 would not learn the identity of the subjects. In certain embodiments the asthma ADMs may be trial-specific ADMs or ADM-EDCs (though not specifically indicated as such in FIG. 20).

Medical researcher 2062 in FIG. 20 is interested in studying Type II diabetes. To this end, Medical Researcher 2062 may access Type II diabetes ADMs in ADM Database 2000 (e.g., Patientl ADM1). Medical Researcher 2062 may, for example, have access to thousands or millions of Type II diabetes ADMs in ADM Database 2000 and may, for example, analyze data from these ADMs identify correlations, test hypotheses, investigate drug efficacy, drug interactions, etc. As discussed herein, ADM data may be de-identified, thus Medical Researcher 2062 would not learn the identity of the patients. The ADMs may be assigned a number, and Medical Researcher 2062 may reference them in that manner in publications reporting results obtained at least in part by analyzing the ADM data.

Pharmaceutical company 2064 in FIG. 20 makes an experimental therapy available to patients with thyroid cancer under an expanded access program. The patients may be ineligible or unable to participate in a clinical trial of the therapy. HCP3 may have learned of the availability of the experimental therapy via an Experimental Therapies link in the thyroid cancer ADM and may have requested, e.g., using functions provided by the thyroid cancer ADM, that Patient 2 be permitted to receive the experimental therapy. Pharmaceutical company 2064 may have agreed to make the therapy available in part because HCP3 utilizes an ADM-equipped EMR system. Pharmaceutical company 2064 can access data in Patient 2's thyroid cancer ADM and may thereby gather data that may be useful in further development of the experimental therapy (e.g., in determining future trial criteria, seeking approval from a regulatory agency, etc.)

FIG. 21 shows a schematic diagram of a system (shown in the upper part of the figure) in which an ADM database interfaces with an ADM-equipped EMR system and with an EDC system in accordance with some embodiments. The lower part of the figure shows a scenario in which a conventional “stand-alone” EDC system (2090) that requires entry of data (at least some of which may come from subjects' EMRs) into the EDC system by study personnel is used. CRC1 represents study personnel in this scenario. CRC1 enters data manually into conventional EDC system 2090. Some of the data may be copied by CRC1 from the subjects' EMRs into the conventional EDC system while some data (e.g., certain trial-specific data) may be entered only into the conventional EDC system from data sources outside the EMR. Thus extensive interactions between CRC1 and EMRs and between CRC1 and the conventional EDC System are depicted. Data sources outside the EMR context are represented by solid oval 2200. Such sources may, for example, be tests that are performed specifically for purposes of the trial protocol, results of which may not be entered into the subject's EMR. CRC1 or other study personnel enters such data directly into conventional EDC system 2090. Study personnel at each site may devote significant time to data entry into such conventional EDC systems. Sponsor 2060 (e.g., sponsor personnel responsible for monitoring the trial) can interact with the conventional EDC system 2090 in this scenario but do not have access to data in the patient EMRs. Sponsor's queries (e.g., regarding missing or potentially inaccurate or inconsistent data) are typically handled via study personnel (e.g., CRC1) or through monitoring visits to Trial Site 1. In a multi-site trial, Sponsor 2060 would typically interact with study personnel and EDC systems at each site.

In the scenario shown in the upper part of FIG. 21, EDC system 2080 interfaces with an ADM database (e.g., via a plug-in to the EDC system). ADM Database 2000 typically contains all ADMs for subjects in the trial. (There may or may not also be trial sites that do not use ADM-equipped EMR systems.) It will be understood that ADM Database 2000 ordinarily contains numerous other ADMs (not shown), which may include any of various ADM types, e.g., other ADMs for the same patients, ADMs for other patients, ADM-SCs, ADM-EDCs for other trials, etc. Data entered into the subjects' EMRs is copied by the system into the appropriate ADMs, as described herein. Data may alternately be entered directly into the ADMs in some embodiments and copied from there into EDC System 2080. The EMRs and ADMs may be synchronized so that data entered into either one populates the appropriate fields of both. Data relevant to the trial is copied from the ADMs into the appropriate records in ADM-interfaced EDC system 2080. Thus a substantial portion of the data entry into ADM-interfaced EDC system 2080 that is required for the trial occurs automatically without requiring a separate data entry step on the part of study personnel. Study personnel in this scenario (represented as CRC2) may typically have significantly less interaction with the EMRs and with the EDC system than do study personnel (represented as CRC1) in the scenario in which a stand-alone EDC system is used. The ADMs may perform data checking and quality control function, which may help identify and avoid potential errors before the data is copied to the EDC system. The ADMs may or may not be trial-specific ADMs. CRC2 may enter some trial-relevant data into ADM-interfaced EDC system 2080 from subjects' EMRs (e.g., in the case of trial-relevant data for which a field does not exist in the ADM, e.g., because the ADM is not a trial-specific ADM and the data would not ordinarily be collected as part of standard of care treatment of the disease) and/or from data sources outside the EMR/ADM context (represented by small solid ovals not numbered in FIG. 21). Alternatively (not shown), e.g., if the ADMs are trial-specific ADMs, CRC2 may enter such data directly into the ADMs, from which such data are copied into the ADM-interfaced EDC system (and may be copied into the EMRs also). In such embodiments, e.g., if the ADMs are trial-specific ADMs, all trial-relevant data may typically be captured by the ADMs. In certain embodiments regardless of whether trial-specific ADMs or ADMs that are not trial-specific are used, CRC2 performs significantly less data entry in the scenario in which ADM-interfaced EMR system 2080 is used than does CRC1 in the scenario in which a conventional EDC system 2090 is used. Sponsor 2060 (e.g., sponsor personnel responsible for monitoring the trial) interacts with the ADM-interfaced EDC system 2080. In some embodiments (not shown in FIG. 21), sponsor 2060 may also be able to interact with the ADM database. Sponsor 2060 may engage a CRO (not shown) to perform at least some such interaction(s) and/or other trial-related activities.

FIG. 22 is a schematic diagram of a system in which trial-specific ADMs (ADM-EDCs) are used to perform all electronic data capture functions for a clinical trial at three trial sites in accordance with some embodiments, eliminating the need for a separate EDC system at these sites. ADM Database 2000 typically contains all ADM-EDCs for subjects in the trial. (There may or may not also be trial sites that do not use ADM-equipped EMR systems.) It will be understood that ADM Database 2000 ordinarily contains numerous other ADMs (not shown), which may include any of various ADM types, e.g., non-trial related ADMs for the same patients (e.g., relating to other diseases), ADMs for other patients, ADM-SCs, ADM-EDCs for other trials, etc. The ADM-EDCs capture trial-relevant data as it is entered into the subjects' EMRs in ADM-equipped EMR system 2100a at Trial Site 1, ADM-equipped EMR system 2100b at Trial Site 2, and ADM-equipped EMR system 2100c at Trial Site 3. Data may alternately be entered directly into the ADM-EDCs in some embodiments, e.g., by a HCP in the course of a subject visit. The EMRs and ADM-EDCs may be synchronized so that data entered into either one populates the appropriate fields of both. The three ADM-equipped EMR systems may be different (e.g., from different vendors). Through ADM functionality (e.g., provided via distinct ADM components) each system can use ADMs. CRC1, CRC2, and CRC3 represent study personnel at the three trial sites. CRC1, CRC2, and CRC3 may enter some data into the ADM-EDCs from sources outside the EMR/ADM-EDC context (represented as small solid ovals near the EMR systems of each trial site). However, most trial-relevant data is typically entered directly into the EMRs or ADM-EDCs, thus avoiding a separate data entry step for such data by study personnel. Sponsor 2060 (e.g., sponsor personnel responsible for monitoring the trial) can interact with the ADM database, which contains all of the ADM-EDCs for subjects in the trial. Sponsor 2060 may, for example, access data from ADM-EDCs used in the trial and may perform any function for which a conventional EDC system would be used and, in some embodiments, one or more additional functions. For example, Sponsor 2060 may in some embodiments interact with subjects via the ADM-EDCs (not shown in FIG. 22). Sponsor 2060 may engage a CRO (not shown) to perform at least some such interaction(s) and/or other trial related activities.

It should be understood that the systems shown in FIGS. 20, 21, and 22, and descriptions thereof, are merely exemplary of certain embodiments and are not to be considered limiting in any respect. Details of implementation, operation, interactions, components, features, configurations, functions, uses, etc., may vary. It should also be understood that activities such as transmission, importing, copying of data, etc., may be initiated and/or executed at least in part by any of various components, modules, processes, devices, etc., in various embodiments, and descriptions thereof herein should not be considered limiting.

In some embodiments of any aspect(s) hereof, database updates, feedback, or response may be performed or provided in a timely manner. In some aspects an average time for providing feedback or response to a HCP or updating an EMR or ADM may be selected so as to not substantially interfere with or delay normal workflow of the HCP. In some aspects an average response or update time may be selected to be below a predetermined value. In some embodiments a predetermined value may be equal to or less than 1, 2, 5, 10, 15, 20, 30, 40, 45, 50, or 60 seconds for, e.g., at least some classes of actions. In some embodiments an alert pertaining at least in part to time anticipated to be required for response or update may be provided, said alert comprising, e.g., an estimated time, an indication that a response or update may take more than a predetermined time, an option to abort an update or query, etc. In some embodiments, database accesses, updates, or queries are at least in part prioritized. Prioritization may take into account, e.g., factors such as the user (e.g., whether the user is a contributor or subscriber), the nature of the action, prior response times to the user or during a session, etc. For example, an action performed in response to a contributor, e.g., a HCP, may be assigned a higher average priority than an action performed by a non-contributor.

Applications for Portable Electronic Device

In some aspects, the disclosure may provide an application for an electronic device, wherein the application is operative to interface with a computer that maintains user account data as described above (“user account application”). The user account application provides the user with access to at least a portion of his or her user account data. For example, in some embodiments in which the business entity issues shares as incentives to contributors, the user account application provides the user with access to at least a portion of his or her share account data held in the share database (described above). For example, the application informs the user (e.g., upon request of the user) of the number of shares owned by or earned by the user and/or the value of said shares or current share price (e.g., if the company is a public company). The user account application may display the information in any suitable format. For example, the share data may be displayed as a graph. In some embodiments the user account application allows the user to purchase and sell shares in the business entity and, e.g., track the share price over time. In some embodiments, only users (or non-user share holders) who own or have the possibility of owning shares may be provided with the share account application. The user account application may not permit the user to change the content of the user account database (although features may be provided that allow the user to change certain content such as UserID or password). The user account application may not provide access to certain content of the user account database. For example, the user account database may include information assembled by the business entity regarding the user's usage of the EMR system. In some embodiments at least some such information may not be accessible to the user.

The electronic device may comprise any suitable type of electronic device. For example, the electronic device may comprise a portable electronic device that a user may hold in his or her hand, such as a portable digital assistant (PDA), also referred to as a portable data assistant, a smartphone, a tablet computer, etc. The electronic device may be a larger portable electronic device, such as a laptop computer. As known in the art, PDAs are small, e.g., hand-held, computers, that are frequently used for tasks such as maintaining a calendar, list of contacts (e.g., email addresses), and other information. PDAs may contain application programs such as word processing programs, web browsers, PDF viewers, etc. As used herein, a “smartphone” may be an electronic device that combines the functions of a wireless phone and a PDA within a single unit. A tablet computer may be a computer that is may be somewhat larger than a mobile phone or personal digital assistant, comprises a flat touch screen, and is primarily operated by touching the screen. It may use an onscreen virtual keyboard.

Often a portable electronic device may weigh under about 1-2 pounds, e.g., between about 3 ounces and about 1.5 pounds. For example, a smartphone or PDA may weigh between about 3 ounces and about 6 ounces and height and width dimensions in the range of less than about 7×5 inches and depth less than about 0.5-1.0 inch, though smaller or larger weight and/or dimensioned devices may be used. Exemplary portable electronic devices include, e.g., a PDA such as an iTouch (Apple, Inc.), a smartphone such as an iPhone (Apple, Inc.) or Galaxy phone (Samsung), or a tablet computer such as the iPad or iPad mini (Apple, Inc.). In some embodiments a portable electronic device may be wearable, e.g., as a wristwatch, armband, etc.

A portable electronic device may include components that may be found in such devices, e.g., control circuitry, storage/memory, input/output circuitry, communications circuitry, processing circuitry, etc. In some embodiments, one or more of such components of the device may be combined or omitted. In some embodiments, the portable electronic device may include other components such as, for example, a proximity sensor, a power supply such as a battery, a display, a positioning system, a camera, an accelerometer, an ambient light sensor, other sensors, an input mechanism, etc.) or multiple instances of one or more such components. In many embodiments, the portable electronic device may possess wireless connectivity. For example, the device may have Bluetooth, Wi-Fi wireless network connectivity, and/or the ability to connect to wireless Wide Area Networks, such as those provided by cellular telecommunications companies.

In some aspects, the disclosure may provide an application for a portable electronic device (PED), wherein the application is operative to interface with a computer that maintains the EMR database. The application may consist of a single application or may comprise multiple applications that provide different functions. For purposes of convenience, the one or more applications will be referred to herein as the “EMR application”. In some embodiments, the EMR application provides a user with full functionality of the EMR system. For example, a contributor may create, view, update, or otherwise use EMRs or ADMs of his or her patients, enter orders, or perform other activities (e.g., data analysis activities such as those described herein) in a similar or essentially identical manner as could be done using a standard notebook or desktop computer. In some embodiments only a subset of the EMR functions may be supported, which subset may depend at least in part on the capabilities of the particular portable electronic device and/or its operating system. For example, the user may be able to view but not update EMRs in some embodiments. It will be understood that details of the applications described herein may be customized for any particular portable electronic device platform of interest. In some embodiments, a EMR application running on the portable electronic device may synchronize with corresponding application(s) running on other electronic devices used by the user so that a seamless and integrated user experience is provided.

In some embodiments, a user may receive health-related offers or information through the EMR application. In some embodiments a user may be offered an opportunity to opt out of or discontinue receiving offers or information or would receive them only upon affirmatively requesting to do so. In some embodiments the offers or information may be tailored to a particular user. For example, a user may perform a search on the Internet for a particular disease or may look up the disease in an information resource provided by the EMR system. In response to the user researching this particular disease, the EMR application may provide offers associated with the researched disease. For example, the EMR application may access a database of clinical trials and search for trials that are recruiting subjects who have the disease. The EMR application may notify the user regarding such clinical trials or may inform the user of recent research studies relating to the disease.

In some embodiments the EMR application may comprise or interface with one or more additional health-related applications for an electronic device, e.g., a portable electronic device. Such application may be, for example, a medication tracking application, an exercise tracking application (e.g., a pedometer application), a weight tracking application, a calorie counting application, a heart or blood pressure monitoring application, etc.

In some aspects, the disclosure relates to ADM networks, ADM communities, and/or applications (“apps”), e.g., patient apps, that may, for example, facilitate participation in or formation of one or more ADM networks and/or ADM communities and/or may facilitate disease management. In some aspects, the disclosure provides methods of creating, maintaining, offering access to, providing, hosting, updating, and/or using ADM networks, ADM communities, and/or applications that facilitate participation in or formation of one or more ADM networks and/or ADM communities and/or may facilitate disease management. In some aspects, computer-executable instructions, apparatus, and/or systems for creating, maintaining, offering access to, providing, hosting, updating, and/or using ADM networks, ADM communities, and/or applications that facilitate participation in or formation of one or more ADM networks and/or ADM communities and/or may facilitate disease management, are provided. In some aspects, computer-readable media comprising any such computer-executable instructions are provided. In some aspects, methods of, and computer-executable instructions useful for, updating, maintaining, adding to, accessing, and/or using ADMs or an ADM database via patient apps are provided. In some aspects, methods of performing or using, and computer-executable instructions that, when executed, perform, one or more functions of a patient app are provided.

In some aspects, “patient ADM network” refers to the set of patients that have at least one confirmed ADM. In some embodiments patient ADM network is composed of patients that have at least one active ADM (“active members”), e.g., patients all of whose ADMs have become inactive may be removed from the network (with a possibility to be reinstated if at least one ADM becomes active again or a new active ADM is added). In some aspects, “patient ADM network” for a particular disease refers to the set of patients that have an ADM for that disease. In some embodiments patient ADM network for a particular disease is composed of patients that have an active ADM for the particular disease (“active members”), e.g., patients whose ADM for the disease has become inactive may be removed from the network (with a possibility to be reinstated if the ADM becomes active again or a new ADM for the disease is created and confirmed). “HCP ADM network” refers to HCPs who have at least one patient with a confirmed ADM and/or, in some embodiments, may comprise HCPs who use an ADM-equipped EMR system or have agreed to use ADMs for the disease. In some embodiments a HCP ADM network is composed of HCPs who have at least one patient with a confirmed active ADM. “HCP ADM network” for a particular disease refers to HCPs who have at least one patient with a confirmed ADM for the particular disease and/or, in some embodiments, may comprise HCPs who use an ADM-equipped EMR system or have agreed to use ADMs for the disease. In some embodiments HCP ADM network is composed of HCPs who have at least one patient with a confirmed active ADM for the particular disease. In some aspects, “HCO ADM network” refers to HCOs that use or have agreed to use ADMs, e.g., in their ordinary medical record keeping and/or in regard to conducting or offering clinical trials, managed access programs, or repositioned therapies. In some aspects, “HCO ADM network” comprises or is composed of HCOs that use or have agreed to use an ADM-equipped EMR system. It will be understood that an HCO may not use ADMs for all patients and/or purposes. For example, not all HCPs working at a particular HCO may use an ADM-equipped EMR system. In some aspects, “sponsor ADM network” refers to sponsors who use ADMs for one or more purposes relating to experimental therapies or post-marketing surveillance and, in some embodiments, may comprise sponsors who are set up to do so (e.g., have been granted appropriate access to an ADM system). In some aspects, “payer ADM network” refers to payers who use ADMs for one or more purposes relating to their function as payers and, in some embodiments, may comprise payers who are set up to do so (e.g., have been granted appropriate access to an ADM system). Other ADM networks composed of entities or individuals that use or are set up to use ADMs may exist in certain embodiments. In some aspects, “ADM network” refers to a particular ADM network (e.g., a patient ADM network for a particular disease) or the collective network of “HCP ADM network” and “patient ADM network”, and, in some embodiments, may further include “HCO ADM network”, “sponsor ADM network”, “payer ADM network”, and/or one or more other networks of individuals or entities that use ADMs for one or more purposes. It will be understood that an individual or entity may be a member of one or more ADM network(s) and may use ADMs for one or more purposes. In some embodiments an opportunity, option, activity, app, device, function, product, service, etc., that may be offered or available to members of an ADM network may be said to be offered or available “through the ADM network”. For example, an experimental therapy that may be offered by or available from HCPs and/or HCOs in an HCP ADM network or HCO ADM network (e.g., that such HCP and/or HCO may make available to appropriate patients) may be said to be offered “through the ADM network”. In some embodiments an opportunity, option, activity, app, device, function, product, service, etc., may be available only to members of an ADM network. In some embodiments patients in a patient ADM network for a particular disease may have access to one or more functions or opportunities that facilitates their access to experimental therapies for the disease. In some embodiments HCPs in a HCP ADM network for a particular disease may have access to one or more functions or opportunities that facilitates their ability to offer experimental therapies for the disease to their patients, e.g., as described herein. In some embodiments “patient ADM community” refers to patients that have joined an ADM community via, e.g., a Dx app (described further below). In some embodiments an ADM community, e.g., a patient ADM community may overlap with or have one or more functions or features of an ADM-related social network, e.g., any of the ADM-related social network or social media functions or features described herein. In some embodiments an ADM community comprises patients that may interact with one another, participate in or view discussion fora, or engage in various other social media type activities. . In some embodiments “HCP ADM community” refers to HCPs that have joined an ADM community via, e.g., an ADM-related social network or via an ADM-equipped EMR system. In some embodiments a HCP ADM community comprises HCPs that may interact with one another, participate in or view discussion fora, or engage in various other social media type activities.

In some aspects, described herein is an application for a portable electronic device (PED), e.g., a smartphone or tablet, wherein the application provides one or more functions of use to or for use by an individual who is a patient or prospective patient. In some embodiments such an app may be referred to as a “patient app”. A prospective patient may be an individual who may become a patient, e.g., an individual who has one or more diseases or is suspected (e.g., by the individual and/or by a HCP whom the individual has consulted) of having one or more diseases or who is at increased risk of developing one or more diseases, e.g., within a selected time period, or who is otherwise in need of or may benefit from medical care or attention.

In some aspects described herein is a “Diagnoses app” or “Dx app” that provides information and functionality relating to one or more diseases (“diagnoses”) that an individual has or is suspected of having or is at increased risk of developing, wherein at least some of the information and functionality is organized on a disease-specific basis. For purposes of description an individual who installs or uses a Dx app for one or more purposes relating to his or her health or health care (whether himself or herself or through someone else who acts on his/her behalf such as a caregiver), may be referred to as a “patient”, whether the individual is a patient or prospective patient. In some embodiments a “Dx app” may serve as a central or “master” app of a patient app package, which package may include one or more apps relating to health promotion, one or more disease-specific apps, or others. In some embodiments a Dx app comprises a disease list, which may provide access to one or more functions or information relating to diseases in the list, such functions or information may be organized or provided at least in part on a disease-specific basis. In some embodiments such functions and/or information are provided by one or more disease-specific apps. In some aspects, a Dx app may, e.g., through a disease-specific app, assist a patient having a disease to better understand and/or manage the disease, locate HCPs capable of treating the disease, provide an opportunity to interact with other patients with the disease, and/or facilitate the patient gaining information about and/or access to therapies, e.g., experimental therapies, for the disease. In some aspects, a Dx app may, e.g., through one or more disease-specific apps, motivate a patient to contribute positively to their own health or disease management.

In some aspects, a variety of patient apps are described herein. In some embodiments at least some patient apps may interact or interface with one another and/or function together as part of a patient app package. It should be understood that a patient app or patient app package may operate across multiple computers. For example, a patient may access and use an app from a PED, desktop computer, notebook computer, or any appropriate computer. Information may be synchronized across devices so that, for example, data entered into a first device (e.g., a PED) is available essentially immediately across the various devices on which the app is installed. It should also be understood that where reference is made to “installing” or “downloading” (e.g., downloading or installing a component such as an app, update, etc.), such terms may be used interchangeably and may refer to both downloading and installing, activating a pre-installed component such as an app or update, or taking other action that makes a component available for use with a particular device (e.g., a PED). Embodiments in which one or more apps are provided to a patient on a computer-readable medium such as a “thumb drive” or DVD are provided. It should also be understood that in some embodiments at least some computer-executable instructions of a component such as an app may be executed remotely, e.g., on one or more remote servers (e.g., in the cloud), rather than on the device on which the component is being used by a user (e.g., a patient). It should also be understood that data entered into a device, e.g., via an app, may be stored remotely, on a user's device, or at least in part on both.

A Dx app may interact with one or more additional apps and/or may comprise one or more components (which may be referred to as “sub-apps”) that collectively comprise a patient app package. In some embodiments a Dx app may interact with an EMR system or EMR database, e.g., an ADM database. A Dx app used by a particular patient may interact with one or more ADMs of that patient, which ADMs may be associated with a single EMR or may be distributed among multiple EMRs of that patient. In some embodiments a Dx app may transmit information to an ADM (e.g., information entered by a patient or gathered by a patient monitoring device (which monitoring device may be a PED or a device that interacts with a PED), obtain information from an ADM, process information obtained from an ADM, and/or display information obtained from an ADM or generated by processing such information. Where reference is made herein to a function, feature, activity, or option that may be performed or offered by a Dx app, such function, feature, activity, or option may be performed or offered by or through any one or more apps of a patient app package in various embodiments.

In some embodiments a Dx app provides one or more functions that may be useful to a patient in managing one or more of the patient's disease(s). In some embodiments a Dx app allows a patient to access one or more of his or her ADMs (see discussion below). In some embodiments a patient who installs a Dx app may be asked whether he or she wants to also install one or more apps that pertain to patient behaviors or habits relevant to health, e.g., diet (“Diet app”), exercise (“Ex app”), or medications (“Rx app”). Such apps may be referred to as “health promotion apps”. A patient app package may comprise a Dx app and, in some embodiments, one or more health promotion apps or monitoring apps described herein. A patient app package may further comprise one or more disease-specific apps, which may be selected by a patient and/or based on a patient's ADMs or diseases. Thus in some embodiments a patient app package may be individualized for a particular patient.

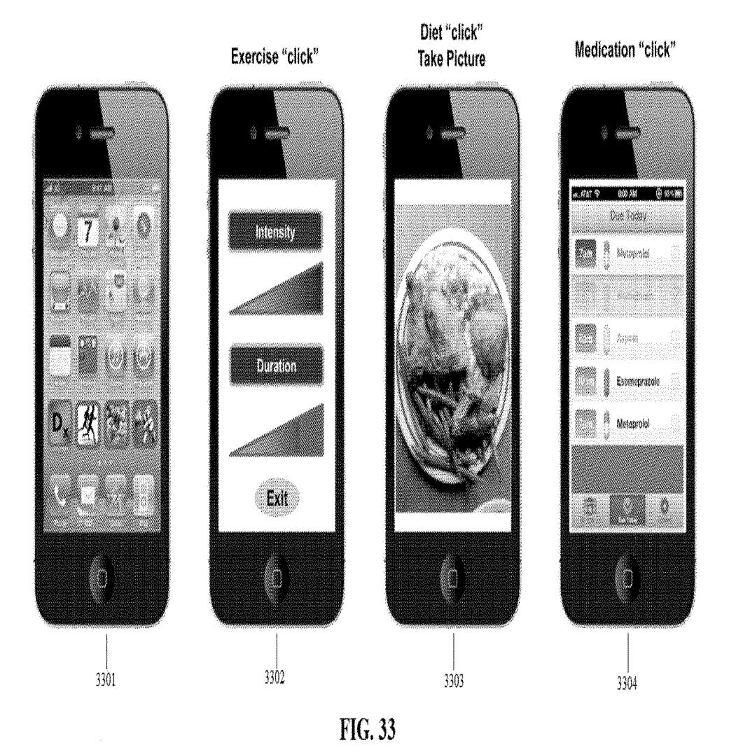

A health promotion app may provide a monitoring function, reminding function, alerting function, may offer suggestions or advice, may interact with a Dx app, may interact with a disease-specific app, and/or may interact with one or more ADMs. Monitoring capability may include providing means whereby a patient can keep track of an activity or habit via the app. Reminding function may provide a reminder to a patient to undertake certain activities on a timely basis. Alerting function may inform a patient when action should be taken to avoid an undesired or potentially harmful consequence or to convey information outside of or in addition to that provided by the reminding function. “Diet” refers generally to the type and amount of foods and beverages that a person consumes. It should be understood that “diet” may be referred to as “food”, “nutrition”, “eating habits” or other similar terms. It will be understood that names of apps provided herein are generally representative of suitable names and are not to be considered limiting. Other names that appropriately convey or are associated with the purpose or content of the app may be used. For example, a “Diet app” (see e.g., FIG. 33 interface 3303, which shows a screen of a “Diet app”) may be referred to as a “Food app”, “Eating habits app” or “Nutrition app”. Exercise may be used interchangeably with physical activity. For example, an “Exercise app” (see, e.g., FIG. 33, interface 3302, which shows a screen of an “Exercise app”) may be referred to as an “Activity app”. Examples include sports, running, jogging, swimming, biking, aerobics, walking, weightlifting, exercise machines, dancing, and activities that may be part of daily life that provide physical activity, such as walking, climbing stairs, housework, or others. In some embodiments multiple versions of an Exercise app may be offered that may vary, e.g., depending on a patient's baseline physical condition. In some embodiments if appropriate an “Activities of Daily Living” app may be provided; such activities may include, e.g., dressing, bathing, eating, etc. Similarly, multiple versions of a Diet app and/or Medication app (see e.g., FIG. 33, interface 3304, which shows a screen of a Medication app) may be offered depending, e.g., on patient characteristics. “Medications” refers generally to those therapeutic agents (typically pharmaceutical agents) that an individual takes (e.g., on a regular or as-needed basis), e.g., for treatment of a disease. In some embodiments medications are those therapies that a patient takes or uses outside the health care setting, which the patient and/or patient's caregiver is generally responsible for ensuring that the patient adheres to the appropriate schedule and/or other complies with other instructions, if any (e.g., timing with regard to food consumption, etc.). In some embodiments medications maybe limited to pharmacological therapies. Pharmacological therapies may include medications prescribed by a HCP, which may be, for example, in the form of tablets, pills, capsules, caplets, inhalers, injections (e.g., subcutaneous). creams, liquids, ointments, gels, etc. In some embodiments medications may include pharmacological therapies to be taken or used on a regular basis, e.g., one or more times per day, every other day, etc. In some embodiments medications that a patient may take on an as-needed (PRN) basis, e.g., on prescription or advice of a physician, may be included. In some embodiments over-the-counter medications (e.g., OTC medications taken on advice of a HCP) may be included. In some embodiments medications may include one or more non-pharmacological therapies such as physiotherapy (e.g., exercises performed under advice of a physician, physiotherapist, etc.). In some embodiments medications may include dressing changes or other patient care activities. In some embodiments one or more separate apps may be provided for non-pharmacological therapies and/or patient care activities. In some embodiments an app may comprise one or more functions or extensions relevant for a particular transient condition or state, e.g., pregnancy, post-surgical, etc. In some embodiments an app, e.g., a Dx app, may be represented by an icon, which may be visible on a home screen or main screen of a PED (see FIG. 33, interface 3301). A “home screen” is often a screen that is seen when a PED is turned on by a user for the first time. (It will be understood that a passcode may be required before the home screen is accessible. It will also be understood that if the phone has been turned off or entered an inactive or sleep mode while a function or app is being used, the phone may return to such function or app rather than to the home screen when it is turned back on. It will also be understood that in some embodiments a home screen may occupy more than one physical screen on a device. For example, a user may swipe to the left or right or zoom out to view portions of the home screen that may not all fit conveniently on a single physical screen.) A home screen often provides an access point for basic functions of the device (e.g., phone, email, text message) as well as for at least some of the applications that are installed. A home screen often displays icons for basic functions of the device as well as for at least some of the installed apps. A PED may have a “home” button that can be pressed to return to the home screen, e.g., if the user is using an application. In some embodiments tapping on or otherwise selecting an icon “opens” the app, e.g., makes app functionality available. An icon may contain one or more letters, numbers, words, abbreviations, symbols, images, or combinations thereof. In some embodiments an icon may contain the letters “Dx”, the phrase “My Dx”. In some embodiments an icon for a Dx app may contain a Greek letter, e.g., a delta (Δ or δ). In some embodiments an icon for a Dx app may contain an image or symbol that may be associated with health or health care, such as a rod of Asclepius. In some embodiment a Diet app, Ex app, and/or Rx app may be installed automatically if a patient installs a Dx app. In some embodiments a Dx app comprises a Diet app, Ex app, and/or Rx app. In some embodiments, icons allowing access to the functions of the Diet app, Ex app, and/or Rx apps are visible, e.g., on the home screen, after installation. In some embodiments such icon(s) may be associated with the particular app. For example, a Diet app may be represented by an image of food, a Rx app may be represented by “Rx” or by an image of pills, an Ex app may be represented by an image of an individual engaged in exercise or by “Ex”, by way of example. In some embodiments at least some icons for health-related apps (e.g., Dx app, health promotion apps) may be located near each other on a home screen, e.g., adjacent to each other. In some embodiments icons for health-related apps are located in one or more folders on a home screen. For example, a folder may be named “Health” or “H” and may contain a Dx app and one or more health promotion or monitoring apps, e.g., a Diet app, an Ex app, and a Rx app. It should be understood that depictions and descriptions of a smartphone screen herein are exemplary. Such depictions and descriptions may represent a screen of any device, e.g., a tablet, notebook computer, or other computer. In certain embodiments such device is a PED.

In some embodiments an app of a patient app package may interact with one or more apps provided by a third party app provider. In some embodiments such an app may be used by a patient instead of or in addition to a particular health monitoring app that may be provided as part of a patient app package. For example, a patient may select a particular third party app to use for monitoring food intake, exercise, medications. In some embodiments data from a third party app is used by a Dx app or sub-app or transmitted to an ADM.

In some embodiments an app may issue an alert (which term may be used interchangeably with “notification” or “notice”). An alert may, for example, notify a patient that an experimental therapy has become available, that a health-related action is upcoming, is due, should be taken or has been omitted. In some embodiments when an alert is issued to a patient, an indicator appears on or near the Dx icon or the icon changes in appearance. The indicator may be, e.g., a number in a circle (which may be colored, e.g., yellow, orange, or red), where the number indicates the number of alerts that the patient has not yet viewed or listened to or otherwise been made aware of the contents by an app (“pending alerts”). In some embodiments, when the patient selects the Dx icon, alerts are listed and/or the patient is asked whether he or she wants to hear or view alerts, e.g., pending alerts. In some embodiments a patient may designate one or more other individuals e.g., a caregiver of the patient, to receive or access alerts in addition to or instead of the patient. In some embodiments an alert warns a patient of a potentially health-threatening event or situation. For example, if a body monitoring function detects a potentially significant medical issue or problem or an important medication has not been taken on schedule, an alert may be issued. In some embodiments a HCP or HCO may be notified as well. In some embodiments an alert may be or comprise a reminder. A reminder may be issued in any of a variety of circumstances. A reminder may be issued to remind a patient of an upcoming action to be taken by the patient (e.g., an upcoming visit to a HCP). A reminder may be issued upon failure of a recurring action (e.g., taking a medication that is to be taken daily) to take place on schedule. A reminder may be issued if a patient has not entered data for a health promotion app on a given day or over a selected time period. In some embodiments reminders may be indicated differently to other alerts. A patient may select what type of reminders to receive and/or their schedule and/or may disable reminders in some embodiments.

In some embodiments, anyone with an appropriate PED or computer can download a Dx app and, in some embodiments, a Diet app, Ex app, Rx app, and/or other patient apps. In some embodiments, a Dx app and, in some embodiments a Diet app, Ex app, Rx app, and/or other patient app, may be available through the same channels by which apps are ordinarily available for the particular PED. For example, apps for Apple devices such as iPhone or iPad may be available from the iTunes App Store (http://www.apple.com/itunes/); apps for devices running an Android operating system may be available from Google Play (http://www.android.com/apps/#); apps for BlackBerrydevices may be available from BlackBerry® World™ (http://us.blackberry.com/apps/blackberry-world.html). In some embodiments, a Dx app and, in some embodiments a Diet app, Ex app, Rx app, and/or other patient app, may be available through a channel specific for such apps in addition to or instead of a general app store. For example, there may be a website that offers the apps for downloading. The website may be operated by or on behalf of an entity that at least in part owns, controls, makes, sells, or provides an app, ADM template, ADM component, ADM database, ADM system, or ADM-equipped EMR system. (Similarly, any websites discussed herein in relation to activities associated with patient apps (e.g., diagnosis confirmation), may in some embodiments be operated by or on behalf of an entity that at least in part owns, controls, makes, sells, or provides an ADM template, ADM component, ADM database or ADM system.) In some embodiments a Dx app, Diet app, Ex app, and/or Rx app is free. In some embodiments a fee may be charged for a Dx app, Diet app, Ex app, and/or Rx app. In some embodiments a fee may be waived or refunded upon full activation of a patient's Dx app. In some embodiments a Dx app may be considered fully activated if its disease list includes at least one disease for which the patient has an ADM with a confirmed diagnosis. In some embodiments a device, e.g., a PED or monitoring device, may be offered, e.g., for a fee or for free, to a patient for using one or more patient apps. In some embodiments a fee may be waived or refunded upon full activation of a patient's Dx app. In some embodiments, receiving a patient app and/or device capable of running a patient app or a monitoring device, may serve as an incentive for a patient to use such app, participate in an ADM network, ADM community, seek medical care from a HCP who is a member of a HCP ADM network.

An ADM with a confirmed diagnosis may be created in a variety of different ways in various embodiments. If a patient has a HCP who uses an ADM-equipped EMR system, an ADM may be created and/or a diagnosis may be confirmed in the ordinary course of the patient's health care record keeping by the HCP. In some embodiments a patient downloads a Dx app and is requested to download data from his/her EMR(s). In some embodiments the Dx app provides appropriate instructions to the patient so that the patient is able to download data from his or her EMR(s). Such data may be downloaded, e.g., via patient portals linked to patients' EMRs, a Blue Button feature on a patient's personal health record (PHR) software that is integrated with or capable of accessing the patient's EMR(s), or other software that may integrate with or access an EMR (which software may be provided by on behalf of an entity that at least in part owns, controls, makes, sells, or provides an app, ADM template, ADM component, ADM database or ADM system). In some embodiments EMR data are sent (e.g., electronically) to one or more remote location(s), e.g. one or more centralized location(s), and the data are used to populate (automatically, computer-assisted with human input, or by human input, or any combination thereof) one or more ADMs for the patient Optical character recognition and/or appropriate software to extract data from the format in which it is provided may be used in some embodiments. If the content of a resulting ADM is sufficient to confirm a diagnosis (e.g., if all data fields required for a diagnosis are occupied with data consistent with the diagnosis), the ADM may be submitted to a HCP, e.g., a physician, for confirmation, and, if confirmed, becomes an, active ADM. The patient then enters the ADM network for that disease and may enter the ADM community for that particular disease. In some embodiments a HCP, e.g., a physician, or in some embodiments, a data coordinator on-site (e.g., wherever a patient's medical records are available) completes and confirms (activates) the ADM (which activation may in some embodiments be subject to final confirmation by a physician), which enters the patient in the ADM network for that disease and allows the patient to enter the patient ADM community for that disease. In some embodiments a patient may ask their HCP, e.g., physician, to complete and/or confirm an ADM for the patient even if the HCP does not use an ADM-equipped EMR system. The HCP may be asked to visit a specified website that provides the ADM template. The ADM template may be at least partly filled in, e.g., based on the content of a patient's EMR that the patient has provided, e.g., to an entity that at least in part owns, controls, makes, sells, or provides the ADM template, ADM component, ADM database, or ADM system. A HCP may be asked to authenticate himself or appropriately, e.g., by entering a license number, prescriber number, user ID, and/or password. Other means of having a patient's HCP complete and/or confirm an ADM are within the scope of the present disclosure. In some embodiments completion and confirmation of an ADM may be sufficient to allow a patient to obtain access to experimental therapies available through an ADM network (assuming the patient otherwise qualifies for such therapies). In some embodiments an ADM that is completed and confirmed but not further updated becomes inactive after a specified time period.

In some embodiments a patient who does not have a confirmed ADM for a particular disease may become a member of a patient ADM community for that disease, provided that the patient has a diagnosis of the disease that has been confirmed by a HCP e.g., a physician. Confirmation of a diagnosis may be obtained or performed in a variety of ways. In some embodiments, merely by way of example, a patient may visit a specified website, select or enter a name of a disease name, and select or enter the name of a HCP who will confirm the diagnosis. In some embodiments a patient may provide the name of the HCP via email, text message, or selecting or entering the name into the PED in response to a request from the disease-specific app. In some embodiments a patient may provide contact information, e.g., email address and/or phone number of the HCP. The patient or system may then request the HCP to visit a specified website (which may be the same or different to the website that the patient visited). On visiting the website the HCP may be asked to enter his or her name and/or an appropriate identifier (e.g., license number). The HCP may then be presented with a list of patients who have indicated that the HCP will confirm a diagnosis for them, along with the corresponding diagnoses. The HCP may confirm or reject the diagnosis for each patient. In some embodiments an HCP is asked to authenticate themselves, e.g., by entering appropriate identifying information to verify their status, such as a license number or prescriber number when initially accessing the confirmation website or thereafter, in order for his or her confirmations to be valid. In some embodiments an HCP may have a userID and/or password for the website. In some embodiments the HCP may be asked to or offered an opportunity to view diagnostic criteria for the diagnosis applied by an ADM for the disease. In some embodiments the HCP may be asked to or offered an opportunity to view or fill out an ADM for a patient. In some embodiments the HCP may be offered an opportunity to obtain further information about ADMs, ADM-equipped EMR systems, ADM plug-ins, ADM-assisted clinical trials, expanded access programs, or repositioned therapies, Dx app, disease-specific app, or other aspects described herein. It should be understood that there are many ways HCP confirmation of a diagnosis could be obtained or performed so as to effectively restrict membership in a patient ADM community to individuals who have or are reasonably certain to have an HCP-confirmed diagnosis of the disease. In some embodiments a patient with a confirmed diagnosis but not having an ADM may join an ADM patient community for the disease but such membership may have a limited duration and/or may have limited functionality associated with it. In some embodiments, for example, membership may be limited to a defined time period such as 1 or 2 years, by which time the patient must have an active ADM in order to continue as a member of the ADM community.

In some embodiments, a Dx app has a disease list associated therewith. A disease list may be composed of a list of one or more diseases that a patient has or may have. In some embodiments, after accessing a Dx app (e.g., by tapping on a Dx icon) a patient is presented with a disease list. For example, if a patient has been diagnosed with multiple myeloma, rheumatoid arthritis, and hypertension and has confirmed ADMs for these diseases, a list of one, more than one, or all of these diseases may appear. A disease list may include any number of diseases, e.g., 1, 2, 3, 4, 5, 6, 7, 8, 9, 10, or more. A disease list may change over time, e.g., diseases may be added or may be removed (e.g., if a disease is cured or resolves.) A disease list may be organized in any of a variety of ways. For example, it may be alphabetical or may be chronological according to the dates that diseases were added to the disease list, dates of creation of ADMs for the diseases, dates of confirmation of the diagnoses (either starting at the earliest date and progressing to more recent dates or in the reverse order). In some embodiments the order may be selectable by the user. In some embodiments the Dx app may select the order based on any of a variety of parameters, such as frequency of access by the patient. A disease list may be populated with disease names in a variety of ways, e.g., as discussed further below. In some embodiments diseases in a disease list may be organized or displayed as icons or using any symbol or character sufficient to identify the disease, or arranged in any manner over one or more screens.

In some embodiments a Dx app comprises or has associated with it one or more disease-specific apps. In some embodiments one or more diseases in a disease list has a disease-specifc app associated with it. In some embodiments a disease list provides access to one or more disease-specific apps. In some embodiments disease-specific apps may be available for one or more diseases within a specialty, e.g., oncology, neurology, cardiology,ophthalmology. It will be understood that disease-specific app may not be available for every disease. In some embodiments if a disease-specific app is not available for a disease selected by a patient, a patient may be informed accordingly. In some embodiments a patient may be notified when such a disease-specific app becomes available. In some embodiments a disease-specific app may be available for at least 5, 10, 20, 30, 50, 100, 200, 300 diseases or more. In some embodiments access to one or more disease-specific apps may be provided in any appropriate manner. In some embodiments a disease-specific app provides one or more functions. For example such functions may include one, more than one, or all of the following functions: “Learn About”, “Find Patients”, “Find Physicians”, “Therapies”, and “Experimental Therapies”. Such functions may be referred to as “core functions” of a disease-specific app. In general, details of and/or content provided by the functions provided by a disease-specific app relate to a particular disease. For example, a “Learn About” function associated with a particular disease-specific app would offer a patient an opportunity to learn about that disease, while a Learn About function for a second disease would offer a patient an opportunity to learn about the second disease. A “Find Patients” function associated with a particular disease-specific app may offer a patient an opportunity to locate and/or communicate with other patients that have the same disease, e.g., are part of the same ADM network or ADM community for that disease. A “Find Physicians” function associated with a particular disease-specific app may offer a patient an opportunity to locate physicians that treat patients who have that disease. A “Therapies” function associated with a particular disease-specific app may provide patients with information about therapies that may be of use to treat the disease. Therapies may include therapies that are accepted by those in the medical profession as appropriate for treating at least some patients with the disease (which may be referred to as “standard” therapies, experimental therapies, and alternative therapies. Therapies may be grouped according to whether they are standard, experimental, or alternative. Information about each type of therapy may be provided by a separate function. In some embodiments a “Therapies” function includes at least a “Standard Therapies” function and an “Experimental Therapies” function. Information may include, e.g., name of a therapy, whether a therapy is approved for the particular disease, side effects, frequency of side effects, whether therapy is available over-the-counter or by prescription only, routes of administration available (e.g., oral, subcutaneous, intravenous, topical), package insert, warnings, contraindications, drug interactions. Alternative therapies may include, for example, herbal remedies, traditional medicines, nutritional supplements suggested (e.g., by their manufacturers) as useful for the disease, therapies suggested by members of the ADM community, or any therapy that may not be considered a standard therapy. Information provided about an alternative therapy may include, e.g., a description of the therapy, patient comments, physician comments, links to studies done on the therapy, contraindications, warnings (e.g., warning the patient, if appropriate, that the therapy has not been shown to be safe or effective, warning the patient, if appropriate, that the therapy has been shown to be deleterious or associated with one or more risks, etc.) Experimental therapies may include clinical trials, managed access programs (e.g., expanded access programs), and/or repositioned therapies. An “Experimental Therapies” function may be provided as part of a “Therapies” function or separately. An “Experimental Therapies” function may provide patients with information about experimental therapies that may be available for the disease. A function related to a particular disease may be referred to as a “disease-related function”. It should be understood that disease-related functions for different diseases may overlap or be similar in one or more respects but will generally not be identical. For example, a physician who treats a number of different diseases may appear in the physician list for each of these diseases. The name of a disease-related function may include the name of the disease or an abbreviation therefor. For example, a “Learn About” function for multiple myeloma may be named “Learn About Multiple Myeloma” or “Learn About MM”. In some embodiments a disease-specific app includes at least an “Experimental Therapies” function. In some embodiments a disease-specific app includes at least an “Experimental Therapies” function and a “Learn About” function. In some embodiments a disease-specific app includes at least an “Experimental Therapies” function, a “Learn About” function, and a “Find Physicians” function. In some embodiments a disease-specific app includes at least an “Experimental Therapies” function and a “Find Physicians” function. In some embodiments a disease-specific app includes a “Therapies” function.

A disease list may be populated in a variety of ways, e.g., as discussed further below. In some embodiments a patient who has a Dx app installed on his or her PED downloads a disease-specific app for one or more diseases. The disease-specific app(s) may interface with and operate together with the Dx app, e.g., as a plug-in. In some embodiments a Dx app comprises a plurality of disease-specific components, each of which provides appropriate functionality for a specific disease. The patient may activate one or more of the disease-specific components, which act as disease-specific apps. For purposes of description disease-specific functions will be generally described in terms of disease-specific apps that provide such functions, but it should be understood that the disease-specific functions may be provided or grouped in any suitable manner. Apps or portions thereof may be updated from time to time or replaced with updated versions. New apps, e.g., for additional diseases, may be added.

In some embodiments a Dx app comprises a function that allows the patient to select or change settings for the Dx app and/or for one or more diseases. In some embodiments a patient is provided an opportunity to select settings (which may be termed “preferences”) when they open a Dx app for the first time or when they first add a disease to their disease list or when they download or activate the app for that disease. In some embodiments, for example, a screen with a disease list includes a “Settings” button that, when tapped, provides a patient with an opportunity to change settings for the Dx app as a whole and/or for one or more diseases that the patient may select. In some embodiments a patient app comprises a “Settings” menu from which setting can be selected. In some embodiments a “Settings” button or menu may be provided within a disease-specific app to allow a patient to set or change settings for that particular app. A setting may be a general setting, which may typically be of general applicability to the Dx app as a whole, e.g., font size or language, or may be a setting for which a patient may have different preferences for different diseases. A patient may choose that a setting be applied to one, more than one, or all diseases. For example, in some embodiments a patient may choose to make his or her contact information or profile visible to other users who are part of the patient ADM community for a particular disease that the patient has or may choose to update or remove such information. A patient may select “always”, “never” or “ask me” for the general setting “Make Me Visible”. If the patient chooses “always”, the patient's profile and contact information is automatically made visible to other members of the networks for the diseases listed on that patient's disease list, while if the patient selects “never”, his or her profile and contact information are not made available. If the patient selects “ask me”, the system asks the patient to indicate a preference when a new disease is added to the patient's disease list. In some embodiments a patient may choose different visibility settings for different diseases. A general setting could be changed from within a disease app as applied to that particular disease. For example, a patient with five diseases may select “never” as a general setting for “Make Me Visible” but may alter that setting as applied to a particular disease for which that patient prefers to be made visible to others in the patient ADM community for that disease. In some embodiments the visibility settings (and all other settings), can be changed in the future by visiting the settings menu.

In some embodiments a Dx app comprises a function that allows the patient to add a disease to his or her disease list and add (e.g., download) or activate a corresponding disease-specific app for that disease. For example, in some embodiments a screen with a disease list includes a display element that provides access to such functions. The display element may, for example, take the form of an “Add Dx” or “New Dx” button, a symbol such as plus sign, or any other suitable element. For purposes of description an element that allows a patient to add a disease will be referred to as an “Add Disease” icon. An Add Disease icon may be located anywhere on the screen, e.g., in the upper right corner. In some embodiments, if a patient taps an Add Disease icon, a list of available diseases appears. The patient may scroll through the list. Upon reaching a disease that the patient wishes to add the disease list, the patient selects the disease, e.g., by tapping on it. In some embodiments a patient may enter a disease name using a keypad. The disease name may autocomplete or offer a short list of alternatives after a sufficient number of letters have been entered to allow doing so. Selecting a disease may result in downloading of a disease-specific app for that disease or activation of a pre-existing disease-specific component of the Dx app. The disease is added to the disease list and the functionality of the disease-specific app for that disease is available to the patient. In some embodiments a patient may visit a website and download a disease-specific app. Downloading the disease-specific app causes the disease to be added to the disease list.

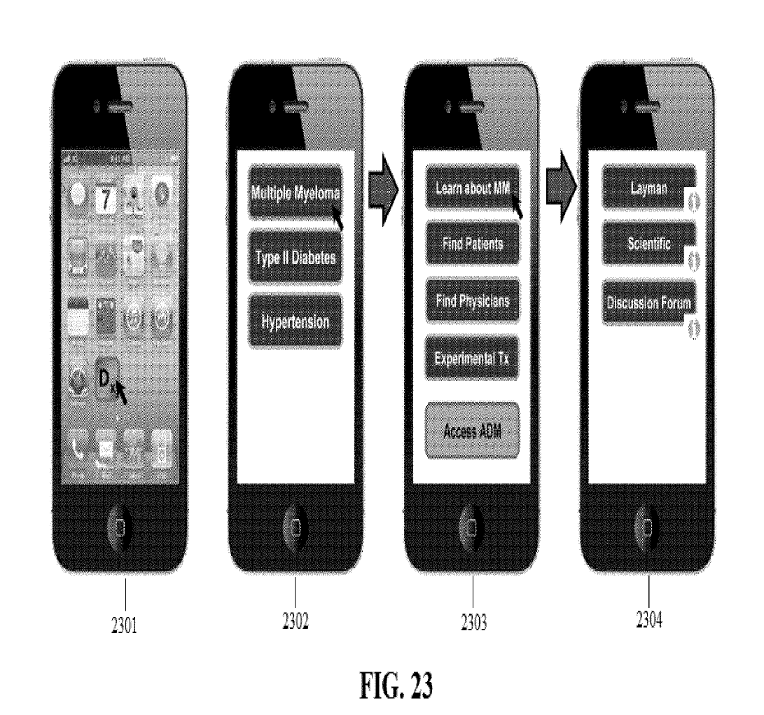

FIG. 23 interface 2301 shows that a Dx app may be accessed via an icon, which may be present on a home screen of a smartphone. FIG. 23 interface 2302 shows that a disease list becomes visible after a patient selects the Dx app (e.g., by tapping on it). FIG. 23 interface 2303 shows that after a patient selects a particular disease, various functions related to that disease become available to the patient (provided by the disease-specific app for that disease). In accordance with some embodiments such functions may include an option that allows the patient to access his or her ADM for that disease. FIG. 23 interface 2304 shows that, in accordance with some embodiments, selecting a “Learn About” function within a disease-specific app provides a patient options to access information of various types, e.g., information suitable for a layperson (layman), scientific information (scientific), and/or discussion forum. A list of information-related options may be provided on a separate screen after a “Learn About” function is selected. In some embodiments a “Learn About” function or a type of information or option to access information may be indicated with a lower case letter “i”, which may be in the form of the ISO graphical symbol for “information” and/or which may be enclosed in a circle (“information symbol”). A “layman” function may provide one or more links to respected publicly available websites that provide information about diseases at a level that may be intended to be understandable by a layperson, such as the Mayo Clinic Diseases and Conditions website, Merck Manual Home Health Manual website, National Institutes of Health Health Information websites, etc. A link may take the patient directly to the relevant information about the particular disease. In some embodiments a link to a Wikipedia article about the disease is provided. In some embodiments information provided by a layman function comprises a news report relating to the disease (e.g., from a newspaper, news program, or other media). In some embodiments information provided by a layman function comprises interview(s) with experts in diagnosis or treatment of the disease, researchers studying the disease or seeking treatments for the disease, wherein the information may be provided at a level intended to be understandable by a layperson. Information provided by a “scientific” function may comprise scientific articles that report on research findings regarding the disease, review articles relevant to the disease, textbook chapters relevant to the disease, or other forms of information of the type that a scientist or physician engaged in research or treatment relating to the disease or similar diseases may ordinarily consult. In some embodiments a scientific article or review article has been published in or accepted for publication in a scientific journal (which may be an online journal). In some embodiments a link to an online database such as PubMed, MedScape, online edition of a textbook, or other source of scientific/medical information is provided. In some embodiments information provided by a scientific function comprises interview(s) with experts in diagnosis or treatment of the disease, researchers studying the disease or seeking treatments for the disease. In some embodiments information is provided in written format, images, audio, video, or a combination thereof. In some embodiments at least some material accessible through a “Learn About” function is at least in part proprietary to an entity that that at least in part owns, controls, makes, sells, or provides the Dx app, ADM component, ADM database, or ADM system. For example, the material may be commissioned or prepared specifically for distribution via the Dx app or may be licensed by the entity for such purpose. A “discussion forum” may comprise one or more online patient discussion fora regarding topics relevant to the disease. Discussion fora may, for example, allow patients to share their experiences with a disease, therapy, etc., with other patients who have the same disease, inform other patients about events or findings relevant to the disease, etc. Patients who participate in a discussion forum may comment on or respond to contributions of other patients. In some embodiments patients may contribute to a discussion forum anonymously or using a pseudonym. In some embodiments a discussion forum may be moderated, e.g., to maintain a focus on topics relevant to the disease.

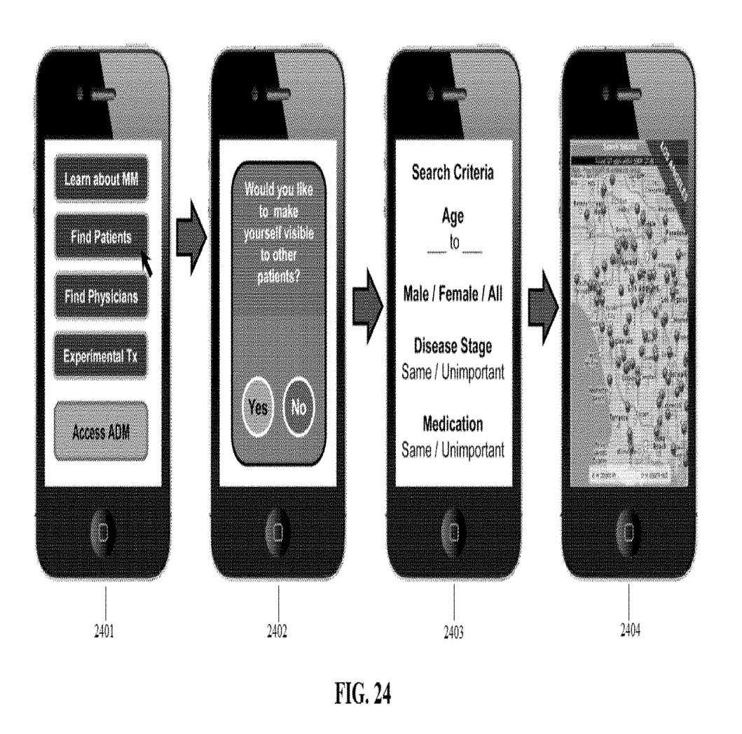

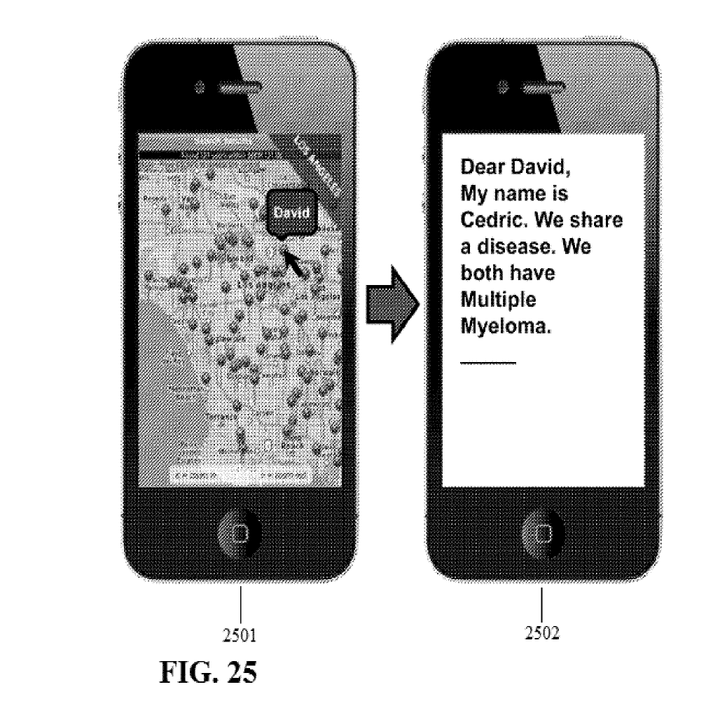

A “Find Patients” function may allow patients who have a particular disease with the ability to locate and/or communicate with other patients that have the same disease, e.g., patients who are members of the ADM network or ADM community for that disease. FIG. 24 interface 2401 shows selection of a “Find Patients” function. FIG. 24, interfaces 2402, 2403 and 2404 show subsequent screens which could be presented to a user of that function. FIG. 25 interface 2501 shows selecting a patient found by the Find Patients app. FIG. 25 interface 2502 shows sending a message to the patient. In some embodiments “Find Patients” function for a particular disease is only available to patients who are members of the ADM network for the disease. In some embodiments, upon selecting “Find Patients”, a patient is asked whether he or she wishes to make themselves visible to other patients (i.e., other patients with the same disease, e.g., within the ADM network or ADM community for that disease). By “make visible” in this context, is meant that at least some information about the patient is made available through the “Find Patients” function to other individuals in the disease network for that disease. In some embodiments a patient may select which information is to be made visible. Patient information may, for example, comprise or consist of one or more of the following: patient's name, patient's email address, patient's phone number(s), patient's home address, patient's gender, patient's age, stage of patient's disease, patient's medication(s), patient's HCP, patient's HCO, and/or patient's profile. Patients electing to be made visible may be asked to confirm their selection(s) before they are put into effect. The option to select whether or not to be made visible (and, if so, the option to select the information to be made visible) may be presented the first time a patient selects “Find Patients” and may not be presented thereafter or may be presented only some of the times that a patient selects “Find Patients”. In some embodiments a patient may change his or her selection(s) via an “Options” button. In some embodiments a patient may be reminded of their selection from time to time and, in some embodiments, may be asked whether he or she wishes to change it. In some embodiments a patient using the “Find Patients” function may be offered an option to enter search criteria to be applied to determine which patients are to be “made visible” to the patient. Search criteria may, for example, comprise or consist of any one or more of the following: age (e.g., an age range), gender, stage of disease, medication(s), geographic region, physician, HCO. In some embodiments a patient may request use of search criteria that would identify patients similar to himself or herself (e.g., same medication, same disease stage, etc.) or may specify that such criteria not be used. In some embodiments a Find Patients function provides a list or graphical display of addresses of other patients with the disease. In some embodiments addresses are displayed as pins on a map. In some embodiments a patient may select a particular address. In some embodiments a patient may then be offered an option to communicate with the patient having that address, e.g., by email or text message.

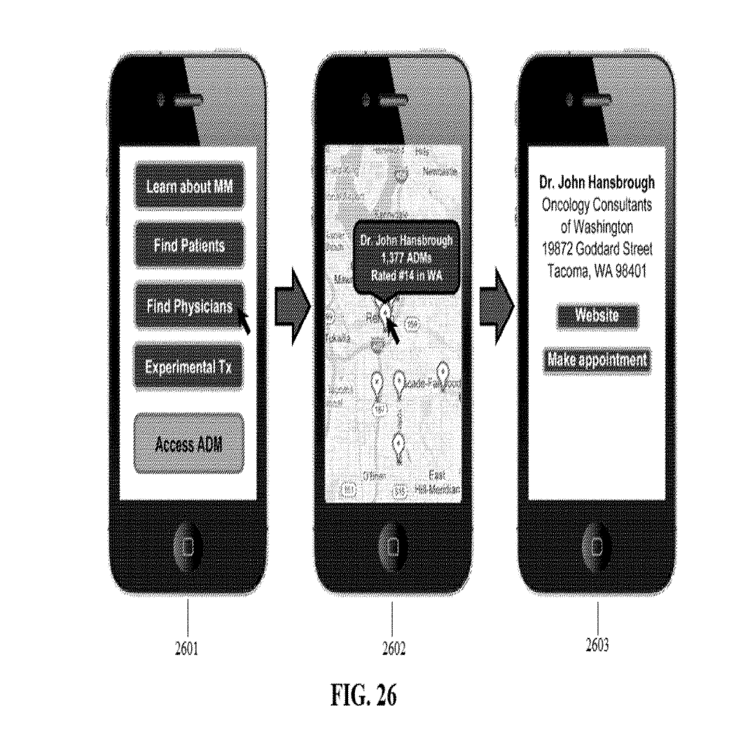

A “Find Physicians” function may allow patients who have a particular disease to locate physicians (and in some embodiments, other HCPs) who treat patients with that disease or who specialize in the medical or surgical specialty that encompasses managing patients with that disease. FIG. 26 interface 2601 shows selection of a “Find Physicians” function. FIG. 26 interface 2602 shows a map presenting the location of various physicians and that selecting a particular physician results in display of the physician's name and rating. FIG. 26 interface 2603 shows the physician's address and affiliation and shows providing options to visit the physician's website or make an appointment with the physician. In some embodiments a Find Physicians function provides a list or graphical display of addresses of such physicians, which may include the name of a HCO with which the physician is affiliated or by which the physician is employed. In some embodiments addresses are displayed as pins on a map. In some embodiments a “Find Physicians” function finds only physicians that are members of the HCP ADM network for that disease. In some embodiments a “Find Physicians” function may additionally find at least some physicians that are not members of the HCP ADM network for that disease. In some embodiments a “Find Physicians” function finds only physicians that are known to use EMRs or verify that they use EMRs. In some embodiments a “Find Physicians” function may additionally find at least some physicians that are not known to use EMRs or do not verify that they use EMRs. In some embodiments one or more additional items of information relating to a physician may be displayed. Such items(s) may include any one or more of the following: a rating of the physician, an indicator of the amount of experience that the physician has in treating patients with that disease, the number of ADMs for that disease that the physician has activated, the number of patients with activated ADMs for that disease that are under care of the physician. A ranking may be a performance-based ranking, a ranking based on the number of ADMs for that disease that the physician has activated, the number of patients with activated ADMs for that disease that are under care of the physician, or a combination thereof. In some embodiments a physician is ranked as compared with other physicians within the same geographic area and/or within the same specialty. In some embodiments criteria on which ratings are based are provided to the patient, e.g. via the Dx app, or made publicly available, e.g., on the Web. In some embodiments a patient may select one or more search criteria for physicians. Such criteria may comprise or consist of one or more of the following: geographic region, whether the physician is a member of the HCP ADM network for that disease, whether the physician is known to or verifies use of EMRs , whether the physician is accepting new patients, type of insurance accepted, languages spoken. In some embodiments a patient may select a particular physician's name and/or address from a list or map. In some embodiments a patient may then be offered an option to visit a website of the physician having that address (or a website of a HCO at which the physician works) and/or to make an appointment with the physician. In some embodiments an appointment may be made using the PED, e.g., by phone, text message, or email.

In certain embodiments a Dx app comprises an “Experimental Therapies” function. In some embodiments, when a patient selects “Experimental Therapies” the patients is asked whether he or she wishes to be notified when experimental therapies become available. In some embodiments if patient selects ‘no” they can still see the experimental therapies upon accessing Experimental Therapies but would not be notified as new experimental therapies become available. In some embodiments a patient who selects “yes” may be notified, e.g., via phone, text message, email, alert, when an experimental therapy becomes available, e.g., when a new clinical trial opens for enrollment or is scheduled to open for enrollment.

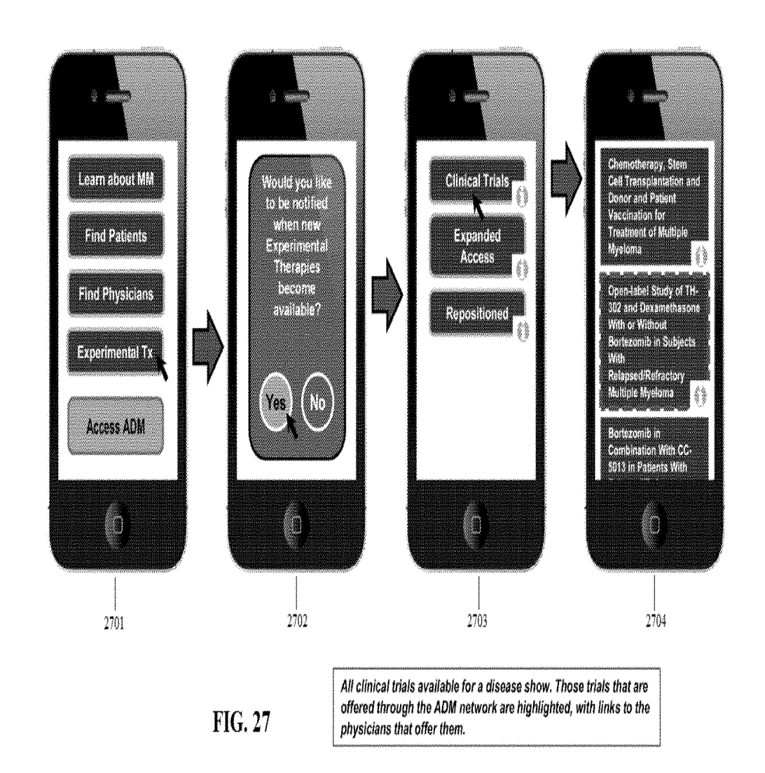

In some embodiments essentially all experimental therapies, e.g., all clinical trials, available for a disease may be shown. (“Essentially all” in this context should be understood as meaning that the list is not limited to experimental therapies offered through the ADM network, e.g., it may encompass at least some of those experimental therapies known or reasonably believed by the creators or providers of the list of experimental therapies or app containing it to be available. It will be understood that such creators or providers may not be aware of each and every experimental therapy available or may omit one or more such therapies in their discretion for any reason or no reason. In some embodiments the list may comprise those experimental therapies offered through the ADM network and those listed on a publicly available registry and/or those that have otherwise come to the attention of the creators or providers of the experimental therapies list or app containing it. In some embodiments sponsors and/or investigators offering experimental therapies may submit information about such therapies to an entity that at least in part owns, controls, makes, sells, or provides an app, ADM template, ADM component, ADM database or ADM system for inclusion in the list. In some embodiments a list may be limited to experimental therapies offered within a particular jurisdiction. For example, clinical trials or expanded access programs available in the US may be listed. In some embodiments a list may be tailored for the particular jurisdiction in which a patient lives.) In some embodiments those experimental therapies, e.g., trials, that are offered through the ADM network are highlighted or otherwise indicated. In some embodiments, if a patient selects a particular experimental therapy, additional information relating to the experimental therapy may be provided. Such information may include any one or more of the following, e.g., as appropriate for the experimental therapy: contact information for or links to one or more physicians and/or sites that offer the experimental therapy, inclusion and/or exclusion criteria, a summary of the trial protocol, information about the intervention being studied, a copy of the informed consent form or a portion thereof (e.g., a portion explaining the intervention and what the patient may experience or be asked to undertake), sponsor name, any information provided about the trial that is listed in a public trial registry such as ClinicalTrials.gov. By way of example, in FIG. 27 interface 2701 a patient who has selected multiple myelomna from their disease list selects the disease-related function Experimental Therapies. FIG. 27 interface 2702 shows that following such selection a patient may be asked whether they would like to be notified when new experimental therapies become available for the disease. In some embodiments this screen appears the first time the patient accesses the Experimental Therapies function. If the patient selects “Yes” this screen may not appear again unless the patient changes the Experimental Therapies setting. FIG. 27 interface 2703 shows a screen that lists various types of experimental therapies from which a patient may select. The figure indicates that a patient makes the selection Clinical Trials. FIG. 27 interface 2704 shows a list of clinical trials available for the disease multiple myeloma. Upon selecting a particular clinical trial a patient may be presented with additional information about the trial, e.g., address or contact information for one or more site(s) or investigators(s) or sponsor(s) conducting or sponsoring the trial, details of the therapy being tested, etc. In some embodiments a patient may be asked whether he or she wishes to contact a site or investigator. For example, the patient may be offered an option to phone the site. Expanded Access (which may be used interchangeably with managed access) may provide a list of therapies for which the patient may be eligible under an expanded access program. Repositioned may provide a list of therapies that may be appropriate for the patient and are available under a Repositioned Therapies program. Under a Repositioned Therapies program a patient may receive a commercially available therapy at no charge or at a reduced charge as compared with the price that the patient or his or her payer would typically otherwise pay for that therapy. If a patient has an active ADM for the disease, a clinical trial list, expanded access therapies list, or repositioned therapies list may be personalized to include only those trials, expanded access programs, or repositioned therapies programs, respectively, for which the patient appears potentially eligible based on the information in his or her ADM. If the patient does not have an active ADM for that disease, the clinical trial list may include one, more than one, or all clinical trials that are recruiting subjects (or will soon be recruiting subjects) that are being conducted by investigators or sponsors within the ADM network for that disease. Similarly, if the patient does not have an active ADM for that disease, the expanded access list or repositioned therapies may include one, more than one, or all expanded access programs or repositioned therapies programs, respectively, that are available within the ADM network for that disease.

In some embodiments an indication is provided as to whether the patient appears eligible or potentially eligible for the experimental therapy based on information in their ADM for that disease. In some embodiments if a patient appears potentially eligible, an explanation of what additional information may be needed to determine eligibility may be provided. In some embodiments if a patient appears not to be eligible, a reason may be provided. In some embodiments at least some information is provided only for those experimental therapies that are offered through the ADM network. For example, in some embodiments contract information for or links to one or more physicians and/or sites that offer the experimental therapy are provided only for those experimental therapies that are offered through the ADM network. In some embodiments a patient may contact a site via the PED. For example, the patient may place a phone call, send a text message or email to the site or to one or more study personnel, e.g., to a CRC or investigator. In some embodiments a patient may contact a sponsor or CRO and, in some embodiments, may receive a response, via the Dx app, in a de-identified manner. In some embodiments information relating to an experimental therapy may be accessed via an “i” icon. In some embodiments a patient may select one or more search criteria to be applied to identify experimental therapies. Search criteria may include any one or more of the following: geographic region, phase of a trial, filter or rank based on likelihood of eligibility. In some embodiments a system may keep track of the number of patients who contact a study personnel or site via a Dx app or after viewing the name of the experimental therapy using a Dx app. In some embodiments a system may keep track of the number of patients who are screened to receive an experimental therapy, e.g., screened for a trial, and/or who enroll in a trial or receive an experimental therapy after viewing the name of the experimental therapy using a Dx app or after contacting study personnel or site via a Dx app.

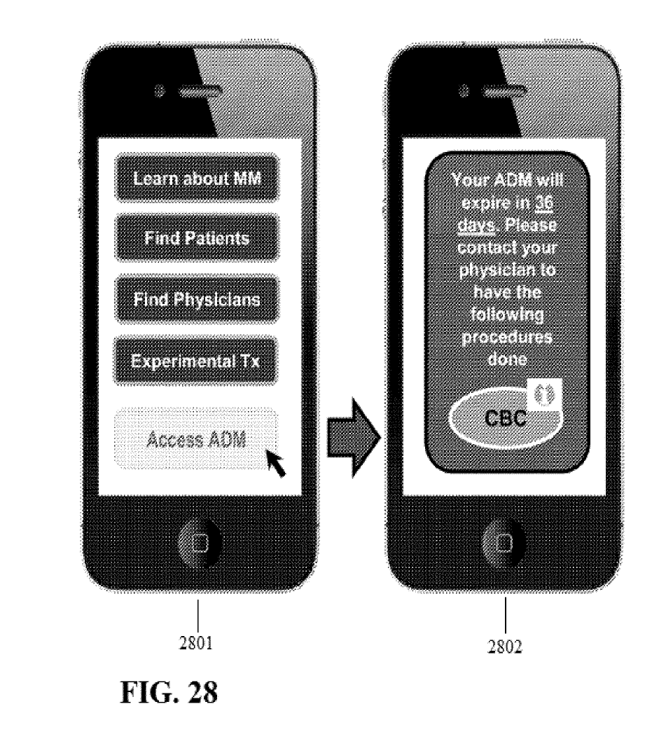

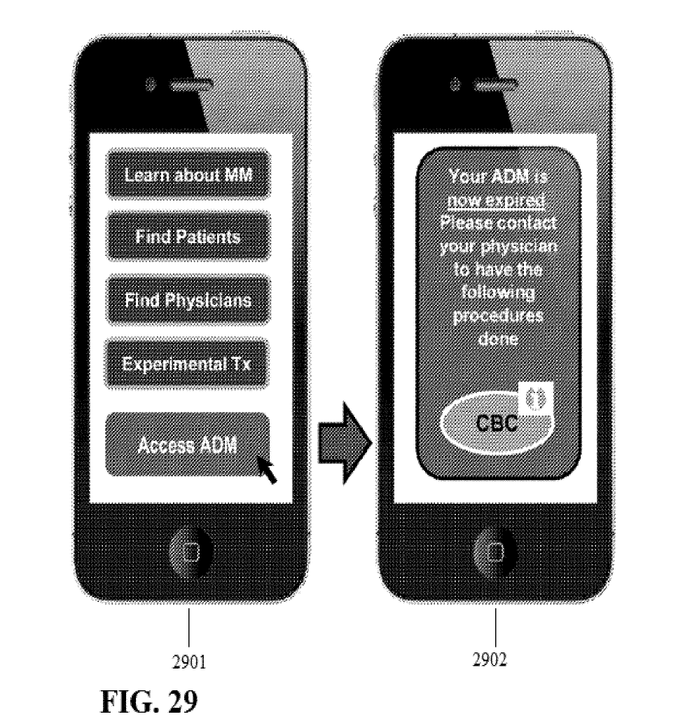

In some embodiments a Dx app provides an “Access ADM” function, whereby patients may access at least some information in, about, or generated from their ADM(s) for one or more diseases, e.g., one or more of diseases listed in their disease list (see, e.g., FIG. 28 interface 2801. An Access ADM function may be active only for those patients who have an ADM for the disease. In some embodiments a patient may access such information via an “Access ADM” button or icon (“Access ADM button”). In some embodiments information regarding the status of an ADM may be provided at least in part by the color of the Access ADM button for that ADM. For example a green button may indicate that the ADM is active and that there are no alerts for that ADM. A yellow button may indicate that the ADM has an alert. An alert may be a pending alert or an alert that the patient has already viewed or listened to or otherwise been made aware of its contents via the app. A red button may indicate that the ADM has become inactive (sometimes referred to as “expired”). In some embodiments a patient may be presented with one or more alerts upon selecting an Access ADM button (see, e.g., FIG. 28 interface 2802). For example, a patient may be informed that the ADM is scheduled to become inactive (sometimes termed “expired”) at a certain time or on a certain date unless one or more actions is taken. Such action(s) may include, for example, visiting a HCP, having one or more tests or procedures performed, etc. In some embodiments an alert may inform the patient what action(s) must be taken in order to avoid expiration of the ADM, may assist the patient in making an appointment to visit a HCP in order that such action(s) may be taken, and/or may provide the patient with an option to view information about the action. For example, information may be provided about a test or procedure. In some embodiments a patient may be informed that an ADM has expired (see, e.g., FIG. 29 interface 2901, which shows accessing an ADM that has expired). In some embodiments an alert may inform the patient what action(s) must be taken in order to reactivate the ADM (see, e.g., FIG. 29 interface 2902, which shows an alert notifying the patient that the ADM has expired and advising the patient how to have the ADM reactivated), may assist the patient in making an appointment to visit a HCP in order that such action(s) may be taken, and/or may provide the patient with an option to view information about the action.

In some embodiments, if a patient does not have an ADM for a particular disease that is in their disease list, the Access ADM button may, for example, be gray or shaded or otherwise different in appearance to apps or functions that are available for use. Upon selecting the Access ADM button a message may be presented informing the patient that the function is not available. An explanation, e.g., indicating that the patient does not have an ADM for the disease, may be provided. The patient may be asked if they wish to view a list of HCPs who treat patients with the disease and use ADMs or may be directed to the Find Physicians function, or otherwise informed how they can obtain an active ADM for the disease. Similarly, in the case of other functions or sub-apps that use or rely on ADMs, such function(s) may not be available to patients who do not have such ADMs or may have limited functionality to such patients. Upon attempting to use such functions or sub-apps, a patient may be informed that the function is not available or has limited functionality. The unavailability of a function or sub-app may be indicated by its name or icon being gray or shaded.

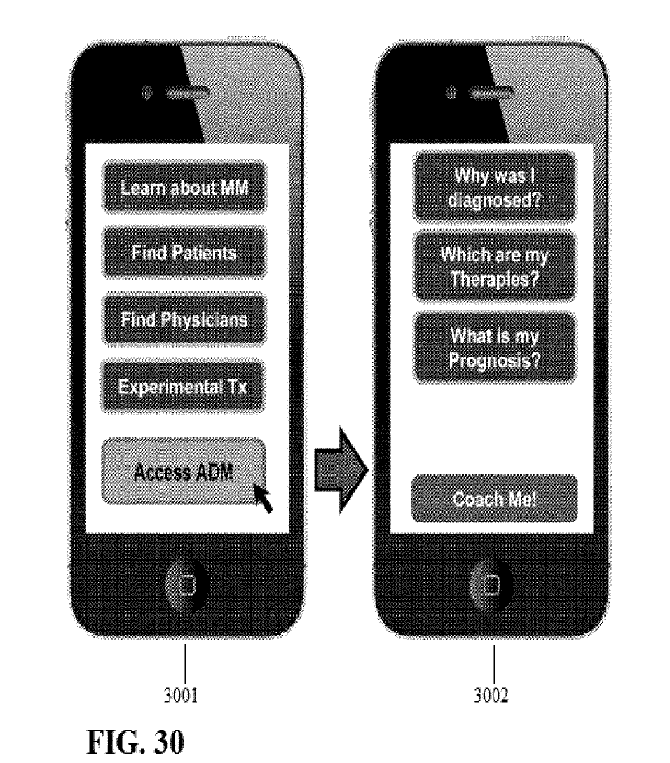

In some embodiments selecting an Access ADM function for a particular disease allows the patient to select from one or more of the following options: “Why was I diagnosed?”, “What are my therapies?”, “What is my prognosis?” or “Coach Me” (see FIG. 30 interface 3001, which shows accessing an active ADM, and interface 3002, which shows options presented after “Access ADM” is selected). In some embodiments, a “Why was I diagnosed?” function provides an explanation of the reasons why the patient was diagnosed with the disease is presented, which explanation is based at least in part on the contents of the patient's ADM. For example, the symptoms, signs, test results, or other criteria that resulted in a confirmed diagnosis as entered in the ADM may be presented. In some embodiments “Why was I diagnosed” may provide a full description of the diagnosis. For example, a disease may be referred to generally “multiple myeloma” but it might more specifically be “CD28+CD15− type A stage 4 multiple myeloma”. In some embodiments, explanations of the signs, tests, biomarkers, molecular markers, criteria (e.g., grading or staging criteria), etc., may be presented, e.g., the patient may be able to obtain such information via an “i” icon presented in association with the symptom, sign, test result, biomarker, molecular marker, or other criteria. In some embodiments, a “What are my therapies?” function provides a list of the therapies that the patient has been recommended or prescribed for the disease is presented, which list is based at least in part on the contents of the patient's ADM. For example, the medications prescribed or recommended by a patient's HCP for the disease, as entered in the ADM, may be presented. Such medications may include both those medications a patient takes at home and those that a patient may receive in a health care setting, e.g., a HCP office. In some embodiments, information about the therapies may be presented, e.g., the patient may be able to obtain such information via an “i” icon presented in association with the name of a therapy. In some embodiments, a “What is my prognosis?” function provides information regarding the patient's prognosis, wherein the information is based at least in part on contents of the patient's ADM for that disease. In some embodiments the information is generated by applying a prognostic algorithm to information in the ADM. For example, in the case of a cancer patient, such information may include the cancer grade, stage, molecular markers, and/or other information typically used by those of ordinary skill in the art to generate a prognosis. The patient may be informed that the prognosis is an approximation and may not take into account all factors relevant to the individual patient. In some embodiments, a patient who selects a “What is my prognosis” function and views a prognosis may be presented with an option to select “How can I improve my prognosis?” Upon selecting such a function, a patient may be offered suggestions as to how they may be able to improve their prognosis, such as by modifying habits or behaviors that affect the prognosis. For example, a patient with cardiovascular disease may be presented with a suggestion to exercise more or modify his or her diet. Such suggestions may be based at least in part on data in the patient's ADM and/or data entered into or gathered by the Dx app or a health promotion app, e.g., by the patient or a monitoring device.

In some aspects, the present disclosure relates to generating, providing, or using a score to establish, reflect, or convey (e.g., to a patient or HCP) how well the behavior of a patient matches what may be understood or believed to be the optimal or a preferred behavior to minimize the symptoms or the progression of a disease. In some aspects, such score may be referred to as a “patient disease management score”. In some aspects, computer-executable instructions are provided that, when executed, compute such a score. In some embodiments such a score may combine aspects of any one or more of exercise, nutrition, monitoring (biomarkers, physiological variables), treatment compliance, and/or other relevant factors (e.g., relevant factors that may be at least in part under control of or modifiable by the patient). In some embodiments individual scores for at least some such behaviors may be computed, Methods for computing a score may be based on art-accepted criteria regarding behaviors that are helpful or detrimental for managing symptoms or progression of a disease. Application of such methods may be individualized for a particular patient based, e.g., on information in a patient ADM or input by the patient. Such information may include, e.g., patient age, other diseases, medications, etc. Behavior may refer to behavior over time, e.g., behavior over a period of at least 1, 2, 4, 6, or 8 weeks. In some embodiments a score may be determined on a daily basis and may in some embodiments be averaged over a period of time. In some aspects, a patient disease management score may be computed based at least in part using data input via a patient app. In some aspects, a patient disease management score may be provided to a patient, HCP, or ADM via a patient app. In some embodiments incentives, e.g., rewards, may be provided to a patient based on improvement or stabilization of a patient disease management score. Such incentives may comprise, e.g., reduced insurance premium, cash or gift certificate rewards, etc. In some aspects, a patient disease management score or computer-executable instructions for generating or providing such score may be used independently or together with one or more other aspects described herein.

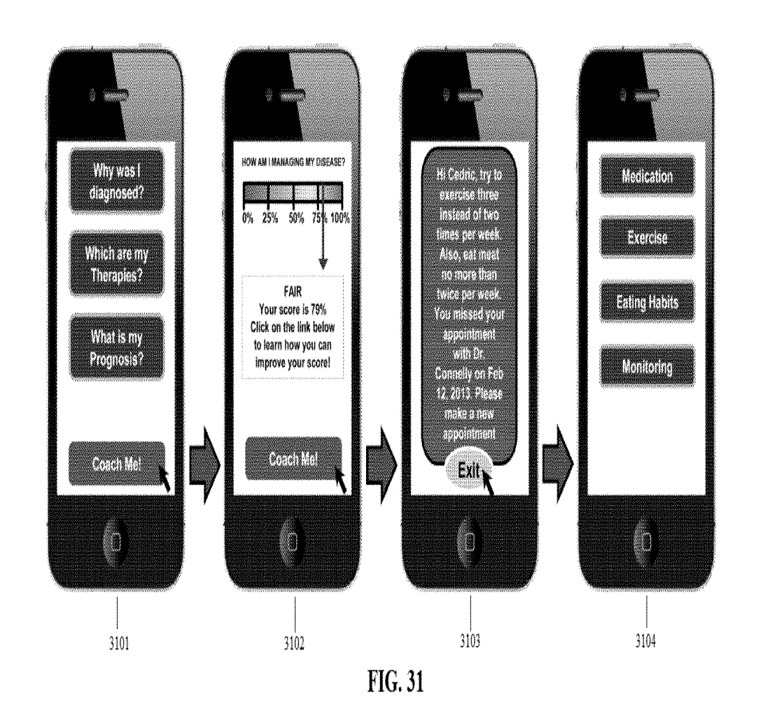

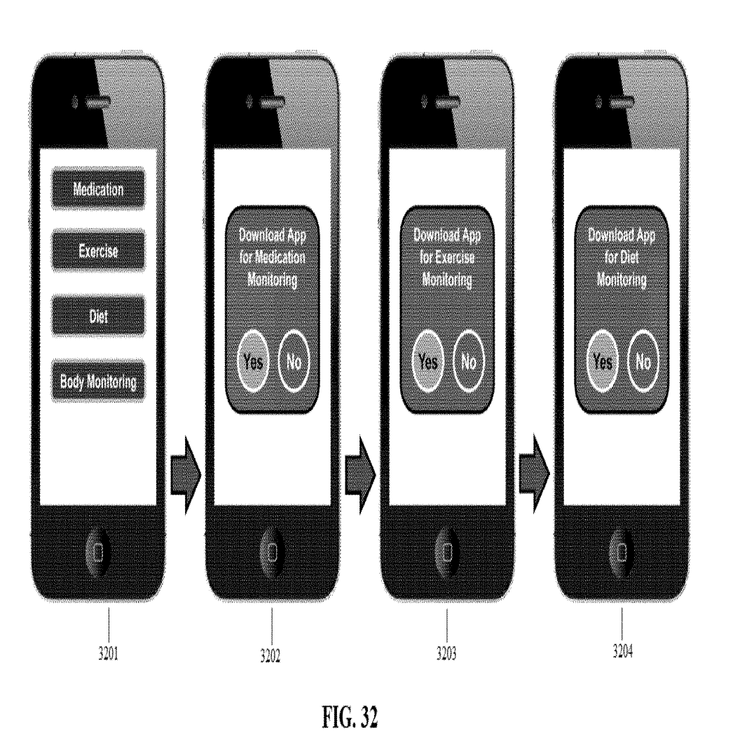

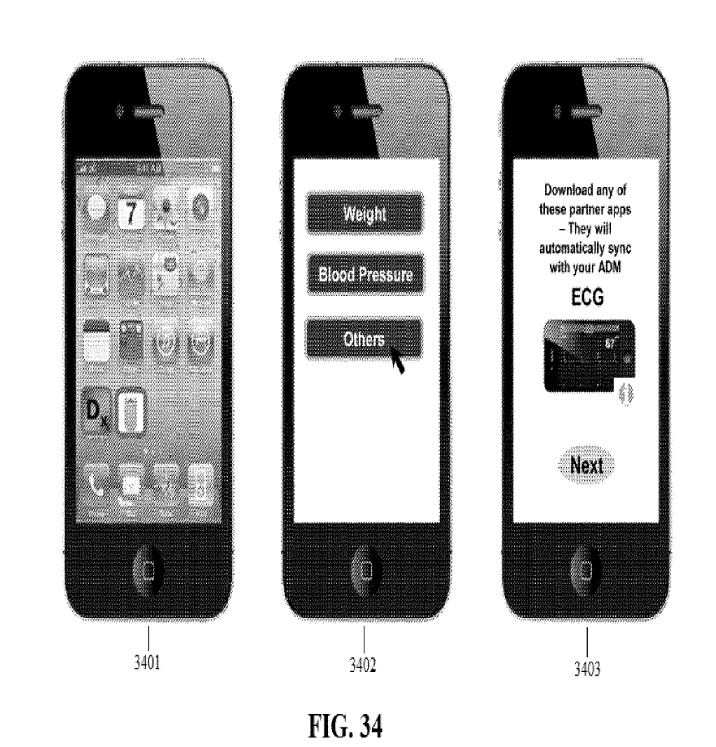

In some embodiments a Coach Me function provides a patient with information about how well (or poorly) they are managing their disease (see, e.g., FIG. 31 interfaces 3101-3104). A Coach Me function may provide a numerical score, percentage, letter score, graphical representation, or other means of providing a patient with an indication of how well they are managing their disease (“patient disease management score”). See, e.g., FIG. 31 interface 3102. A patient disease management score may reflect any one or more of the following factors: the extent to which the patient adheres to one or more therapeutic regimens prescribed or suggested by their HCP (as indicated in the ADM), the extent to which the patient adheres to recommendations pertaining to diet, exercise, and/or other health promotion/maintenance behaviors (e.g., as indicated in the ADM or generated by the Dx app), the extent to which the patient keeps appointments (e.g., for follow-up visits) with their HCPs, etc. In some embodiments a score is assigned to each of one or more such factors. Such scores may be presented individually and/or may be combined to provide an overall score. Different factors may be weighted equally or accorded different weights. In some embodiments a Coach Me function may provide a patient with suggestions as to how they may improve their patient disease management score(s) (see, e.g., FIG. 31, interface 3103). For example, a patient may be advised to reduce or increase consumption of one or more foods, may be informed that they missed a medical appointment and advised to reschedule, etc. In some embodiments a Coach Me function provides a patient with access to one or more of the following apps: Medications (Rx), Exercise (Ex), Eating Habits, Body Monitoring. See, e.g., FIG. 31, interface 3104. Body monitoring may include, e.g., monitoring of heart rate, blood pressure, blood sugar, weight, sleep, EKG, ECG, and/or other physiological variables and/or biomarkers (see, e.g., FIG. 34, interface 3402, which shows weight and blood pressure body monitoring apps, and FIG. 34 interface 3403, which shows that upon selecting “other” from interface B, a patient is offered a chance to download an ECG app). Data relating to body monitoring may be entered by a patient or may be entered directly from a body monitoring device, which in some embodiments may be or comprise a sensor, a wearable device, or an implanted device. In some embodiments it is envisioned that a patient app communicates with a wearable monitoring device, which may be applied as a patch to the skin, which may comprise one or more sensors or detectors. In some embodiments a patient may be invited to download one or more body monitoring apps upon selecting a button with the appropriate name (see, e.g., FIG. 32 interface 3201, which could be used to select an app, and interfaces 3202, 3203 and 3204, which could be used, respectively, to download medication monitoring, exercise monitoring and diet monitoring apps). Upon subsequent accesses of the Coach Me function, selecting such button would launch the app. In some embodiments such apps may be accessed via an icon on the home screen (see, e.g., FIG. 34 interface 3401). In some embodiments a user, e.g., a patient, may be able to access a health promotion app via the “Coach Me” section of a disease-specific app. In some embodiments a health promotion app or disease-specific app may be accessible from a separate icon on the home screen in addition to or instead of via the Dx app. For example, a user may be able to directly access a health promotion app or particular disease-specific app from the home screen without needing to first access the Dx app. A health promotion, monitoring, or Coach Me app may provide tools such as graphical displays that assist a patient in understanding how their behaviors and/or score changes over time. In some embodiments a Coach Me app may provide real-time feedback to a patient, which may be based at least in part on data input into the patient app, e.g., by the patient or a monitoring device. For example, a patient may be provided with a suggestion to increase their exercise intensity.

In some embodiments data entered into or gathered by a health promotion app or monitoring app may be transmitted to a patient's ADM(s) or EMR(s) and/or to a remote location that processes the data and, if appropriate, transmits at least some such information to a patient's ADM(s) or EMR(s). In some embodiments data may be transmitted to one or more ADMs to which the data is particularly relevant and/or for which a field for the data exists. For example, monitoring of blood sugar may be transmitted to a diabetes ADM. Monitoring of blood pressure may be transmitted to a hypertension ADM. Medication data may be transmitted to an ADM for a disease for which the medication has been prescribed or recommended. In some embodiments an ADM may have one or more fields or sections dedicated at least in part to data entered by a patient or via a patient app.

In some embodiments a patient app may comprise one or more data checking functions. For example, entry of implausible, out of range, or inconsistent data may be identified. An alert or other feedback may be provided to a patient, caregiver, HCP, etc. In some embodiments at least some patient input is time and/or date stamped or recorded (e.g., in the case of voice input). In some embodiments entry of at least some data may only be accepted within a defined time window. For example, a patient may not be permitted to complete a symptom diary entry for a given day before 5 PM on that day. In some embodiments data transmitted or received by a PED, e.g., via a patient app, may be encrypted. In some embodiments limitations or requirements may be specified in order to comply with or facilitate a clinical trial or MAP protocol. In some embodiments a patient app is used to collect a patient-reported outcome (e.g., pain, symptom, side effect, etc.) in a clinical trial or MAP. In some embodiments a patient app may interface with an interactive voice response system, e.g., in the context of a clinical trial, MAP, or repositioned therapies program. In some embodiments a patient app may interface with an ADM, ADM-SC, or ADM-EDC that may at least in part perform or provide one or more functions of an IVRS, e.g., in the context of a clinical trial, MAP, or repositioned therapies program.

In some aspects, it may take on average no more than 1, 2, 5, 10, 15, or 20 minutes a day for a patient to enter data into a Diet app, Ex app, and Rx app (or any one or more such apps used by the patient). Interface and/or data entry means may be selected to make it simple, convenient, and/or quick to use an app. In some embodiments, for example, camera or voice input may be used for at least some entry instead of selection from a list or typing. For example a photo of food or medication may suffice to enter data for a Diet app or Rx app, optionally with patient and/or caregiver confirmation that the food or medication is to be consumed or taken or was consumed or taken. In some embodiments interface and/or data entry means may be suitable for or readily usable by patients with limited vision and/or physical dexterity. In some embodiments instructions in use of an app or device may be provided, e.g., in writing, audio, video, or via in-person classes. In some embodiments an average refers to average for a particular patient (e.g., over the course of a period of at least a week, month, or year). In some embodiments an average refers to an average across multiple patients (e.g., at least 10 patients), e.g., over such time period. A patient or patients may be randomly selected from the set of patients that use an app. A time may be measured after a patient has become familiar with the use of the app, e.g., after the patient has been using the app on a regular basis for at least a month.

In some embodiments an app may issue an alert (which term may be used interchangeably with “notification” or “notice”). An alert may, for example, notify a patient that an experimental therapy has become available, that a health-related action is upcoming, is due, should be taken or has been omitted. In some embodiments when an alert is issued to a patient a device emits an intermittent or continuous sound or vibration. The sound may be a distinctive sound or vibration associated with a Dx app and/or associated with a particular type of alert. A sound may be a verbal message, which may inform the patient that an alert is pending and may, in some embodiments, inform the patient of at least some of the content. A sound may be a tone or sequence of tones. In some embodiments when an alert is issued to a patient, an indicator appears on or near the Dx icon or the icon changes in appearance. The indicator may be, e.g., a number in a circle (which may be colored, e.g., yellow, orange, or red), where the number indicates the number of alerts that the patient has not yet viewed or listened to or otherwise been made aware of the contents by an app (“pending alerts”). In some embodiments, when the patient selects the Dx icon, alerts are listed and/or the patient is asked whether he or she wants to hear or view alerts, e.g., pending alerts. In some embodiments a patient may designate one or more other individuals e.g., a caregiver of the patient, to receive or access alerts in addition to or instead of the patient.

In some embodiments a patient app may comprise an extension or one or more additional function(s) relating to one or more experimental therapies. For example, if a patient enrolls in a clinical trial, managed access program, or receives a repositioned therapy through the ADM network, a patient app may include additional data entry or monitoring functions that may not be included in a standard patient app, e.g., a standard disease-specific app for a particular disease for which the therapy is used. A patient may, for example, perform additional monitoring, may keep a symptom diary, answer a questionnaire, confirm having taken or used the relevant experimental therapy, etc. In some embodiments versions of a patient app, e.g., a health promotion app or disease-specific patient app, may be tailored for particular experimental therapies purposes. Functions such as ability to communicate with and/or receive communications from a sponsor or site, e.g., communications relating to the experimental therapy, may be provided.

Implementation

This section includes discussion of certain aspects relating to implementation of the present invention. It should be understood that aspects pertaining to implementation are also discussed elsewhere herein. In general, the present invention may be implemented with any suitable combination of hardware and software in various embodiments. If implemented as a computer-implemented apparatus, the present invention is implemented using means for performing those steps and functions described herein that have been selected for the particular embodiment of the invention being implemented.

The present invention may be included in an article of manufacture (e.g., one or more computer program products) comprising, for instance, computer useable media. The media has embodied therein, for instance, computer readable program code means for providing and facilitating the mechanisms of the present invention. The article of manufacture may be included as part of a computer system or sold separately.

In general, the EMR database may be implemented using any suitable database management system (DBMS). In some embodiments a relational database management system (RDBMS) is used. Various RDBMS software packages are available, e.g., from Microsoft (e.g., Microsoft SQL Server), Oracle (e.g., MySQL), Informix, and IBM (e.g., DB2). Non-SQL based DBMSs, e.g., object database management systems, may be used in various embodiments of the invention. It should be understood that the data may be stored in multiple distinct databases, which may be stored in different locations. Data may be stored and retrieved using standard approaches. It will be understood that data may be stored in the EMR database in any suitable manner. The EMR database may contain references, e.g., pointers, to the data itself, which data may be stored within the EMR system or externally. For example, a EMR for a particular patient may contain a reference to a medical image, which medical image may be stored in a medical image database. In some embodiments the content of the EMR database is digitally watermarked.

It will be understood that the invention may be implemented using one or more computer systems, which may each comprise one or more computers. A computer system of use in the present invention may be a general-purpose computer system that is programmable using a high-level computer programming language. A computer system may be implemented at least in part using specially programmed, special purpose hardware. In general, a computer system includes a processor, which may be a commercially available processor in various embodiments. Such a processor usually executes an operating system which may be, for example, a Windows operating system (Microsoft), MAC OS (Apple), Linux available from various sources, UNIX available from various sources, etc. Many other operating systems may be used. It will be understood that portable electronic devices may use different operating systems from those running on larger devices, e.g., iOS (Apple), Android (Open Handset Alliance), etc. A processor and operating system together provide a computer platform for which application programs in high-level programming languages are written. It should be understood that the invention is not limited to a particular computer system platform, processor, operating system, or network. It would be apparent to those skilled in the art that the present invention could be implemented using any of a wide variety of programming languages or computer systems. It should be appreciated that the invention is not limited to any particular architecture, network, or communication protocol. Various embodiments of the invention may be implemented as programmed or non-programmed elements, or any combination thereof. Various embodiments of the present invention may be programmed using an object-oriented programming language, such as Java or C++. Other object-oriented programming languages may also be used. Functional, scripting, and/or logical programming languages may be used. One or more elements of the invention or aspects thereof may include one or more application programming interfaces (APIs). Such APIs may, for example, facilitate communication between existing electronic medical record systems and a system of the present invention. One or more elements of the invention or aspects thereof may be implemented as or using a “Web service” (which term refers to a software system designed to support interoperable machine-to-machine interaction over a network). One or more elements of the invention or aspects thereof may be implemented using a document description language or environment (e.g., a markup language such as XML, or HTML). One of ordinary skill in the art will understand that numerous domain-specific markup languages exist. In some aspects the invention may modify or develop a domain-specific markup language for carrying out at least some functions of the invention. For example, such language may incorporate tags for items of medical data such as images (e.g., X-rays, CT scans, MRI scans, PET scans, etc.), EKGs, EEGs, or other types of health information.

It will be understood that a computer system may include various standard components such as one or more peripheral devices, e.g., one or more input devices (e.g., keyboard, mouse, etc.), one or more output devices (e.g., a display), data storage/memory component(s) (e.g., random access memory, read only memory), communications circuitry, etc. It will be understood that different users may employ computer systems having any of a wide variety of different components or configurations. For example, HCPs or patients may often interact with the EMR system using standard personal computers in their place of work or home.

One or more components of an inventive system may be distributed across one or more computer systems, one or more of which may be coupled to a communications network. For example, various embodiments of the invention or components thereof may be distributed among one or more computer systems configured to provide a service (e.g., servers) to one or more client computers, or to perform a task as part of a distributed system. For example, various embodiments of the invention or components thereof may be performed on a client-server system that includes components distributed among one or more server systems that perform various functions according to various embodiments of the invention. These components may communicate over one or more communication networks using a communication protocol. It would be appreciated that the business entity may or may not own the computer system or components thereof. In some embodiments at least some functions of the system may be outsourced. In some embodiments cloud computing and/or cloud storage may be used at least in part. In some embodiments, EMRs are at least in part stored at a site where medical information is generated or entered (“local storage”), e.g., at a health care organization. In some embodiments, multiple copies of EMRs are stored. For example, at least one copy may be stored by the business entity (e.g., on computer-readable medium owned or controlled by the business entity) and at least one copy may be stored locally and accessible by the business entity. Synchronization may be provided so that all copies remain the same or equivalent at most or essentially all times. References to a “network” or “communication network”, unless otherwise indicated or specified, may include one or more intranets or the Internet.

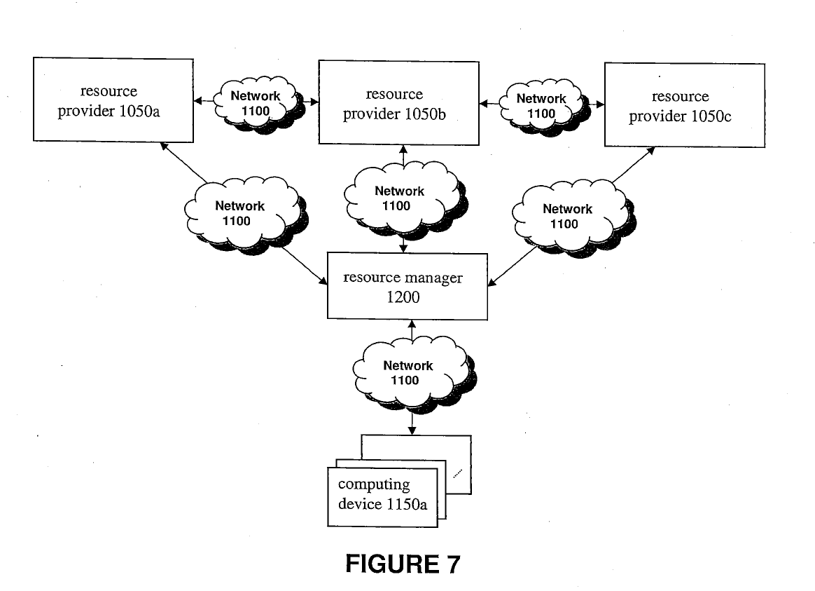

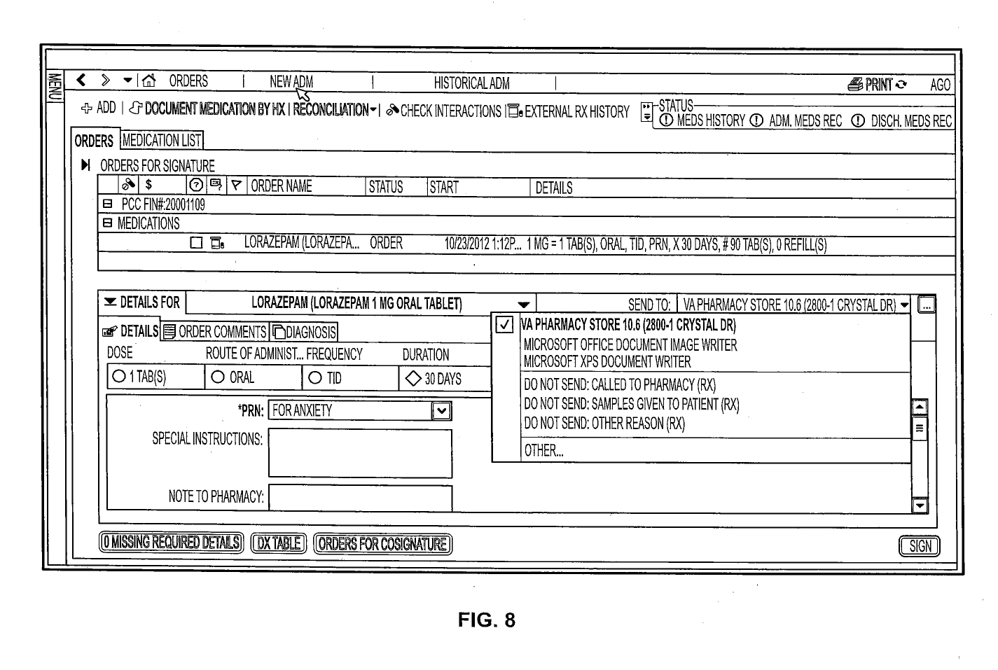

Referring now to FIG. 7, a block diagram of an exemplary cloud computing environment 1000 in which various embodiments may be implemented is shown and described. The cloud computing environment 1000 may include one or more resource providers 1050a, 1050b, 1050c (collectively, 1050). Each resource provider 1050 may include computing resources. In some implementations, computing resources may include any hardware and/or software used to process data. For example, computing resources may include hardware and/or software capable of executing algorithms, computer programs, and/or computer applications. In some implementations, exemplary computing resources may include application servers and/or databases with storage and retrieval capabilities. Each resource provider 1050 may be connected to any other resource provider 1050 in the cloud computing environment 1000. In some implementations, the resource providers 1050 may be connected over a computer network 1100. Each resource provider 1050 may be connected to one or more computing device 1150a, 1150b, 1150c (collectively, 1150), over a computer network 1100.

The cloud computing environment 1000 may include a resource manager 1200. The resource manager 1200 may be connected to the resource providers 1050 and the computing devices 1150 over the computer network 1100. In some implementations, the resource manager 1200 may facilitate the provision of computing resources by one or more resource providers 1050 to one or more computing devices 1150. The resource manager 1200 may receive a request for a computing resource from a computing device 1150. The resource manager 1200 may identify one or more resource providers 1050 capable of providing the computing resource requested by the computing device 1150. The resource manager 1200 may select a resource provider 1050 to provide the computing resource. The resource manager 1200 may facilitate a connection between the resource provider 1050 and the computing device 1150. In some implementations, the resource manager 1200 may establish a connection between a resource provider 1050 and a computing device 1150. In some implementations, the resource manager 1200 may redirect a computing device 1150 to a resource provider 1050 with the requested computing resource.

In some embodiments, all or substantially all health information in an EMR and/or in the EMR database may be stored on computer-readable media within the jurisdiction and/or within the geographical borders (optionally including ocean territory) of a selected country or union. In some embodiments, at least all or substantially all personally identifiable health information in an EMR and/or in the EMR database may be stored on computer-readable media within the jurisdiction and/or within the geographical borders (optionally including ocean territory) of a selected country or union. In some embodiments, all or substantially all health information received and stored (e.g., pertaining to a patient) may be stored on computer-readable media within the jurisdiction and/or within the geographical borders (optionally including ocean territory) of a selected country or union. In some embodiments, at least all or substantially all health information regarding a patient, or at least all or substantially all personally identifiable health information regarding a patient, may be stored in a country or union in which the patient resides or in which the patient seeks health care. In some embodiments, at least all or substantially all personally identifiable health information regarding a patient may be transmitted only within a selected country or union, e.g., a country or union in which the patient resides or in which the patient seeks health care.

In some embodiments of any aspect herein, a country may be the U.S. In some embodiments of any aspect herein, a country may be a country other than the U.S., which country may be any country in the world in various embodiments, e.g., Argentina, Australia, Belgium, Brazil, Canada, Chile, China, Egypt, France, India, Israel, Italy, Japan, Mexico, Netherlands, Norway, Pakistan, Poland, New Zealand, Philippines, Russia, South Africa, South Korea, Spain, Switzerland, Turkey, the United Kingdom. In some embodiments of any aspect herein, a union may be the European Union. In some embodiments of any aspect herein, a country or union may be a country or union in which the patient resides or seeks health care or in which a HCP practices or is registered to practice. In some embodiments of any aspect herein, a country or union may be a country or union in which the business entity is incorporated or its headquarters are physically located.

In some embodiments, an ADM template or other element of an EMR may be generated at least in part by crowd-sourcing or using at least some crowdsourcing principles, which may comprise sourcing the generation of rules and/or code to a group of people or community (crowd) through an open call, e.g., a task may be broadcast (e.g., by posting on a web page) to an unknown group of solvers in the form of an open call for solutions. The open call may be completely open or may be restricted at least in part (e.g., a solver or team may need to comprise at least one physician or medical student, in some embodiments). The task(s) may include generating criteria, generating sets of predetermined options, generating any aspect of an ADM template, writing computer code for any aspect of an EMR system or ADM template, etc. Broadcasting a task may comprise at least providing a task description. Broadcasting a task may comprise providing a set of guidelines (e.g., for diagnosis and/or management of a disease) and, e.g., a sample or example ADM template, and/or code therefor. ADM template(s) or other task responses proposed by the crowd may be at least in part owned by the business entity, the proposer(s), or both. The crowd may vote on a proposed ADM template or set of rules, criteria, or options to be adopted. Voting may be limited to HCPs, e.g., physicians. Selection of a “winner” may be at the discretion of the business entity and/or may be approved by or with advice of a professional organization or board (e.g., in a relevant discipline). Prize(s) may be provided, which may, e.g., comprise a share in revenue generated through use of an adopted ADM template. In some embodiments, an ADM template or other element of an EMR may be generated at least in part by posting task(s) for bidding and awarding a contract for such task(s) to a selected bidder or bidders. In some embodiments, teams of physicians, programmers, and/or physician-programmers may be engaged or may participate. In some embodiments HCPs who utilize an ADM-equipped EMR system may contribute suggestions for inclusion of one or more data items in an ADM. For example, a HCP may suggest that a particular diagnostic test be included as a criterion for arriving at a definitive diagnosis or may suggest monitoring of a particular test result. HCPs using the system may be permitted to vote on whether such data item should be included in an ADM. In some embodiments voting may be restricted to HCPs who have created at least a specified number of ADMs and/or have at least a specified number of patients whose EMRs include an ADM for that disease. In some embodiments an ADM template or ADM component is made available in an open source manner, in which the source code is available to HCPs and/or to the general public for use and/or modification from its original design. In some embodiments it is required that any such use or modification is made available at no cost and for any purpose to an entity that at least in part owns, controls, makes, sells, or provides such ADM template or ADM component.

Referring now to FIG. 7, a block diagram of an exemplary cloud computing environment 1000 is shown and described. The cloud computing environment 1000 may include one or more resource providers 1050a, 1050b, 1050c (collectively, 1050). Each resource provider 1050 may include computing resources. In some implementations, computing resources may include any hardware and/or software used to process data. For example, computing resources may include hardware and/or software capable of executing algorithms, computer programs, and/or computer applications. In some implementations, exemplary computing resources may include application servers and/or databases with storage and retrieval capabilities. Each resource provider 1050 may be connected to any other resource provider 1050 in the cloud computing environment 1000. In some implementations, the resource providers 1050 may be connected over a computer network 1100. Each resource provider 1050 may be connected to one or more computing device 1150a, 1150b, 1150c (collectively, 1150), over a computer network 1100.

The cloud computing environment 1000 may include a resource manager 1200. The resource manager 1200 may be connected to the resource providers 1050 and the computing devices 1150 over the computer network 1100. In some implementations, the resource manager 1200 may facilitate the provision of computing resources by one or more resource providers 1050 to one or more computing devices 1150. The resource manager 1200 may receive a request for a computing resource from a computing device 1150. The resource manager 1200 may identify one or more resource providers 1050 capable of providing the computing resource requested by the computing device 1150. The resource manager 1200 may select a resource provider 1050 to provide the computing resource. The resource manager 1200 may facilitate a connection between the resource provider 1050 and the computing device 1150. In some implementations, the resource manager 1200 may establish a connection between a resource provider 1050 and a computing device 1150. In some implementations, the resource manager 1200 may redirect a computing device 1150 to a resource provider 1050 with the requested computing resource.

It is expressly contemplated that each of the various aspects, embodiments, and features thereof described herein may be freely combined with any or all other aspects, embodiments, and features. The resulting aspects and embodiments (e.g., products and methods) are within the scope of the invention. It should be understood that headings herein are provided for purposes of convenience and do not imply any limitation on content included below such heading or the use of such content in combination with content included below other headings.

All articles, books, patent applications, patents, other publications, websites, and databases mentioned in this application are incorporated herein by reference. In the event of a conflict between the specification and any of the incorporated references the specification (including any amendments thereto) shall control. Unless otherwise indicated, art-accepted meanings of terms and abbreviations are used herein.

Those skilled in the art will recognize, or be able to ascertain using no more than routine experimentation, many equivalents to the specific embodiments of the invention described herein. The scope of the present invention is not intended to be limited to the above Description, but rather is as set forth in the appended claims. In the claims articles such as “a”, “an” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. It is to be understood that the invention encompasses all variations, combinations, and permutations in which one or more limitations, elements, clauses, descriptive terms, etc., from one or more of the listed claims is introduced into another claim. For example, any claim that is dependent on another claim may be modified to include one or more elements, limitations, clauses, or descriptive terms, found in any other claim that is dependent on the same base claim. Furthermore, where the claims recite a product (e.g., an apparatus or device or computer-readable medium), it is to be understood that methods of using the product according to any of the methods disclosed herein, and methods of making the product, are included within the scope of the invention, unless otherwise indicated or unless it would be evident to one of ordinary skill in the art that a contradiction or inconsistency would arise. For example, methods comprising executing computer-readable instructions to perform one or more acts or steps relating to an ADM, EMR, or database, such as accessing, retrieving, or analyzing one or more data elements therein, are provided. Any method may comprise a step of receiving a transmission, which transmission may comprise a query. Any method may comprise a step of analyzing a transmission, which transmission may comprise a query. Any method may comprise a step of transmitting (e.g., following receipt of a query), which transmission may comprise a response to a query. An apparatus may comprise one or more computer-readable media (e.g., memory). A memory may comprise one or more non-transitory computer-readable media. In some embodiments a memory may comprise at least a first medium and a second medium, wherein the first medium comprises a database and the second medium comprises the instructions. A database, or instructions, or both, may be stored on or divided among any number of computer-readable media, in various embodiments. An apparatus may comprise one or more processors. An apparatus may comprise one or more computer-readable media and one or more processors. A system may comprise an apparatus, which may itself comprise one or more systems or apparatuses. A claim expressed at least in part in terms a system may be expressed at least in part in terms of an apparatus (or apparatuses), or vice versa. Where a contributor or an act performed by a contributor are described, such contributor may in at least some embodiments be a designee of the contributor, and/or such act may be performed by a designee of the contributor, e.g., under direction of the contributor. Where an incentive is provided to a contributor, such incentive may in at least some embodiments be provided to a contributor's designee.

Where elements are presented as lists, it is to be understood that each subgroup of the elements is also disclosed, and any element(s) may be removed from the group. The invention provides all such embodiments.

The terms “approximately” or “about” in reference to a number generally include numbers that fall within ±10%, in some embodiments ±5%, in some embodiments ±1%, in some embodiments ±0.5% of the number unless otherwise stated or otherwise evident from the context (except where such number would impermissibly exceed 100% of a possible value). Where ranges are given, endpoints are included. Furthermore, it is to be understood that unless otherwise indicated or otherwise evident from the context and understanding of one of ordinary skill in the art, values that are expressed as ranges may assume any specific value or subrange within the stated ranges in different embodiments of the invention, to the tenth of the unit of the lower limit of the range, unless the context clearly dictates otherwise. Any one or more embodiment(s), element(s), feature(s), aspect(s), component(s) etc., of the present invention may be explicitly excluded from any one or more of the claims.

1. An apparatus comprising non-transitory computer-readable medium encoded with computer-executable instructions for performing a method comprising:
(a) sending, to a computer, a set of data configured to cause the computer to display a first template for structured collection of health information, wherein the first template comprises fields for entry of health information pertaining to one or more diagnostics, therapeutics, key symptoms, signs, complications, or outcomes of a disease;
(b) receiving, from a contributor, a dataset comprising health information regarding an individual, the health information having been entered into the first template using the computer;
(c) analyzing the dataset to determine whether the health information fulfills a set of predetermined criteria for eligibility in one or more clinical trials or managed access programs for an experimental therapy for the disease;
(d) storing at least some of the health information in a database in association with an identifier of the individual;
(e) determining based on said health information that the individual is potentially eligible for at least one of said clinical trials or managed access programs;
(f) sending, to the computer, a set of data configured to cause the computer to display a second template comprising one or more fields for entry of screening data for one or more of said clinical trials or managed access programs; and
(g) receiving, from the contributor, screening data that was entered into the second template;
(h) analyzing the screening data to determine whether, given the screening data, the individual is still potentially eligible for one or more of said clinical trials or managed access programs (b) providing an incentive to the contributor or the contributor's designee.

2-9. (canceled)

10. The apparatus of claim 1, wherein the contributor is a physician and the individual is a patient of the physician.

11. (canceled)

12. The apparatus of claim 1, wherein the method further comprises de-identifying the dataset.

13-15. (canceled)

16. The apparatus of claim 1, the method further comprising: (a) receiving additional data regarding the individual from a second contributor, wherein the second contributor may or may not be the same as the contributor of step (a) and, optionally, adding at least some of the additional data to the EMR.

17-23. (canceled)

24. The apparatus of claim 1, the method further comprising: maintaining in one or more computers, account data of the datasets received from contributors.

25. (canceled)

26. The apparatus of claim 24, the method further comprising: electronically providing to the contributor account data regarding said contributor's account.

27. (canceled)

28. The apparatus of claim 24, the method further comprising: electronically receiving a request for account information from the contributor; and electronically providing to the contributor account data regarding said contributor's account in response to the request.

29-30. (canceled)

31. The apparatus of claim 1, the method further comprising: receiving a request from a subscriber; and providing de-identified data from the database to the subscriber in response to the request.

32-119. (canceled)

120. The apparatus of claim 1, wherein the method further comprises: (i) generating or providing a list of clinical trials or managed access programs (MAPs) for which a patient is eligible or potentially eligible; (ii) completing or assisting a HCP to complete at least a portion of a form to request that the patient be permitted to participate in a clinical trial or MAP; (iii) transmitting, to a sponsor or regulatory agency, a request that the patient be permitted to participate in the clinical trial or MAP; (iv) receiving, from the sponsor or regulatory agency, an indication whether the patient is permitted to participate in the clinical trial or MAP; (v) providing a third template, wherein the third template is designated for the clinical trial or MAP, wherein the third template comprises fields for entry of health information pertaining to one or more diagnostics, therapeutics, key symptoms, signs, complications, or outcomes of a disease; (vi) complying or assisting an investigator or sponsor to comply with a law or regulation pertaining to the clinical trial or MAP; (vii) collecting or analyzing data pertaining to the patient participating in the clinical trial or MAP; and/or (viii) transmitting data generated in the clinical trial or MAP to the sponsor or regulatory agency.

121-126. (canceled)

127. The apparatus of claim 1, wherein the one or more clinical trials or managed access programs comprise a trial set consisting of trials available within a particular geographic region.

128. The apparatus of claim 1, wherein the one or more clinical trials or managed access programs comprise a trial set consisting of one or more trials sponsored by a particular sponsor or sponsors.

129-136. (canceled)

137. A apparatus comprising an EMR system that comprises non-transitory computer-readable medium encoded with computer-executable instructions for performing a method comprising:
(a) sending, to a computer, a set of data configured to cause the computer to display a template for structured collection of health information, wherein the template comprises fields for entry of health information pertaining to one or more diagnostics, therapeutics, key symptoms, signs, complications, or outcomes of a disease;
(b) receiving, from a contributor, a dataset comprising health information regarding an individual, the health information having been entered into the template using the computer, and
(c) storing at least some of the health information in a database in association with an identifier of the individual,
wherein the EMR system further comprises an environment or module that provides one or more functions pertaining to experimental therapies.

138. The apparatus of claim 137, wherein the one or more functions facilitate clinical trial eligibility determination, subject enrollment, and/or electronic data capture.

139. The apparatus of claim 137, wherein the apparatus comprises means for entering the environment or module from within an EMR, wherein said means optionally comprises a link.

140. The apparatus of claim 137, wherein entry into the environment or module results in display of a distinctive identifier informing a user that the experimental therapies environment or module has been entered, wherein the distinctive identifier optionally comprises a ribbon appearing at or near the top, bottom, or side of a screen.

141-170. (canceled)

171. A apparatus comprising computer-executable instructions that, when executed, (a) determine whether an individual has been diagnosed with a particular disease by (i) accessing a database that stores health information regarding the individual or (ii) receiving confirmation that the individual has been diagnosed as having the disease; and (b) permit the individual or a caregiver of the individual to access a computer program product that provides one or more health-related applications or functions that relate to the particular disease.

172. The apparatus of claim 171 wherein the computer program product is suitable for execution on a portable electronic device.

173. The apparatus of claim 171, wherein the computer program product comprises a Body Monitoring application.

174-175. (canceled)

176. The apparatus of claim 171, wherein the computer program product comprises an Experimental Therapies function.

177-188. (canceled)

189. A method of conducting a clinical trial, managed access program, or repositioned therapies program, the method comprising providing access to an apparatus as described in claim 171; and (i) providing information regarding a clinical trial, managed access program, or repositioned therapies program to one or more of said patients using the apparatus; (ii) screening or enrolling one or more of said patients who has used the apparatus such use optionally comprising viewing name of or information about a clinical trial, managed access program, or repositioned therapies program; or (iii) collecting data relevant to the trial or program via the apparatus.

190-196. (canceled)
